SEQUENCE LISTING (36 sequences)

SEQ ID NO: 1 - DNA, 1474 bp, Bacteroides caccae
tggctcaggatgaacgctagctacaggcttaacacatgcaagtcgaggggcatcagtttggtttgcttgcaaaccaaagctggcgaccggcgcacgggtgagtaacacgtatccaacctgcctcatactcggggatagcctttcgaaagaaagattaatatccgatagcatatatttcccgcatgggttttatattaaagaaattcggtatgagatggggatgcgttccattagtttgttgggggggtaacggcccaccaagactacgatggataggggttctgagaggaaggtcccccacattggaactgagacacggtccaaactcctacgggaggcagcagtgaggaatattggtcaatggacgcgagtctgaaccagccaagtagcgtgaaggatgactgccctatgggttgtaaacttcttttatatgggaataaagttgtccacgtgtggatttttgtatgtaccatatgaataaggatcggctaactccgtgccagcagccgcggtaatacggaggatccgagcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagccactggagtgtaactgacgctgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaattatggggaaacccataggccgcaaggcatttgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggccgctacctggtgacaggatgccaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggta

SEQ ID NO: 2 - DNA, 1473 bp, Unknown
  Bacteroides cellulosilyticus/Bacteroides intestinalis
agagtttgatcctggctcaggatgaacgctagctacaggcttaacacatgcaagtcgaggggcagcatgacctagcaataggttgatggcgaccggcgcacgggtgagtaacacgtatccaacctaccggttattccgggatagcctttcgaaagaaagattaataccggatagtataacgagaaggcatctttttgttattaaagaatttcgataaccgatggggatgcgttccattagtttgttggcggggtaacggcccaccaagacatcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtgagccacgtgtggctttttgtatgtaccatacgaataaggatcggctaactccgtgccagcagccgcggtaatacggaggatccgagcgttatccggatttattgggtttaaagggagcgtaggcggactattaagtcagctgtgaaagtttgcggctcaaccgtaaaattgcagttgatactggtcgtcttgagtgcagtagaggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgtttgcgatatacggcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaatatagtggaaacattatagccgcaaggcatttgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacacagcgatgtgatgctaatcccaaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtccgtaaccgtaaggagcggcctagggtaaaactggtaattggggctaagtcgta

SEQ ID NO: 3 - DNA, 1472 bp, Bacteroides faecis
ctcaggatgaacgctagctacaggcttaacacatgcaagtcgaggggcagcatttcagtttgcttgcaaactggagatggcgaccggcgcacgggtgagtaacacgtatccaacctgccgataactcggggatagcctttcgaaagaaagattaatacccgatggcataatagaaccgcatggtttttttattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagtcgtaagacaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggta

SEQ ID NO: 4 - DNA, 929 bp, Bacteroides ovatus
cgatatccggatttattggagtttaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggtkatgctgaggactctagagagactgccgtcgtaagatgtgaggaaggtggggatgacgtcaaatcagcacggcccttacgtccggggctacacacgtgttacaatggggggtacagaaggcsgctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctaggggtaaaactggtaattggggc

SEQ ID NO: 5 - DNA, 873 bp, Bacteroides thetaiotaomicron
tttaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccwagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcawwtgaatawwytggaaacagkwtagycgyaagrcawwtgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgca

SEQ ID NO: 6 - DNA, 1474 bp, Bacteroides uniformis
ctggctcaggatgaacgctagctacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatccctaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcgaggagcgccctagggtaaaactggtgattggggctaagtcgtaacaaggta

SEQ ID NO: 7 - DNA, 1503 bp, Bacteroides vulgatus
atgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagttcacatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatcccaaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaag

SEQ ID NO: 8 - DNA, 887 bp, Bifidobacterium adolescentis
gggctcgtagkcggttcgtcgcgtccggtgtgaaagtccaycgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacactgtgaggtggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccatcctttttgggg

SEQ ID NO: 9 - DNA, 1495 bp, Bifidobacterium longum
tggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctgg

SEQ ID NO: 10 - DNA, 689 bp, Bifidobacterium pseudocatenulatum
ggttcgtcgcgtccggtgtgaaagtccatcgtttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgwcggagctaacgcgttaagcatcccgcctggggagtacggccgcaaggctaaaacwmaaakaaattgacgggggcccgcacaagcggcggagcatgcggattaattcgatgcaacgcgaagaaccttacctgggcttgacatgttcccgacagccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgttcagggcttcacgcatgctacaatggccggtacaacgggatgcgacacggcgacgtg

SEQ ID NO: 11 - DNA, 1394 bp, Unknown
  Blautia coccoides/Blautia producta
tgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaacgggggcttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaaggcgggaccgataactggggtgaagtcgtaacaaggta

SEQ ID NO: 12 - DNA, 918 bp, Clostridium citroniae
tccggatttactggagtagtaagggagcgtagacggcgaagcaagtctggagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggtcagtaaagtgacctttccttcgggacaggggagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcgaccctgcgaaggcaagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaa

SEQ ID NO: 13 - DNA, 1307 bp, Clostridium clostridioforme
taataccgcataagcgcacagtgccgcatggcagtgtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccggggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgccgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatccccctgacgggccggtaacgcggcctttccttcgggacaggggagacaggtggtgcatggttgtcgtcagctcgtgtcgtgagatgttgggttaagtcccgcaacgagcgcaacccttatccttagtagccagcaggtagagccgggcactctagggagactgccagggataacctggaggaaggtggggatgacgtcaaatcatcatgccccttatgatttgggctacacacgtgctacaatggcgtaaacaaagggaagcgagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaaggcggggcaggtaactggggtgaagtcgt

SEQ ID NO: 14 - DNA, 1475 bp, Clostridium innocuum
atggagagtttgatcctggctcaggatgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacgatgagaactaagtgttggaggaattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggaaacaaataccctagagatagggggataattatggatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcggccacaaagagcagcgacacagtgatgtgaagcgaatctcataaaggtcgtctcagttcggattgaagtctgcaactcgacttcatgaagtcggaatcgctagtaatcgcagatcagcatgctgcggtgaatacgttctcgggccttgtacacaccgcccgtcaaaccatgggagtcagtaatacccgaagccggtggcataaccgtaaggagtgagccgtcgaaggtaggaccga

SEQ ID NO: 15 - DNA, 1475 bp, Unknown
  Clostridium innocuum/Erysipelotrichaceae bacterium 6_1_45
atggagagtttgatcctggctcaggatgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacgatgagaactaagtgttggaggaattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggaaacaaataccctagagatagggggataattatggatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcgaccacaaagagcagcgacacagtgatgtgaagcgaatctcataaaggtcgtctcagttcggattgaagtctgcaactcgacttcatgaagtcggaatcgctagtaatcgcagatcagcatgctgcggtgaatacgttctcgggccttgtacacaccgcccgtcaaaccatgggagtcagtaatacccgaagccggtggcataaccgtaaggagtgagccgtcgaaggtaggaccga

SEQ ID NO: 16 - DNA, 906 bp, Clostridium sordellii
acacatgcaagtcgagcgaacccttcggggtgagcggcggacgggtgagtaacgcgtgggtaacctgccctgtacacacggataacataccgaaaggtatgctaatacgggatrayatatgagagtcgcatggcttttgtatcaaagctccggcggtacaggatggacccgcgtctgattagctagttggtaaggtaacggcttaccaaggcaacgatcagtagccgacctgagagggtgatcggccacattggaactgagacacggtccaaactcmtacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggcggtctttcaagccagaagtgaaaggctacggctcaaccgtagtaagcttttggaactgtaggacttgagtgcaggagaggagagtggaattcctagtgtagcggtgaaatgcgtagatattaggaggaacaccagtagcgaaggcggctctctggactgtaactgacgctgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttatctaarcttgacatc

SEQ ID NO: 17 - DNA, 380 bp, Coprococcus comes
acrggaggcagcagtggggaatattgcacaatgggggaaaccctgatgcagcgacgccrcgtgagcgaagaagtattkcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaaggcgggggctcccccccggggactgctttggaaactatgcagctagactgtcggacaggtaagtggaattcccagtgtagcggtgaaatgcgtagatattgggaggaacagcaktgggtaaggctscttacaggacrat

SEQ ID NO: 18 - DNA, 1317 bp, Dorea longicatena
taacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagcttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaagg

SEQ ID NO: 19 - DNA, 808 bp, Erysipelatoclostridium ramosum
gagggagcaggcggcagcaagggtctgtggtgaaagcctgaagttaaacttcagtaagccatagaaaccaggcagctagagtgcaggagaggakcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactragtgttggatgtcaaagttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatactcataaaggctccagagatggagagatagctatatgagatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaargcggggatgacgtcaaatcatcatgccccttatgacctgggctacacacgtgctacaatggatggtgcagagggaagcgaagccgcgaggtgaagcaaaacccataaaaccattctcagttcggattgtagtctgcarctcgactacatgaagttggaatcgctagtaatcgcgaatcarcatgtcgcgatgaatamgttctcgggcctt

SEQ ID NO: 20 - DNA, 1521 bp, Eubacterium rectale
agagtttgatcctggctcaggatgaacgctggcggcgtgcttaacacatgcaagtcgaacgaagcactttatttgatttccttcgggactgattattttgtgactgagtggcggacgggtgagtaacgcgtgggtaacctgccttgtacagggggataacagttggaaacggctgctaataccgcataagcgcacggcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtaccttctcttcggagcaggagtgacaggtggtgcatggttgtcgtcagctcgtgtcgtgagatgttgggttaagtcccgcaacgagcgcaacccttatctttagtagccagcggttcggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaaggaaggagctgtcgaaggcaggctcgataactggggtgaagtcgtaacaaggtagccgtatcggaaggtgcggctggatcacct

SEQ ID NO: 21 - DNA, 1482 bp, Odoribacter splanchnicus
agagtttgatcctggctcaggatgaacgctagcgacaggcttaacacatgcaagtcgaggggcatcatgaggtagcaataccttgatggcgaccggcgcacgggtgagtaacgcgtatgcaacctgcccgataccggggtatagcccatggaaacgtggattaacaccccatagtacttttatcctgcatgggatgtgagttaaatgtttaaggtatcggatgggcatgcgtcctattagttagttggcggggtaacagcccaccaagacgatgataggtaggggttctgagaggaaggtcccccacattggaactgagacacggtccaaactcctacgggaggcagcagtgaggaatattggtcaatggacgagagtctgaaccagccaagtcgcgtgagggaagactgccctatgggttgtaaacctcttttataagggaagaataagttctacgtgtagaatgatgcctgtaccttatgaataagcatcggctaactccgtgccagcagccgcggtaatacggaggatgcgagcgttatccggatttattgggtttaaagggtgcgtaggcggtttattaagttagtggttaaatatttgagctaaactcaattgtgccattaatactggtaaactggagtacagacgaggtaggcggaataagttaagtagcggtgaaatgcatagatataacttagaactccgatagcgaaggcagcttaccagactgtaactgacgctgatgcacgagagcgtgggtagcgaacaggattagataccctggtagtccacgccgtaaacgatgctcactggttctgtgcgatatattgtacgggattaagcgaaagtattaagtgagccacctggggagtacgtcggcaacgatgaaactcaaaggaattgacgggggcccgcacaagcggaggaacatgtggtttaattcgatgatacgcgaggaaccttacctgggtttaaatgggaaatgtcgtatttggaaacagatattctcttcggagcgtttttcaaggtgctgcatggttgtcgtcagctcgtgccgtgaggtgtcgggttaagtcccataacgagcgcaacccttaccgttagttgctagcatgtaatgatgagcactctaacgggactgccaccgtaaggtgagaggaaggcggggatgacgtcaaatcagcacggcccttacacccagggctacacacgtgttacaatggccggtacagagggccgctaccaggtgactggatgccaatctcaaaagccggtcgtagttcggattggagtctgtaacccgactccatgaagttggattcgctagtaatcgcgcatcagccatggcgcggtgaatacgttcccgggccttgtacacaccgcccgtcaagccatggaagccgggggtgcctgaagtccgtaaccgcgaggatcggcctagggcaaaactggtaactggggctaagtcgtaacaaggta

SEQ ID NO: 22 - DNA, 738 bp, Parabacteroides distasonis
cgaggggcagcrcaggagttagcaataccsggtggcgaccggcgcacgggtgagtaacgcgtatgcaacttrcctatcagagggggataacccggcgaaagtcggactaataccgcatgaagcagggatyccgcatgggratatttgctaaagattcatcgctgatagataggcatgcgttccattaggcagttggcggggtaacrgcccaccaaaccgacgatggataggggttctgagaggaaggtcccccacattggtactgagacacggaccaaactcctacgggaggcagcagtgaggaatattggtcaatggscgwragsctgaaccagccaagtcgcgtgagggatgaaggttctatggatcgtaaacctcttttataagggaataaagtgcgggacgtgtccyrttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgwttgcgaaggcagcctgccaagccgtaactgacgcggatgcacgaaagcgtggggatcaaacaggattagataccctggta

SEQ ID NO: 23 - DNA, 521 bp, Parabacteroides merdae
catgcaagtcgaggggcagcatgatttgtagcaatacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttckgagaggaaggtcccccacattggtactgagacacggaccaaactcctacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgmsarcmgccgcgggaatacggaagatgcagagcgttatccggatwtattggggtta

SEQ ID NO: 24 - DNA, 1368 bp, Bacteroides xylanisolvens
catgcaagtcgaggggcagcattttagtttgcttgcaaactaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatctggaaacaggttagccgcaaggcaaatgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggg

SEQ ID NO: 25 - DNA, 1399 bp, Blautia obeum
ggcgtgcttaacacatgcaagtcgaacgggaaaccttttattgaagcttcggcagatttagctggtttctagtggcggacgggtgagtaacgcgtgggtaacctgccttatacagggggataacaaccagaaatggttgctaataccgcataagcgcacaggaccgcatggtccggtgtgaaaaactccggtggtataagatggacccgcgttggattagctagttggcagggtaacggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgaccgttccttaaccggaactttccttcgggacaggggagacaggtggtgcatggttgtcgtcagctcgtgtcgtgagatgttgggttaagtcccgcaacgagcgcaacccctatccccagtagccagcagtccggctgggcactctgaggagactgccagggataacctggaggaaggcggggatgacgtcaaatcatcatgccccttatgatttgggctacacacgtgctacaatggcgtaaacaaagggaagcaagcctgcgaaggtaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaactgc

SEQ ID NO: 26 - DNA, 1485 bp, Alistipes putredinis
agagtttgatcctggctcaggatgaacgctagcggcaggcttaacacatgcaagtcgaggggcagcataatggatagcaatatctatggtggcgaccggcgcacgggtgcgtaacgcgtatgcaacctacctttaacagggggataacactgagaaattggtactaataccccataatatcatagaaggcatcttttatggttgaaaattccgatggttagagatgggcatgcgttgtattagctagttggtggggtaacggctcaccaaggcgacgatacatagggggactgagaggttaaccccccacactggtactgagacacggaccagactcctacgggaggcagcagtgaggaatattggtcaatggacgcaagtctgaaccagccatgccgcgtgcaggatgacggctctatgagttgtaaactgcttttgtacgagggtaaacgcagatacgtgtatctgtctgaaagtatcgtacgaataaggatcggctaactccgtgccagcagccgcggtaatacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagcgacgattcttgaaagaggatttcccttcggggcgcgaaactaggtgctgcatggttgtcgtcagctcgtgccgtgaggtgtcgggttaagtcccataacgagcgcaacccctaccgttagttgccatcaggtgaagctgggcactctggcgggactgccggtgtaagccgagaggaaggtggggatgacgtcaaatcagcacggcccttacgtccggggctacacacgtgttacaatggtaggtacagagggcagctacccagcgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatgggagccgggggtgcctgaagttcgtgaccgcaaggagcgacctagggcaaaactggtgactggggctaagtcgtaacaaggta

SEQ ID NO: 27 - DNA, 1464 bp, Collinsella aerofaciens
agagttcgatcctggctcaggatgaacgctggcggcgcgcctaacacatgcaagtcgaacggcacctatcttcggatagaagcgagtggcgaacggctgagtaacacgtggagaacctgccccctcccccgggatagccgcccgaaaggacgggtaataccggataccccggggtgccgcatggcaccccggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagtggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacaggattagataccctggtagtcccagccgtaaacgatggacgctaggtgtggggggacgatccccccgtgccgcagccaacgcattaagcgtcccgcctggggagtacggccgcaaggctaaaactcaaaggaattgacgggggcccgcacaagcagcggagcatgtggcttaattcgaagcaacgcgaagaaccttaccagggcttgacatatgggtgaagcgggggagaccccgtggccgagaggagcccatacaggtggtgcatggctgtcgtcagctcgtgtcgtgagatgttgggttaagtcccgcaacgagcgcaacccccgccgcgtgttgccatcgggtgatgccgggaacccacgcgggaccgccgccgtcaaggcggaggagggcggggacgacgtcaagtcatcatgccccttatgccctgggctgcacacgtgctacaatggccggtacagagggatgccaccccgcgagggggagcggatcccggaaagccggccccagttcggattgggggctgcaacccgcccccatgaagtcggagttgctagtaatcgcggatcagcatgccgcggtgaatgcgttcccgggccttgtacacaccgcccgtcacaccacccgagtcgtctgcacccgaagtcgccggcccaaccgagaggggggaggcgccgaaggtgtggagggtgaggggggtgaagtcgtaacaaggta

SEQ ID NO: 28 - DNA, 1472 bp, Unknown
  Bacteroides faecis/Eubacterium hallii
ctcaggatgaacgctagctacaggcttaacacatgcaagtcgaggggcagcattccagtttgcttgcaaactggagatggcgaccggcgcacgggtgagtaacacgtatccaacctgccgataactcggggatagcctttcgaaagaaagattaatacccgatggcataatagaaccgcatggtttgattattaaagaatttcggttatcgatggggatgcgttccattaggcagttggtggggtaacggcccaccaaaccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtggtccacgtgtggatttttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggcgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagtcgtaagacaaatgtgaaggtgctgcatggttgtcgtcagctcgtgccgtgaggtgtcggcttaagtgccataacgagcgcaacccttatctttagttactaacaggtcatgctgaggactctggagagactgccgtcgtaagatgtgaggaaggtggggatgacgtcaaatcagcacggcccttacgtccggggctacacacgtgttacaatggggggtacagaaggccgctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggta

SEQ ID NO: 29 - DNA, 317 bp, Alistipes shahii
acatagggggwstgwkaggttwrccscccacattsrtactgagmcatgawcmaactctmtacgggargsagsagtgaggaatattggtcrrtggacgcaagtctgaaccagccatgccgsgtgcrggaagacggctckatgagtkgkaaactgcttttgtacrarrgtaaacgctcttacgtgtaagagcctgaaagtatsgtacraatgaggatcggctaactccgtgccagcagccgcggtaatacggaggatccaagcgttatccggatttattgggtttaaagggtgcgtaggcgggttgataaagttagrgg

SEQ ID NO: 30 - DNA, 811 bp, Anaerostipes caccae
gcttacacatgcaagtcgaacgaagcatttargattgaagttttcggatggatttcctatatgactgagtggcggacgggtgagtaacgcgtggggaacctgccctatacagggggataacagctggaaacggctgctaataccgcataagcgcacagaatcgcatgattcagtgtgaaaagccctggcagtataggatggtcccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaacagacggtacctgactaagaagccccggctaactacgtgccagcagccgcggtaatacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctagtgtagcggtgaaatgcgtagatattaggaggaacaccagtggcgaaggcggcttactggactgtcactgacactgatgcacgaaagcgtggggagcaaacaggattagataccctggtagtccacgccgtaaacgatgaatactaggtgtcggggccgtagaggcttcggtgccgcagcaaa

SEQ ID NO: 31 - DNA, 728 bp, Phascolarctobacterium faecium
cggagaattttcatttcggtagaattcttagtggcgaacgggtgagtaacgcgtaggcaacctgccctttagacggggacaacattccgaaaggagtgctaataccggatgtgatcatcgtgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacgggaggcagcagtggggaatcttccgcaatggacgaaagtctgacagagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtttgacgctgagatgcgaaagccagggtagc

SEQ ID NO: 32 - DNA, 707 bp, Agathobaculum butyriciproducens
tagtggcggacgggtgagtaacgcgtgagcaatctgcctttaagagggggataacagtcggaaacggctgctaataccgcataaagcattgaattcgcatgttttcgatgccaaaggagcaatccgcttttagatgagctcgcgtctgattagctagttggcggggtaacggcccaccaaggcgacgatcagtagccggactgagaggttgaacggccacattgggactgagacacggcccagactcctacgggaggcagcagtggggaatattgcgcaatgggggraaccctgacgcagcaacgccgcgtgattgaagaaggccttcgggttgtaaagatctttaatcagggacgaaamatgacggtacctgaagaataagctccggctaactacgtgccagcagccgcggtaatacgtagggagcaagcgttatccggatttactgggtgtaaagggcgcgcaggcgggccggcaagttggaagtgaaatccgggggcttaacccccgaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagataccctggtagtccacgccgtaaacgatggata

SEQ ID NO: 33 - DNA, 1525 bp, Bacteroides fragilis
atgaagagtttgatcctggctcaggatgaacgctagctacaggcttaacacatgcaagtcgaggggcatcaggaagaaagcttgctttctttgctggcgaccggcgcacgggtgagtaacacgtatccaacctgccctttactcggggatagcctttcgaaagaaagattaatacccgatggcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaagcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggatcgtcctagggtaaaactggtgactggggctaagtcgtaacaaggtagccgtaccggaaggtgcggctggaacacctcctt

SEQ ID NO: 34 - DNA, 1022 bp, Fusobacterium mortiferum
tggctcaggatgaacgctgacagaatgcttaacacatgcaagtctacttgatccttcgggtgatggtggcggacgggtgagtaacgcgtaaagaacttgccctgcagtctgggacaacatttggaaacgaatgctaataccggatattatgtatttctcgcatgagttttacatgaaagctatatgcgctgcaggagagctttgcgtcctattagctagttggtgaggtaacggctcaccaaggccatgataggtagccggcctgagagggtgaacggccacaaggggactgagacacggcccttactcctacgggaggcagcagtggggaatattggacaatggaccaaaagtctgatccagcaattctgtgtgcacgatgaagtttttcggaatgtaaagtgctttcagttgggacgaagtaagtgacggtaccaacagaagaagcgacggctaaatacgtgccagcagccgcggtaatacgtatgtcgcaagcgttatccggatttattgggcgtaaagcgcgtctaggcggtttggtaagtctgatgtgaaaatgcggggctcaactccgtattgcgttggaaactgctaaactagagtactggagaggtgggcggaactacaagtgtagaggtgaaattcgtagatatttgtaggaatgccgatggggaagccagcccactggacagatactgacgctaaagcgcgaaagcgtgggtagcaaacaggattagataccctggtagtccacgccgtaaacgatgattactaggtgttgggggtcgaacctcagcgcccaagctaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcctaagaaattagcagagatgcttttgtgccccttcgggggaacttagtgacaggtggtgcatggctgtcgtcagctcgtgt

SEQ ID NO: 35 - DNA, 1490 bp, Unknown
  Paraclostridium benzoelyticum/Clostridium bifermantans
agagtttgatcctggctcaggatgaacgctggcggcgtgcctaacacatgcaagtcgagcgatctcttcggagagagcggcggacgggtgagtaacgcgtgggtaacctgccctgtacacacggataacataccgaaaggtatactaatacgggataacatacgaaagtcgcatggcttttgtatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggttttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatcccactgacctctccctaatcggagatttcccttcggggacagtggtgacaggtggtgcatggttgtcgtcagctcgtgtcgtgagatgttgggttaagtcccgcaacgagcgcaacccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgcgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatggaagttgggggcgcccgaagccggttagctaaccttttaggaagcggccgtcgaaggtgaaaccaatgactggggtgaagtcgtaacaaggtagccgtatcggaaggtgcggctggatcacct

SEQ ID NO: 36 - DNA, 1538 bp, Unknown
  Escherichia fergusonii/Escherichia coli
ttgaagagtttgatcatggctcagattgaacgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

(19) United States
(12) Patent Application Publication
(10) Pub. No.: US 2022/0409674 A1
(43) Pub. Date: Dec. 29, 2022

(54) COMPOSITIONS AND METHODS FOR SUPPRESSING PATHOGENIC ORGANISMS

(71) Applicant: Vedanta Biosciences, Inc., Cambridge, MA (US)

(72) Inventor: Silvia Caballero, Cambridge, MA (US)

(73) Assignee: Vedanta Biosciences, Inc., Cambridge, MA (US)

(21) Appl. No.: 17/633,930

(22) PCT Filed: Aug. 7, 2020
(86) PCT No.: PCT/US2020/045442

§ 371 Date: Feb. 8, 2022

Related U.S. Application Data
(60) Provisional application No. 62/884,917, filed on Aug. 9, 2019.
(60) Provisional application No. 62/901,206, filed on Sep. 16, 2019.
(60) Provisional application No. 62/947,517, filed on Dec. 12, 2019.
(60) Provisional application No. 63/031,299, filed on May 28, 2020.

(51) Int. Cl. A61K 35/741; A61K 35/742; A61K 35/745

(52) U.S. Cl. CPC A61K 35/741; A61K 35/742; A61K 35/745; A23L 33/135

(57) ABSTRACT

Provided herein are compositions and methods for suppressing pathogenic organisms. Also provided herein are compositions and methods for inducing regulatory T-cells in response to pathogenic organisms and methods of treating a disease or disorder associated with bacterial colonization. Also provided herein are methods of suppressing colonization of the intestine of a subject with oral microbiome bacteria.

### RELATED APPLICATIONS

This application claims the benefit under 35 U.S.C. § 119(e) of U.S. provisional application No. 62/884,917, filed Aug. 9, 2019; U.S. provisional application No. 62/901,206, filed Sep. 16, 2019; U.S. provisional application No. 62/947,517, filed Dec. 12, 2019; and U.S. provisional application No. 63/031,299, filed May 28, 2020. The entire contents of each of these referenced applications are incorporated by reference herein.

### GOVERNMENT SUPPORT

This invention was made with government support under Grant No. 4500003237 awarded by the U.S. Department of Health and Human Services Office of the Assistant Secretary for Preparedness and Response (HHS/ASPR). The government has certain rights in the invention.

### FIELD

Provided herein are compositions and methods for suppressing pathogenic organisms. Provided herein are compositions and methods for treating diseases or disorders associated with bacterial colonization or treating diseases or disorders associated with an immune response induced by bacteria. Also provided herein are compositions and methods for inducing regulatory T cell and short chain fatty acid production in response to pathogenic organisms.

### BACKGROUND

Multidrug resistant organisms (MDROs; “superbugs”), microorganisms that have developed resistance to one or more classes of antimicrobial agents, such as antibiotics, are emerging as serious global health threat. It is estimated that over 2 million people in the United States contract serious bacterial infections that are resistant to one or more antibiotics each year (CDC, Antibiotic Resistance Threats in the United States, 2013. Publication No. CS239559-B). Treatment options for subjects with MDROs are extremely limited; prevention of transmission is critical. The most important factor contributing to the generation and propagation of MDROs is the use and overuse/misuse of antibiotics and it is thought that the problem will increase in severity as further pathogenic organisms with antibiotic resistance arise (CDC, 2013; WHO 2017).

### SUMMARY

In some aspects, the present disclosure provides compositions comprising one or more purified bacterial strains belonging to the phylum Firmicutes or Bacteroidetes, and one or more purified bacterial strains selected from the group consisting of an Escherichia species and a Fusobacterium species.

In some embodiments, the one or more purified bacterial strains belonging to the phylum Firmicutes are bacterial strains belonging to the Clostridiaceae family. In some embodiments, the one or more purified bacterial strains belonging to the phylum Firmicutes are bacterial strains belonging to Clostridium cluster IV and/or Clostridium cluster XIVa. In some embodiments, the one or more purified bacterial strains belonging to the phylum Bacteroidetes are bacterial strains belonging to the Bacteroidaceae family.

In some embodiments, the one or more purified bacterial strains belonging to the phylum Firmicutes or Bacteroidetes are bacterial strains that produce short chain fatty acids. In some embodiments, the short chain fatty acids are butyrate.

In some aspects, the present disclosure provides compositions comprising a purified bacterial strain belonging to Escherichia species, a purified bacterial strain belonging to Fusobacterium species, and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis.

In some aspects, the present disclosure provides compositions comprising a purified bacterial strain belonging to Escherichia species, a purified bacterial strain belonging to Fusobacterium species, and one or more purified bacterial strains of species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii.

In some aspects, the present disclosure provides compositions comprising a purified bacterial strain belonging to Escherichia species, a purified bacterial strain belonging to Fusobacterium species, and one or more purified bacterial strains of species selected from the group consisting of Collinsella aerofaciens, Bifidobacterium longum, Bacteroides ovatus, Bacteroides vulgatus, Alistipes putredinis, Clostridium citroniae, Clostridium clostridioforme, Erysipelatoclostridium ramosum, Erysipelotrichaceae bacterium 6_1_45, Blautia obeum, Blautia producta, Dorea longicatena, and Phascolarctobacterium faecium.

In some aspects, the present disclosure provides compositions comprising Clostridium bolteae, Anaerotruncus colihominis, Sellimonas intestinalis, Clostridium symbiosum, Blautia producta, Dorea longicatena, Erysipelotrichaceae bacterium, Flavinofractor plautii, a purified bacterial strain belonging to Escherichia species, and a purified bacterial strain belonging to Fusobacterium species.

In some aspects, the present disclosure provides compositions comprising Anaerotruncus colihominis, Sellimonas intestinalis, Clostridium symbiosum, Dorea longicatena, Erysipelotrichaceae bacterium, Flavinofractor plautii, a purified bacterial strain belonging to Escherichia species, and a purified bacterial strain belonging to Fusobacterium species.

In some aspects, the present disclosure provides compositions comprising Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), a purified bacterial strain belonging to Escherichia species, and a purified bacterial strain belonging to Fusobacterium species.

In some aspects, the present disclosure provides compositions comprising Clostridium bolteae, Anaerotruncus colihominis, Sellimonas intestinalis, Clostridium symbiosum, Blautia producta, Erysipelotrichaceae bacterium, Flavinofractor plautii, a purified bacterial strain belonging to Escherichia species, and a purified bacterial strain belonging to Fusobacterium species.

In some aspects, the present disclosure provides compositions comprising Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecis, a purified bacterial strain belonging to Escherichia species, and a purified bacterial strain belonging to Fusobacterium species.

In some aspects, the present disclosure provides compositions comprising Collinsella aerofaciens, Bacteroides ovatus, Bacteroides thetaiotamicron, Bacteroides xyalinosolvens, Bacteroides cellulosilyticus, Bacteroides uniformis, Bacteroides vulgatus, Parabacteroides distasonis, Alistipes putredinis, Alistipes shahii, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium rectale, Anaerostipes caccae, Blautia obeum, Blautia producta, Coprococcus comes, Dorea longicatena, Agathobaculum butyriciproducens, Phascolarctobacterium faecis, a purified bacterial strain belonging to Escherichia species, and a purified bacterial strain belonging to Fusobacterium species.

In some aspects, the present disclosure provides compositions comprising Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, a purified bacterial strain belonging to Escherichia species, and a purified bacterial strain belonging to Fusobacterium species.

In some aspects, the present disclosure provides compositions comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In some aspects, the present disclosure provides compositions comprising Bacteroides cellulosilyticus, Bacteroides ovatus, Bacteroides thetaiotaomicron, Bacteroides uniformis, Bacteroides vulgatus, Bifidobacterium adolescentis, Bifidobacterium longum, Blautia producta, Clostridium citroniae, Clostridium clostridioforme, Clostridium innocuum, Clostridium innocuum, Coprococcus comes, Dorea longicatena, Erysipelatoclostridium ramosum, Eubacterium rectale, Parabacteroides distasonis, Bacteroides xylanisolvens, Blautia obeum, Alistipes putredinis, Collinsella aerofaciens, Eubacterium hallii, Anaerostipes caccae, Phascolarctobacterium faecium, Agathobaculum butyriciproducens, Fusobacterium mortiferum, and Escherichia coli.

In some aspects, the present disclosure provides compositions comprising Bacteroides caccae, Bacteroides cellulosilyticus, Bacteroides ovatus, Bacteroides thetaiotaomicron, Bacteroides uniformis, Bacteroides vulgatus, Bifidobacterium longum, Blautia producta, Clostridium clostridioforme, Clostridium innocuum, Coprococcus comes, Dorea longicatena, Erysipelatoclostridium ramosum, Clostridium citroniae, Odoribacter splanchnicus, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Agathobaculum butyriciproducens, Bacteroides fragilis, Fusobacterium mortiferum, and Escherichia coli.

In some aspects, the present disclosure provides compositions comprising Bacteroides ovatus, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Clostridium innocuum, Paeniclostridium sordellii, Coprococcus comes, Dorea longicatena, Erysipelatoclostridium ramosum, Eubacterium rectale, Odoribacter splanchnicus, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, Phascolarctobacterium faecium, Agathobaculum butyriciproducens, Fusobacterium mortiferum, Paraclostridium bifermentans, and Escherichia coli.

In some aspects, the present disclosure provides compositions comprising Bacteroides ovatus, Bacteroides vulgatus, Bifidobacterium longum, Blautia producta, Clostridium citroniae, Clostridium clostridioforme, Clostridium innocuum, Coprococcus comes, Dorea longicatena, Erysipelatoclostridium ramosum, Odoribacter splanchnicus, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Fusobacterium mortiferum, and Escherichia coli.

In some aspects, the present disclosure provides compositions comprising Bacteroides faecis, Bacteroides ovatus, Bacteroides vulgatus, Bifidobacterium adolescentis, Bifidobacterium longum, Blautia producta, Clostridium citroniae, Clostridium clostridioforme, Clostridium innocuum, Clostridium innocuum, Paeniclostridium sordellii, Dorea longicatena, Erysipelatoclostridium ramosum, Odoribacter splanchnicus, Blautia obeum, Alistipes putredinis, Collinsella aerofaciens, Eubacterium hallii, Anaerostipes caccae, Phascolarctobacterium faecium, Agathobaculum butyriciproducens, Bacteroides fragilis, Fusobacterium mortiferum, Paraclostridium bifermentans, and Escherichia coli.

In some aspects, the present disclosure provides compositions comprising Bacteroides faecis, Bacteroides ovatus, Bacteroides vulgatus, Bifidobacterium longum, Blautia producta, Clostridium citroniae, Clostridium clostridioforme, Clostridium innocuum, Dorea longicatena, Erysipelatoclostridium ramosum, Odoribacter splanchnicus, Alistipes putredinis, Collinsella aerofaciens, Blautia obeum, Anaerostipes caccae, Phascolarctobacterium faecium, Agathobaculum butyriciproducens, Fusobacterium mortiferum, and Escherichia coli.

In some aspects, the present disclosure provides compositions comprising Bacteroides vulgatus, Clostridium citroniae, and Phascolarctobacterium faecium, and one or more purified bacterial strains of species selected from the group consisting of Bacteroides faecis, Bacteroides ovatus, Bifidobacterium longum, Blautia producta, Clostridium clostridioforme, Clostridium innocuum, Dorea longicatena, Erysipelatoclostridium ramosum, Odoribacter splanchnicus, Alistipes putredinis, Collinsella aerofaciens, Blautia obeum, Anaerostipes caccae, Agathobaculum butyriciproducens, Fusobacterium mortiferum, and Escherichia coli

In some aspects, the present disclosure provides compositions comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Clostridium clostridioforme, Blautia obeum, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In some aspects, the present disclosure provides compositions comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Blautia obeum, Clostridium innocuum, Agathobaculum butyriciproducens, Clostridium innocuum, Dorea longicatena, Collinsella aerofaciens, Eubacterium hallii, Bacteroides faecis, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In some aspects, the present disclosure provides compositions comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Paraclostridium bifermentans, Clostridium citroniae, Anaerostipes caccae, Fusobacterium mortiferum, Blautia obeum, Clostridium innocuum, Agathobaculum butyriciproducens, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In some aspects, the present disclosure provides compositions comprising Collinsella aerofaciens, Bifidobacterium longum, Bacteroides ovatus, Bacteroides faecis, Bacteroides vulgatus, Odoribacter splanchnicus, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Dorea longicatena, Agathobaculum butyriciproducens, Phascolarctobacterium faecium, Escherichia coli, and Fusobacterium mortiferum.

In some aspects, the present disclosure provides compositions comprising Collinsella aerofaciens, Bifidobacterium longum, Bacteroides ovatus, Bacteroides vulgatus, Parabacteroides distasonis, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Agathobaculum butyriciproducens, Phascolarctobacterium faecium, Escherichia coli, and Fusobacterium mortiferum.

In some aspects, the present disclosure provides compositions comprising Collinsella aerofaciens, Bifidobacterium longum, Bacteroides ovatus, Bacteroides vulgatus, Alistipes putredinis, Clostridium citroniae, Clostridium clostridioforme, Erysipelatoclostridium ramosum, Erysipelotrichaceae bacterium 6_1_45, Blautia obeum, Blautia producta, Dorea longicatena, Phascolarctobacterium faecium, Escherichia coli, and Fusobacterium mortiferum.

In some embodiments, the composition comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 purified bacterial strains.

In some embodiments, the Escherichia species is Escherichia coli. In some embodiments, the Escherichia coli is Escherichia sp. 3_2_53FAA. In some embodiments, the Fusobacterium species is Fusobacterium mortiferum.

In some embodiments, the Escherichia coli encodes one or more genes associated with bacteriocin production. In some embodiments, the Escherichia coli does not encode or more genes associated with plasmid uptake.

In some embodiments, the composition comprises bacterial strains that originate from more than one human donor.

In some embodiments, the composition is effective in suppressing the replication, survival, and/or colonization of one or more pathogenic organisms. In some embodiments, the composition is effective in treating an infection by a pathogenic organism in a subject. In some embodiments, the pathogenic organism is susceptible to antibiotics.

In some embodiments, the pathogenic organism is resistant to one or more antibiotics. In some embodiments, the pathogenic organism is a multi-drug resistant organism. In some embodiments the multi-drug resistant organism is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, or Clindamycin resistant Group B Streptococcus.

In some embodiments, the composition is effective in inducing the production of regulatory T cells (Tregs) in the intestine. In some embodiments, the composition is effective in inducing the production of short chain fatty acids (SCFAs) in the intestine. In some embodiments, the composition is effective in suppressing the replication, survival, and/or colonization of the intestine by one or more bacteria associated with induction of a Th1 immune response. In some embodiments, the composition is effective in suppressing the replication, survival, and/or colonization of the intestine by one or more oral microbiome bacteria.

In some embodiments, the bacterial strains are lyophilized. In some embodiments, the bacterial strains are spray-dried. In some embodiments, the one or more bacterial strains are in spore form. In some embodiments, each of the bacterial strains is in spore form. In some embodiments, the one or more bacterial strains are in vegetative form. In some embodiments, each of the bacterial strains is in vegetative form.

In some aspects, the prevent disclosure provides pharmaceutical compositions comprising any of the compositions described herein. In some embodiments, the pharmaceutical composition further comprises a pharmaceutically acceptable excipient. In some embodiments, the pharmaceutical composition is formulated for oral delivery. In some embodiments, the pharmaceutical composition is formulated for rectal delivery. In some embodiments, the pharmaceutical composition is formulated for delivery to the intestine. In some embodiments, the pharmaceutical composition is formulated for delivery to the colon.

In some embodiments, the pharmaceutical composition is administered as one dose. In some embodiments, the pharmaceutical composition is administered as multiple doses. In some embodiments, each dose of the pharmaceutical composition comprises the administration of multiple capsules.

In some aspects, the present disclosure provides methods of suppressing infection by a pathogenic organism in a subject, comprising administering a therapeutically effective amount of any of the compositions or food products described herein. In some aspects, the present disclosure provides any of the compositions or food products described herein for use as a medicament. In some aspects, the present disclosure provides any of the compositions or food products described herein for use in the suppression of infection by a pathogenic organism. In some aspects, the present disclosure provides use of any of the compositions or food products described herein in the preparation of a medicament for the suppression of infection by a pathogenic organism. In some embodiments, the pathogenic organism is susceptible to antibiotics. In some embodiments, the pathogenic organism is resistant to antibiotics. In some embodiments, the pathogenic organism is a Clostridium difficile. In some embodiments, the pathogenic organism is a multi-drug resistant organism.

In some embodiments, the pathogenic organism is Klebsiella pneumoniae. In some embodiments, the Klebsiella pneumoniae is multi-drug resistant. In some embodiments, the multi-drug resistant Klebsiella pneumoniae is carbapenem-resistant Klebsiella pneumoniae.

In some embodiments, the Klebsiella pneumoniae induces a Th1 response. In some embodiments, the Klebsiella pneumoniae is strain BAA-2552, strain KP-1, strain 700721, strain 13882, strain 34E1, strain BAA-1705, strain 700603, or strain Kp-2H7. In some embodiments, the Klebsiella pneumoniae is strain Kp-2H7.

In some embodiments the subject is a human. In some embodiments, the composition is administered to the subject more than once. In some embodiments, the composition is administered to the subject by oral administration. In some embodiments, the composition is administered to the subject by rectal administration.

In some embodiments, the administering suppresses the replication, survival, and/or colonization of the pathogenic organism. In some embodiments, the pathogenic organism is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, or Clindamycin resistant Group B Streptococcus.

In some embodiments, the method further comprises administering one or more additional compositions comprising bacteria. In some embodiments, administration of the pharmaceutical composition is not preceded by administration of an antibiotic. In some embodiments, administration of the pharmaceutical composition is not preceded by administration of vancomycin. In some embodiments, the method further comprises administering an antibiotic to the subject prior to administration of the pharmaceutical composition. In some embodiments, the antibiotic is vancomycin.

In some aspects, the present disclosure provides methods of treating infection by a pathogenic organism in a subject, comprising administering a therapeutically effective amount of any of the compositions or food products described herein. In some aspects, the present disclosure provides any of the compositions or food products described herein for use as a medicament. In some aspects, the present disclosure provides any of the compositions or food products described herein for use in the treatment of infection by a pathogenic organism. In some aspects, the present disclosure provides use of any of the compositions or food products described herein in the preparation of a medicament for the treatment of infection by a pathogenic organism. In some embodiments, the pathogenic organism is susceptible to antibiotics. In some embodiments, the pathogenic organism is resistant to antibiotics. In some embodiments, the pathogenic organism is a Clostridium difficile. In some embodiments, the pathogenic organism is a multi-drug resistant organism.

In some embodiments, the pathogenic organism is Klebsiella pneumoniae. In some embodiments, the Klebsiella pneumoniae is multi-drug resistant. In some embodiments, the multi-drug resistant Klebsiella pneumoniae is carbapenem-resistant Klebsiella pneumoniae.

In some embodiments, the Klebsiella pneumoniae induces a Th1 response. In some embodiments, the Klebsiella pneumoniae is strain BAA-2552, strain KP-1, strain 700721, strain 13882, strain 34E1, strain BAA-1705, strain 700603, or strain Kp-2H7. In some embodiments, the Klebsiella pneumoniae is strain Kp-2H7.

In some embodiments the subject is a human. In some embodiments, the composition is administered to the subject more than once. In some embodiments, the composition is administered to the subject by oral administration. In some embodiments, the composition is administered to the subject by rectal administration.

In some embodiments, the administering suppresses the replication, survival, and/or colonization of the pathogenic organism. In some embodiments, the pathogenic organism is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, or Clindamycin resistant Group B Streptococcus.

In some embodiments, the method further comprises administering one or more additional compositions comprising bacteria. In some embodiments, administration of the pharmaceutical composition is not preceded by administration of an antibiotic. In some embodiments, administration of the pharmaceutical composition is not preceded by administration of vancomycin. In some embodiments, the method further comprises administering an antibiotic to the subject prior to administration of the pharmaceutical composition. In some embodiments, the antibiotic is vancomycin.

In some aspects, the present disclosure provides methods of treating a disease or disorder associated with bacterial colonization in a subject, comprising administering a therapeutically effective amount of any of the compositions or pharmaceutical compositions described herein. In some aspects, the present disclosure provides any of the compositions or food products described herein for use as a medicament. In some aspects, the present disclosure provides any of the compositions or food products described herein for use in the treatment of a disease or disorder associated with bacterial colonization. In some aspects, the present disclosure provides use of any of the compositions or food products described herein in the preparation of a medicament for the treatment of a disease or disorder associated with bacterial colonization. In some embodiments, the pathogenic organism is susceptible to antibiotics. In some embodiments, the pathogenic organism is resistant to antibiotics. In some embodiments, the pathogenic organism is a Clostridium difficile. In some embodiments, the pathogenic organism is a multi-drug resistant organism.

In some embodiments, the pathogenic organism is Klebsiella pneumoniae. In some embodiments, the Klebsiella pneumoniae is multi-drug resistant. In some embodiments, the multi-drug resistant Klebsiella pneumoniae is carbapenem-resistant Klebsiella pneumoniae.

In some embodiments, the Klebsiella pneumoniae induces a Th1 response. In some embodiments, the Klebsiella pneumoniae is strain BAA-2552, strain KP-1, strain 700721, strain 13882, strain 34E1, strain BAA-1705, strain 700603, or strain Kp-2H7. In some embodiments, the Klebsiella pneumoniae is strain Kp-2H7.

In some embodiments the subject is a human. In some embodiments, the composition is administered to the subject more than once. In some embodiments, the composition is administered to the subject by oral administration. In some embodiments, the composition is administered to the subject by rectal administration.

In some embodiments, the administering suppresses the replication, survival, and/or colonization of the pathogenic organism.

In some embodiments, the pathogenic organism is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, or Clindamycin resistant Group B Streptococcus.

In some embodiments, the method further comprises administering one or more additional compositions comprising bacteria. In some embodiments, administration of the pharmaceutical composition is not preceded by administration of an antibiotic. In some embodiments, administration of the pharmaceutical composition is not preceded by administration of vancomycin. In some embodiments, the method further comprises administering an antibiotic to the subject prior to administration of the pharmaceutical composition. In some embodiments, the antibiotic is vancomycin.

In some aspects, the present disclosure provides methods of suppressing colonization of the intestine with oral microbiome bacteria, comprising administering a therapeutically effective amount of any of the compositions or food products described herein. In some aspects, the present disclosure provides any of the compositions or food products described herein for use as a medicament. In some aspects, the present disclosure provides any of the compositions or food products described herein for use in the suppression of colonization of the intestine of a subject with oral microbiome bacteria. In some aspects, the present disclosure provides of use of any of the compositions or food products described herein in the preparation of a medicament for the suppression of colonization of the intestine of a subject with oral microbiome bacteria.

In some embodiments, the oral microbiome bacteria are susceptible to antibiotics. In some embodiments, the oral microbiome bacteria are resistant to antibiotics.

In some embodiments the subject is a human. In some embodiments, the composition is administered to the subject more than once. In some embodiments, the composition is administered to the subject by oral administration. In some embodiments, the composition is administered to the subject by rectal administration.

In some embodiments, the administering suppresses the replication, survival, and/or colonization of the oral microbiome bacteria.

In some embodiments, the method further comprises administering one or more additional compositions comprising bacteria. In some embodiments, administration of the pharmaceutical composition is not preceded by administration of an antibiotic. In some embodiments, administration of the pharmaceutical composition is not preceded by administration of vancomycin. In some embodiments, the method further comprises administering an antibiotic to the subject prior to administration of the pharmaceutical composition. In some embodiments, the antibiotic is vancomycin.

### BRIEF DESCRIPTION OF THE DRAWINGS

The accompanying drawings are not intended to be drawn to scale. The figures are illustrative only and are not required for enablement of the disclosure. For purposes of clarity, not every component may be labeled in every drawing. In the drawings:

FIG. 1 presents a timeline of a mouse model of carbapenem-resistant Enterobacteriaceae (“CRE”) colonization experiment.

FIG. 2 shows CRE colonization (colony forming units, “CFU”) levels in fecal samples from mice at 15 days post treatment. From left-to-right: control (PBS), a composition of 36 bacterial strains (“36-mix” or “LBP-1”), 36-mix without E. coli (“36-Ec” or “LBP-2”), 36-mix without Fusobacterium mortiferum (“36-Fuso” or “LBP-3”), 36-mix without both E. coli and Fusobacterium (“36-Ec&Fuso” or “LBP-4”), and stool fraction treatment (“SFL”). L.o.D. is Limit of Detection.

FIGS. 3A and 3B show CRE colonization (CFU) levels in fecal samples from mice at 15 days post treatment. From left-to-right: control (PBS), a composition of 36 bacterial strains (“36-mix” or “LBP-1”), a composition of 33 bacterial strains (“33-mix B” or “LBP-5”), a composition of 32 bacterial strains (“32-mix” or “LBP-6”), a composition of 27 bacterial strains (“27-mix” or “LBP-7”), a composition of 23 bacterial strains (“23-mix” or “LBP-8”), and stool fraction treatment (“SFL”). L.o.D. is Limit of Detection. FIG. 3B is a table showing strains of the indicated compositions and corresponding anti-CRE efficacy. “+” indicates strains included in the indicated composition.

FIG. 4 is a table showing the bacterial strains present in the 36-mix or LBP-1, 27-mix or LBP-7, and 23-mix or LBP-8 assessed in FIG. 3.

FIG. 5 is a table showing the bacterial strains present in the 36-mix or LBP-1, 33-mix B or LBP-5, and 32-mix or LBP-6 assessed in FIG. 3.

FIG. 6 shows results of in vitro competition experiments with the indicated bacterial strains and Campylobacter jejuni 81-176 (C. jejuni). Cultures of C. jejuni were incubated in the presence of controls: Lactococcus sp. (JL17), Escherichia coli ATCC 25922 (Ec 25922), Campylobacter in Thioglycollate broth (Campy thio), Campylobacter in Thioglycollate broth supplemented with chopped meat (Campy CM-thio), Campylobacter in Thioglycollate broth plus succinate (Campy thio+S); or the indicated individual bacterial strains. Strains 8, 9, 18, and 19 were considered “active,” and strains 15, 27, and 36 were considered “weakly active” in reducing the number of C. jejuni colony forming units (CFU/mL) compared to controls. CFU is colony forming units, (+) denotes active strains (≥3 log reduction), (*) denotes weakly active strains (1.5 to 2.9 log reduction), and (∘) denotes controls. “Not active” strains had <1.5 log reduction.

FIG. 7 shows results of in vitro competition experiments with the indicated bacterial strains and Campylobacter jejuni 81-176 (C. jejuni). Cultures of C. jejuni were incubated in the presence of controls: Lactococcus sp. (JL17), Escherichia coli ATCC 25922 (Ec 25922), strain 6, Campylobacter in Thioglycollate broth (Campy thio), Campylobacter in Thioglycollate broth supplemented with chopped meat (Campy CM-thio); or the indicated individual bacterial strains. VE303 strain 6 (VE303-06) was found to be active in reducing the number of C. jejuni colony forming units (CFU/mL). CFU is colony forming units, (+) denotes active strains (≥3 log reduction), (*) denotes weakly active strains (1.5 to 2.9 log reduction), and (∘) denotes controls. “Not active” strains had <1.5 log reduction.

FIG. 8 shows results of in vitro competition experiments with the indicated bacterial strains and Shigella flexneri 2457T (S. flexneri). Cultures of S. flexneri were incubated in the presence of controls: Lactococcus sp. (JL17), Escherichia coli ATCC 25922 (Ec 25922), Shigella in peptone-yeast-glucose medium (Shigella PYG), Shigella in chopped meat with carbohydrates broth (Shigella CMC), Shigella in yeast casitone fatty acids with carbohydrates broth (Shigella YCFAC), Shigella in peptone-yeast-glucose medium with succinate (Shigella PYG+S); or the indicated individual bacterial strains. Strains 8, 9, 11, 13, 16, 21, 35, and 36 were considered “active,” and strains 12, 15, 25, 30, and 34 were considered “weakly active” in reducing the S. flexneri colony forming units (CFU/mL) compared to controls. CFU is colony forming units, (+) denotes active strains (≥4 log reduction), (*) denotes weakly active strains (2 to 3.9 log reduction), and (∘) denotes controls. “Not active” strains had <2 log reduction.

FIG. 9 shows results of in vitro competition experiments with the indicated bacterial strains and Shigella flexneri 2457T (S. flexneri). Cultures of S. flexneri were incubated in the presence of controls: Lactococcus sp. (JL17), Escherichia coli ATCC 25922 (Ec 25922), Shigella in peptone-yeast-glucose medium (Shigella PYG), Shigella in chopped meat with carbohydrates broth (Shigella CMC), strain 24; or the indicated individual bacterial strains. VE303 strains 1 (VE303-01), 2 (VE303-02), 4 (VE303-04), and 5 (VE303-05) were “weakly active” in reducing the S. flexneri colony forming units (CFU/mL). CFU is colony forming units, (+) denotes active strains (≥4 log reduction), (*) denotes weakly active strains (2 to 3.9 log reduction), and (∘) denotes controls. “Not active” strains had <2 log reduction.

FIG. 10 is a table showing a summary of the pathogen-inhibition activity of the indicated bacterial strains.

FIG. 11 shows a workflow diagram for measuring the survival of the Campylobacter jejuni 81-176 strain. The diagram at the right indicates the oxygen concentration gradient used and where facultative anaerobic, aerobic, microaerophilic, and anaerobic bacterial species are expected to grow in thioglycollate broth.

FIG. 12 shows a workflow diagram adapting broth competition assays for measuring the growth suppression of Shigella flexneri 2457T strain (S. flexneri 2457T), Enteroaggregative Escherichia coli (EAEC), and Klebsiella pneumoniae ATCC BAA-2814 (CRE) strains.

FIG. 13 shows results of in vitro broth competition experiments with the indicated bacterial strains and Campylobacter jejuni 81-176 (C. jejuni). Cultures of C. jejuni were incubated in the presence of C. jejuni (Campy alone) or the indicated individual bacterial strains. VE303 strain 6 (VE303-06) was found to be active, and VE303 strains 1 and 7 (VE303-01 and VE303-07, respectively) were found to be weakly active in reducing the number of C. jejuni colony forming units (CFU/mL). CFU is colony forming units, (+) denotes active strains (≥3 log reduction), (*) denotes weakly active strains (1.5 to 2.9 log reduction), and (∘) denotes control. Not active strains had <1.5 log reduction.

FIG. 14 shows a workflow diagram adapting a soft agar overlay assay for measuring the pathogen killing and/or suppression of the Shigella, EAEC, CRE, and Campylobacter strains.

FIG. 15 shows exemplary photographs of a soft agar overlay assay. The final activity of a strain was based on a consensus across all replicates. Strains that are considered “active” produced a zone of inhibition in ≥66% of replicates; strains that are considered “weakly active” produced a zone of inhibition in 34-65% of replicates; and strains that are considered “not active” produced a zone of inhibition in ≤33% in replicates.

FIG. 16 shows the number of active strains and their targets from broth competition assays and soft agar overlay assays for measuring the growth suppression and/or killing of C. jejuni (Campylobacter), S. flexneri (Shigella), and EAEC strains.

FIG. 17 is a table showing a summary of the pathogen inhibition activity of the indicated bacterial strains in the broth competition assays and soft agar overlay assays against S. flexneri, EAEC, or C. jejuni.

FIG. 18 is a table showing a summary of the pathogen inhibition activity of the indicated bacterial strains in the broth competition assays and soft agar overlay assays against S. flexneri, EAEC, or C. jejuni.

FIG. 19 is a table showing a summary of the pathogen inhibition activity of the indicated bacterial strains in the broth competition assays and soft agar overlay assays against S. flexneri, EAEC, or C. jejuni.

FIG. 20 is a table showing a summary of the pathogen inhibition activity of the indicated bacterial strains in the broth competition assays and soft agar overlay assays against S. flexneri, EAEC, or C. jejuni.

FIG. 21 is a table showing a summary of the pathogen inhibition activity of the indicated bacterial strains in the broth competition assays and soft agar overlay assays against S. flexneri, EAEC, or C. jejuni.

FIG. 22 shows CRE colonization (colony forming units, “CFU”) levels in fecal samples from mice at 15 days post treatment with the indicated live bacterial compositions. From left-to-right: control (PBS), a composition of 36 bacterial strains (“LBP-1”), a composition of 31 bacterial strains (“LBP-9”), a composition of 33 bacterial strains (“LBP-10”), a composition of 35 bacterial strains (“LBP-11”), a composition of 35 bacterial strains (“LBP-12”), a composition of 30 bacterial strains (“LBP-13”), a composition of 21 bacterial strains (“LBP-14”), a composition of 23 bacterial strains (“LBP-15”), a composition of 26 bacterial strains (“LBP-16”), a composition of 22 bacterial strains (“LBP-17”), a composition of 21 bacterial strains (“LBP-18”), a composition of 20 bacterial strains (“LBP-19”), a composition of 34 bacterial strains (“LBP-20”), a composition of 33 bacterial strains (“LBP-21”), a composition of 27 bacterial strains (“LBP-22”), a composition of 25 bacterial strains (“LBP-23”), a composition of 27 bacterial strains (“LBP-24”), a composition of 30 bacterial strains (“LBP-25”), a composition of 27 bacterial strains (“LBP-26”), a composition of 22 bacterial strains (“LBP-27”), a composition of 26 bacterial strains (“LBP-28”), a composition of 22 bacterial strains (“LBP-29”), a composition of 17 bacterial strains (“LBP-30”), a composition of 25 bacterial strains (“LBP-31”), a composition of 19 bacterial strains (“LBP-32”), and a composition of 17 bacterial strains (“LBP-33”). L.o.D. is Limit of Detection. The strain composition of each of the LBPs can also be found in FIG. 35.

FIG. 23 shows CRE colonization (colony forming units, “CFU”) levels in fecal samples from mice at 0 (D0), 8 (D8), 15 (D15), and 22 (D22) days post treatment. At each time point, the treatment corresponds to, from left to right: control (PBS), a composition of 30 bacterial strains (“30-mix”), and a composition of 36 bacterial strains (“36-mix”). The 36-mix corresponds to “LBP-1” and the 30-mix corresponds to “LBP-25”. L.o.D. is Limit of Detection.

FIGS. 24A and 24B show assessment of exclusion of various strains on CRE (Klebsiella pneumoniae “Kpn”) decolonization. FIG. 24A shows CRE colonization (colony forming units, “CFU”) levels in fecal samples from mice at days 14-17 post treatment (D14-17 post Tx). L.o.D. is Limit of Detection. FIG. 24B is a table showing strains excluded from the indicated compositions and corresponding anti-CRE efficacy. “+” indicates strains included in the indicated composition.

FIGS. 25A and 25B show assessment of exclusion of Bacteroides species on CRE decolonization. FIG. 25A is a table showing the bacterial species present in LBP-32 (19-mix). “+” indicates strains included in the indicated composition. FIG. 25B shows CRE (Klebsiella pneumoniae “Kpn”) colonization (colony forming units, “CFU”) levels in fecal samples from mice at 0 (D0), 3 (D3), 8 (D8), 14 (D14), 17 (D17), and 22 (D22) days post treatment. At each time point, the treatment corresponds to, from left to right: control (PBS), composition LBP-32 (19-mix), and composition LBP-1 (36-mix). L.o.D. is Limit of Detection.

FIGS. 26A and 26B show assessment of modified versions of live bacterial product LBP-32 (19-mix) on CRE (Klebsiella pneumoniae “Kpn”) decolonization. FIG. 26A is a table showing the bacterial species present in the 19-mix (“LBP-32”) and a modified 17-mix (“LBP-30”). “+” indicates strains included in the indicated composition. Arrows indicate Bacteroides vulgatus, Clostridium citroniae, and Phascolarctobacterium faecis, which are excluded from the 17-mix (“LBP-30”). FIG. 26B shows CRE colonization (colony forming units, “CFU”) levels in fecal samples from mice at 15 days post treatment (D8 post Tx) with the indicated live bacterial product: LBP-30 (17-mix), LBP-32 (19-mix), LBP-24 (27-mix), LBP-1 (36-mix), or control (PBS). L.o.D. is Limit of Detection.

FIG. 27 shows CRE colonization (colony forming units, “CFU”) levels in small intestine samples from mice at 22 days post treatment (Post Tx) with control (PBS) or the live bacterial product LBP-32 (19-mix). L.o.D. is limit of detection.

FIGS. 28A and 28B show optimization strategies for developing live bacterial products. FIG. 28A shows a schematic of a single model optimization strategy in which a live biotherapeutic product (“LBP”) is administered to mice colonized with Klebsiella pneumoniae (CRE; “Anti-CRE LBP”) or mice colonized with ESBL; “Anti-ESBL LBP”). Then, the Anti-CRE LBP and Anti-ESBL LBP are combined and administered to mice colonized with CRE and mice colonized with ESBL, and candidate LBP are identified. FIG. 28B shows a schematic of a dual model optimization strategy in which a mouse model of CRE and ESBL colonization is administered both Anti-CRE-LBP and Anti-ESBL-LBP, and candidate LBP are identified.

FIG. 29 presents a timeline of a mouse model of a single challenge with carbapenem-resistant Enterobacteriaceae (“CRE”) or extended spectrum beta-lactamase (“ESBL”)-producing Enterobacteriaceae colonization experiment, or a co-challenge CRE and ESBL colonization experiment.

FIGS. 30A and 30B shows colonization levels of mice in the single challenge (mono-colonized) or co-colonized challenge models shown in FIG. 29. FIG. 30A shows extended spectrum beta-lactamase (“ESBL”) producing Enterobacteriaceae colonization (colony forming units, “CFU”) levels in fecal samples from mice at 3 (D3), 6 (D6), 11 (D11), and 17 (D17) post-challenge. Mice were either challenged with ESBL (“mono-colonized,” light gray) or co-challenged with ESBL and CRE (“co-colonized,” dark gray). FIG. 30B shows CRE colonization (colony forming units, “CFU”) levels in fecal samples from mice at 3 (D3), 6 (D6), 11 (D11), and 17 (D17) days post-challenge. Mice were either challenged with CRE (“mono-colonized,” light gray) or co-challenged with CRE and ESBL (“co-colonized,” dark gray). Each group of mice contained 8 mice.

FIGS. 31A and 31B show colonization of mice in the single challenge (mono-colonized) or co-colonized challenge models. FIG. 31A presents a timeline of a mouse model of a single challenge with carbapenem-resistant Enterobacteriaceae (“CRE”) or extended spectrum beta-lactamase (“ESBL”) producing Enterobacteriaceae colonization experiment, or a co-challenge CRE and ESBL colonization experiment on day −3 after 7 days of antibiotics administration. The 36-mix (LBP-1) or control (“PBS”) was then administered on days −2, −1, and 0. FIG. 31B shows colonization levels of mice in the single-challenge (“mono”) or co-colonized (“co”) challenge models shown in FIG. 31A. Mice were either challenged with CRE (“CRE Mono”), ESBL (“ESBL Mono”), co-challenged with CRE and ESBL (“CRE Co”), or co-challenged with ESBL and CRE (“ESBL Co”). Colonization (colonization forming units, “CFU”) levels in fecal samples are shown at 14 days post-treatment with the 36-mix or control (PBS).

FIG. 32 is a table showing a summary of the pathogen inhibition activity of the indicated bacterial strains in the broth competition assays and soft agar overlay assays against S. flexneri, EAEC, or C. jejuni.

FIG. 33 is a table showing a summary of the pathogen inhibition activity of the indicated bacterial strains in the broth competition assays and soft agar overlay assays against S. flexneri, EAEC, or C. jejuni.

FIGS. 34A and 34B show the number of active strains and their targets from broth competition assays and soft agar overlay assays for measuring the growth suppression and/or killing of C. jejuni (“Campylobacter”), S. flexneri (“Shigella”), and EAEC strains.

FIG. 35 is a table showing the bacterial strains present in each of the bacterial compositions assessed in FIG. 22: LBP-1, LBP-9, LBP-10, LBP-11, LBP-12, LBP-13, LBP-14, LBP-15, LBP-16, LBP-17, LBP-18, LBP-19, LBP-20, LBP-21, LBP-22, LBP-23, LBP-24, LBP-25, LBP-26, LBP-27, LBP-28, LBP-29, LBP-30, LBP-31, LBP-32, LBP-33, and LBP-34. The number of bacterial strains in each LBP composition is indicated in parentheses.

FIG. 36 shows results of in vitro competition experiments with the indicated bacterial strains and carbapenem-resistant Klebsiella pneumoniae ATCC BAA-2814 (CRE). Cultures of CRE were incubated in the presence of controls: Lactococcus sp. (JL17), Escherichia coli ATCC 25922 (Ec 25922), VE303 strain 6 (VE303-06), CRE only (CRE alone); or the indicated individual bacterial strains. Strains 16, 36, 9, 10, and 8 were considered “active,” and strains 11 and 34 were considered “weakly active” in reducing the number of CRE colony forming units (CFU/mL) compared to controls. CFU is colony forming units, (+) denotes active strains (≥4.0 log reduction), (*) denotes weakly active strains (2.0 to 3.9 log reduction), and (∘) denotes controls. “Not active” strains had <2.0 log reduction.

FIG. 37 shows results of in vitro competition experiments with the indicated bacterial strains and carbapenem-resistant Klebsiella pneumoniae ATCC BAA-2814 (CRE). Cultures of CRE were incubated in the presence of controls: Lactococcus sp. (JL17), Escherichia coli ATCC 25922 (Ec 25922), CRE only (CRE alone); or the indicated individual bacterial strains. VE303 strain 1 (VE303-01) and VE303 strain 4 (VE303-04) were found to be active in reducing the number of CRE colony forming units (CFU/mL). CFU is colony forming units, (+) denotes active strains (≥4.0 log reduction), (*) denotes weakly active strains (2.0 to 3.9 log reduction), and (∘) denotes controls. “Not active” strains had <2.0 log reduction.

FIG. 38 is a table showing a summary of the pathogen inhibition activity of the indicated bacterial strains in the broth competition assays and soft agar overlay assays against CRE.

FIG. 39 shows carbapenem-resistant Klebsiella pneumoniae (CRE) colonization (colony forming units, “CFU”) levels in samples from mice at 14 days post treatment (Post Tx) with control (PBS), the live bacterial product LBP-32, or the live bacterial product LBP-34. L.o.D. is limit of detection.

### DETAILED DESCRIPTION

Suppressing or preventing undesired bacteria in a subject or suppressing or preventing colonization of bacteria in a particular region of the body can be challenging. Bacterial colonization may induce immune responses (local or systemic) in the subject, which may lead to serious disease. In particular, for multi-drug resistant organisms, or organisms that have acquired antibiotic resistance, elimination with many conventional therapeutics, such as antibiotics, may not be possible due to resistance or tolerance to the therapeutic. Additionally, it has been recently appreciated that intestinal colonization by bacteria of the oral microbiome may influence the immune environment of the intestine, such as induce Th1-dominated immune responses and lead to chronic inflammation and inflammatory conditions (see, e.g., Atarashi et al. Science (2017) 358 (359-365)). Normal bacterial colonization of different regions of the body, such as the oral cavity, may provide a reservoir of bacteria that can migrate and colonize other regions, such as the intestines.

Provided herein are compositions and methods for suppressing colonization by pathogenic organisms, such as multi-drug resistant organisms. Also provided herein are compositions and methods for suppressing colonization of the intestine of subject with bacteria of the oral microbiome. Provided herein are compositions and methods for treating diseases or disorders associated with bacterial colonization or treating diseases or disorders associated with an immune response induced by bacteria. Also provided herein are compositions and methods for inducing regulatory T cell (Treg) production in response to pathogenic organisms. Provided herein are compositions and methods for inducing production of short chain fatty acids (SCFAs) in response to pathogenic organisms.

In some embodiments, the one or more of the bacterial strains of the compositions provided herein colonize or recolonize the intestinal tract or parts of the intestinal tract (e.g., the colon or the cecum) of the subject. Such colonization or recolonization may also be referred to as grafting. In some embodiments, the one or more of the bacterial strains of the compositions recolonize the intestinal tract (e.g., the colon or the cecum) of the subject, for example after another organism or population of organisms has been partially or completely removed (e.g., by antibiotic treatment). In some embodiments, one or more of the bacterial strains of the compositions recolonize the intestinal tract (e.g., the colon or the cecum) after one or more pathogenic organisms or other organism (e.g., bacteria that induce an immune response) has been removed. In some embodiments, the recolonization of the intestinal tract or parts thereof by the bacterial strains of the compositions described herein prevents or suppresses colonization by undesired organisms (e.g., pathogenic organisms, multi-drug resistant organisms, oral microbiome bacteria, bacteria that induce immune responses, pathobionts, bacteria that reduce Treg production, bacteria that reduce SCFA production).

In some embodiments, the one or more of the bacterial strains of the compositions can “outgrow” a pathogen or undesired bacteria, such as a pathogenic organism, multi-drug resistant organism, oral microbiome bacteria, bacteria that induce immune responses, bacteria that reduce Treg production, or bacteria that reduce SCFA production. Thus, in some embodiments, if a pathogen or undesired bacteria (e.g., pathogenic organisms, multi-drug resistant organism, oral microbiome bacteria, bacteria that induce immune responses bacteria that reduce Treg production, bacteria that reduce SCFA production) and one or more bacteria of compositions provided herein are both present in the intestinal tract (e.g., the colon or the cecum), the one or more bacteria of compositions provided herein grow faster (e.g., have a shorter doubling time) than the pathogen, thereby preventing the pathogen from accumulating in the intestinal tract (e.g., the colon or the cecum). In some embodiments, the one or more bacteria of compositions provided herein grow faster than the pathogen in an otherwise intact or complete microbiome. In some embodiments, the one or more bacteria of compositions provided herein grow faster than the pathogen in a depleted microbiome (e.g., following antibiotic treatment). In some embodiments, the faster growth results because the one or more bacteria of the compositions provided herein are better at grafting in the intestinal tract (e.g., the colon or the cecum). In some embodiments, the faster growth results because the one or more bacteria of the compositions provided herein are better at metabolizing nutrients present in the intestinal tract (e.g., the colon or the cecum). In some embodiments, the compositions of bacterial strains provided herein prevent or inhibit replication of the pathogen. In some embodiments, the compositions of bacterial strains provided herein induce death of (kill) the pathogen. In some embodiments, the bacterial strains of the compositions provided herein can treat pathogenic infections, because of the synergy between the bacterial strains.

In some embodiments, the bacterial compositions described herein prevent recolonization by a pathogen or undesired bacteria (e.g., pathogenic organisms, multi-drug resistant organisms, oral microbiome bacteria, bacteria that induce immune responses, pathobionts, bacteria that reduce Treg production, bacteria that reduce SCFA production). For example, in some embodiments, the pathogen or undesired bacteria have been reduced or eliminated from the subject, for example, using a first therapeutic agent (such as an antibiotic), and the bacterial compositions described herein are administered to prevent recolonization of the subject. In some embodiments, the bacterial compositions described herein reduce or eliminate a pathogen or undesired bacteria from the subject and prevent recolonization of the subject.

In some embodiments, the combination of bacterial strains of the compositions provided herein is superior in the use of nutrients when compared to the pathogen or undesired bacteria, thereby suppressing the growth of the pathogen or undesired bacteria. In some embodiments, the combination of bacterial strains of the compositions provided herein is superior in grafting when compared to the pathogen or undesired bacteria, thereby suppressing the growth of the pathogen or undesired bacteria. In some embodiments, the combination of bacterial strains of the compositions provided herein is superior in the use of nutrients and in grafting when compared to the pathogen or undesired bacteria, thereby suppressing the growth of the pathogen or undesired bacteria. In some embodiments, the combination of bacterial strains of the compositions provided herein inhibits the growth, survival, and/or colonization of the pathogen or undesired bacteria.

In some embodiments, the combination of bacterial strains of the compositions provided herein has antagonizing or inhibitory activity towards the pathogen or undesired bacteria, thereby inhibiting the growth, survival, and/or colonization of the pathogen or undesired bacteria. In some embodiments, at least one bacterial strain of the compositions provided herein has antagonizing or inhibitory activity towards the pathogen or undesired bacteria, thereby inhibiting the growth, survival, and/or colonization of the pathogen or undesired bacteria.

In some embodiments, the synergistic effect is provided by the capacity of the combination to colonize specific niches in the intestinal tract (e.g., the colon or the cecum). In some embodiments, the synergistic effect is provided by the capacity of the combination to metabolize specific nutrients. In some embodiments, the synergistic effect is provided by the capacity of the combination to provide specific metabolites to the environment.

The bacterial strains used in the compositions provided herein generally are isolated from the microbiome of healthy individuals. In some embodiments, the compositions include strains originating from a single individual. In some embodiments, the compositions include strains originating from multiple individuals. In some embodiments, the bacterial strains are obtained from multiple individuals, isolated, and grown up individually. The bacterial compositions that are grown up individually may subsequently be combined to provide the compositions of the disclosure. It should be appreciated that the origin of the bacterial strains of the compositions provided herein is not limited to the human microbiome from a healthy individual. In some embodiments, the bacterial strains originate from a human with a microbiome in dysbiosis. In some embodiments, the bacterial strains originate from non-human animals or the environment (e.g., soil or surface water). In some embodiments, the combinations of bacterial strains provided herein originate from multiple sources (e.g., human and non-human animals).

In some embodiments, the bacteria of the compositions provided herein are anaerobic bacteria. In some embodiments, the bacteria of the compositions provided herein are obligate anaerobic bacteria. In some embodiments, the bacteria of the compositions provided herein are clostridia. Clostridia may be classified into phylogenetic clusters with other closely related strains and species. (See e.g., Rajilic-Stojanovic, M., and de Vos, W. M. FEMS Microbiol Rev 38, (2014) 996-1047). In general, clostridia are classified as belonging to a specific cluster based on their 16S rRNA (or 16S rDNA) nucleic acid sequence. Methods for determining the identity of specific bacterial species based on their 16S rRNA (or 16S rDNA) nucleic acid sequence are well known in the art (See, e.g., Jumpstart Consortium Human Microbiome Project Data Generation Working, G. PLoS One (2012) 7, e39315).

In one aspect, the disclosure provides compositions comprising one or more purified bacterial strains belonging to the phylum Firmicutes and one or more purified bacterial strains selected from the group consisting of an Escherichia species (e.g., Escherichia sp. 3_2_53FAA) and a Fusobacterium species (e.g., Fusobacterium mortiferum). In one aspect, the disclosure provides compositions consisting of one or more purified bacterial strains belonging to the phylum Firmicutes and one or more purified bacterial strains selected from the group consisting of an Escherichia species (e.g., Escherichia sp. 3_2_53FAA) and a Fusobacterium species (e.g., Fusobacterium mortiferum). In one aspect, the disclosure provides compositions consisting essentially of one or more purified bacterial strains belonging to the phylum Firmicutes and one or more purified bacterial strains selected from the group consisting of an Escherichia species (e.g., Escherichia sp. 3_2_53FAA) and a Fusobacterium species (e.g., Fusobacterium mortiferum). In some embodiments, consisting essentially of, as used herein, refers to compositions that do not include any additional therapeutically active bacterial strains. In one aspect, the disclosure provides compositions consisting of one or more purified bacterial strains belonging to the phylum Firmicutes and one or more purified bacterial strains selected from the group consisting of an Escherichia species (e.g., Escherichia sp. 3_2_53FAA) and a Fusobacterium species (e.g., Fusobacterium mortiferum).

In one aspect, the disclosure provides compositions comprising one or more purified bacterial strains belonging to the phylum Firmicutes and one or more purified bacterial strains of Escherichia species, such as E. coli (e.g., Escherichia sp. 3_2_53FAA). In one aspect, the disclosure provides compositions consisting of one or more purified bacterial strains belonging to the phylum Firmicutes and one or more purified bacterial strains of Escherichia species, such as E. coli (e.g., Escherichia sp. 3_2_53FAA). In one aspect, the disclosure provides compositions consisting essentially of one or more purified bacterial strains belonging to the phylum Firmicutes and one or more purified bacterial strains of Escherichia species, such as E. coli (e.g., Escherichia sp. 3_2_53FAA).

In one aspect, the disclosure provides compositions comprising one or more purified bacterial strains belonging to the phylum Firmicutes and one or more purified bacterial strains of Fusobacterium species (e.g., Fusobacterium mortiferum). In one aspect, the disclosure provides compositions consisting of one or more purified bacterial strains belonging to the phylum Firmicutes and one or more purified bacterial strains of Fusobacterium species (e.g., Fusobacterium mortiferum). In one aspect, the disclosure provides compositions consisting essentially of one or more purified bacterial strains belonging to the phylum Firmicutes and one or more purified bacterial strains of Fusobacterium species (e.g., Fusobacterium mortiferum).

The phylum Firmicutes are characterized by low-guanosine and cytosine content DNA. Firmicutes make up the largest portion of the mouse and human microbiomes, where they are involved in energy resorption and the development of diabetes and obesity. Classes of Firmicute bacteria include the Bacilli (e.g., Bacillales, Lactobacillales, Mollicutes, Erysipelotrichia) and the Clostridia (e.g., Clostridales, Halanaerobiales, Natranaerobiales, Thermoanaerobacterales, and Negativicutes), and Thermolithobacteria.

In some embodiments, one or more bacterial strains of the phylum Firmicutes belong to the family Clostridiaceae. In some embodiments, the compositions described herein contain one or more bacterial strains belonging to the genus Clostridium. In some embodiments, the compositions described herein contain one or more bacterial strains belonging to Clostridium cluster IV and/or XIVa.

In some embodiments, one or more bacterial strains of the phylum Firmicutes produces short chain fatty acids (SCFAs), such as any of the SCFAs described herein. In some embodiments, one or more bacterial strains of the phylum Firmicutes produces butyrate. Butyrate is primarily produced by Firmicutes and Bacteriodetes in the gastrointestinal tract. In some embodiments, one or more of the bacterial strains that belong to the Firmicutes are butyrate producers, such as Anaerotruncus colihominis, Sellimonas intestinalis (Ruminococcus torques), Clostridium symbiosum, Dorea longicatena, Erysipelotrichaceae bacterium, and Flavinofractor plautii (Subdoligranulum spp).

In one aspect, the disclosure provides compositions comprising a purified bacterial strain belonging to Escherichia species, such as E. coli (e.g., Escherichia sp. 3_2_53FAA), a purified bacterial strain belonging to Fusobacterium species (e.g., Fusobacterium mortiferum) and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis.

In one aspect, the disclosure provides compositions consisting of a purified bacterial strain belonging to Escherichia species, such as E. coli (e.g., Escherichia sp. 3_2_53FAA), a purified bacterial strain belonging to Fusobacterium species (e.g., Fusobacterium mortiferum) and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis.

In one aspect, the disclosure provides compositions consisting essentially of a purified bacterial strain belonging to Escherichia species, such as E. coli (e.g., Escherichia sp. 3_2_53FAA), a purified bacterial strain belonging to Fusobacterium species (e.g., Fusobacterium mortiferum) and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis. In some embodiments, consisting essentially of, as used herein, refers to compositions that do not include any additional therapeutically active bacterial strains.

In one aspect, the bacterial composition comprises a purified bacterial strain comprising a 16S rDNA sequence having at least 97% sequence identity with the nucleic acid sequence set forth as SEQ ID NO: 36, a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 8-20, 25, 28, 30-32, and 35.

In one aspect, the bacterial composition consists of a purified bacterial strain comprising a 16S rDNA sequence having at least 97% sequence identity with the nucleic acid sequence set forth as SEQ ID NO: 36, a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 8-20, 25, 28, 30-32, and 35.

In one aspect, the bacterial composition consists essentially of a purified bacterial strain comprising a 16S rDNA sequence having at least 97% sequence identity with the nucleic acid sequence set forth as SEQ ID NO: 36, a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 8-20, 25, 28, 30-32, and 35.

In one aspect, the disclosure provides compositions comprising a purified bacterial strain belonging to Escherichia species, such as E. coli (e.g., Escherichia sp. 3_2_53FAA) and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis.

In one aspect, the disclosure provides compositions consisting of a purified bacterial strain belonging to Escherichia species, such as E. coli (e.g., Escherichia sp. 3_2_53FAA) and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis.

In one aspect, the disclosure provides compositions consisting essentially of a purified bacterial strain belonging to Escherichia species, such as E. coli (e.g., Escherichia sp. 3_2_53FAA) and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis. In some embodiments, consisting essentially of, as used herein, refers to compositions that do not include any additional therapeutically active bacterial strains.

In one aspect, the bacterial composition comprises a purified bacterial strain comprising a 16S rDNA sequence having at least 97% sequence identity with the nucleic acid sequence set forth as SEQ ID NO: 36, and one or more purified bacterial strains comprising a 16S rDNA sequence having at least 97% sequence identity with the nucleic acid sequence set forth as SEQ ID NOs: 8-20, 25, 28, 30-32, and 35.

In one aspect, the bacterial composition consists of a purified bacterial strain comprising a 16S rDNA sequence having at least 97% sequence identity with the nucleic acid sequence set forth as SEQ ID NO: 36, and one or more purified bacterial strains comprising a 16S rDNA sequence having at least 97% sequence identity with the nucleic acid sequence set forth as SEQ ID NOs: 8-20, 25, 28, 30-32, and 35.

In one aspect, the bacterial composition consists essentially of a purified bacterial strain comprising a 16S rDNA sequence having at least 97% sequence identity with the nucleic acid sequence set forth as SEQ ID NO: 36, and one or more purified bacterial strains comprising a 16S rDNA sequence having at least 97% sequence identity with the nucleic acid sequence set forth as SEQ ID NOs: 8-20, 25, 28, 30-32, and 35.

In one aspect, the disclosure provides compositions comprising a purified bacterial strain belonging to Fusobacterium species (e.g., Fusobacterium mortiferum) and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis.

In one aspect, the disclosure provides compositions consisting of a purified bacterial strain belonging to Fusobacterium species (e.g., Fusobacterium mortiferum) and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis.

In one aspect, the disclosure provides compositions consisting essentially of a purified bacterial strain belonging to Fusobacterium species (e.g., Fusobacterium mortiferum) and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis. In some embodiments, consisting essentially of, as used herein, refers to compositions that do not include any additional therapeutically active bacterial strains.

In one aspect, the bacterial composition comprises a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 8-20, 25, 28, 30-32, and 35.

In one aspect, the bacterial composition consists of a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 8-20, 25, 28, 30-32, and 35.

In one aspect, the bacterial composition consists essentially of a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 8-20, 25, 28, 30-32, and 35.

In one aspect, the disclosure provides compositions comprising bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, and Agathobaculum butyriciproducens.

In one aspect, the disclosure provides compositions consisting of bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, and Agathobaculum butyriciproducens.

In one aspect, the disclosure provides compositions consisting essentially of bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, and Agathobaculum butyriciproducens. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 1-30 and 32. In one aspect, the bacterial composition consists of purified bacterial strains comprising a 16S rDNA sequence having at least 97% sequence identity with the nucleic acid sequence set forth as SEQ ID NOs: 1-30 and 32. In one aspect, the bacterial composition consists essentially of purified bacterial strains comprising a 16S rDNA sequence having at least 97% sequence identity with the nucleic acid sequence set forth as SEQ ID NOs: 1-30 and 32.

In one aspect, the disclosure provides compositions comprising bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Bacteroides fragilis, Paraclostridium bifermentans, Escherichia coli, and Fusobacterium mortiferum.

In one aspect, the disclosure provides compositions consisting of bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Bacteroides fragilis, Paraclostridium bifermentans, Escherichia coli, and Fusobacterium mortiferum.

In one aspect, the disclosure provides compositions consisting essentially of bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Bacteroides fragilis, Paraclostridium bifermentans, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 1-29 and 33-36. In one aspect, the bacterial composition consists of purified bacterial strains comprising a 16S rDNA sequence having at least 97% sequence identity with the nucleic acid sequence set forth as SEQ ID NOs: 1-29 and 33-36. In one aspect, the bacterial composition consists essentially of purified bacterial strains comprising a 16S rDNA sequence having at least 97% sequence identity with the nucleic acid sequence set forth as SEQ ID NOs: 1-29 and 33-36.

In one aspect, the disclosure provides compositions comprising bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, Phascolarctobacterium faecium, Agathobaculum butyriciproducens, Bacteroides fragilis, Fusobacterium mortiferum, and Paraclostridium bifermentans.

In one aspect, the disclosure provides compositions consisting of bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, Phascolarctobacterium faecium, Agathobaculum butyriciproducens, Bacteroides fragilis, Fusobacterium mortiferum, and Paraclostridium bifermentans.

In one aspect, the disclosure provides compositions consisting essentially of bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, Phascolarctobacterium faecium, Agathobaculum butyriciproducens, Bacteroides fragilis, Fusobacterium mortiferum, and Paraclostridium bifermentans. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 1-35. In one aspect, the bacterial composition consists of purified bacterial strains comprising a 16S rDNA sequence having at least 97% sequence identity with the nucleic acid sequence set forth as SEQ ID NOs: 1-35. In one aspect, the bacterial composition consists essentially of purified bacterial strains comprising a 16S rDNA sequence having at least 97% sequence identity with the nucleic acid sequence set forth as SEQ ID NOs: 1-35.

In one aspect, the disclosure provides compositions comprising bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Phascolarctobacterium faecium, Anaerostipes caccae, Agathobaculum butyriciproducens, Bacteroides fragilis, Paraclostridium bifermentans, and E. coli.

In one aspect, the disclosure provides compositions consisting of bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Phascolarctobacterium faecium, Anaerostipes caccae, Agathobaculum butyriciproducens, Bacteroides fragilis, Paraclostridium bifermentans, and E. coli.

In one aspect, the disclosure provides compositions consisting essentially of bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Phascolarctobacterium faecium, Anaerostipes caccae, Agathobaculum butyriciproducens, Bacteroides fragilis, Paraclostridium bifermentans, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 1-33, 35, and 36. In one aspect, the bacterial composition consists of purified bacterial strains comprising a 16S rDNA sequence having at least 97% sequence identity with the nucleic acid sequence set forth as SEQ ID NOs: 1-33, 35, and 36. In one aspect, the bacterial composition consists essentially of purified bacterial strains comprising a 16S rDNA sequence having at least 97% sequence identity with the nucleic acid sequence set forth as SEQ ID NOs: 1-33, 35, and 36.

In one aspect, the disclosure provides compositions comprising bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Fusobacterium mortiferum, and E. coli.

In one aspect, the disclosure provides compositions consisting of bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Fusobacterium mortiferum, and E. coli.

In one aspect, the disclosure provides compositions consisting essentially of bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 1-15, 17-29, 34, and 36. In one aspect, the bacterial composition consists of purified bacterial strains comprising a 16S rDNA sequence having at least 97% sequence identity with the nucleic acid sequence set forth as SEQ ID NOs: 1-15, 17-29, 34, and 36. In one aspect, the bacterial composition consists essentially of purified bacterial strains comprising a 16S rDNA sequence having at least 97% sequence identity with the nucleic acid sequence set forth as SEQ ID NOs: 1-15, 17-29, 34, and 36.

In one aspect, the disclosure provides compositions comprising bacterial strains of species Bacteroides intestinalis, Bacteroides faecis, Bacteroides ovatus, Bacteroides uniformis, Bacteroides vulgatus, Bifidobacterium longum, Blautia producta, Clostridium citroniae, Clostridium clostridioforme, Erysipelotrichaceae bacterium 6_1_45, Dorea longicatena, Erysipelatoclostridium ramosum, Eubacterium rectale, Odoribacter sp. UNK.MGS-12, Parabacteroides distasonis, Bacteroides xylanisolvens, Blautia obeum, Alistipes putredinis, Eubacterium hallii, Fusobacterium mortiferum, and E. coli.

In one aspect, the disclosure provides compositions consisting of bacterial strains of species Bacteroides intestinalis, Bacteroides faecis, Bacteroides ovatus, Bacteroides uniformis, Bacteroides vulgatus, Bifidobacterium longum, Blautia producta, Clostridium citroniae, Clostridium clostridioforme, Erysipelotrichaceae bacterium 6_1_45, Dorea longicatena, Erysipelatoclostridium ramosum, Eubacterium rectale, Odoribacter sp. UNK.MGS-12, Parabacteroides distasonis, Bacteroides xylanisolvens, Blautia obeum, Alistipes putredinis, Eubacterium hallii, Fusobacterium mortiferum, and E. coli. In one aspect, the disclosure provides compositions consisting essentially of bacterial strains of species Bacteroides intestinalis, Bacteroides faecis, Bacteroides ovatus, Bacteroides uniformis, Bacteroides vulgatus, Bifidobacterium longum, Blautia producta, Clostridium citroniae, Clostridium clostridioforme, Erysipelotrichaceae bacterium 6_1_45, Dorea longicatena, Erysipelatoclostridium ramosum, Eubacterium rectale, Odoribacter sp. UNK.MGS-12, Parabacteroides distasonis, Bacteroides xylanisolvens, Blautia obeum, Alistipes putredinis, Eubacterium hallii, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 2-4, 6, 7, 9, 11-13, 15, 18-22, 24-26, 28, 34, and 36. In one aspect, the bacterial composition consists of purified bacterial strains comprising a 16S rDNA sequence having at least 97% sequence identity with the nucleic acid sequence set forth as SEQ ID NOs: 2-4, 6, 7, 9, 11-13, 15, 18-22, 24-26, 28, 34, and 36. In one aspect, the bacterial composition consists essentially of purified bacterial strains comprising a 16S rDNA sequence having at least 97% sequence identity with the nucleic acid sequence set forth as SEQ ID NOs: 2-4, 6, 7, 9, 11-13, 15, 18-22, 24-26, 28, 34, and 36.

In one aspect, the disclosure provides compositions comprising bacterial strains of species Bacteroides caccae, Bacteroides intestinalis, Bacteroides faecis, Bacteroides thetaiotaomicron, Bacteroides uniformis, Bacteroides vulgatus, Bacteroides ovatus, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Bacteroides xylanisolvens, Blautia obeum, Alistipes putredinis, Eubacterium hallii, Alistipes shahii, Fusobacterium mortiferum, and E. coli.

In one aspect, the disclosure provides compositions consisting of bacterial strains of species Bacteroides caccae, Bacteroides intestinalis, Bacteroides faecis, Bacteroides thetaiotaomicron, Bacteroides uniformis, Bacteroides vulgatus, Bacteroides ovatus, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Bacteroides xylanisolvens, Blautia obeum, Alistipes putredinis, Eubacterium hallii, Alistipes shahii, Fusobacterium mortiferum, and E. coli.

In one aspect, the disclosure provides compositions consisting essentially of bacterial strains of species Bacteroides caccae, Bacteroides intestinalis, Bacteroides faecis, Bacteroides thetaiotaomicron, Bacteroides uniformis, Bacteroides vulgatus, Bacteroides ovatus, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Bacteroides xylanisolvens, Blautia obeum, Alistipes putredinis, Eubacterium hallii, Alistipes shahii, Fusobacterium mortiferum, and E. coli. In some embodiments, consisting essentially of, as used herein, refers to compositions that do not include any additional therapeutically active bacterial strains. In one aspect, the bacterial composition comprises purified bacterial strains comprising a 16S rDNA sequence having at least 97% sequence identity with the nucleic acid sequence set forth as SEQ ID NOs: 1-3, 5-8, 11-15, 17-20, 24-26, 28, 29, 34, and 36. In one aspect, the bacterial composition consists of purified bacterial strains comprising a 16S rDNA sequence having at least 97% sequence identity with the nucleic acid sequence set forth as SEQ ID NOs: 1-3, 5-8, 11-15, 17-20, 24-26, 28, 29, 34, and 36. In one aspect, the bacterial composition consists essentially of purified bacterial strains comprising a 16S rDNA sequence having at least 97% sequence identity with the nucleic acid sequence set forth as SEQ ID NOs: 1-3, 5-8, 11-15, 17-20, 24-26, 28, 29, 34, and 36.

In one aspect, the disclosure provides compositions comprising bacterial strains of species Bacteroides caccae, Bacteroides intestinalis, Bacteroides faecis, Bacteroides ovatus, Bacteroides thetaiotamicron, Bacteroides uniformis, Bacteroides vulgatus, Bifidobacterium adolescentis, Bifidobacterium longum, Bifidobacterium pseudocatenulatum, Clostridium innocuum, Erysipelotrichaceae bacterium 6_1_45, Paeniclostridium sordellii, Erysipelatoclostridium ramosum, Odoribacter sp. UNK.MGS-12, Parabacteroides distasonis, Parabacteroides merdae, Bacteroides xylanisolvens, Alistipes putredinis, Collinsella aerofaciens, Alistipes shahii, Phascolarctobacterium faecium, Bacteroides fragilis, Paraclostridium bifermentans, Fusobacterium mortiferum, and E. coli.

In one aspect, the disclosure provides compositions consisting of bacterial strains of species Bacteroides caccae, Bacteroides intestinalis, Bacteroides faecis, Bacteroides ovatus, Bacteroides thetaiotamicron, Bacteroides uniformis, Bacteroides vulgatus, Bifidobacterium adolescentis, Bifidobacterium longum, Bifidobacterium pseudocatenulatum, Clostridium innocuum, Erysipelotrichaceae bacterium 6_1_45, Paeniclostridium sordellii, Erysipelatoclostridium ramosum, Odoribacter sp. UNK.MGS-12, Parabacteroides distasonis, Parabacteroides merdae, Bacteroides xylanisolvens, Alistipes putredinis, Collinsella aerofaciens, Alistipes shahii, Phascolarctobacterium faecium, Bacteroides fragilis, Paraclostridium bifermentans, Fusobacterium mortiferum, and E. coli.

In one aspect, the disclosure provides compositions consisting essentially of bacterial strains of species Bacteroides caccae, Bacteroides intestinalis, Bacteroides faecis, Bacteroides ovatus, Bacteroides thetaiotamicron, Bacteroides uniformis, Bacteroides vulgatus, Bifidobacterium adolescentis, Bifidobacterium longum, Bifidobacterium pseudocatenulatum, Clostridium innocuum, Erysipelotrichaceae bacterium 6_1_45, Paeniclostridium sordellii, Erysipelatoclostridium ramosum, Odoribacter sp. UNK.MGS-12, Parabacteroides distasonis, Parabacteroides merdae, Bacteroides xylanisolvens, Alistipes putredinis, Collinsella aerofaciens, Alistipes shahii, Phascolarctobacterium faecium, Bacteroides fragilis, Paraclostridium bifermentans, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 1-10, 14-16, 19, 21-27, 29, 31, and 33-36. In one aspect, the bacterial composition consists of purified bacterial strains comprising a 16S rDNA sequence having at least 97% sequence identity with the nucleic acid sequence set forth as SEQ ID NOs: 1-10, 14-16, 19, 21-27, 29, 31, and 33-36. In one aspect, the bacterial composition consists essentially of purified bacterial strains comprising a 16S rDNA sequence having at least 97% sequence identity with the nucleic acid sequence set forth as SEQ ID NOs: 1-10, 14-16, 19, 21-27, 29, 31, and 33-36.

In one aspect, the disclosure provides compositions comprising bacterial strains of species Bacteroides caccae, Bacteroides intestinalis, Bacteroides faecis, Bacteroides ovatus, Bacteroides thetaiotamicron, Bacteroides uniformis, Bacteroides vulgatus, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Bacteroides xylanisolvens, Blautia obeum, Alistipes putredinis, Eubacterium hallii, Alistipes shahii, and E. coli.

In one aspect, the disclosure provides compositions consisting of bacterial strains of species Bacteroides caccae, Bacteroides intestinalis, Bacteroides faecis, Bacteroides ovatus, Bacteroides thetaiotamicron, Bacteroides uniformis, Bacteroides vulgatus, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Bacteroides xylanisolvens, Blautia obeum, Alistipes putredinis, Eubacterium hallii, Alistipes shahii, and E. coli.

In one aspect, the disclosure provides compositions consisting essentially of bacterial strains of species Bacteroides caccae, Bacteroides intestinalis, Bacteroides faecis, Bacteroides ovatus, Bacteroides thetaiotamicron, Bacteroides uniformis, Bacteroides vulgatus, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Bacteroides xylanisolvens, Blautia obeum, Alistipes putredinis, Eubacterium hallii, Alistipes shahii, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 1-7, 11-15, 17-20, 24-26, 28, 29, and 36. In one aspect, the bacterial composition consists of purified bacterial strains comprising a 16S rDNA sequence having at least 97% sequence identity with the nucleic acid sequence set forth as SEQ ID NOs: 1-7, 11-15, 17-20, 24-26, 28, 29, and 36. In one aspect, the bacterial composition consists essentially of purified bacterial strains comprising a 16S rDNA sequence having at least 97% sequence identity with the nucleic acid sequence set forth as SEQ ID NOs: 1-7, 11-15, 17-20, 24-26, 28, 29, and 36.

In one aspect, the disclosure provides compositions comprising bacterial strains of species Bacteroides caccae, Bacteroides intestinalis, Bacteroides faecis, Bacteroides ovatus, Bacteroides thetaiotamicron, Bacteroides uniformis, Bacteroides vulgatus, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Bacteroides xylanisolvens, Blautia obeum, Alistipes putredinis, Eubacterium hallii, and Alistipes shahii.

In one aspect, the disclosure provides compositions consisting of bacterial strains of species Bacteroides caccae, Bacteroides intestinalis, Bacteroides faecis, Bacteroides ovatus, Bacteroides thetaiotamicron, Bacteroides uniformis, Bacteroides vulgatus, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Bacteroides xylanisolvens, Blautia obeum, Alistipes putredinis, Eubacterium hallii, and Alistipes shahii.

In one aspect, the disclosure provides compositions consisting essentially of bacterial strains of species Bacteroides caccae, Bacteroides intestinalis, Bacteroides faecis, Bacteroides ovatus, Bacteroides thetaiotamicron, Bacteroides uniformis, Bacteroides vulgatus, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Bacteroides xylanisolvens, Blautia obeum, Alistipes putredinis, Eubacterium hallii, and Alistipes shahii. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 1-7, 11-15, 17-20, 24-26, 28, and 29. In one aspect, the bacterial composition consists of purified bacterial strains comprising a 16S rDNA sequence having at least 97% sequence identity with the nucleic acid sequence set forth as SEQ ID NOs: 1-7, 11, 13-15, 17, 19, 24-26, 28, 29, 34, and 36. In one aspect, the bacterial composition consists essentially of purified bacterial strains comprising a 16S rDNA sequence having at least 97% sequence identity with the nucleic acid sequence set forth as SEQ ID NOs: 1-7, 11, 13-15, 17, 19, 24-26, 28, 29, 34, and 36.

In one aspect, the disclosure provides compositions comprising bacterial strains of species Bacteroides caccae, Bacteroides intestinalis, Bacteroides faecis, Bacteroides ovatus, Bacteroides thetaiotamicron, Bacteroides uniformis, Bacteroides vulgatus, Blautia producta, Clostridium clostridioforme, Clostridium innocuum, Erysipelotrichaceae bacterium 6_1_45, Coprococcus comes, Erysipelatoclostridium ramosum, Bacteroides xylanisolvens, Blautia obeum, Alistipes putredinis, Eubacterium hallii, Alistipes shahii, Fusobacterium mortiferum, and E. coli.

In one aspect, the disclosure provides compositions consisting of bacterial strains of species Bacteroides caccae, Bacteroides intestinalis, Bacteroides faecis, Bacteroides ovatus, Bacteroides thetaiotamicron, Bacteroides uniformis, Bacteroides vulgatus, Blautia producta, Clostridium clostridioforme, Clostridium innocuum, Erysipelotrichaceae bacterium 6_1_45, Coprococcus comes, Erysipelatoclostridium ramosum, Bacteroides xylanisolvens, Blautia obeum, Alistipes putredinis, Eubacterium hallii, Alistipes shahii, Fusobacterium mortiferum, and E. coli.

In one aspect, the disclosure provides compositions consisting essentially of bacterial strains of species Bacteroides caccae, Bacteroides intestinalis, Bacteroides faecis, Bacteroides ovatus, Bacteroides thetaiotamicron, Bacteroides uniformis, Bacteroides vulgatus, Blautia producta, Clostridium clostridioforme, Clostridium innocuum, Erysipelotrichaceae bacterium 6_1_45, Coprococcus comes, Erysipelatoclostridium ramosum, Bacteroides xylanisolvens, Blautia obeum, Alistipes putredinis, Eubacterium hallii, Alistipes shahii, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 1-7, 11, 13-15, 17, 19, 24-26, 28, 29, 34, and 36. In one aspect, the bacterial composition consists of purified bacterial strains comprising a 16S rDNA sequence having at least 97% sequence identity with the nucleic acid sequence set forth as SEQ ID NOs: 1-7, 11, 13-15, 17, 19, 24-26, 28, 29, 34, and 36. In one aspect, the bacterial composition consists essentially of purified bacterial strains comprising a 16S rDNA sequence having at least 97% sequence identity with the nucleic acid sequence set forth as SEQ ID NOs: 1-7, 11, 13-15, 17, 19, 24-26, 28, 29, 34, and 36.

In one aspect, the disclosure provides compositions comprising bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordelli, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, Phascolarctobacterium faecium, Agathobaculum butyriciproducens, Bacteroides fragilis, and Paraclostridium bifermentans. In one aspect, the disclosure provides compositions consisting of bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordelli, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, Phascolarctobacterium faecium, Agathobaculum butyriciproducens, Bacteroides fragilis, and Paraclostridium bifermentans.

In one aspect, the disclosure provides compositions consisting essentially of bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordelli, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, Phascolarctobacterium faecium, Agathobaculum butyriciproducens, Bacteroides fragilis, and Paraclostridium bifermentans. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 1-33 and 35. In one aspect, the bacterial composition consists of purified bacterial strains comprising a 16S rDNA sequence having at least 97% sequence identity with the nucleic acid sequence set forth as SEQ ID NOs: 1-33 and 35. In one aspect, the bacterial composition consists essentially of purified bacterial strains comprising a 16S rDNA sequence having at least 97% sequence identity with the nucleic acid sequence set forth as SEQ ID NOs: 1-33 and 35.

In one aspect, the disclosure provides compositions comprising bacterial strains of species Bacteroides intestinalis, Bacteroides ovatus, Bacteroides thetaiotaomicron, Bacteroides uniformis, Bacteroides vulgatus, Bifidobacterium adolescentis, Bifidobacterium longum, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Parabacteroides distasonis, Bacteroides xylanisolvens, Blautia obeum, Alistipes putredinis, Collinsella aerofaciens, Eubacterium hallii, Anaerostipes caccae, Phascolarctobacterium faecium, Agathobaculum butyriciproducens, Fusobacterium mortiferum, and E. coli.

In one aspect, the disclosure provides compositions consisting of bacterial strains of species Bacteroides intestinalis, Bacteroides ovatus, Bacteroides thetaiotaomicron, Bacteroides uniformis, Bacteroides vulgatus, Bifidobacterium adolescentis, Bifidobacterium longum, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Parabacteroides distasonis, Bacteroides xylanisolvens, Blautia obeum, Alistipes putredinis, Collinsella aerofaciens, Eubacterium hallii, Anaerostipes caccae, Phascolarctobacterium faecium, Agathobaculum butyriciproducens, Fusobacterium mortiferum, and E. coli.

In one aspect, the disclosure provides compositions consisting essentially of bacterial strains of species Bacteroides intestinalis, Bacteroides ovatus, Bacteroides thetaiotaomicron, Bacteroides uniformis, Bacteroides vulgatus, Bifidobacterium adolescentis, Bifidobacterium longum, Blautia producta, Clostridium citroniae, Clostridium clostridioforme, Clostridium innocuum, Erysipelotrichaceae bacterium 6_1_45, Coprococcus comes, Dorea longicatena, Erysipelatoclostridium ramosum, Eubacterium rectale, Parabacteroides distasonis, Bacteroides xylanisolvens, Blautia obeum, Alistipes putredinis, Collinsella aerofaciens, Eubacterium hallii, Anaerostipes caccae, Phascolarctobacterium faecium, Agathobaculum butyriciproducens, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 2, 4-9, 11-15, 17-20, 22, 24-28, 30-32, 34, and 36. In one aspect, the bacterial composition consists of purified bacterial strains comprising a 16S rDNA sequence having at least 97% sequence identity with the nucleic acid sequence set forth as SEQ ID NOs: 2, 4-9, 11-15, 17-20, 22, 24-28, 30-32, 34, and 36. In one aspect, the bacterial composition consists essentially of purified bacterial strains comprising a 16S rDNA sequence having at least 97% sequence identity with the nucleic acid sequence set forth as SEQ ID NOs: 2, 4-9, 11-15, 17-20, 22, 24-28, 30-32, 34, and 36.

In one aspect, the disclosure provides compositions comprising bacterial strains of species Bacteroides intestinalis, Bacteroides ovatus, Bacteroides thetaiotaomicron, Bacteroides uniformis, Bacteroides vulgatus, Bifidobacterium longum, Blautia producta, Clostridium clostridioforme, Clostridium innocuum, Erysipelotrichaceae bacterium 6_1_45, Dorea longicatena, Erysipelatoclostridium ramosum, Clostridium citroniae, Parabacteroides distasonis, Bacteroides xylanisolvens, Blautia obeum, Alistipes putredinis, Collinsella aerofaciens, Eubacterium hallii, Anaerostipes caccae, Phascolarctobacterium faecium, Agathobaculum butyriciproducens, Fusobacterium mortiferum, and E. coli.

In one aspect, the disclosure provides compositions consisting of bacterial strains of species Bacteroides intestinalis, Bacteroides ovatus, Bacteroides thetaiotaomicron, Bacteroides uniformis, Bacteroides vulgatus, Bifidobacterium longum, Blautia producta, Clostridium clostridioforme, Clostridium innocuum, Erysipelotrichaceae bacterium 6_1_45, Dorea longicatena, Erysipelatoclostridium ramosum, Clostridium citroniae, Parabacteroides distasonis, Bacteroides xylanisolvens, Blautia obeum, Alistipes putredinis, Collinsella aerofaciens, Eubacterium hallii, Anaerostipes caccae, Phascolarctobacterium faecium, Agathobaculum butyriciproducens, Fusobacterium mortiferum, and E. coli.

In one aspect, the disclosure provides compositions consisting essentially of bacterial strains of species Bacteroides intestinalis, Bacteroides ovatus, Bacteroides thetaiotaomicron, Bacteroides uniformis, Bacteroides vulgatus, Bifidobacterium longum, Blautia producta, Clostridium clostridioforme, Clostridium innocuum, Erysipelotrichaceae bacterium 6_1_45, Dorea longicatena, Erysipelatoclostridium ramosum, Clostridium citroniae, Parabacteroides distasonis, Bacteroides xylanisolvens, Blautia obeum, Alistipes putredinis, Collinsella aerofaciens, Eubacterium hallii, Anaerostipes caccae, Phascolarctobacterium faecium, Agathobaculum butyriciproducens, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 2, 4-7, 9, 11, 13-15, 18-20, 22, 24-28, 30-32, 34, and 36. In one aspect, the bacterial composition consists of purified bacterial strains comprising a 16S rDNA sequence having at least 97% sequence identity with the nucleic acid sequence set forth as SEQ ID NOs: 2, 4-7, 9, 11, 13-15, 18-20, 22, 24-28, 30-32, 34, and 36. In one aspect, the bacterial composition consists essentially of purified bacterial strains comprising a 16S rDNA sequence having at least 97% sequence identity with the nucleic acid sequence set forth as SEQ ID NOs: 2, 4-7, 9, 11, 13-15, 18-20, 22, 24-28, 30-32, 34, and 36.

In one aspect, the disclosure provides compositions comprising bacterial strains of species Bacteroides caccae, Bacteroides intestinalis, Bacteroides ovatus, Bacteroides thetaiotaomicron, Bacteroides uniformis, Bacteroides vulgatus, Bifidobacterium longum, Blautia producta, Clostridium clostridioforme, Clostridium innocuum, Coprococcus comes, Dorea longicatena, Erysipelatoclostridium ramosum, Clostridium citroniae, Odoribacter sp. UNK.MGS-12, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Agathobaculum butyriciproducens, Bacteroides fragilis, Fusobacterium mortiferum, and E. coli.

In one aspect, the disclosure provides compositions consisting of bacterial strains of species Bacteroides caccae, Bacteroides intestinalis, Bacteroides ovatus, Bacteroides thetaiotaomicron, Bacteroides uniformis, Bacteroides vulgatus, Bifidobacterium longum, Blautia producta, Clostridium clostridioforme, Clostridium innocuum, Coprococcus comes, Dorea longicatena, Erysipelatoclostridium ramosum, Clostridium citroniae, Odoribacter sp. UNK.MGS-12, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Agathobaculum butyriciproducens, Bacteroides fragilis, Fusobacterium mortiferum, and E. coli.

In one aspect, the disclosure provides compositions consisting essentially of bacterial strains of species Bacteroides caccae, Bacteroides intestinalis, Bacteroides ovatus, Bacteroides thetaiotaomicron, Bacteroides uniformis, Bacteroides vulgatus, Bifidobacterium longum, Blautia producta, Clostridium clostridioforme, Clostridium innocuum, Coprococcus comes, Dorea longicatena, Erysipelatoclostridium ramosum, Clostridium citroniae, Odoribacter sp. UNK.MGS-12, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Agathobaculum butyriciproducens, Bacteroides fragilis, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 1, 2, 4-7, 9, 11, 13, 14, 17-22, 24-27, 29-34, and 36. In one aspect, the bacterial composition consists of purified bacterial strains comprising a 16S rDNA sequence having at least 97% sequence identity with the nucleic acid sequence set forth as SEQ ID NOs: 1, 2, 4-7, 9, 11, 13, 14, 17-22, 24-27, 29-34, and 36. In one aspect, the bacterial composition consists essentially of purified bacterial strains comprising a 16S rDNA sequence having at least 97% sequence identity with the nucleic acid sequence set forth as SEQ ID NOs: 1, 2, 4-7, 9, 11, 13, 14, 17-22, 24-27, 29-34, and 36.

In one aspect, the disclosure provides compositions comprising bacterial strains of species Bacteroides ovatus, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, Phascolarctobacterium faecium, Agathobaculum butyriciproducens, Paraclostridium bifermentans, Fusobacterium mortiferum, and E. coli.

In one aspect, the disclosure provides compositions consisting of bacterial strains of species Bacteroides ovatus, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, Phascolarctobacterium faecium, Agathobaculum butyriciproducens, Paraclostridium bifermentans, Fusobacterium mortiferum, and E. coli.

In one aspect, the disclosure provides compositions consisting essentially of bacterial strains of species Bacteroides ovatus, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Erysipelotrichaceae bacterium 6_1_45, Paeniclostridium sordellii, Coprococcus comes, Dorea longicatena, Erysipelatoclostridium ramosum, Eubacterium rectale, Odoribacter sp. UNK.MGS-12, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, Phascolarctobacterium faecium, Agathobaculum butyriciproducens, Paraclostridium bifermentans, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 4, 7-32, and 34-36. In one aspect, the bacterial composition consists of purified bacterial strains comprising a 16S rDNA sequence having at least 97% sequence identity with the nucleic acid sequence set forth as SEQ ID NOs: 4, 7-32, and 34-36. In one aspect, the bacterial composition consists essentially of purified bacterial strains comprising a 16S rDNA sequence having at least 97% sequence identity with the nucleic acid sequence set forth as SEQ ID NOs: 4, 7-32, and 34-36.

In one aspect, the disclosure provides compositions comprising bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium longum, Blautia producta, Clostridium clostridioforme, Clostridium innocuum, Erysipelotrichaceae bacterium 6_1_45, Paeniclostridium sordelli, Dorea longicatena, Erysipelatoclostridium ramosum, Clostridium citroniae, Parabacteroides distasonis, Parabacteroides merdae, Bacteroides xylanisolvens, Blautia obeum, Alistipes putredinis, Alistipes shahii, Anaerostipes caccae, Phascolarctobacterium faecium, Agathobaculum butyriciproducens, Bacteroides fragilis, Paraclostridium bifermentans, Fusobacterium mortiferum, and E. coli.

In one aspect, the disclosure provides compositions consisting of bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium longum, Blautia producta, Clostridium clostridioforme, Clostridium innocuum, Erysipelotrichaceae bacterium 6_1_45, Paeniclostridium sordelli, Dorea longicatena, Erysipelatoclostridium ramosum, Clostridium citroniae, Parabacteroides distasonis, Parabacteroides merdae, Bacteroides xylanisolvens, Blautia obeum, Alistipes putredinis, Alistipes shahii, Anaerostipes caccae, Phascolarctobacterium faecium, Agathobaculum butyriciproducens, Bacteroides fragilis, Paraclostridium bifermentans, Fusobacterium mortiferum, and E. coli.

In one aspect, the disclosure provides compositions consisting essentially of bacterial strains of species Bacteroides caccae, Bacteroides intestinalis, Bacteroides faecis, Bacteroides ovatus, Bacteroides thetaiotaomicron, Bacteroides uniformis, Bacteroides vulgatus, Bifidobacterium longum, Blautia producta, Clostridium clostridioforme, Clostridium innocuum, Erysipelotrichaceae bacterium 6_1_45, Paeniclostridium sordelli, Dorea longicatena, Erysipelatoclostridium ramosum, Clostridium citroniae, Parabacteroides distasonis, Parabacteroides merdae, Bacteroides xylanisolvens, Blautia obeum, Alistipes putredinis, Alistipes shahii, Anaerostipes caccae, Phascolarctobacterium faecium, Agathobaculum butyriciproducens, Bacteroides fragilis, Paraclostridium bifermentans, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 1-7, 9, 11, 13-16, 18-20, 22-26, and 29-36. In one aspect, the bacterial composition consists of purified bacterial strains comprising a 16S rDNA sequence having at least 97% sequence identity with the nucleic acid sequence set forth as SEQ ID NOs: 1-7, 9, 11, 13-16, 18-20, 22-26, and 29-36. In one aspect, the bacterial composition consists essentially of purified bacterial strains comprising a 16S rDNA sequence having at least 97% sequence identity with the nucleic acid sequence set forth as SEQ ID NOs: 1-7, 9, 11, 13-16, 18-20, 22-26, and 29-36.

In one aspect, the disclosure provides compositions comprising bacterial strains of species Bacteroides caccae, Bacteroides ovatus, Bacteroides vulgatus, Bifidobacterium longum, Blautia producta, Clostridium citroniae, Clostridium clostridioforme, Clostridium innocuum, Coprococcus comes, Dorea longicatena, Erysipelatoclostridium ramosum, Odoribacter sp. UNK.MGS-12, Parabacteroides distasonis, Bacteroides xylanisolvens, Blautia obeum, Alistipes putredinis, Alistipes shahii, Anaerostipes caccae, Phascolarctobacterium faecium, Agathobaculum butyriciproducens, Fusobacterium mortiferum, and E. coli.

In one aspect, the disclosure provides compositions consisting of bacterial strains of species Bacteroides caccae, Bacteroides ovatus, Bacteroides vulgatus, Bifidobacterium longum, Blautia producta, Clostridium citroniae, Clostridium clostridioforme, Clostridium innocuum, Coprococcus comes, Dorea longicatena, Erysipelatoclostridium ramosum, Odoribacter sp. UNK.MGS-12, Parabacteroides distasonis, Bacteroides xylanisolvens, Blautia obeum, Alistipes putredinis, Alistipes shahii, Anaerostipes caccae, Phascolarctobacterium faecium, Agathobaculum butyriciproducens, Fusobacterium mortiferum, and E. coli.

In one aspect, the disclosure provides compositions consisting essentially of bacterial strains of species Bacteroides caccae, Bacteroides ovatus, Bacteroides vulgatus, Bifidobacterium longum, Blautia producta, Clostridium citroniae, Clostridium clostridioforme, Clostridium innocuum, Coprococcus comes, Dorea longicatena, Erysipelatoclostridium ramosum, Odoribacter sp. UNK.MGS-12, Parabacteroides distasonis, Bacteroides xylanisolvens, Blautia obeum, Alistipes putredinis, Alistipes shahii, Anaerostipes caccae, Phascolarctobacterium faecium, Agathobaculum butyriciproducens, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 1, 4, 7, 9, 11-14, 17-19, 21, 22, 24-26, 29-32, 34, and 36. In one aspect, the bacterial composition consists of purified bacterial strains comprising a 16S rDNA sequence having at least 97% sequence identity with the nucleic acid sequence set forth as SEQ ID NOs: 1, 4, 7, 9, 11-14, 17-19, 21, 22, 24-26, 29-32, 34, and 36. In one aspect, the bacterial composition consists essentially of purified bacterial strains comprising a 16S rDNA sequence having at least 97% sequence identity with the nucleic acid sequence set forth as SEQ ID NOs: 1, 4, 7, 9, 11-14, 17-19, 21, 22, 24-26, 29-32, 34, and 36.

In one aspect, the disclosure provides compositions comprising bacterial strains of species Bacteroides caccae, Bacteroides intestinalis, Bacteroides ovatus, Bacteroides thetaiotaomicron, Bacteroides uniformis, Bacteroides vulgatus, Bifidobacterium longum, Blautia producta, Clostridium citroniae, Clostridium clostridioforme, Clostridium innocuum, Coprococcus comes, Dorea longicatena, Erysipelatoclostridium ramosum, Odoribacter sp. UNK.MGS-12, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Agathobaculum butyriciproducens, Fusobacterium mortiferum, and E. coli.

In one aspect, the disclosure provides compositions consisting of bacterial strains of species Bacteroides caccae, Bacteroides intestinalis, Bacteroides ovatus, Bacteroides thetaiotaomicron, Bacteroides uniformis, Bacteroides vulgatus, Bifidobacterium longum, Blautia producta, Clostridium citroniae, Clostridium clostridioforme, Clostridium innocuum, Coprococcus comes, Dorea longicatena, Erysipelatoclostridium ramosum, Odoribacter sp. UNK.MGS-12, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Agathobaculum butyriciproducens, Fusobacterium mortiferum, and E. coli.

In one aspect, the disclosure provides compositions consisting essentially of bacterial strains of species Bacteroides caccae, Bacteroides intestinalis, Bacteroides ovatus, Bacteroides thetaiotaomicron, Bacteroides uniformis, Bacteroides vulgatus, Bifidobacterium longum, Blautia producta, Clostridium citroniae, Clostridium clostridioforme, Clostridium innocuum, Coprococcus comes, Dorea longicatena, Erysipelatoclostridium ramosum, Odoribacter sp. UNK.MGS-12, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Agathobaculum butyriciproducens, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 1, 2, 4-7, 9, 11-14, 17-19, 21, 22, 24-27, 29-32, 34, and 36. In one aspect, the bacterial composition consists of purified bacterial strains comprising a 16S rDNA sequence having at least 97% sequence identity with the nucleic acid sequence set forth as SEQ ID NOs: 1, 2, 4-7, 9, 11-14, 17-19, 21, 22, 24-27, 29-32, 34, and 36. In one aspect, the bacterial composition consists essentially of purified bacterial strains comprising a 16S rDNA sequence having at least 97% sequence identity with the nucleic acid sequence set forth as SEQ ID NOs: 1, 2, 4-7, 9, 11-14, 17-19, 21, 22, 24-27, 29-32, 34, and 36.

In one aspect, the disclosure provides compositions comprising bacterial strains of species Bacteroides ovatus, Bacteroides vulgatus, Bifidobacterium longum, Blautia producta, Clostridium citroniae, Clostridium clostridioforme, Clostridium innocuum, Coprococcus comes, Dorea longicatena, Erysipelatoclostridium ramosum, Odoribacter sp. UNK.MGS-12, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Fusobacterium mortiferum, and E. coli.

In one aspect, the disclosure provides compositions consisting of bacterial strains of species Bacteroides ovatus, Bacteroides vulgatus, Bifidobacterium longum, Blautia producta, Clostridium citroniae, Clostridium clostridioforme, Clostridium innocuum, Coprococcus comes, Dorea longicatena, Erysipelatoclostridium ramosum, Odoribacter sp. UNK.MGS-12, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Fusobacterium mortiferum, and E. coli.

In one aspect, the disclosure provides compositions consisting essentially of bacterial strains of species Bacteroides ovatus, Bacteroides vulgatus, Bifidobacterium longum, Blautia producta, Clostridium citroniae, Clostridium clostridioforme, Clostridium innocuum, Coprococcus comes, Dorea longicatena, Erysipelatoclostridium ramosum, Odoribacter sp. UNK.MGS-12, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 4, 7, 9, 11-14, 17-19, 21, 22, 24-27, 29-32, 34, and 36. In one aspect, the bacterial composition consists of purified bacterial strains comprising a 16S rDNA sequence having at least 97% sequence identity with the nucleic acid sequence set forth as SEQ ID NOs: 4, 7, 9, 11-14, 17-19, 21, 22, 24-27, 29-32, 34, and 36. In one aspect, the bacterial composition consists essentially of purified bacterial strains comprising a 16S rDNA sequence having at least 97% sequence identity with the nucleic acid sequence set forth as SEQ ID NOs: 4, 7, 9, 11-14, 17-19, 21, 22, 24-27, 29-32, 34, and 36.

In one aspect, the disclosure provides compositions comprising bacterial strains of species Bacteroides ovatus, Bifidobacterium longum, Blautia producta, Clostridium clostridioforme, Clostridium innocuum, Coprococcus comes, Dorea longicatena, Erysipelatoclostridium ramosum, Odoribacter sp. UNK.MGS-12, Bacteroides xylanisolvens, Alistipes putredinis, Collinsella aerofaciens, Blautia obeum, Anaerostipes caccae, Agathobaculum butyriciproducens, Fusobacterium mortiferum, and E. coli.

In one aspect, the disclosure provides compositions consisting of Bacteroides ovatus, Bifidobacterium longum, Blautia producta, Clostridium clostridioforme, Clostridium innocuum, Coprococcus comes, Dorea longicatena, Erysipelatoclostridium ramosum, Odoribacter sp. UNK.MGS-12, Bacteroides xylanisolvens, Alistipes putredinis, Collinsella aerofaciens, Blautia obeum, Anaerostipes caccae, Agathobaculum butyriciproducens, Fusobacterium mortiferum, and E. coli.

In one aspect, the disclosure provides compositions consisting essentially of Bacteroides ovatus, Bifidobacterium longum, Blautia producta, Clostridium clostridioforme, Clostridium innocuum, Coprococcus comes, Dorea longicatena, Erysipelatoclostridium ramosum, Odoribacter sp. UNK.MGS-12, Bacteroides xylanisolvens, Alistipes putredinis, Collinsella aerofaciens, Blautia obeum, Anaerostipes caccae, Agathobaculum butyriciproducens, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 4, 9, 11, 13, 14, 17-19, 21, 24, 26, 27, 29, 30, 32, 34, and 36. In one aspect, the bacterial composition consists of purified bacterial strains comprising a 16S rDNA sequence having at least 97% sequence identity with the nucleic acid sequence set forth as SEQ ID NOs: 4, 9, 11, 13, 14, 17-19, 21, 24, 26, 27, 29, 30, 32, 34, and 36. In one aspect, the bacterial composition consists essentially of purified bacterial strains comprising a 16S rDNA sequence having at least 97% sequence identity with the nucleic acid sequence set forth as SEQ ID NOs: 4, 9, 11, 13, 14, 17-19, 21, 24, 26, 27, 29, 30, 32, 34, and 36.

In one aspect, the disclosure provides compositions comprising bacterial strains of species Bacteroides faecis, Bacteroides ovatus, Bacteroides vulgatus, Bifidobacterium adolescentis, Bifidobacterium longum, Blautia producta, Clostridium citroniae, Clostridium clostridioforme, Clostridium innocuum, Erysipelotrichaceae bacterium 6_1_45, Paeniclostridium sordellii, Dorea longicatena, Erysipelatoclostridium ramosum, Odoribacter sp. UNK.MGS-12, Blautia obeum, Alistipes putredinis, Collinsella aerofaciens, Eubacterium hallii, Anaerostipes caccae, Phascolarctobacterium faecium, Agathobaculum butyriciproducens, Bacteroides fragilis, Paraclostridium bifermentans, Fusobacterium mortiferum, and E. coli.

In one aspect, the disclosure provides compositions consisting of bacterial strains of species Bacteroides faecis, Bacteroides ovatus, Bacteroides vulgatus, Bifidobacterium adolescentis, Bifidobacterium longum, Blautia producta, Clostridium citroniae, Clostridium clostridioforme, Clostridium innocuum, Erysipelotrichaceae bacterium 6_1_45, Paeniclostridium sordellii, Dorea longicatena, Erysipelatoclostridium ramosum, Odoribacter sp. UNK.MGS-12, Blautia obeum, Alistipes putredinis, Collinsella aerofaciens, Eubacterium hallii, Anaerostipes caccae, Phascolarctobacterium faecium, Agathobaculum butyriciproducens, Bacteroides fragilis, Paraclostridium bifermentans, Fusobacterium mortiferum, and E. coli.

In one aspect, the disclosure provides compositions consisting essentially of bacterial strains of species Bacteroides faecis, Bacteroides ovatus, Bacteroides vulgatus, Bifidobacterium adolescentis, Bifidobacterium longum, Blautia producta, Clostridium citroniae, Clostridium clostridioforme, Clostridium innocuum, Erysipelotrichaceae bacterium 6_1_45, Paeniclostridium sordellii, Dorea longicatena, Erysipelatoclostridium ramosum, Odoribacter sp. UNK.MGS-12, Blautia obeum, Alistipes putredinis, Collinsella aerofaciens, Eubacterium hallii, Anaerostipes caccae, Phascolarctobacterium faecium, Agathobaculum butyriciproducens, Bacteroides fragilis, Paraclostridium bifermentans, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 3, 4, 7-9, 11-16, 18, 19, 21, 25-28, and 30-36. In one aspect, the bacterial composition consists of purified bacterial strains comprising a 16S rDNA sequence having at least 97% sequence identity with the nucleic acid sequence set forth as SEQ ID NOs: 3, 4, 7-9, 11-16, 18, 19, 21, 25-28, and 30-36. In one aspect, the bacterial composition consists essentially of purified bacterial strains comprising a 16S rDNA sequence having at least 97% sequence identity with the nucleic acid sequence set forth as SEQ ID NOs: 3, 4, 7-9, 11-16, 18, 19, 21, 25-28, and 30-36.

In one aspect, the disclosure provides compositions comprising bacterial strains of species Bacteroides faecis, Bacteroides ovatus, Bacteroides vulgatus, Bifidobacterium longum, Blautia producta, Clostridium citroniae, Clostridium clostridioforme, Clostridium innocuum, Dorea longicatena, Erysipelatoclostridium ramosum, Odoribacter sp. UNK.MGS-12, Alistipes putredinis, Collinsella aerofaciens, Blautia obeum, Anaerostipes caccae, Phascolarctobacterium faecium, Agathobaculum butyriciproducens, Fusobacterium mortiferum, and E. coli.

In one aspect, the disclosure provides compositions consisting of bacterial strains of species Bacteroides faecis, Bacteroides ovatus, Bacteroides vulgatus, Bifidobacterium longum, Blautia producta, Clostridium citroniae, Clostridium clostridioforme, Clostridium innocuum, Dorea longicatena, Erysipelatoclostridium ramosum, Odoribacter sp. UNK.MGS-12, Alistipes putredinis, Collinsella aerofaciens, Blautia obeum, Anaerostipes caccae, Phascolarctobacterium faecium, Agathobaculum butyriciproducens, Fusobacterium mortiferum, and E. coli.

In one aspect, the disclosure provides compositions consisting essentially of bacterial strains of species Bacteroides faecis, Bacteroides ovatus, Bacteroides vulgatus, Bifidobacterium longum, Blautia producta, Clostridium citroniae, Clostridium clostridioforme, Clostridium innocuum, Dorea longicatena, Erysipelatoclostridium ramosum, Odoribacter sp. UNK.MGS-12, Alistipes putredinis, Collinsella aerofaciens, Blautia obeum, Anaerostipes caccae, Phascolarctobacterium faecium, Agathobaculum butyriciproducens, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 3, 4, 7, 9, 11-14, 18, 19, 21, 26, 27, 29-32, 34, and 36. In one aspect, the bacterial composition consists of purified bacterial strains comprising a 16S rDNA sequences having at least 97% sequence identity with the nucleic acid sequence set forth as SEQ ID NOs: 3, 4, 7, 9, 11-14, 18, 19, 21, 26, 27, 29-32, 34, and 36. In one aspect, the bacterial composition consists essentially of purified bacterial strains comprising a 16S rDNA sequence having at least 97% sequence identity with the nucleic acid sequence set forth as SEQ ID NOs: 3, 4, 7, 9, 11-14, 18, 19, 21, 26, 27, 29-32, 34, and 36.

In one aspect, the disclosure provides compositions comprising purified bacterial strains related to the following species Bacteroides ovatus, Bacteroides vulgatus, Bifidobacterium longum, Blautia producta, Clostridium citroniae, Clostridium clostridioforme, Clostridium innocuum, Erysipelatoclostridium ramosum, Parabacteroides distasonis, Alistipes putredinis, Collinsella aerofaciens, Blautia obeum, Anaerostipes caccae, Phascolarctobacterium faecium, Agathobaculum butyriciproducens, Fusobacterium mortiferum, and E. coli.

In one aspect, the disclosure provides compositions consisting of purified bacterial strains related to the following species Bacteroides ovatus, Bacteroides vulgatus, Bifidobacterium longum, Blautia producta, Clostridium citroniae, Clostridium clostridioforme, Clostridium innocuum, Erysipelatoclostridium ramosum, Parabacteroides distasonis, Alistipes putredinis, Collinsella aerofaciens, Blautia obeum, Anaerostipes caccae, Phascolarctobacterium faecium, Agathobaculum butyriciproducens, Fusobacterium mortiferum, and E. coli.

In one aspect, the disclosure provides compositions consisting essentially of purified bacterial strains related to the following species Bacteroides ovatus, Bacteroides vulgatus, Bifidobacterium longum, Blautia producta, Clostridium citroniae, Clostridium clostridioforme, Clostridium innocuum, Erysipelatoclostridium ramosum, Parabacteroides distasonis, Alistipes putredinis, Collinsella aerofaciens, Blautia obeum, Anaerostipes caccae, Phascolarctobacterium faecium, Agathobaculum butyriciproducens, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 4, 7, 9, 11-14, 19, 22, 26, 27, 29-32, 34, and 36. In one aspect, the bacterial composition consists of purified bacterial strains comprising a 16S rDNA sequence having at least 97% sequence identity with the nucleic acid sequence set forth as SEQ ID NOs: 4, 7, 9, 11-14, 19, 22, 26, 27, 29-32, 34, and 36. In one aspect, the bacterial composition consists essentially of purified bacterial strains comprising a 16S rDNA sequence having at least 97% sequence identity with the nucleic acid sequence set forth as SEQ ID NOs: 4, 7, 9, 11-14, 19, 22, 26, 27, 29-32, 34, and 36.

In one aspect, the disclosure provides compositions comprising purified bacterial strains related to the following species Bacteroides ovatus, Bacteroides vulgatus, Bifidobacterium longum, Blautia producta, Clostridium citroniae, Clostridium clostridioforme, Erysipelotrichaceae bacterium 6_1_45, Dorea longicatena, Erysipelatoclostridium ramosum, Blautia obeum, Alistipes putredinis, Collinsella aerofaciens, Phascolarctobacterium faecium, Fusobacterium mortiferum, and E. coli.

In one aspect, the disclosure provides compositions consisting of purified bacterial strains related to the following species Bacteroides ovatus, Bacteroides vulgatus, Bifidobacterium longum, Blautia producta, Clostridium citroniae, Clostridium clostridioforme, Erysipelotrichaceae bacterium 6_1_45, Dorea longicatena, Erysipelatoclostridium ramosum, Blautia obeum, Alistipes putredinis, Collinsella aerofaciens, Phascolarctobacterium faecium, Fusobacterium mortiferum, and E. coli.

In one aspect, the disclosure provides compositions consisting essentially of purified bacterial strains related to the following species Bacteroides ovatus, Bacteroides vulgatus, Bifidobacterium longum, Blautia producta, Clostridium citroniae, Clostridium clostridioforme, Erysipelotrichaceae bacterium 6_1_45, Dorea longicatena, Erysipelatoclostridium ramosum, Blautia obeum, Alistipes putredinis, Collinsella aerofaciens, Phascolarctobacterium faecium, Fusobacterium mortiferum, and E. coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 4, 7, 9, 11-13, 15, 18, 19, 25-27, 31, 34, and 36. In one aspect, the bacterial composition consists of purified bacterial strains comprising a 16S rDNA sequence having at least 97% sequence identity with the nucleic acid sequence set forth as SEQ ID NOs: 4, 7, 9, 11-13, 15, 18, 19, 25-27, 31, 34, and 36. In one aspect, the bacterial composition consists essentially of purified bacterial strains comprising a 16S rDNA sequence having at least 97% sequence identity with the nucleic acid sequence set forth as SEQ ID NOs: 4, 7, 9, 11-13, 15, 18, 19, 25-27, 31, 34, and 36.

In one aspect, the disclosure provides compositions comprising purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting essentially of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum. In some embodiments, consisting essentially of, as used herein, refers to compositions that do not include any additional therapeutically active bacterial strains.

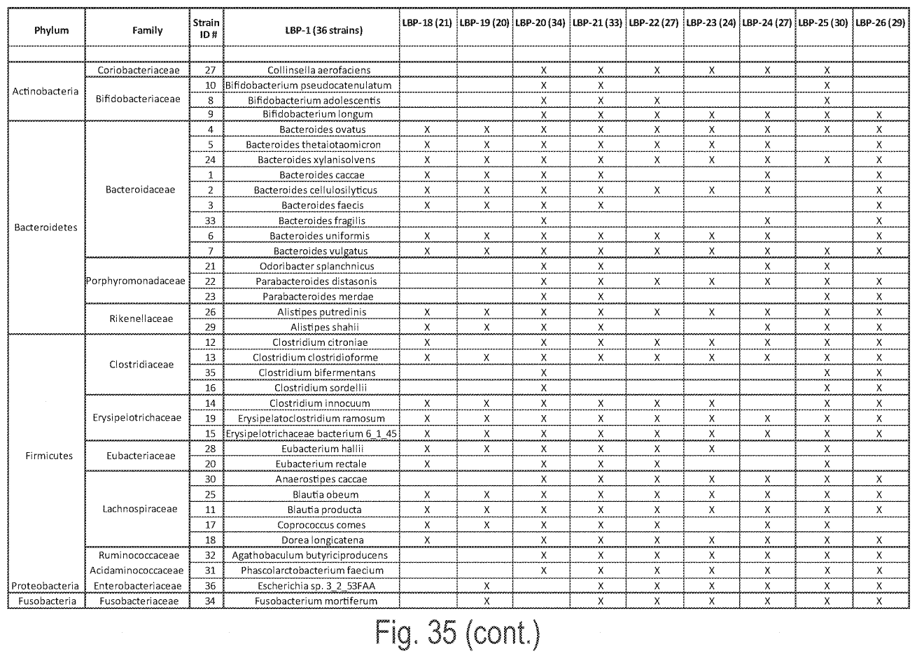

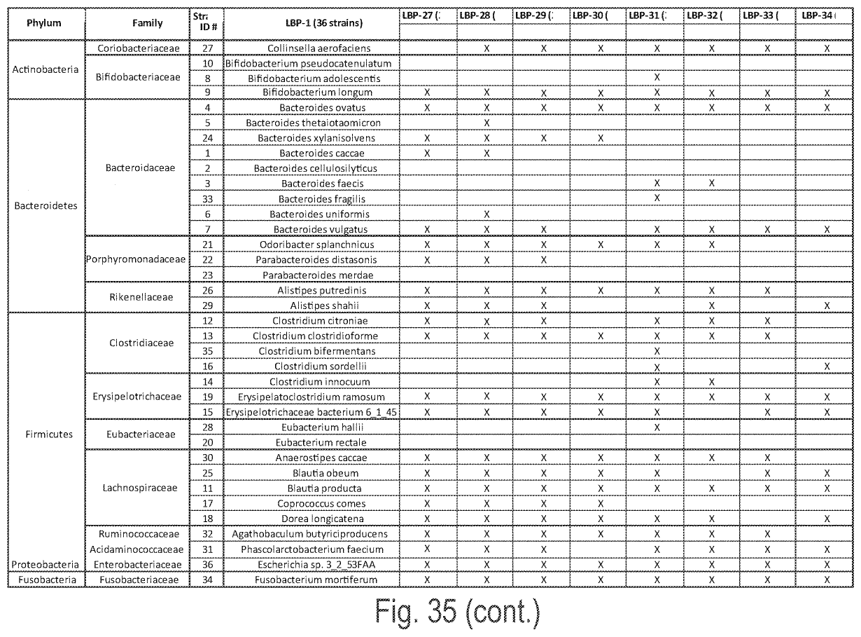

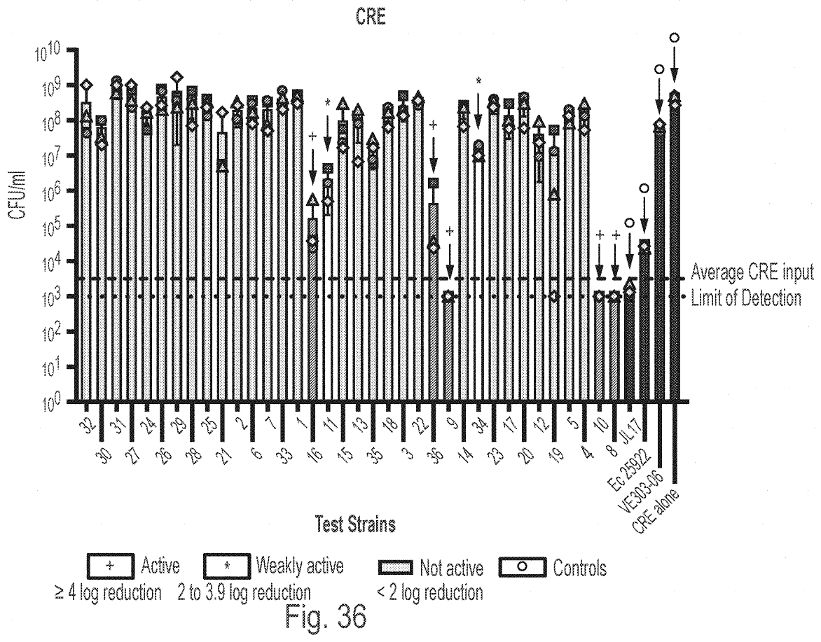

In one aspect, the bacterial composition comprises purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 35, 16, 14, 19, 15, 28, 20, 30, 25, 11, 17, 18, 32, 31, 36, and 34. In one aspect, the bacterial composition consists of purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 35, 16, 14, 19, 15, 28, 20, 30, 25, 11, 17, 18, 32, 31, 36, and 34. In one aspect, the bacterial composition consist essentially of purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 35, 16, 14, 19, 15, 28, 20, 30, 25, 11, 17, 18, 32, 31, 36, and 34. In one aspect, the bacterial consortium is LBP-1 (See, e.g., FIG. 35).

In one aspect, the disclosure provides compositions comprising purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, and Agathobaculum butyriciproducens.

In one aspect, the disclosure provides compositions consisting of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, and Agathobaculum butyriciproducens.

In one aspect, the disclosure provides compositions consisting essentially of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, and Agathobaculum butyriciproducens. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 10, 8, 9, 4, 5, 24, 1, 2, 3, 6, 7, 21, 22, 23, 26, 29, 12, 13, 16, 14, 19, 15, 28, 20, 30, 25, 11, 17, 18, and 32. In one aspect, the bacterial composition consists of purified bacterial strains comprising a 16S rDNA sequence having at least 97% sequence identity with the nucleic acid sequence set forth as SEQ ID Nos: 27, 10, 8, 9, 4, 5, 24, 1, 2, 3, 6, 7, 21, 22, 23, 26, 29, 12, 13, 16, 14, 19, 15, 28, 20, 30, 25, 11, 17, 18, and 32. In one aspect, the bacterial composition consist essentially of purified bacterial strains comprising a 16S rDNA sequence having at least 97% sequence identity with the nucleic acid sequence set forth as SEQ ID NOs: 27, 10, 8, 9, 4, 5, 24, 1, 2, 3, 6, 7, 21, 22, 23, 26, 29, 12, 13, 16, 14, 19, 15, 28, 20, 30, 25, 11, 17, 18, and 32. In one aspect, the bacterial consortium is LBP-9 (See e.g., FIG. 35).

In one aspect, the disclosure provides compositions comprising purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, Escherichia coli, and Fusobacterium mortiferum.

In one aspect, the disclosure provides compositions consisting of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, Escherichia coli, and Fusobacterium mortiferum.

In one aspect, the disclosure provides compositions consisting essentially of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 35, 16, 14, 19, 15, 28, 20, 25, 11, 17, 18, 36, and 34. In one aspect, the bacterial composition consists of purified bacterial strains comprising a 16S rDNA sequence having at least 97% sequence identity with the nucleic acid sequence set forth as SEQ ID Nos: 27, 10, 8, 9, 4, 5, 24, 1, 2, 3, 33, 6, 7, 21, 22, 23, 26, 29, 12, 13, 35, 16, 14, 19, 15, 28, 20, 25, 11, 17, 18, 36, and 34. In one aspect, the bacterial composition consist essentially of purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 35, 16, 14, 19, 15, 28, 20, 25, 11, 17, 18, 36, and 34. In one aspect, the bacterial consortium is LBP-10 (See e.g., FIG. 35).

In one aspect, the disclosure provides compositions comprising purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, and Fusobacterium mortiferum.

In one aspect, the disclosure provides compositions consisting of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, and Fusobacterium mortiferum.

In one aspect, the disclosure provides compositions consisting essentially of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 35, 16, 14, 19, 15, 28, 20, 30, 25, 11, 17, 18, 32, 31, and 34. In one aspect, the bacterial composition consists of purified bacterial strains comprising a 16S rDNA sequence having at least 97% sequence identity with the nucleic acid sequence set forth as SEQ ID Nos: 27, 10, 8, 9, 4, 5, 24, 1, 2, 3, 33, 6, 7, 21, 22, 23, 26, 29, 12, 13, 35, 16, 14, 19, 15, 28, 20, 30, 25, 11, 17, 18, 32, 31, and 34. In one aspect, the bacterial composition consist essentially of purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 35, 16, 14, 19, 15, 28, 20, 30, 25, 11, 17, 18, 32, 31, and 34. In one aspect, the bacterial consortium is LBP-11 (See e.g., FIG. 35).

In one aspect, the disclosure provides compositions comprising purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, and Escherichia coli.

In one aspect, the disclosure provides compositions consisting of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, and Escherichia coli.

In one aspect, the disclosure provides compositions consisting essentially of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, and Escherichia coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 35, 16, 14, 19, 15, 28, 20, 30, 25, 11, 17, 18, 32, 31, and 36. In one aspect, the bacterial composition consists of purified bacterial strains comprising a 16S rDNA sequence having at least 97% sequence identity with the nucleic acid sequence set forth as SEQ ID Nos: 27, 10, 8, 9, 4, 5, 24, 1, 2, 3, 33, 6, 7, 21, 22, 23, 26, 29, 12, 13, 35, 16, 14, 19, 15, 28, 20, 30, 25, 11, 17, 18, 32, 31, and 36. In one aspect, the bacterial composition consist essentially of purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 35, 16, 14, 19, 15, 28, 20, 30, 25, 11, 17, 18, 32, 31, and 36. In one aspect, the bacterial consortium is LBP-12 (See e.g., FIG. 35).

In one aspect, the disclosure provides compositions comprising purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, Escherichia coli, and Fusobacterium mortiferum.

In one aspect, the disclosure provides compositions consisting of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, Escherichia coli, and Fusobacterium mortiferum.

In one aspect, the disclosure provides compositions consisting essentially of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 10, 8, 9, 4, 5, 24, 1, 2, 3, 6, 7, 21, 22, 23, 26, 29, 12, 13, 14, 19, 15, 28, 20, 25, 11, 17, 18, 36, and 34. In one aspect, the bacterial composition consists of purified bacterial strains comprising a 16S rDNA sequence having at least 97% sequence identity with the nucleic acid sequence set forth as SEQ ID Nos: 27, 10, 8, 9, 4, 5, 24, 1, 2, 3, 6, 7, 21, 22, 23, 26, 29, 12, 13, 14, 19, 15, 28, 20, 25, 11, 17, 18, 36, and 34. In one aspect, the bacterial composition consist essentially of purified bacterial strains comprising a 16S rDNA sequence having at least 97% sequence identity with the nucleic acid sequence set forth as SEQ ID NOs: 27, 10, 8, 9, 4, 5, 24, 1, 2, 3, 6, 7, 21, 22, 23, 26, 29, 12, 13, 14, 19, 15, 28, 30, 25, 11, 17, 18, 36, and 34. In one aspect, the bacterial consortium is LBP-13 (See e.g., FIG. 35).

In one aspect, the disclosure provides compositions comprising purified bacterial strains related to the following species Bifidobacterium longum, Bacteroides ovatus, Bacteroides xylanisolvens, Bacteroides cellulosilyticus, Bacteroides faecis, Bacteroides uniformis, Bacteroides vulgatus, Odoribacter splanchnicus, Parabacteroides distasonis, Alistipes putredinis, Clostridium citroniae, Clostridium clostridioforme, Clostridium innocuum, Erysipelatoclostridium ramosum, Eubacterium hallii, Eubacterium rectale, Blautia obeum, Blautia producta, Dorea longicatena, Escherichia coli, and Fusobacterium mortiferum.

In one aspect, the disclosure provides compositions consisting of purified bacterial strains related to the following species Bifidobacterium longum, Bacteroides ovatus, Bacteroides xylanisolvens, Bacteroides cellulosilyticus, Bacteroides faecis, Bacteroides uniformis, Bacteroides vulgatus, Odoribacter splanchnicus, Parabacteroides distasonis, Alistipes putredinis, Clostridium citroniae, Clostridium clostridioforme, Clostridium innocuum, Erysipelatoclostridium ramosum, Eubacterium hallii, Eubacterium rectale, Blautia obeum, Blautia producta, Dorea longicatena, Escherichia coli, and Fusobacterium mortiferum.

In one aspect, the disclosure provides compositions consisting essentially of purified bacterial strains related to the following species Bifidobacterium longum, Bacteroides ovatus, Bacteroides xylanisolvens, Bacteroides cellulosilyticus, Bacteroides faecis, Bacteroides uniformis, Bacteroides vulgatus, Odoribacter splanchnicus, Parabacteroides distasonis, Alistipes putredinis, Clostridium citroniae, Clostridium clostridioforme, Clostridium innocuum, Erysipelatoclostridium ramosum, Eubacterium hallii, Eubacterium rectale, Blautia obeum, Blautia producta, Dorea longicatena, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 9, 4, 24, 2, 3, 6, 7, 21, 22, 26, 12, 13, 14, 19, 28, 20, 25, 11, 18, 36, and 34. In one aspect, the bacterial composition consists of purified bacterial strains comprising a 16S rDNA sequence having at least 97% sequence identity with the nucleic acid sequence set forth as SEQ ID Nos: 9, 4, 24, 2, 3, 6, 7, 21, 22, 26, 12, 13, 14, 19, 28, 20, 25, 11, 18, 36, and 34. In one aspect, the bacterial composition consist essentially of purified bacterial strains comprising a 16S rDNA sequence having at least 97% sequence identity with the nucleic acid sequence set forth as SEQ ID NOs: 9, 4, 24, 2, 3, 6, 7, 21, 22, 26, 12, 13, 14, 19, 28, 20, 25, 11, 18, 36, and 34. In one aspect, the bacterial consortium is LBP-14 (See e.g., FIG. 35).

In one aspect, the disclosure provides compositions comprising purified bacterial strains related to the following species Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Parabacteroides merdae, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, Escherichia coli, and Fusobacterium mortiferum.

In one aspect, the disclosure provides compositions consisting of purified bacterial strains related to the following species Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Parabacteroides merdae, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, Escherichia coli, and Fusobacterium mortiferum.

In one aspect, the disclosure provides compositions consisting essentially of purified bacterial strains related to the following species Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Parabacteroides merdae, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 4, 5, 24, 1, 2, 3, 6, 7, 26, 29, 12, 13, 14, 19, 15, 28, 20, 25, 11, 17, 18, 36, and 34. In one aspect, the bacterial composition consists of purified bacterial strains comprising a 16S rDNA sequence having at least 97% sequence identity with the nucleic acid sequence set forth as SEQ ID NOs: 4, 5, 24, 1, 2, 3, 6, 7, 26, 29, 12, 13, 14, 19, 15, 28, 20, 25, 11, 17, 18, 36, and 34. In one aspect, the bacterial composition consists essentially of purified bacterial strains comprising a 16S rDNA sequence having at least 97% sequence identity with the nucleic acid sequence set forth as SEQ ID NOs: 4, 5, 24, 1, 2, 3, 6, 7, 26, 29, 12, 13, 14, 19, 15, 28, 20, 25, 11, 17, 18, 36, and 34. In one aspect, the bacterial consortium is LBP-15 (See e.g., FIG. 35).

In one aspect, the disclosure provides compositions comprising purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium bifermentans, Clostridium sordellii, Clostridium innocuum, Erysipelatoclostridium ramosum, Erysipelotrichaceae bacterium 6_1_45, Phascolarctobacterium faecium, Escherichia coli, and Fusobacterium mortiferum.

In one aspect, the disclosure provides compositions consisting of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium bifermentans, Clostridium sordellii, Clostridium innocuum, Erysipelatoclostridium ramosum, Erysipelotrichaceae bacterium 6_1_45, Phascolarctobacterium faecium, Escherichia coli, and Fusobacterium mortiferum.

In one aspect, the disclosure provides compositions consisting essentially of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium bifermentans, Clostridium sordellii, Clostridium innocuum, Erysipelatoclostridium ramosum, Erysipelotrichaceae bacterium 6_1_45, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 35, 16, 14, 19, 15, 31, 36, and 34. In one aspect, the bacterial composition consists of purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 35, 16, 14, 19, 15, 31, 36, and 34. In one aspect, the bacterial composition consists essentially of purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 35, 16, 14, 19, 15, 31, 36, and 34. In one aspect, the bacterial consortium is LBP-16 (See e.g., FIG. 35).

In one aspect, the disclosure provides compositions comprising purified bacterial strains related to the following species Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, and Escherichia coli.

In one aspect, the disclosure provides compositions consisting of purified bacterial strains related to the following species Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, and Escherichia coli.

In one aspect, the disclosure provides compositions consisting essentially of purified bacterial strains related to the following species Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, and Escherichia coli. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 4, 5, 24, 1, 2, 3, 6, 7, 26, 29, 12, 13, 14, 19, 15, 28, 20, 25, 11, 17, 18 and 36. In one aspect, the bacterial composition consists of purified bacterial strains comprising a 16S rDNA sequence having at least 97% sequence identity with the nucleic acid sequence set forth as SEQ ID Nos: 4, 5, 24, 1, 2, 3, 6, 7, 26, 29, 12, 13, 14, 19, 15, 28, 20, 25, 11, 17, 18 and 36. In one aspect, the bacterial composition consist essentially of purified bacterial strains comprising a 16S rDNA sequence having at least 97% sequence identity with the nucleic acid sequence set forth as SEQ ID NOs: 4, 5, 24, 1, 2, 3, 6, 7, 26, 29, 12, 13, 14, 19, 15, 28, 20, 25, 11, 17, 18 and 36. In one aspect, the bacterial consortium is LBP-17 (See e.g., FIG. 35).

In one aspect, the disclosure provides compositions comprising purified bacterial strains related to the following species Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, and Dorea longicatena.

In one aspect, the disclosure provides compositions consisting of purified bacterial strains related to the following species Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, and Dorea longicatena.

In one aspect, the disclosure provides compositions consisting essentially of purified bacterial strains related to the following species Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, and Dorea longicatena. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 4, 5, 24, 1, 2, 3, 6, 7, 26, 29, 12, 13, 14, 19, 15, 28, 20, 25, 11, 17, and 18. In one aspect, the bacterial composition consists of purified bacterial strains comprising a 16S rDNA sequence having at least 97% sequence identity with the nucleic acid sequence set forth as SEQ ID NOs: 4, 5, 24, 1, 2, 3, 6, 7, 26, 29, 12, 13, 14, 19, 15, 28, 20, 25, 11, 17, and 18. In one aspect, the bacterial composition consists essentially of purified bacterial strains comprising a 16S rDNA sequence having at least 97% sequence identity with the nucleic acid sequence set forth as SEQ ID NOs: 4, 5, 24, 1, 2, 3, 6, 7, 26, 29, 12, 13, 14, 19, 15, 28, 20, 25, 11, 17, and 18. In one aspect, the bacterial consortium is LBP-18 (See e.g., FIG. 35).

In one aspect, the disclosure provides compositions comprising purified bacterial strains related to the following species Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Alistipes putredinis, Alistipes shahii, Clostridium clostridioforme, Clostridium innocuum, Erysipelatoclostridium ramosum, Erysipelotrichaceae bacterium 6_1_45, Eubacterium hallii, Blautia obeum, Blautia producta, Coprococcus comes, Escherichia coli, and Fusobacterium mortiferum.

In one aspect, the disclosure provides compositions consisting of purified bacterial strains related to the following species Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Alistipes putredinis, Alistipes shahii, Clostridium clostridioforme, Clostridium innocuum, Erysipelatoclostridium ramosum, Erysipelotrichaceae bacterium 6_1_45, Eubacterium hallii, Blautia obeum, Blautia producta, Coprococcus comes, Escherichia coli, and Fusobacterium mortiferum.

In one aspect, the disclosure provides compositions consisting essentially of purified bacterial strains related to the following species Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Alistipes putredinis, Alistipes shahii, Clostridium clostridioforme, Clostridium innocuum, Erysipelatoclostridium ramosum, Erysipelotrichaceae bacterium 6_1_45, Eubacterium hallii, Blautia obeum, Blautia producta, Coprococcus comes, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 4, 5, 24, 1, 2, 3, 6, 7, 26, 29, 13, 14, 19, 15, 28, 25, 11, 17, 34, and 36. In one aspect, the bacterial composition consists of purified bacterial strains comprising a 16S rDNA sequence having at least 97% sequence identity with the nucleic acid sequence set forth as SEQ ID NOs: 4, 5, 24, 1, 2, 3, 6, 7, 26, 29, 13, 14, 19, 15, 28, 25, 11, 17, 34, and 36. In one aspect, the bacterial composition consists essentially of purified bacterial strains comprising a 16S rDNA sequence having at least 97% sequence identity with the nucleic acid sequence set forth as SEQ ID NOs: 4, 5, 24, 1, 2, 3, 6, 7, 26, 29, 13, 14, 19, 15, 28, 25, 11, 17, 34, and 36. In one aspect, the bacterial consortium is LBP-19 (See e.g., FIG. 35).

In one aspect, the disclosure provides compositions comprising purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecium.

In one aspect, the disclosure provides compositions consisting of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecium.

In one aspect, the disclosure provides compositions consisting essentially of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecium. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 35, 16, 14, 19, 15, 28, 20, 30, 25, 11, 17, 18, 32, and 31. In one aspect, the bacterial composition consists of purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 35, 16, 14, 19, 15, 28, 20, 30, 25, 11, 17, 18, 32, and 31. In one aspect, the bacterial composition consists essentially of purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 35, 16, 14, 19, 15, 28, 20, 30, 25, 11, 17, 18, 32, and 31. In one aspect, the bacterial consortium is LBP-20 (See e.g., FIG. 35).

In one aspect, the disclosure provides compositions comprising purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting essentially of purified bacterial strains related to the following species Collinsella aerofaciens, Bifidobacterium pseudocatenulatum, Bifidobacterium adolescentis, Bifidobacterium longum, Bacteroides ovatus, Bacteroides thetaiotaomicron, Bacteroides xylanisolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 14, 19, 15, 28, 20, 30, 25, 11, 17, 18, 32, 31, 36, and 34. In one aspect, the bacterial composition consists of purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 14, 19, 15, 28, 20, 30, 25, 11, 17, 18, 32, 31, 36, and 34. In one aspect, the bacterial composition consists essentially of purified bacterial strains comprising a 16S rDNA sequence having at least 97% sequence identity with the nucleic acid sequence set forth as SEQ ID NOs: 27, 10, 8, 9, 4, 5, 24, 1, 2, 3, 33, 6, 7, 21, 22, 23, 26, 29, 12, 13, 14, 19, 15, 28, 20, 30, 25, 11, 17, 18, 32, 31, 36, and 34. In one aspect, the bacterial consortium is LBP-21 (See e.g., FIG. 35).

In one aspect, the disclosure provides compositions comprising purified bacterial strains related to the following species Collinsella aerofaciens, Bifidobacterium adolescentis, Bifidobacterium longum, Bacteroides ovatus, Bacteroides thetaiotaomicron, Bacteroides xylanisolvens, Bacteroides cellulosilyticus, Bacteroides uniformis, Bacteroides vulgatus, Parabacteroides distasonis, Alistipes putredinis, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting of purified bacterial strains related to the following species Collinsella aerofaciens, Bifidobacterium adolescentis, Bifidobacterium longum, Bacteroides ovatus, Bacteroides thetaiotaomicron, Bacteroides xylanisolvens, Bacteroides cellulosilyticus, Bacteroides uniformis, Bacteroides vulgatus, Parabacteroides distasonis, Alistipes putredinis, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting essentially of purified bacterial strains related to the following species Collinsella aerofaciens, Bifidobacterium adolescentis, Bifidobacterium longum, Bacteroides ovatus, Bacteroides thetaiotaomicron, Bacteroides xylanisolvens, Bacteroides cellulosilyticus, Bacteroides uniformis, Bacteroides vulgatus, Parabacteroides distasonis, Alistipes putredinis, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 8, 9, 4, 5, 24, 2, 6, 7, 22, 26, 12, 13, 14, 19, 15, 28, 20, 30, 25, 11, 17, 18, 32, 31, 36, and 34. In one aspect, the bacterial composition consists of purified bacterial strains comprising a 16S rDNA sequence having at least 97% sequence identity with the nucleic acid sequence set forth as SEQ ID NOs: 27, 8, 9, 4, 5, 24, 2, 6, 7, 22, 26, 12, 13, 14, 19, 15, 28, 20, 30, 25, 11, 17, 18, 32, 31, 36, and 34. In one aspect, the bacterial composition consists essentially of purified bacterial strains comprising a 16S rDNA sequence having at least 97% sequence identity with the nucleic acid sequence set forth as SEQ ID NOs: 27, 8, 9, 4, 5, 24, 2, 6, 7, 22, 26, 12, 13, 14, 19, 15, 28, 20, 30, 25, 11, 17, 18, 32, 31, 36, and 34. In one aspect, the bacterial consortium is LBP-22 (See e.g., FIG. 35).

In one aspect, the disclosure provides compositions comprising purified bacterial strains related to the following species Collinsella aerofaciens, Bifidobacterium longum, Bacteroides ovatus, Bacteroides thetaiotaomicron, Bacteroides xylanisolvens, Bacteroides cellulosilyticus, Bacteroides uniformis, Bacteroides vulgatus, Parabacteroides distasonis, Alistipes putredinis, Clostridium citroniae, Clostridium clostridioforme, Clostridium innocuum, Erysipelatoclostridium ramosum, Erysipelotrichaceae bacterium 6_1_45, Eubacterium hallii, Anaerostipes caccae, Blautia obeum, Blautia producta, Dorea longicatena, Agathobaculum butyriciproducens, Phascolarctobacterium faecium, Escherichia coli, and Fusobacterium mortiferum.

In one aspect, the disclosure provides compositions consisting of purified bacterial strains related to the following species Collinsella aerofaciens, Bifidobacterium longum, Bacteroides ovatus, Bacteroides thetaiotaomicron, Bacteroides xylanisolvens, Bacteroides cellulosilyticus, Bacteroides uniformis, Bacteroides vulgatus, Parabacteroides distasonis, Alistipes putredinis, Clostridium citroniae, Clostridium clostridioforme, Clostridium innocuum, Erysipelatoclostridium ramosum, Erysipelotrichaceae bacterium 6_1_45, Eubacterium hallii, Anaerostipes caccae, Blautia obeum, Blautia producta, Dorea longicatena, Agathobaculum butyriciproducens, Phascolarctobacterium faecium, Escherichia coli, and Fusobacterium mortiferum.

In one aspect, the disclosure provides compositions consisting essentially of purified bacterial strains related to the following species Collinsella aerofaciens, Bifidobacterium longum, Bacteroides ovatus, Bacteroides thetaiotaomicron, Bacteroides xylanisolvens, Bacteroides cellulosilyticus, Bacteroides uniformis, Bacteroides vulgatus, Parabacteroides distasonis, Alistipes putredinis, Clostridium citroniae, Clostridium clostridioforme, Clostridium innocuum, Erysipelatoclostridium ramosum, Erysipelotrichaceae bacterium 6_1_45, Eubacterium hallii, Anaerostipes caccae, Blautia obeum, Blautia producta, Dorea longicatena, Agathobaculum butyriciproducens, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 9, 4, 5, 24, 2, 6, 7, 22, 26, 12, 13, 14, 19, 15, 28, 30, 25, 11, 18, 32, 31, 36, and 24. In one aspect, the bacterial composition consists of purified bacterial strains comprising a 16S rDNA sequence having at least 97% sequence identity with the nucleic acid sequence set forth as SEQ ID NOs: 27, 9, 4, 5, 24, 2, 6, 7, 22, 26, 12, 13, 14, 19, 15, 28, 30, 25, 11, 18, 32, 31, 36, and 24. In one aspect, the bacterial composition consists essentially of purified bacterial strains comprising a 16S rDNA sequence having at least 97% sequence identity with the nucleic acid sequence set forth as SEQ ID NOs: 27, 9, 4, 5, 24, 2, 6, 7, 22, 26, 12, 13, 14, 19, 15, 28, 30, 25, 11, 18, 32, 31, 36, and 24. In one aspect, the bacterial consortium is LBP-23 (See e.g., FIG. 35).

In one aspect, the disclosure provides compositions comprising purified bacterial strains related to the following species Collinsella aerofaciens, Bifidobacterium longum, Bacteroides ovatus, Bacteroides thetaiotaomicron, Bacteroides xylanisolvens, Bacteroides caccae, Bacteroides cellulosilyticus, Bacteroides fragilis, Bacteroides uniformis, Bacteroides vulgatus, Odoribacter splanchnicus, Parabacteroides distasonis, Alistipes putredinis, Alistipes shahii, Clostridium citroniae, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting of purified bacterial strains related to the following species Collinsella aerofaciens, Bifidobacterium longum, Bacteroides ovatus, Bacteroides thetaiotaomicron, Bacteroides xylanisolvens, Bacteroides caccae, Bacteroides cellulosilyticus, Bacteroides fragilis, Bacteroides uniformis, Bacteroides vulgatus, Odoribacter splanchnicus, Parabacteroides distasonis, Alistipes putredinis, Alistipes shahii, Clostridium citroniae, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting essentially of purified bacterial strains related to the following species Collinsella aerofaciens, Bifidobacterium longum, Bacteroides ovatus, Bacteroides thetaiotaomicron, Bacteroides xylanisolvens, Bacteroides caccae, Bacteroides cellulosilyticus, Bacteroides fragilis, Bacteroides uniformis, Bacteroides vulgatus, Odoribacter splanchnicus, Parabacteroides distasonis, Alistipes putredinis, Alistipes shahii, Clostridium citroniae, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 9, 4, 5, 24, 1, 2, 33, 6, 7, 21, 22, 26, 29, 12, 13, 19, 15, 30, 25, 11, 17, 18, 32, 31, 36, and 34. In one aspect, the bacterial composition consists of purified bacterial strains comprising a 16S rDNA sequence having at least 97% sequence identity with the nucleic acid sequence set forth as SEQ ID NOs: 27, 9, 4, 5, 24, 1, 2, 33, 6, 7, 21, 22, 26, 29, 12, 13, 19, 15, 30, 25, 11, 17, 18, 32, 31, 36, and 34. In one aspect, the bacterial composition consists essentially of purified bacterial strains comprising a 16S rDNA sequence having at least 97% sequence identity with the nucleic acid sequence set forth as SEQ ID NOs: 27, 9, 4, 5, 24, 1, 2, 33, 6, 7, 21, 22, 26, 29, 12, 13, 19, 15, 30, 25, 11, 17, 18, 32, 31, 36, and 34. In one aspect, the bacterial consortium is LBP-24 (See e.g., FIG. 35).

In one aspect, the disclosure provides compositions comprising purified bacterial strains related to the following species Collinsella aerofaciens, Bifidobacterium pseudocatenulatum, Bifidobacterium adolescentis, Bifidobacterium longum, Bacteroides ovatus, Bacteroides xylanisolvens, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting of purified bacterial strains related to the following species Collinsella aerofaciens, Bifidobacterium pseudocatenulatum, Bifidobacterium adolescentis, Bifidobacterium longum, Bacteroides ovatus, Bacteroides xylanisolvens, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting essentially of purified bacterial strains related to the following species Collinsella aerofaciens, Bifidobacterium pseudocatenulatum, Bifidobacterium adolescentis, Bifidobacterium longum, Bacteroides ovatus, Bacteroides xylanisolvens, Bacteroides vulgatus, Odoribacter splanchnicus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 10, 8, 9, 4, 24, 7, 21, 22, 23, 26, 29, 12, 13, 35, 16, 14, 19, 15, 28, 20, 30, 25, 11, 17, 18, 32, 31, 36, and 34. In one aspect, the bacterial composition consists of purified bacterial strains comprising a 16S rDNA sequence having at least 97% sequence identity with the nucleic acid sequence set forth as SEQ ID NOs: 27, 10, 8, 9, 4, 24, 7, 21, 22, 23, 26, 29, 12, 13, 35, 16, 14, 19, 15, 28, 20, 30, 25, 11, 17, 18, 32, 31, 36, and 34. In one aspect, the bacterial composition consists essentially of purified bacterial strains comprising a 16S rDNA sequence having at least 97% sequence identity with the nucleic acid sequence set forth as SEQ ID NOs: 27, 10, 8, 9, 4, 24, 7, 21, 22, 23, 26, 29, 12, 13, 35, 16, 14, 19, 15, 28, 20, 30, 25, 11, 17, 18, 32, 31, 36, and 34. In one aspect, the bacterial consortium is LBP-25 (See e.g., FIG. 35).

In one aspect, the disclosure provides compositions comprising purified bacterial strains related to the following species Bifidobacterium longum, Bacteroides ovatus, Bacteroides thetaiotamicron, Bacteroides xylanisolvens, Bacteroides caccae, Bacteroides cellulosilyticus, Bacteroides faecis, Bacteroides fragilis, Bacteroides uniformis, Bacteroides vulgatus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Anaerostipes caccae, Blautia obeum, Blautia producta, Dorea longicatena, Agathobaculum butyriciproducens, Phascolarctobacterium faecium, Escherichia coli, and Fusobacterium mortiferum.

In one aspect, the disclosure provides compositions consisting of purified bacterial strains related to the following species Bifidobacterium longum, Bacteroides ovatus, Bacteroides thetaiotamicron, Bacteroides xylanisolvens, Bacteroides caccae, Bacteroides cellulosilyticus, Bacteroides faecis, Bacteroides fragilis, Bacteroides uniformis, Bacteroides vulgatus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Anaerostipes caccae, Blautia obeum, Blautia producta, Dorea longicatena, Agathobaculum butyriciproducens, Phascolarctobacterium faecium, Escherichia coli, and Fusobacterium mortiferum.

In one aspect, the disclosure provides compositions consisting essentially of purified bacterial strains related to the following species Bifidobacterium longum, Bacteroides ovatus, Bacteroides thetaiotamicron, Bacteroides xylanisolvens, Bacteroides caccae, Bacteroides cellulosilyticus, Bacteroides faecis, Bacteroides fragilis, Bacteroides uniformis, Bacteroides vulgatus, Parabacteroides distasonis, Parabacteroides merdae, Alistipes putredinis, Alistipes shahii, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Anaerostipes caccae, Blautia obeum, Blautia producta, Dorea longicatena, Agathobaculum butyriciproducens, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 9, 4, 5, 24, 1, 2, 3, 33, 6, 7, 22, 23, 26, 29, 12, 13, 35, 16, 14, 19, 15, 30, 25, 11, 18, 32, 31, 36, and 34. In one aspect, the bacterial composition consists of purified bacterial strains comprising a 16S rDNA sequence having at least 97% sequence identity with the nucleic acid sequence set forth as SEQ ID NOs: 9, 4, 5, 24, 1, 2, 3, 33, 6, 7, 22, 23, 26, 29, 12, 13, 35, 16, 14, 19, 15, 30, 25, 11, 18, 32, 31, 36, and 34. In one aspect, the bacterial composition consists essentially of purified bacterial strains comprising a 16S rDNA sequence having at least 97% sequence identity with the nucleic acid sequence set forth as SEQ ID NOs: 9, 4, 5, 24, 1, 2, 3, 33, 6, 7, 22, 23, 26, 29, 12, 13, 35, 16, 14, 19, 15, 30, 25, 11, 18, 32, 31, 36, and 34. In one aspect, the bacterial consortium is LBP-26 (See e.g., FIG. 35).

In one aspect, the disclosure provides compositions comprising purified bacterial strains related to the following species Bifidobacterium longum, Bacteroides ovatus, Bacteroides xylanisolvens, Bacteroides caccae, Bacteroides vulgatus, Odoribacter splanchnicus, Parabacteroides distasonis, Alistipes putredinis, Alistipes shahii, Clostridium citroniae, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting of purified bacterial strains related to the following species Bifidobacterium longum, Bacteroides ovatus, Bacteroides xylanisolvens, Bacteroides caccae, Bacteroides vulgatus, Odoribacter splanchnicus, Parabacteroides distasonis, Alistipes putredinis, Alistipes shahii, Clostridium citroniae, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting essentially of purified bacterial strains related to the following species Bifidobacterium longum, Bacteroides ovatus, Bacteroides xylanisolvens, Bacteroides caccae, Bacteroides vulgatus, Odoribacter splanchnicus, Parabacteroides distasonis, Alistipes putredinis, Alistipes shahii, Clostridium citroniae, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 9, 4, 5, 24, 1, 7, 21, 22, 26, 29, 12, 13, 19, 15, 30, 25, 11, 17, 18, 32, 31, 36, and 34. In one aspect, the bacterial composition consists of purified bacterial strains comprising a 16S rDNA sequence having at least 97% sequence identity with the nucleic acid sequence set forth as SEQ ID NOs: 9, 4, 5, 24, 1, 7, 21, 22, 26, 29, 12, 13, 19, 15, 30, 25, 11, 17, 18, 32, 31, 36, and 34. In one aspect, the bacterial composition consists essentially of purified bacterial strains comprising a 16S rDNA sequence having at least 97% sequence identity with the nucleic acid sequence set forth as SEQ ID NOs: 9, 4, 5, 24, 1, 7, 21, 22, 26, 29, 12, 13, 19, 15, 30, 25, 11, 17, 18, 32, 31, 36, and 34. In one aspect, the bacterial consortium is LBP-27 (See e.g., FIG. 35).

In one aspect, the disclosure provides compositions comprising purified bacterial strains related to the following species Collinsella aerofaciens, Bifidobacterium longum, Bacteroides ovatus, Bacteroides thetaiotamicron, Bacteroides xylanisolvens, Bacteroides caccae, Bacteroides cellulosilyticus, Bacteroides ovatus, Bacteroides vulgatus, Odoribacter splanchnicus, Parabacteroides distasonis, Alistipes putredinis, Alistipes shahii, Clostridium citroniae, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting of purified bacterial strains related to the following species Collinsella aerofaciens, Bifidobacterium longum, Bacteroides ovatus, Bacteroides thetaiotamicron, Bacteroides xylanisolvens, Bacteroides caccae, Bacteroides cellulosilyticus, Bacteroides ovatus, Bacteroides vulgatus, Odoribacter splanchnicus, Parabacteroides distasonis, Alistipes putredinis, Alistipes shahii, Clostridium citroniae, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting essentially of purified bacterial strains related to the following species Collinsella aerofaciens, Bifidobacterium longum, Bacteroides ovatus, Bacteroides thetaiotamicron, Bacteroides xylanisolvens, Bacteroides caccae, Bacteroides cellulosilyticus, Bacteroides ovatus, Bacteroides vulgatus, Odoribacter splanchnicus, Parabacteroides distasonis, Alistipes putredinis, Alistipes shahii, Clostridium citroniae, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 9, 4, 5, 24, 1, 2, 6, 7, 21, 22, 26, 29, 12, 13, 19, 15, 30, 25, 11, 17, 18, 32, 31, 36, and 34. In one aspect, the bacterial composition consists of purified bacterial strains comprising a 16S rDNA sequence having at least 97% sequence identity with the nucleic acid sequence set forth as SEQ ID NOs: 27, 9, 4, 5, 24, 1, 2, 6, 7, 21, 22, 26, 29, 12, 13, 19, 15, 30, 25, 11, 17, 18, 32, 31, 36, and 34. In one aspect, the bacterial composition consists essentially of purified bacterial strains comprising a 16S rDNA sequence having at least 97% sequence identity with the nucleic acid sequence set forth as SEQ ID NOs: 27, 9, 4, 5, 24, 1, 2, 6, 7, 21, 22, 26, 29, 12, 13, 19, 15, 30, 25, 11, 17, 18, 32, 31, 36, and 34. In one aspect, the bacterial consortium is LBP-28 (See e.g., FIG. 35).

In one aspect, the disclosure provides compositions comprising purified bacterial strains related to the following species Collinsella aerofaciens, Bifidobacterium longum, Bacteroides ovatus, Bacteroides xylanisolvens, Bacteroides vulgatus, Odoribacter splanchnicus, Parabacteroides distasonis, Alistipes putredinis, Alistipes shahii, Clostridium citroniae, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting of purified bacterial strains related to the following species Collinsella aerofaciens, Bifidobacterium longum, Bacteroides ovatus, Bacteroides xylanisolvens, Bacteroides vulgatus, Odoribacter splanchnicus, Parabacteroides distasonis, Alistipes putredinis, Alistipes shahii, Clostridium citroniae, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting essentially of purified bacterial strains related to the following species Collinsella aerofaciens, Bifidobacterium longum, Bacteroides ovatus, Bacteroides xylanisolvens, Bacteroides vulgatus, Odoribacter splanchnicus, Parabacteroides distasonis, Alistipes putredinis, Alistipes shahii, Clostridium citroniae, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 9, 4, 24, 7, 21, 22, 26, 29, 12, 13, 19, 15, 30, 25, 11, 17, 18, 32, 31, 36, and 34. In one aspect, the bacterial composition consists of purified bacterial strains comprising a 16S rDNA sequence having at least 97% sequence identity with the nucleic acid sequence set forth as SEQ ID NOs: 27, 9, 4, 24, 7, 21, 22, 26, 29, 12, 13, 19, 15, 30, 25, 11, 17, 18, 32, 31, 36, and 34. In one aspect, the bacterial composition consists essentially of purified bacterial strains comprising a 16S rDNA sequence having at least 97% sequence identity with the nucleic acid sequence set forth as SEQ ID NOs: 27, 9, 4, 24, 7, 21, 22, 26, 29, 12, 13, 19, 15, 30, 25, 11, 17, 18, 32, 31, 36, and 34. In one aspect, the bacterial consortium is LBP-29 (See e.g., FIG. 35).

In one aspect, the disclosure provides compositions comprising purified bacterial strains related to the following species Collinsella aerofaciens, Bifidobacterium longum, Bacteroides ovatus, Bacteroides xylanisolvens, Odoribacter splanchnicus, Alistipes putredinis, Alistipes shahii, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting of purified bacterial strains related to the following species Collinsella aerofaciens, Bifidobacterium longum, Bacteroides ovatus, Bacteroides xylanisolvens, Odoribacter splanchnicus, Alistipes putredinis, Alistipes shahii, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum.

In one aspect, the disclosure provides compositions consisting essentially of purified bacterial strains related to the following species Collinsella aerofaciens, Bifidobacterium longum, Bacteroides ovatus, Bacteroides xylanisolvens, Odoribacter splanchnicus, Alistipes putredinis, Alistipes shahii, Clostridium clostridioforme, Erysipelatoclostridium ramosum, Erysipelotrichaceae bacterium 6_1_45, Anaerostipes caccae, Blautia obeum, Blautia producta, Coprococcus comes, Dorea longicatena, Agathobaculum butyriciproducens, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 9, 4, 24, 21, 26, 13, 19, 15, 30, 25, 11, 17, 18, 32, 36, and 34. In one aspect, the bacterial composition consists of purified bacterial strains comprising a 16S rDNA sequence having at least 97% sequence identity with the nucleic acid sequence set forth as SEQ ID NOs: 27, 9, 4, 24, 21, 26, 13, 19, 15, 30, 25, 11, 17, 18, 32, 36, and 34. In one aspect, the bacterial composition consists essentially of purified bacterial strains comprising a 16S rDNA sequence having at least 97% sequence identity with the nucleic acid sequence set forth as SEQ ID NOs: 27, 9, 4, 24, 21, 26, 13, 19, 15, 30, 25, 11, 17, 18, 32, 36, and 34. In one aspect, the bacterial consortium is LBP-30 (See e.g., FIG. 35).

In one aspect, the disclosure provides compositions comprising purified bacterial strains related to the following species Collinsella aerofaciens, Bifidobacterium adolescentis, Bifidobacterium longum, Bacteroides ovatus, Bacteroides faecis, Bacteroides fragilis, Bacteroides vulgatus, Odoribacter splanchnicus, Alistipes putredinis, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Anaerostipes caccae, Blautia obeum, Blautia producta, Dorea longicatena, Agathobaculum butyriciproducens, Phascolarctobacterium faecium, Escherichia coli, and Fusobacterium mortiferum.

In one aspect, the disclosure provides compositions consisting of purified bacterial strains related to the following species Collinsella aerofaciens, Bifidobacterium adolescentis, Bifidobacterium longum, Bacteroides ovatus, Bacteroides faecis, Bacteroides fragilis, Bacteroides vulgatus, Odoribacter splanchnicus, Alistipes putredinis, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Anaerostipes caccae, Blautia obeum, Blautia producta, Dorea longicatena, Agathobaculum butyriciproducens, Phascolarctobacterium faecium, Escherichia coli, and Fusobacterium mortiferum.

In one aspect, the disclosure provides compositions consisting essentially of purified bacterial strains related to the following species Collinsella aerofaciens, Bifidobacterium adolescentis, Bifidobacterium longum, Bacteroides ovatus, Bacteroides faecis, Bacteroides fragilis, Bacteroides vulgatus, Odoribacter splanchnicus, Alistipes putredinis, Clostridium citroniae, Clostridium clostridioforme, Clostridium bifermentans, Clostridium sordellii, Clostridium innocuum, Erysipelatoclostridium ramosum, Erysipelotrichaceae bacterium 6_1_45, Anaerostipes caccae, Blautia obeum, Blautia producta, Dorea longicatena, Agathobaculum butyriciproducens, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 8, 9, 4, 3, 33, 7, 21, 26, 12, 13, 35, 16, 14, 19, 15, 28, 30, 25, 11, 18, 32, 31, 36, 34. In one aspect, the bacterial composition consists of purified bacterial strains comprising a 16S rDNA sequence having at least 97% sequence identity with the nucleic acid sequence set forth as SEQ ID NOs: 27, 8, 9, 4, 3, 33, 7, 21, 26, 12, 13, 35, 16, 14, 19, 15, 28, 30, 25, 11, 18, 32, 31, 36, 34. In one aspect, the bacterial composition consists essentially of purified bacterial strains comprising a 16S rDNA sequence having at least 97% sequence identity with the nucleic acid sequence set forth as SEQ ID NOs: 27, 8, 9, 4, 3, 33, 7, 21, 26, 12, 13, 35, 16, 14, 19, 15, 28, 30, 25, 11, 18, 32, 31, 36, 34. In one aspect, the bacterial consortium is LBP-31 (See e.g., FIG. 35).

In one aspect, the disclosure provides compositions comprising purified bacterial strains related to the following species Collinsella aerofaciens, Bifidobacterium longum, Bacteroides ovatus, Bacteroides faecis, Bacteroides vulgatus, Odoribacter splanchnicus, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Dorea longicatena, Agathobaculum butyriciproducens, Phascolarctobacterium faecium, Escherichia coli, and Fusobacterium mortiferum.

In one aspect, the disclosure provides compositions consisting of purified bacterial strains related to the following species Collinsella aerofaciens, Bifidobacterium longum, Bacteroides ovatus, Bacteroides faecis, Bacteroides vulgatus, Odoribacter splanchnicus, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Dorea longicatena, Agathobaculum butyriciproducens, Phascolarctobacterium faecium, Escherichia coli, and Fusobacterium mortiferum.

In one aspect, the disclosure provides compositions consisting essentially of purified bacterial strains related to the following species Collinsella aerofaciens, Bifidobacterium longum, Bacteroides ovatus, Bacteroides faecis, Bacteroides vulgatus, Odoribacter splanchnicus, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Dorea longicatena, Agathobaculum butyriciproducens, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 9, 4, 3, 7, 21, 26, 12, 13, 19, 15, 30, 25, 11, 18, 32, 31, 36, and 34. In one aspect, the bacterial composition consists of purified bacterial strains comprising a 16S rDNA sequence having at least 97% sequence identity with the nucleic acid sequence set forth as SEQ ID NOs: 27, 9, 4, 3, 7, 21, 26, 12, 13, 19, 15, 30, 25, 11, 18, 32, 31, 36, and 34. In one aspect, the bacterial composition consists essentially of purified bacterial strains comprising a 16S rDNA sequence having at least 97% sequence identity with the nucleic acid sequence set forth as SEQ ID NOs: 27, 9, 4, 3, 7, 21, 26, 12, 13, 19, 15, 30, 25, 11, 18, 32, 31, 36, and 34. In one aspect, the bacterial consortium is LBP-32 (See e.g., FIG. 35).

In one aspect, the disclosure provides compositions comprising purified bacterial strains related to the following species Collinsella aerofaciens, Bifidobacterium longum, Bacteroides ovatus, Bacteroides vulgatus, Parabacteroides distasonis, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Agathobaculum butyriciproducens, Phascolarctobacterium faecium, Escherichia coli, and Fusobacterium mortiferum.

In one aspect, the disclosure provides compositions consisting of purified bacterial strains related to the following species Collinsella aerofaciens, Bifidobacterium longum, Bacteroides ovatus, Bacteroides vulgatus, Parabacteroides distasonis, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Agathobaculum butyriciproducens, Phascolarctobacterium faecium, Escherichia coli, and Fusobacterium mortiferum.

In one aspect, the disclosure provides compositions consisting essentially of purified bacterial strains related to the following species Collinsella aerofaciens, Bifidobacterium longum, Bacteroides ovatus, Bacteroides vulgatus, Parabacteroides distasonis, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Agathobaculum butyriciproducens, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 9, 4, 7, 22, 26, 12, 13, 19, 15, 30, 25, 11, 32, 31, 36, and 34. In one aspect, the bacterial composition consists of purified bacterial strains comprising a 16S rDNA sequence having at least 97% sequence identity with the nucleic acid sequence set forth as SEQ ID NOs: 27, 9, 4, 7, 22, 26, 12, 13, 19, 15, 30, 25, 11, 32, 31, 36, and 34. In one aspect, the bacterial composition consists essentially of purified bacterial strains comprising a 16S rDNA sequence having at least 97% sequence identity with the nucleic acid sequence set forth as SEQ ID NOs: 27, 9, 4, 7, 22, 26, 12, 13, 19, 15, 30, 25, 11, 32, 31, 36, and 34. In one aspect, the bacterial consortium is LBP-33 (See e.g., FIG. 35).

In one aspect, the disclosure provides compositions comprising purified bacterial strains related to the following species Collinsella aerofaciens, Bifidobacterium longum, Bacteroides ovatus, Bacteroides vulgatus, Alistipes putredinis, Clostridium citroniae, Clostridium clostridioforme, Erysipelatoclostridium ramosum, Erysipelotrichaceae bacterium 6_1_45, Blautia obeum, Blautia producta, Dorea longicatena, Phascolarctobacterium faecium, Escherichia coli, and Fusobacterium mortiferum.

In one aspect, the disclosure provides compositions consisting of purified bacterial strains related to the following species Collinsella aerofaciens, Bifidobacterium longum, Bacteroides ovatus, Bacteroides vulgatus, Alistipes putredinis, Clostridium citroniae, Clostridium clostridioforme, Erysipelatoclostridium ramosum, Erysipelotrichaceae bacterium 6_1_45, Blautia obeum, Blautia producta, Dorea longicatena, Phascolarctobacterium faecium, Escherichia coli, and Fusobacterium mortiferum.

In one aspect, the disclosure provides compositions consisting essentially of purified bacterial strains related to the following species Collinsella aerofaciens, Bifidobacterium longum, Bacteroides ovatus, Bacteroides vulgatus, Alistipes putredinis, Clostridium citroniae, Clostridium clostridioforme, Erysipelatoclostridium ramosum, Erysipelotrichaceae bacterium 6_1_45, Blautia obeum, Blautia producta, Dorea longicatena, Phascolarctobacterium faecium, Escherichia coli, and Fusobacterium mortiferum. In some embodiments, consisting essentially of, as used herein, refers to compositions that do not include any additional therapeutically active bacterial strains.

In one aspect, the bacterial composition comprises purified bacterial strains comprising a 16S rDNA sequence having at least 97% sequence identity with the nucleic acid sequence set forth as SEQ ID NOs: 27, 9, 4, 7, 26, 12, 13, 19, 15, 25, 11, 18, 31, 36, and 34. In one aspect, the bacterial composition consists of purified bacterial strains comprising a 16S rDNA sequence having at least 97% sequence identity with the nucleic acid sequence set forth as SEQ ID NOs: 27, 9, 4, 7, 26, 12, 13, 19, 15, 25, 11, 18, 31, 36, and 34. In one aspect, the bacterial composition consists essentially of purified bacterial strains comprising a 16S rDNA sequence having at least 97% sequence identity with the nucleic acid sequence set forth as SEQ ID NOs: 27, 9, 4, 7, 26, 12, 13, 19, 15, 25, 11, 18, 31, 36, and 34. In one aspect, the bacterial consortium is LBP-34 (See e.g., FIG. 35).

In one aspect, the disclosure provides compositions comprising one or more purified bacterial strains belonging to the phylum Bacteroidetes and one or more purified bacterial strains selected from the group consisting of an Escherichia species (e.g., Escherichia sp. 3_2_53FAA) and a Fusobacterium species (e.g., Fusobacterium mortiferum). In one aspect, the disclosure provides compositions consisting of one or more purified bacterial strains belonging to the phylum Bacteroidetes and one or more purified bacterial strains selected from the group consisting of an Escherichia species (e.g., Escherichia sp. 3_2_53FAA) and a Fusobacterium species (e.g., Fusobacterium mortiferum). In one aspect, the disclosure provides compositions consisting essentially of one or more purified bacterial strains belonging to the phylum Bacteroidetes and one or more purified bacterial strains selected from the group consisting of an Escherichia species (e.g., Escherichia sp. 3_2_53FAA) and a Fusobacterium species (e.g., Fusobacterium mortiferum).

In one aspect, the disclosure provides compositions comprising one or more purified bacterial strains belonging to the phylum Bacteroidetes and one or more purified bacterial strains of Escherichia species, such as E. coli (e.g., Escherichia sp. 3_2_53FAA). In one aspect, the disclosure provides compositions consisting of one or more purified bacterial strains belonging to the phylum Bacteroidetes and one or more purified bacterial strains of Escherichia species, such as E. coli (e.g., Escherichia sp. 3_2_53FAA). In one aspect, the disclosure provides compositions consisting essentially of one or more purified bacterial strains belonging to the phylum Bacteroidetes and one or more purified bacterial strains of Escherichia species, such as E. coli (e.g., Escherichia sp. 3_2_53FAA).

In one aspect, the disclosure provides compositions comprising one or more purified bacterial strains belonging to the phylum Bacteroidetes and one or more purified bacterial strains of Fusobacterium species (e.g., Fusobacterium mortiferum). In one aspect, the disclosure provides compositions consisting of one or more purified bacterial strains belonging to the phylum Bacteroidetes and one or more purified bacterial strains of Fusobacterium species (e.g., Fusobacterium mortiferum). In one aspect, the disclosure provides compositions consisting essentially of one or more purified bacterial strains belonging to the phylum Bacteroidetes and one or more purified bacterial strains of Fusobacterium species (e.g., Fusobacterium mortiferum). In some embodiments, consisting essentially of, as used herein, refers to compositions that do not include any additional therapeutically active bacterial strains.

In some embodiments, the compositions described herein comprise one or more purified bacterial strains belonging to the phylum Bacteroidetes. Bacteroidetes are Gram-negative, non-spore forming, rod-shaped bacteria. Although Bacteroidetes can be opportunistic pathogens, many Bacteroidetes species are symbiotic with the mouse and human gastrointestinal tract, where they degrade proteins and complex sugar polymers for the host. Reduced abundance of Bacteriodetes may be associated with obesity and irritable bowel syndrome, and increased abundance of Bacteriodetes may be associated with type 1 and type 2 diabetes. The phylum Bacteriodetes includes classes Bacteriodia (e.g., Bacteroidales), Balneolia (e.g., Balneolales), Chitinophagia (e.g., Chitinophagales), Cytophagia (e.g., Cytophagales), Flavobacteriia (e.g., Flavobacteriales), Rhodothermia (e.g., Rhodothermales), and Sphingobacteria (e.g., Sphingobacteriales).

In some embodiments, one or more bacterial strains of the phylum Bacteriodetes belongs to the class Bacteriodales. In some embodiments, one or more bacterial strains of the phylum Bacteriodetes belongs to the family Bacteriodaceae.

In some embodiments, one or more bacterial strains of the phylum Bacteroidetes produces short chain fatty acids (SCFAs), such as any of the SCFAs described herein. In some embodiments, one or more bacterial strains of the phylum Bacteroidetes produces butyrate. In some embodiments, the Bacteriodetes bacteria that are butyrate producers are Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii.

In one aspect, the disclosure provides compositions comprising a purified bacterial strain belonging to an Escherichia species (e.g., Escherichia sp. 3_2_53FAA), a Fusobacterium species (e.g., Fusobacterium mortiferum), and one or more (e.g., 1, 2, 3, 4, 5, 6, 7, 8, 9, 10, 11, 12, 13, 14, 15, 16, 17, 18, 19, or 20) purified bacterial strains of species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii.

In one aspect, the disclosure provides compositions consisting of a purified bacterial strain belonging to an Escherichia species (e.g., Escherichia sp. 3_2_53FAA), a Fusobacterium species (e.g., Fusobacterium mortiferum), and one or more (e.g., 1, 2, 3, 4, 5, 6, 7, 8, 9, 10, 11, 12, 13, 14, 15, 16, 17, 18, 19, or 20) purified bacterial strains of species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii.

In one aspect, the disclosure provides compositions consisting essentially of a purified bacterial strain belonging to an Escherichia species (e.g., Escherichia sp. 3_2_53FAA) and a Fusobacterium species (e.g., Fusobacterium mortiferum), and one or more (e.g., 1, 2, 3, 4, 5, 6, 7, 8, 9, 10, 11, 12, 13, 14, 15, 16, 17, 18, 19, or 20) purified bacterial strains of species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii. In some embodiments, consisting essentially of, as used herein, refers to compositions that do not include any additional therapeutically active bacterial strains.

In one aspect, the bacterial composition comprises a purified bacterial strain comprising a 16S rDNA sequence having at least 97% sequence identity with the nucleic acid sequence set forth as SEQ ID NO: 36, a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 1-7, 21-24, 26, 29, and 33.

In one aspect, the bacterial composition consists of a purified bacterial strain comprising a 16S rDNA sequence having at least 97% sequence identity with the nucleic acid sequence set forth as SEQ ID NO: 36, a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 1-7, 21-24, 26, 29, and 33.

In one aspect, the bacterial composition consists essentially of a purified bacterial strain comprising a 16S rDNA sequence having at least 97% sequence identity with the nucleic acid sequence set forth as SEQ ID NO: 36, a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 1-7, 21-24, 26, 29, and 33.

In one aspect, the disclosure provides compositions comprising a purified bacterial strain belonging to an Escherichia species (e.g., Escherichia sp. 3_2_53FAA) and one or more (e.g., 1, 2, 3, 4, 5, 6, 7, 8, 9, 10, 11, 12, 13, 14, 15, 16, 17, 18, 19, or 20) purified bacterial strains of species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii.

In one aspect, the disclosure provides compositions consisting of a purified bacterial strain belonging to an Escherichia species (e.g., Escherichia sp. 3_2_53FAA) and one or more (e.g., 1, 2, 3, 4, 5, 6, 7, 8, 9, 10, 11, 12, 13, 14, 15, 16, 17, 18, 19, or 20) purified bacterial strains of species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii.

In one aspect, the disclosure provides compositions consisting essentially of a purified bacterial strain belonging to an Escherichia species (e.g., Escherichia sp. 3_2_53FAA) and one or more (e.g., 1, 2, 3, 4, 5, 6, 7, 8, 9, 10, 11, 12, 13, 14, 15, 16, 17, 18, 19, or 20) purified bacterial strains of species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii. In some embodiments, consisting essentially of, as used herein, refers to compositions that do not include any additional therapeutically active bacterial strains.

In one aspect, the bacterial composition comprises a purified bacterial strain comprising a 16S rDNA sequence having at least 97% sequence identity with the nucleic acid sequence set forth as SEQ ID NO: 36, and one or more purified bacterial strains comprising a 16S rDNA sequence having at least 97% sequence identity with the nucleic acid sequence set forth as SEQ ID NOs: 1-7, 21-24, 26, 29, and 33.

In one aspect, the bacterial composition consists of a purified bacterial strain comprising a 16S rDNA sequence having at least 97% sequence identity with the nucleic acid sequence set forth as SEQ ID NO: 36, and one or more purified bacterial strains comprising a 16S rDNA sequence having at least 97% sequence identity with the nucleic acid sequence set forth as SEQ ID NOs: 1-7, 21-24, 26, 29, and 33.

In one aspect, the bacterial composition consists essentially of a purified bacterial strain comprising a 16S rDNA sequence having at least 97% sequence identity with the nucleic acid sequence set forth as SEQ ID NO: 36, and one or more purified bacterial strains comprising a 16S rDNA sequence having at least 97% sequence identity with the nucleic acid sequence set forth as SEQ ID NOs: 1-7, 21-24, 26, 29, and 33.

In one aspect, the disclosure provides compositions comprising a purified bacterial strain belonging to a Fusobacterium species (e.g., Fusobacterium mortiferum) and one or more (e.g., 1, 2, 3, 4, 5, 6, 7, 8, 9, 10, 11, 12, 13, 14, 15, 16, 17, 18, 19, or 20) purified bacterial strains of species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii.

In one aspect, the disclosure provides compositions consisting of a purified bacterial strain belonging to a Fusobacterium species (e.g., Fusobacterium mortiferum) and one or more (e.g., 1, 2, 3, 4, 5, 6, 7, 8, 9, 10, 11, 12, 13, 14, 15, 16, 17, 18, 19, or 20) purified bacterial strains of species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii.

In one aspect, the disclosure provides compositions consisting essentially of a purified bacterial strain belonging to a Fusobacterium species (e.g., Fusobacterium mortiferum) and one or more (e.g., 1, 2, 3, 4, 5, 6, 7, 8, 9, 10, 11, 12, 13, 14, 15, 16, 17, 18, 19, or 20) purified bacterial strains of species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii. In some embodiments, consisting essentially of, as used herein, refers to compositions that do not include any additional therapeutically active bacterial strains.

In one aspect, the bacterial composition comprises a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 1-7, 21-24, 26, 29, and 33.

In one aspect, the bacterial composition consists of a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 1-7, 21-24, 26, 29, and 33.

In one aspect, the bacterial composition consists essentially of a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 1-7, 21-24, 26, 29, and 33.

In one aspect, the disclosure provides compositions comprising a purified bacterial strain belonging to Escherichia species (such as E. coli (e.g. Escherichia sp. 3_2_53FAA), a purified bacterial strain belonging to Fusobacterium (e.g., Fusobacterium mortiferum) and one or more bacterial species selected from the group consisting of Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis. See, PCT Publication No. WO 2019/118515, which is incorporated by reference herein in its entirety.

In one aspect, the disclosure provides compositions consisting of a purified bacterial strain belonging to Escherichia species (such as E. coli (e.g. Escherichia sp. 3_2_53FAA), a purified bacterial strain belonging to Fusobacterium (e.g., Fusobacterium mortiferum) and one or more bacterial species selected from the group consisting of Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis.

In one aspect, the disclosure provides compositions consisting essentially of a purified bacterial strain belonging to Escherichia species (such as E. coli (e.g. Escherichia sp. 3_2_53FAA), a purified bacterial strain belonging to Fusobacterium (e.g., Fusobacterium mortiferum) and one or more bacterial species selected from the group consisting of Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis.

In one aspect, the bacterial composition comprises a purified bacterial strain comprising a 16S rDNA sequence having at least 97% sequence identity with the nucleic acid sequence set forth as SEQ ID NO: 36, a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 1-33 and 35.

In one aspect, the bacterial composition consists of a purified bacterial strain comprising a 16S rDNA sequence having at least 97% sequence identity with the nucleic acid sequence set forth as SEQ ID NO: 36, a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 1-33 and 35.

In one aspect, the bacterial composition consists essentially of a purified bacterial strain comprising a 16S rDNA sequence having at least 97% sequence identity with the nucleic acid sequence set forth as SEQ ID NO: 36, a purified bacterial strain comprising a 16S rDNA sequence having at least 97% sequence identity with the nucleic acid sequence set forth as SEQ ID NO: 34, and one or more purified bacterial strains comprising a 16S rDNA sequence having at least 97% sequence identity with the nucleic acid sequence set forth as SEQ ID NOs: 1-33 and 35.

It should be appreciated that any of the compositions described herein may contain multiple strains of a particular bacterial species. For example, in some embodiments, the composition may comprise two strains of Parabacteroides distasonis and/or two strains of Parabacteroides merdae.

The disclosure also encompasses compositions comprising bacterial strains having close sequence identity or homology to and/or fall within the species of Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium bolteae, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecis, Escherichia sp. 3_2_53FAA, and Fusobacterium mortiferum.

In some embodiments, the compositions disclosed herein comprise two or more bacterial strains. In some embodiments, the compositions described herein comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20, at least 21, at least 22, at least 23, at least 24, at least 25, at least 26, at least 27, at least 28, at least 29, at least 30, at least 31, at least 32, at least 33, at least 34, at least 35, at least 36 or more bacterial strains (e.g., purified bacterial strains).

In some embodiments, the composition does not comprise Fusobacterium mortiferum. In some embodiments, the composition does not comprise a bacterial strain that is considered a potential clinical risk. In some embodiments, the composition does not comprise C. bifermentans, B. fragilis, and/or C. sordellii. In some embodiments, the composition does not comprise a bacterial strain belonging to the family Eubacteriaceae, such as Eubacterium hallii and/or Eubacterium rectale.

It should be appreciated that the terms “bacteria” and “bacterial strains” as used herein are interchangeable. The compositions described herein containing multiple purified bacterial strains may also be referred to as “live bacterial products.”

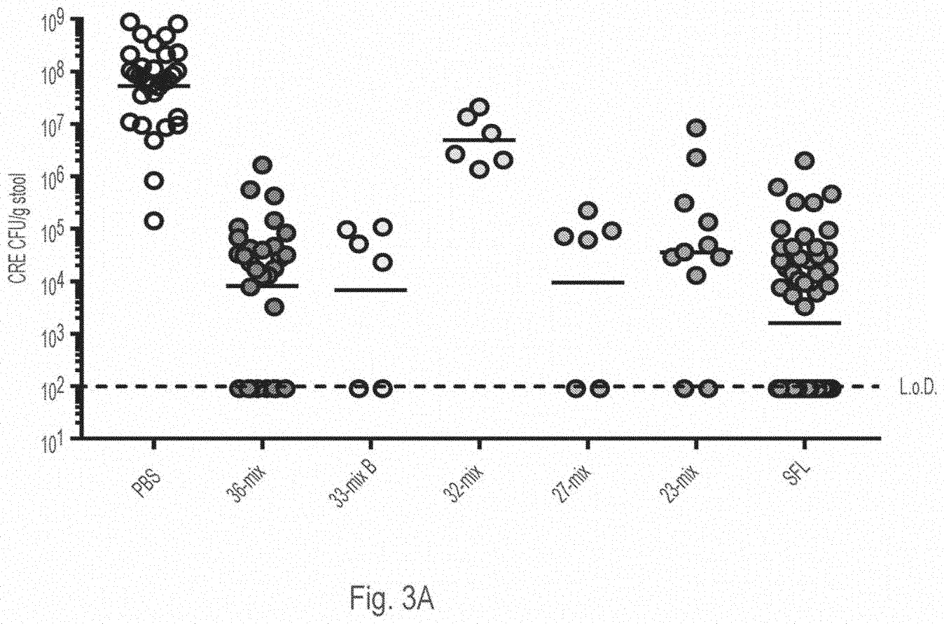

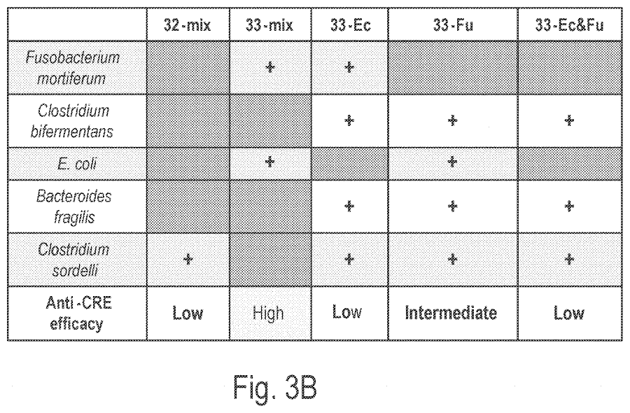

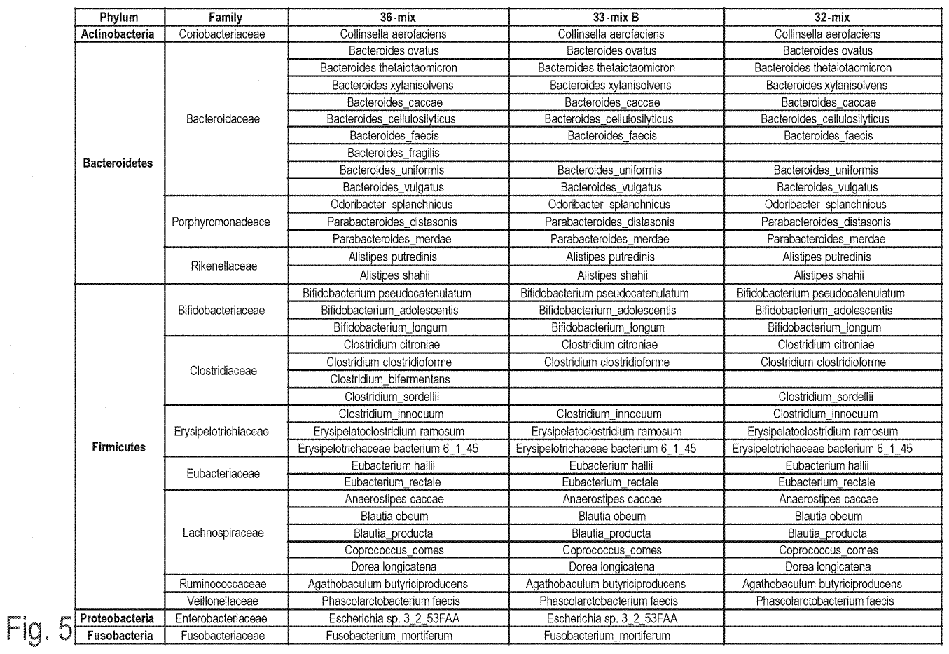

In one aspect, the disclosure provides a live bacterial product referred to as “33-mix B” (see, FIGS. 3 and 5). As shown in FIG. 5, live bacterial product 33-mix B contains 33 bacterial strains related to the following species Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecis, Escherichia sp. 3_2_53FAA, and Fusobacterium mortiferum.

In some embodiments, the compositions comprise two or more (e.g., 2, 3, 4, 5, or more) bacterial species selected from the group consisting of Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecis, Escherichia sp. 3_2_53FAA, and Fusobacterium mortiferum.

In some embodiments, the compositions consists of two or more (e.g., 2, 3, 4, 5, or more) bacterial species selected from the group consisting of Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecis, Escherichia sp. 3_2_53FAA, and Fusobacterium mortiferum.

In some embodiments, the compositions comprise 33 bacterial species: Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecis, Escherichia sp. 3_2_53FAA, and Fusobacterium mortiferum.

In some embodiments, the compositions consist of 33 bacterial species: Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecis, Escherichia sp. 3_2_53FAA, and Fusobacterium mortiferum.

In some embodiments, the compositions consist essentially of 33 bacterial species: Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecis, Escherichia sp. 3_2_53FAA, and Fusobacterium mortiferum. In some embodiments, consisting essentially of, as used herein, refers to compositions that do not include any additional therapeutically active bacterial strains.

In one aspect, the bacterial composition comprises two or more (e.g., 2, 3, 4, 5 or more) purified bacterial strains comprising 16S rDNA sequences having at least 97% sequence identity with the nucleic acid sequence selected from SEQ ID NOs: 1-36. In one aspect, the bacterial composition consists of two or more (e.g., 2, 3, 4, 5 or more) purified bacterial strains comprising 16S rDNA sequences having at least 97% sequence identity with the nucleic acid sequence selected from SEQ ID NOs: 1-36. In one aspect, the bacterial composition consists essentially of two or more (e.g., 2, 3, 4, 5 or more) purified bacterial strains comprising 16S rDNA sequences having at least 97% sequence identity with the nucleic acid sequence selected from SEQ ID NOs: 1-36.

In one aspect, the bacterial composition comprises 33 bacterial strains comprising 16S rDNA sequences having at least 97% sequence identity with the nucleic acid sequence selected from SEQ ID NOs: 1-15, 17-32, 34, and 36. In one aspect, the bacterial composition consists of 33 bacterial strains comprising 16S rDNA sequences having at least 97% sequence identity with the nucleic acid sequence selected from SEQ ID NOs: 1-15, 17-32, 34, and 36. In one aspect, the bacterial composition consists essentially of 33 bacterial strains comprising 16S rDNA sequences having at least 97% sequence identity with the nucleic acid sequence selected from SEQ ID NOs: 1-15, 17-32, 34, and 36.

In one aspect, the disclosure provides a live bacterial product referred to as “27-mix” (see, FIGS. 3 and 5). As shown in FIG. 5, live bacterial product 27-mix contains 27 bacterial strains related to the following species: Collinsella aerofaciens, Bacteroides ovatus, Bacteroides thetaiotamicron, Bacteroides xyalinosolvens, Bacteroides cellulosilyticus, Bacteroides uniformis, Bacteroides vulgatus, Parabacteroides distasonis, Alistipes putredinis, Alistipes shahii, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium rectale, Anaerostipes caccae, Blautia obeum, Blautia producta, Coprococcus comes, Dorea longicatena, Agathobaculum butyriciproducens, Phascolarctobacterium faecis, Escherichia sp. 3_2_53FAA, and Fusobacterium mortiferum.

In some embodiments, the compositions comprise two or more (e.g., 2, 3, 4, 5, or more) bacterial species selected from the group consisting of Collinsella aerofaciens, Bacteroides ovatus, Bacteroides thetaiotamicron, Bacteroides xyalinosolvens, Bacteroides cellulosilyticus, Bacteroides uniformis, Bacteroides vulgatus, Parabacteroides distasonis, Alistipes putredinis, Alistipes shahii, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium rectale, Anaerostipes caccae, Blautia obeum, Blautia producta, Coprococcus comes, Dorea longicatena, Agathobaculum butyriciproducens, Phascolarctobacterium faecis, Escherichia sp. 3_2_53FAA, and Fusobacterium mortiferum.

In some embodiments, the compositions comprise 27 bacterial species: Collinsella aerofaciens, Bacteroides ovatus, Bacteroides thetaiotamicron, Bacteroides xyalinosolvens, Bacteroides cellulosilyticus, Bacteroides uniformis, Bacteroides vulgatus, Parabacteroides distasonis, Alistipes putredinis, Alistipes shahii, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium rectale, Anaerostipes caccae, Blautia obeum, Blautia producta, Coprococcus comes, Dorea longicatena, Agathobaculum butyriciproducens, Phascolarctobacterium faecis, Escherichia sp. 3_2_53FAA, and Fusobacterium mortiferum.

In some embodiments, the compositions consist of 27 bacterial species: Collinsella aerofaciens, Bacteroides ovatus, Bacteroides thetaiotamicron, Bacteroides xyalinosolvens, Bacteroides cellulosilyticus, Bacteroides uniformis, Bacteroides vulgatus, Parabacteroides distasonis, Alistipes putredinis, Alistipes shahii, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium rectale, Anaerostipes caccae, Blautia obeum, Blautia producta, Coprococcus comes, Dorea longicatena, Agathobaculum butyriciproducens, Phascolarctobacterium faecis, Escherichia sp. 3_2_53FAA, and Fusobacterium mortiferum.

In some embodiments, the compositions consist essentially of 27 bacterial species: Collinsella aerofaciens, Bacteroides ovatus, Bacteroides thetaiotamicron, Bacteroides xyalinosolvens, Bacteroides cellulosilyticus, Bacteroides uniformis, Bacteroides vulgatus, Parabacteroides distasonis, Alistipes putredinis, Alistipes shahii, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium rectale, Anaerostipes caccae, Blautia obeum, Blautia producta, Coprococcus comes, Dorea longicatena, Agathobaculum butyriciproducens, Phascolarctobacterium faecis, Escherichia sp. 3_2_53FAA, and Fusobacterium mortiferum. In some embodiments, consisting essentially of, as used herein, refers to compositions that do not include any additional therapeutically active bacterial strains.

In one aspect, the bacterial composition comprises two or more (e.g., 2, 3, 4, 5 or more) purified bacterial strains comprising 16S rDNA sequences having at least 97% sequence identity with the nucleic acid sequence selected from SEQ ID NOs: 2, 4-9, 11-15, 17-20, 22, 24-27, 29-34, and 36. In one aspect, the bacterial composition consists of two or more (e.g., 2, 3, 4, 5 or more) purified bacterial strains comprising 16S rDNA sequences having at least 97% sequence identity with the nucleic acid sequence selected from SEQ ID NOs: 2, 4-9, 11-15, 17-20, 22, 24-27, 29-34, and 36. In one aspect, the bacterial composition consists essentially of two or more (e.g., 2, 3, 4, 5 or more) purified bacterial strains comprising 16S rDNA sequences having at least 97% sequence identity with the nucleic acid sequence selected from SEQ ID NOs: 2, 4-9, 11-15, 17-20, 22, 24-27, 29-34, and 36.

In one aspect, the bacterial composition comprises 27 bacterial strains comprising 16S rDNA sequences having at least 97% sequence identity with the nucleic acid sequence selected from SEQ ID NOs: 2, 4-9, 11-15, 17-20, 22, 24-27, 29-34, and 36. In one aspect, the bacterial composition consists of 27 bacterial strains comprising 16S rDNA sequence having at least 97% sequence identity with the nucleic acid sequences selected from SEQ ID NOs: 2, 4-9, 11-15, 17-20, 22, 24-27, 29-34, and 36. In one aspect, the bacterial composition consists essentially of 27 bacterial strains comprising 16S rDNA sequences having at least 97% sequence identity with the nucleic acid sequence selected from SEQ ID NOs: 2, 4-9, 11-15, 17-20, 22, 24-27, 29-34, and 36.

In one aspect, the disclosure provides a live bacterial product referred to as “23-mix” (see, FIGS. 3 and 5). As shown in FIG. 5, live bacterial product 23-mix contains 23 bacterial strains related to the following species: Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, Escherichia sp. 3_2_53FAA, and Fusobacterium mortiferum.

In some embodiments, the compositions comprise two or more (e.g., 2, 3, 4, 5, or more) bacterial species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, Escherichia sp. 3_2_53FAA, and Fusobacterium mortiferum.

In some embodiments, the compositions comprise 23 bacterial species: Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, Escherichia sp. 3_2_53FAA, and Fusobacterium mortiferum.

In some embodiments, the compositions consist of 23 bacterial species: Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, Escherichia sp. 3_2_53FAA, and Fusobacterium mortiferum.

In some embodiments, the compositions consist essentially of 23 bacterial species: Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, Escherichia sp. 3_2_53FAA, and Fusobacterium mortiferum. In some embodiments, consisting essentially of, as used herein, refers to compositions that do not include any additional therapeutically active bacterial strains.

In one aspect, the bacterial composition comprises two or more (e.g., 2, 3, 4, 5 or more) purified bacterial strains comprising 16S rDNA sequences having at least 97% sequence identity with the nucleic acid sequence selected from SEQ ID NOs: 1-7, 11-15, 17-20, 24-26, 28, 29, 34, and 36. In one aspect, the bacterial composition consists of two or more (e.g., 2, 3, 4, 5 or more) purified bacterial strains comprising 16S rDNA sequences having at least 97% sequence identity with the nucleic acid sequence selected from SEQ ID NOs: 1-7, 11-15, 17-20, 24-26, 28, 29, 34, and 36. In one aspect, the bacterial composition consists essentially of two or more (e.g., 2, 3, 4, 5 or more) purified bacterial strains comprising 16S rDNA sequences having at least 97% sequence identity with the nucleic acid sequence selected from SEQ ID NOs: 1-7, 11-15, 17-20, 24-26, 28, 29, 34, and 36.

In one aspect, the bacterial composition comprises 23 bacterial strains comprising 16S rDNA sequences having at least 97% sequence identity with the nucleic acid sequence selected from SEQ ID NOs: 1-7, 11-15, 17-20, 24-26, 28, 29, 34, and 36. In one aspect, the bacterial composition consists of 23 bacterial strains comprising 16S rDNA sequences having at least 97% sequence identity with the nucleic acid sequence selected from SEQ ID NOs: 1-7, 11-15, 17-20, 24-26, 28, 29, 34, and 36. In one aspect, the bacterial composition consists essentially of 23 bacterial strain comprising 16S rDNA sequences having at least 97% sequence identity with the nucleic acid sequence selected from SEQ ID NOs: 1-7, 11-15, 17-20, 24-26, 28, 29, 34, and 36.

In one aspect, the disclosure provides compositions comprising purified bacterial strains related to the following species Clostridium bolteae, Anaerotruncus colihominis, Sellimonas intestinalis (Ruminococcus torques), Clostridium symbiosum, Blautia producta, Dorea longicatena, Erysipelotrichaceae bacterium, Flavinofractor plautii (Subdoligranulum spp), a purified bacterial strain belonging to Escherichia species (such as E. coli, e.g., Escherichia coli sp. 3_2_53FAA), and a purified bacterial strain belonging to Fusobacterium species (e.g. Fusobacterium mortiferum). The compositions comprising bacterial strains Clostridium bolteae, Anaerotruncus colihominis, Sellimonas intestinalis, Clostridium symbiosum, Blautia producta, Dorea longicatena, Erysipelotrichaceae bacterium, and Flavinofractor plautii are described for instance in PCT Publication No.: WO 2017/218680, which is incorporated herein by reference in its entirety. The strains are also depicted in Tables 1 and 2. It should be appreciated that alternative strain names, e.g., as depicted in Tables 1 and 2, may be used as well. The composition comprising bacterial strains Clostridium bolteae, Anaerotruncus colihominis, Sellimonas intestinalis, Clostridium symbiosum, Blautia producta, Dorea longicatena, Erysipelotrichaceae bacterium, and Flavinofractor plautii is also referred to as VE303. The sequences of the bacterial strains of VE303 are also presented in PCT Publication No. WO 2017/218680, e.g., Table 3.

In one aspect, the disclosure provides compositions consisting of purified bacterial strains related to the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, a purified bacterial strain belonging to Escherichia species (such as E. coli, e.g., Escherichia coli sp. 3_2_53FAA), and a purified bacterial strain belonging to Fusobacterium species (e.g. Fusobacterium mortiferum).

In one aspect, the disclosure provides compositions consisting essentially of purified bacterial strains related to the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, a purified bacterial strain belonging to Escherichia species (such as E. coli, e.g., Escherichia coli sp. 3_2_53FAA), and a purified bacterial strain belonging to Fusobacterium species (e.g., Fusobacterium mortiferum).

In one aspect, the disclosure provides compositions comprising purified bacterial strains comprising 16S rDNA sequences having at least 97% sequence identity with sequences provided in PCT Publication No. WO 2017/218680, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain comprising a 16S rDNA sequence having at least 97% sequence identity with the nucleic acid sequence set forth as SEQ ID NO: 36, and a purified bacterial strain comprising a 16S rDNA sequence having at least 97% sequence identity with the nucleic acid sequence set forth as SEQ ID NO: 34.

In one aspect, the disclosure provides compositions consisting of purified bacterial strains comprising 16S rDNA sequences having at least 97% sequence identity with sequences provided in PCT Publication No. WO 2017/218680, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain comprising a 16S rDNA sequence having at least 97% sequence identity with the nucleic acid sequence set forth as SEQ ID NO: 36, and a purified bacterial strain comprising a 16S rDNA sequence having at least 97% sequence identity with the nucleic acid sequence set forth as SEQ ID NO: 34.

In one aspect, the disclosure provides compositions consisting essentially of purified bacterial strains comprising 16S rDNA sequences having at least 97% sequence identity with sequences provided in PCT Publication No. WO 2017/218680, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain comprising a 16S rDNA sequence having at least 97% sequence identity with the nucleic acid sequence set forth as SEQ ID NO: 36, and a purified bacterial strain comprising a 16S rDNA sequence having at least 97% sequence identity with the nucleic acid sequence set forth as SEQ ID NO: 34.

In one aspect, the disclosure provides compositions comprising purified bacterial strains related to the following species Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In one aspect, the disclosure provides compositions consisting of purified bacterial strains related to the following species Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In one aspect, the disclosure provides compositions consisting essentially of purified bacterial strains related to the following species Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In one aspect, the disclosure provides compositions comprising purified bacterial strains comprising 16S rDNA sequences having at least 97% sequence identity with sequences provided in PCT Publication No. WO 2017/218680, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and purified bacterial strains comprising 16S rDNA sequences having at least 97% sequence identity with the nucleic acid sequence set forth as SEQ ID NOs: 3, 8-14, 16, 18, 19, 27, 28, 32, 35, and 36.

In one aspect, the disclosure provides compositions comprising purified bacterial strains related to the following species Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Clostridium clostridioforme, Blautia obeum, Clostridium innocuum, Agathobaculum butyriciproducens, Erysipelotrichaceae bacterium, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Clostridium innocuum, and Flavonifractor plautii.

In one aspect, the disclosure provides compositions consisting of purified bacterial strains related to the following species Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Clostridium clostridioforme, Blautia obeum, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In one aspect, the disclosure provides compositions consisting essentially of purified bacterial strains related to the following species Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Clostridium clostridioforme, Blautia obeum, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In one aspect, the disclosure provides compositions comprising purified bacterial strains comprising 16S rDNA sequences having at least 97% sequence identity with sequences provided in PCT Publication No. WO 2017/218680, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and purified bacterial strains comprising 16S rDNA sequences having at least 97% sequence identity with the nucleic acid sequence set forth as SEQ ID NOs: 3, 8-16, 18, 19, 25, 27, 28, 30, 32, and 34-36.

In one aspect, the disclosure provides compositions comprising purified bacterial strains related to the following species Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Blautia obeum, Clostridium innocuum, Agathobaculum butyriciproducens, Clostridium innocuum, Dorea longicatena, Collinsella aerofaciens, Eubacterium hallii, Bacteroides faecis, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In one aspect, the disclosure provides compositions consisting of purified bacterial strains related to the following species Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Blautia obeum, Clostridium innocuum, Agathobaculum butyriciproducens, Clostridium innocuum, Dorea longicatena, Collinsella aerofaciens, Eubacterium hallii, Bacteroides faecis, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In one aspect, the disclosure provides compositions consisting essentially of purified bacterial strains related to the following species Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Blautia obeum, Clostridium innocuum, Agathobaculum butyriciproducens, Clostridium innocuum, Dorea longicatena, Collinsella aerofaciens, Eubacterium hallii, Bacteroides faecis, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In one aspect, the disclosure provides compositions comprising purified bacterial strains comprising 16S rDNA sequences having at least 97% sequence identity with sequences provided in PCT Publication No. WO 2017/218680, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and purified bacterial strains comprising 16S rDNA sequences having at least 97% sequence identity with the nucleic acid sequence set forth as SEQ ID NOs: 3, 4, 8-16, 18, 19, 21, 25, 27, 28, 30, and 32-36.

In one aspect, the disclosure provides compositions comprising purified bacterial strains related to the following species Blautia producta, Bifidobacterium longum, Bifidobacterium adolescentis, Paeniclostridium sordellii, Bifidobacterium pseudocatenulatum, Erysipelatoclostridium ramosum, Escherichia coli, Paraclostridium bifermentans, Clostridium citroniae, Anaerostipes caccae, Fusobacterium mortiferum, Blautia obeum, Clostridium innocuum, Agathobaculum butyriciproducens, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In one aspect, the disclosure provides compositions consisting of purified bacterial strains related to the following species Blautia producta, Bifidobacterium longum, Bifidobacterium adolescentis, Paeniclostridium sordellii, Bifidobacterium pseudocatenulatum, Erysipelatoclostridium ramosum, Escherichia coli, Paraclostridium bifermentans, Clostridium citroniae, Anaerostipes caccae, Fusobacterium mortiferum, Blautia obeum, Clostridium innocuum, Agathobaculum butyriciproducens, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In one aspect, the disclosure provides compositions consisting essentially of purified bacterial strains related to the following species Blautia producta, Bifidobacterium longum, Bifidobacterium adolescentis, Paeniclostridium sordellii, Bifidobacterium pseudocatenulatum, Erysipelatoclostridium ramosum, Escherichia coli, Paraclostridium bifermentans, Clostridium citroniae, Anaerostipes caccae, Fusobacterium mortiferum, Blautia obeum, Clostridium innocuum, Agathobaculum butyriciproducens, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

In one aspect, the disclosure provides compositions comprising purified bacterial strains comprising 16S rDNA sequences having at least 97% sequence identity with sequences provided in PCT Publication No. WO 2017/218680, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and purified bacterial strains comprising 16S rDNA sequences having at least 97% sequence identity with the nucleic acid sequence set forth as SEQ ID NOs: 4, 8-12, 15, 16, 19, 21, 27, 30, and 32-36.

TABLE 1
Bacterial strains of composition VE303

| | Closest species based on | | | |
|---|---|---|---|---|
| Closest species based on | Consensus SEQ ID # of 16S | Closest species based on | | |
| Sanger sequencing of 16S | region as compared with 16S | WGS compared versus | Additional closely | Clostridium |
| region | database | WG databases | related sequences | cluster |
| Clostridium bolteae | Clostridium bolteae | Clostridium bolteae 90A9 | | XIVa |
| Anaerotruncus colihominis | Anaerotruncus colihominis | Anaerotruncus colihominis | | IV |
| | | DSM 17241 | | |
| Eubacterium fissicatena | Dracourtella massiliensis | Dracourtella massiliensis | Ruminococcus | XIVa |
| | | GDI | torques; Sellimonas | |
| | | | intestinalis | |
| Clostridium symbiosum | Clostridium symbiosum | Clostridium symbiosum | | XIVa |
| | | WAL-14163 | | |
| Blautia producta | Blautia producta | Clostridium bacterium | Blautia product | XIVa |
| | | UC5.1-1D4 | ATCC 27340 | |
| Dorea longicatena | Dorea longicatena | Dorea longicatena CAG:42 | | XIVa |
| Clostridium innocuum | Clostridium innocuum | Erysipelotrichaceae | | XVII |
| | | bacterium 21_3 | | |
| Flavinofractor plautii | Flavinofractor plautii | Clostridium orbiscindens | Subdolinogranulum | IV |
| | | l_3_50AFAA | | |

TABLE 2
VE303 - Bacterial strains of VE303 and sequence identity to nearest relative

| | | | Nearest Relative | | |
|---|---|---|---|---|---|
| | Clostridium | | (NCBI | FastANI | Genome |
| Strain | Cluster | Nearest Relative (Scientific name) | Tax ID) | Identity (%) | Length (bp) |
| VE303-01 | XIVa | Clostridium bolteae 90A9 | 997894 | 99.7576 | 6071159 |
| VE303-02 | IV | Anaerotruncus colihominis DSM 17241 | 445972 | 99.155 | 3538351 |
| VE303-03 | XIVa | Sellimonas intestinalis | 1653434 | 99.4586 | 3195208 |
| VE303-04 | XIVa | [Clostridium] symbiosum WAL-14163 | 742740 | 99.0591 | 5600243 |
| VE303-05 | XIVa | Clostridia bacterium UC5.1-1D4 | 1697784 | 98.6518 | 5944559 |
| VE303-06 | XIVa | Dorea longicatena CAG:42 | 1263074 | 98.4309 | 3439497 |
| VE303-07 | XVII | Erysipelotrichaceae bacterium 21_3 | 658657 | 99.4812 | 4386760 |
| VE303-08 | IV | Flavonifractor plautii 1_3_50AFAA | 742738 | 98.1231 | 4771756 |

In one aspect, the disclosure provides compositions comprising purified bacterial strains related to the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain belonging to Escherichia species (such as E. coli, e.g., Escherichia coli sp. 3_2_53FAA). In one aspect, the disclosure provides compositions consisting of purified bacterial strains related to the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain belonging to Escherichia species (such as E. coli, e.g., Escherichia coli sp. 3_2_53FAA). In one aspect, the disclosure provides compositions consisting essentially of purified bacterial strains related to the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain belonging to Escherichia species (such as E. coli, e.g., Escherichia coli sp. 3_2_53FAA).

In one aspect, the disclosure provides compositions comprising purified bacterial strains comprising 16S rDNA sequences having at least 97% sequence identity with sequences provided in PCT Publication No. WO 2017/218680, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain comprising a 16S rDNA sequence having at least 97% sequence identity with the nucleic acid sequence set forth as SEQ ID NO: 36.

In one aspect, the disclosure provides compositions consisting of purified bacterial strains comprising 16S rDNA sequences having at least 97% sequence identity with sequences provided in PCT Publication No. WO 2017/218680, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain comprising a 16S rDNA sequence having at least 97% sequence identity with the nucleic acid sequence set forth as SEQ ID NO: 36.

In one aspect, the disclosure provides compositions consisting of purified bacterial strains comprising 16S rDNA sequences having at least 97% sequence identity with sequences provided in PCT Publication No. WO 2017/218680, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain comprising a 16S rDNA sequence having at least 97% sequence identity with the nucleic acid sequence set forth as SEQ ID NO: 36.

In one aspect, the disclosure provides compositions comprising purified bacterial strains related to the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain belonging to Fusobacterium species (e.g., Fusobacterium mortiferum). In one aspect, the disclosure provides compositions consisting of purified bacterial strains related to the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain belonging Fusobacterium species (e.g. Fusobacterium mortiferum). In one aspect, the disclosure provides compositions consisting essentially of purified bacterial strains related to the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain belonging to Fusobacterium species (e.g. Fusobacterium mortiferum).

In one aspect, the disclosure provides compositions comprising purified bacterial strains comprising 16S rDNA sequences having at least 97% sequence identity with sequences provided in PCT Publication No. WO 2017/218680, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain comprising a 16S rDNA sequence having at least 97% sequence identity with the nucleic acid sequence set forth as SEQ ID NO: 34.

In one aspect, the disclosure provides compositions comprising purified bacterial strains comprising 16S rDNA sequences having at least 97% sequence identity with sequences provided in PCT Publication No. WO 2017/218680, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain comprising a 16S rDNA sequence having at least 97% sequence identity with the nucleic acid sequence set forth as SEQ ID NO: 34.

In one aspect, the disclosure provides compositions comprising purified bacterial strains comprising 16S rDNA sequences having at least 97% sequence identity with sequences provided in PCT Publication No. WO 2017/218680, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain comprising a 16S rDNA sequence having at least 97% sequence identity with the nucleic acid sequence set forth as SEQ ID NO: 34.

In one aspect, the disclosure provides compositions comprising purified bacterial strains related to the following species Clostridium bolteae, Anaerotruncus colihominis, Ruminococcus torques, Clostridium symbiosum, Dorea longicatena, Erysipelotrichaceae bacterium, Subdoligranulum spp, a purified bacterial strain belonging to Escherichia species (such as E. coli, e.g., Escherichia coli sp. 3_2_53FAA), and a purified bacterial strain belonging to Fusobacterium species (e.g., Fusobacterium mortiferum). In one aspect, the disclosure provides compositions consisting of purified bacterial strains related to the following species Clostridium bolteae, Anaerotruncus colihominis, Ruminococcus torques, Clostridium symbiosum, Dorea longicatena, Erysipelotrichaceae bacterium, Subdoligranulum spp, a purified bacterial strain belonging to Escherichia species (such as E. coli, e.g., Escherichia coli sp. 3_2_53FAA), and a purified bacterial strain belonging to Fusobacterium species (e.g., Fusobacterium mortiferum). In one aspect, the disclosure provides compositions consisting essentially of purified bacterial strains related to the following species Clostridium bolteae, Anaerotruncus colihominis, Ruminococcus torques, Clostridium symbiosum, Dorea longicatena, Erysipelotrichaceae bacterium, Subdoligranulum spp, a purified bacterial strain belonging to Escherichia species (such as E. coli, e.g., Escherichia coli sp. 3_2_53FAA), and a purified bacterial strain belonging to Fusobacterium species (e.g., Fusobacterium mortiferum). In one aspect, the disclosure provides compositions comprising purified bacterial strains related to the following species Clostridium bolteae, Anaerotruncus colihominis, Ruminococcus torques, Clostridium symbiosum, Dorea longicatena, Erysipelotrichaceae bacterium, Subdoligranulum spp, and a purified bacterial strain belonging to Escherichia species (such as E. coli, e.g., Escherichia coli sp. 3_2_53FAA). In one aspect, the disclosure provides compositions consisting of purified bacterial strains related to the following species Clostridium bolteae, Anaerotruncus colihominis, Ruminococcus torques, Clostridium symbiosum, Dorea longicatena, Erysipelotrichaceae bacterium, Subdoligranulum spp, and a purified bacterial strain belonging to Escherichia species (such as E. coli, e.g., Escherichia coli sp. 3_2_53FAA). In one aspect, the disclosure provides compositions consisting essentially of purified bacterial strains related to the following species Clostridium bolteae, Anaerotruncus colihominis, Ruminococcus torques, Clostridium symbiosum, Dorea longicatena, Erysipelotrichaceae bacterium, Subdoligranulum spp, and a purified bacterial strain belonging to Escherichia species (such as E. coli, e.g., Escherichia coli sp. 3_2_53FAA).

In one aspect, the disclosure provides compositions comprising purified bacterial strains related to the following species Clostridium bolteae, Anaerotruncus colihominis, Ruminococcus torques, Clostridium symbiosum, Dorea longicatena, Erysipelotrichaceae bacterium, Subdoligranulum spp, and a purified bacterial strain belonging to Fusobacterium species (e.g., Fusobacterium mortiferum). In one aspect, the disclosure provides compositions consisting of purified bacterial strains related to the following species Clostridium bolteae, Anaerotruncus colihominis, Ruminococcus torques, Clostridium symbiosum, Dorea longicatena, Erysipelotrichaceae bacterium, Subdoligranulum spp, and a purified bacterial strain belonging to Fusobacterium species (e.g., Fusobacterium mortiferum). In one aspect, the disclosure provides compositions consisting essentially of purified bacterial strains related to the following species Clostridium bolteae, Anaerotruncus colihominis, Ruminococcus torques, Clostridium symbiosum, Dorea longicatena, Erysipelotrichaceae bacterium, Subdoligranulum spp, and a purified bacterial strain belonging to Fusobacterium species (e.g., Fusobacterium mortiferum).

In one aspect, the disclosure provides compositions comprising purified bacterial strains related to the following species Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), a purified bacterial strain belonging to Escherichia species (such as E. coli, e.g., Escherichia coli sp. 3_2_53FAA), and a purified bacterial strain belonging to Fusobacterium species (e.g., Fusobacterium mortiferum).

Compositions comprising bacterial strains Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), and Lachnospiraceae bacterium A4 (Lachnospiraceae bacterium 3_1_57FAA_CT1) are described, for example, in PCT Publication No. WO 2013/080561, which is incorporated herein by reference in its entirety. Such bacterial compositions are also described, for example in Atarashi et al. Science (2011) 331(6015): 337-341 and Atarashi et al. Nature (2013) 500(7361): 232-236.

Compositions comprising bacterial strains Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), and Lachnospiraceae bacterium A4 (Lachnospiraceae bacterium 3_1_57FAA_CT1) may also be referred to as VE202. Sequences of the bacterial strains of VE202 are also presented in PCT Publication No. WO 2013/080561. It should be appreciated that alternative names of the bacterial strains may be used.

In one aspect, the disclosure provides compositions consisting of purified bacterial strains related to the following species Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), a purified bacterial strain belonging to Escherichia species (such as E. coli, e.g., Escherichia coli sp. 3_2_53FAA), and a purified bacterial strain belonging to Fusobacterium species (e.g., Fusobacterium mortiferum).

In one aspect, the disclosure provides compositions consisting essentially of purified bacterial strains related to the following species Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), a purified bacterial strain belonging to Escherichia species (such as E. coli, e.g., Escherichia coli sp. 3_2_53FAA), and a purified bacterial strain belonging to Fusobacterium species (e.g., Fusobacterium mortiferum).

In one aspect, the disclosure provides compositions comprising purified bacterial strains comprising 16S rDNA sequences having at least 97% sequence identity with sequences provided in PCT Publication No. WO 2013/080561, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain comprising a 16S rDNA sequence having at least 97% sequence identity with the nucleic acid sequence set forth as SEQ ID NO: 34, and a purified bacterial strain comprising a 16S rDNA sequence having at least 97% sequence identity with the nucleic acid sequence set forth as SEQ ID NO: 36.

In one aspect, the disclosure provides compositions consisting of purified bacterial strains comprising 16S rDNA sequences having at least 97% sequence identity with sequences provided in PCT Publication No. WO 2013/080561, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain comprising a 16S rDNA sequence having at least 97% sequence identity with the nucleic acid sequence set forth as SEQ ID NO: 34, and a purified bacterial strain comprising a 16S rDNA sequence having at least 97% sequence identity with the nucleic acid sequence set forth as SEQ ID NO: 36.

In one aspect, the disclosure provides compositions consisting essentially of purified bacterial strains comprising 16S rDNA sequences having at least 97% sequence identity with sequences provided in PCT Publication No. WO 2013/080561, for the following species Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain comprising a 16S rDNA sequence having at least 97% sequence identity with the nucleic acid sequence set forth as SEQ ID NO: 34, and a purified bacterial strain comprising a 16S rDNA sequence having at least 97% sequence identity with the nucleic acid sequence set forth as SEQ ID NO: 36.

In one aspect, the disclosure provides compositions comprising purified bacterial strains related to the following species Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), and a purified bacterial strain belonging to Escherichia species (such as E. coli, e.g., Escherichia coli sp. 3_2_53FAA).

In one aspect, the disclosure provides compositions consisting of purified bacterial strains related to the following species Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), and a purified bacterial strain belonging to Escherichia species (such as E. coli, e.g., Escherichia coli sp. 3_2_53FAA).

In one aspect, the disclosure provides compositions consisting essentially of purified bacterial strains related to the following species Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), and a purified bacterial strain belonging to Escherichia species (such as E. coli, e.g., Escherichia coli sp. 3_2_53FAA).

In one aspect, the disclosure provides compositions comprising purified bacterial strains comprising 16S rDNA sequences having at least 97% sequence identity with sequences provided in PCT Publication No. WO 2013/080561, and a purified bacterial strain comprising a 16S rDNA sequence having at least 97% sequence identity with the nucleic acid sequence set forth as SEQ ID NO: 34.

In one aspect, the disclosure provides compositions consisting of purified bacterial strains comprising 16S rDNA sequences having at least 97% sequence identity with sequences provided in PCT Publication No. WO 2013/080561, and a purified bacterial strain comprising a 16S rDNA sequence having at least 97% sequence identity with the nucleic acid sequence set forth as SEQ ID NO: 34.

In one aspect, the disclosure provides compositions consisting essentially of purified bacterial strains comprising 16S rDNA sequences having at least 97% sequence identity with sequences provided in PCT Publication No. WO 2013/080561, and a purified bacterial strain comprising a 16S rDNA sequence having at least 97% sequence identity with the nucleic acid sequence set forth as SEQ ID NO: 34.

In one aspect, the disclosure provides compositions comprising purified bacterial strains related to the following species Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), and a purified bacterial strain belonging to Fusobacterium species (e.g., Fusobacterium mortiferum).

In one aspect, the disclosure provides compositions consisting of purified bacterial strains related to the following species Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), and a purified bacterial strain belonging to Fusobacterium species (e.g., Fusobacterium mortiferum).

In one aspect, the disclosure provides compositions consisting essentially of purified bacterial strains related to the following species Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), and a purified bacterial strain belonging to Fusobacterium species (e.g., Fusobacterium mortiferum).

In one aspect, the disclosure provides compositions comprising purified bacterial strains comprising 16S rDNA sequences having at least 97% sequence identity with sequences provided in PCT Publication No. WO 2013/080561, and a purified bacterial strain comprising a 16S rDNA sequence having at least 97% sequence identity with the nucleic acid sequence set forth as SEQ ID NO: 36.

In one aspect, the disclosure provides compositions consisting of purified bacterial strains comprising 16S rDNA sequences having at least 97% sequence identity with sequences provided in PCT Publication No. WO 2013/080561, and a purified bacterial strain comprising a 16S rDNA sequence having at least 97% sequence identity with the nucleic acid sequence set forth as SEQ ID NO: 36.

In one aspect, the disclosure provides compositions consisting essentially of purified bacterial strains comprising 16S rDNA sequences having at least 97% sequence identity with sequences provided in PCT Publication No. WO 2013/080561, and a purified bacterial strain comprising a 16S rDNA sequence having at least 97% sequence identity with the nucleic acid sequence set forth as SEQ ID NO: 36.

In one aspect, the disclosure provides compositions comprising purified bacterial strains related to the following species Clostridium bolteae, Anaerotruncus colihominis, Ruminococcus torques, Clostridium symbiosum, Blautia producta, Erysipelotrichaceae bacterium, Subdoligranulum spp, a purified bacterial strain belonging to Escherichia species (such as E. coli, e.g., Escherichia coli sp. 3_2_53FAA), and a purified bacterial strain belonging to Fusobacterium species (e.g., Fusobacterium mortiferum). The compositions comprising bacterial strains Clostridium bolteae, Anaerotruncus colihominis, Ruminococcus torques, Clostridium symbiosum, Blautia producta, Erysipelotrichaceae bacterium, and Subdoligranulum spp, are described, for example, in PCT Publication No. WO 2019/094837, which is incorporated by reference herein in its entirety. Sequences of the bacterial strains are also presented in PCT Publication No. WO 2019/094837, e.g., Table 1. It should be appreciated that alternative names may be used to refer to the bacterial strains.

In one aspect, the disclosure provides compositions consisting of purified bacterial strains related to the following species Clostridium bolteae, Anaerotruncus colihominis, Ruminococcus torques, Clostridium symbiosum, Blautia producta, Erysipelotrichaceae bacterium, Subdoligranulum spp, a purified bacterial strain belonging to Escherichia species (such as E. coli, e.g., Escherichia coli sp. 3_2_53FAA), and a purified bacterial strain belonging to Fusobacterium species (e.g., Fusobacterium mortiferum). In one aspect, the disclosure provides compositions consisting essentially of purified bacterial strains related to the following species Clostridium bolteae, Anaerotruncus colihominis, Ruminococcus torques, Clostridium symbiosum, Blautia producta, Erysipelotrichaceae bacterium, Subdoligranulum spp, a purified bacterial strain belonging to Escherichia species (such as E. coli, e.g., Escherichia coli sp. 3_2_53FAA), and a purified bacterial strain belonging to Fusobacterium species (e.g., Fusobacterium mortiferum).

In one aspect, the disclosure provides compositions comprising purified bacterial strains comprising 16S rDNA sequences having at least 97% sequence identity with sequences provided in PCT Publication No. WO 2019/094837, for strains Clostridium bolteae, Anaerotruncus colihominis, Ruminococcus torques, Clostridium symbiosum, Blautia producta, Erysipelotrichaceae bacterium, and Subdoligranulum spp, and a purified bacterial strain comprising a 16S rDNA sequence having at least 97% sequence identity with the nucleic acid sequence set forth as SEQ ID NO: 36, and a purified bacterial strain comprising a 16S rDNA sequence having at least 97% sequence identity with the nucleic acid sequence set forth as SEQ ID NO: 34.

In one aspect, the disclosure provides compositions consisting of purified bacterial strains comprising 16S rDNA sequences having at least 97% sequence identity with sequences provided in PCT Publication No. WO 2019/094837, for strains Clostridium bolteae, Anaerotruncus colihominis, Ruminococcus torques, Clostridium symbiosum, Blautia producta, Erysipelotrichaceae bacterium, and Subdoligranulum spp, and a purified bacterial strain comprising a 16S rDNA sequence having at least 97% sequence identity with the nucleic acid sequence set forth as SEQ ID NO: 36, and a purified bacterial strain comprising a 16S rDNA sequence having at least 97% sequence identity with the nucleic acid sequence set forth as SEQ ID NO: 34.

In one aspect, the disclosure provides compositions consisting essentially of purified bacterial strains comprising 16S rDNA sequences having at least 97% sequence identity with sequences provided in PCT Publication No. WO 2019/094837, for strains Clostridium bolteae, Anaerotruncus colihominis, Ruminococcus torques, Clostridium symbiosum, Blautia producta, Erysipelotrichaceae bacterium, and Subdoligranulum spp, and a purified bacterial strain comprising a 16S rDNA sequence having at least 97% sequence identity with the nucleic acid sequence set forth as SEQ ID NO: 36, a purified bacterial strain comprising a 16S rDNA sequence having at least 97% sequence identity with the nucleic acid sequence set forth as SEQ ID NO: 34.

In one aspect, the disclosure provides compositions comprising purified bacterial strains related to the following species Clostridium bolteae, Anaerotruncus colihominis, Ruminococcus torques, Clostridium symbiosum, Blautia producta, Erysipelotrichaceae bacterium, Subdoligranulum spp, and a purified bacterial strain belonging to Escherichia species (such as E. coli, e.g., Escherichia coli sp. 3_2_53FAA). In one aspect, the disclosure provides compositions consisting of purified bacterial strains related to the following species Clostridium bolteae, Anaerotruncus colihominis, Ruminococcus torques, Clostridium symbiosum, Blautia producta, Erysipelotrichaceae bacterium, Subdoligranulum spp, and a purified bacterial strain belonging to Escherichia species (such as E. coli, e.g., Escherichia coli sp. 3_2_53FAA). In one aspect, the disclosure provides compositions consisting essentially of purified bacterial strains related to the following species Clostridium bolteae, Anaerotruncus colihominis, Ruminococcus torques, Clostridium symbiosum, Blautia producta, Erysipelotrichaceae bacterium, Subdoligranulum spp, and a purified bacterial strain belonging to Escherichia species (such as E. coli, e.g., Escherichia coli sp. 3_2_53FAA).

In one aspect, the disclosure provides compositions comprising purified bacterial strains comprising 16S rDNA sequences having at least 97% sequence identity with sequences provided in PCT Publication No. WO 2019/094837, for strains Clostridium bolteae, Anaerotruncus colihominis, Ruminococcus torques, Clostridium symbiosum, Blautia producta, Erysipelotrichaceae bacterium, and Subdoligranulum spp, and a purified bacterial strain comprising a 16S rDNA sequence having at least 97% sequence identity with the nucleic acid sequence set forth as SEQ ID NO: 36.

In one aspect, the disclosure provides compositions consisting of purified bacterial strains comprising 16S rDNA sequences having at least 97% sequence identity with sequences provided in PCT Publication No. WO 2019/094837, for strains Clostridium bolteae, Anaerotruncus colihominis, Ruminococcus torques, Clostridium symbiosum, Blautia producta, Erysipelotrichaceae bacterium, and Subdoligranulum spp, and a purified bacterial strain comprising a 16S rDNA sequence having at least 97% sequence identity with the nucleic acid sequence set forth as SEQ ID NO: 36.

In one aspect, the disclosure provides compositions consisting essentially of purified bacterial strains comprising 16S rDNA sequences having at least 97% sequence identity with sequences provided in PCT Publication No. WO 2019/094837, for strains Clostridium bolteae, Anaerotruncus colihominis, Ruminococcus torques, Clostridium symbiosum, Blautia producta, Erysipelotrichaceae bacterium, and Subdoligranulum spp, and a purified bacterial strain comprising a 16S rDNA sequence having at least 97% sequence identity with the nucleic acid sequence set forth as SEQ ID NO: 36.

In one aspect, the disclosure provides compositions comprising purified bacterial strains related to the following species Clostridium bolteae, Anaerotruncus colihominis, Ruminococcus torques, Clostridium symbiosum, Blautia producta, Erysipelotrichaceae bacterium, Subdoligranulum spp, and a purified bacterial strain belonging to Fusobacterium species (e.g., Fusobacterium mortiferum). In one aspect, the disclosure provides compositions consisting of purified bacterial strains related to the following species Clostridium bolteae, Anaerotruncus colihominis, Ruminococcus torques, Clostridium symbiosum, Blautia producta, Erysipelotrichaceae bacterium, Subdoligranulum spp, and a purified bacterial strain belonging to Fusobacterium species (e.g., Fusobacterium mortiferum). In one aspect, the disclosure provides compositions consisting essentially of purified bacterial strains related to the following species Clostridium bolteae, Anaerotruncus colihominis, Ruminococcus torques, Clostridium symbiosum, Blautia producta Erysipelotrichaceae bacterium, Subdoligranulum spp, and a purified bacterial strain belonging to Fusobacterium species (e.g., Fusobacterium mortiferum).

In one aspect, the disclosure provides compositions comprising purified bacterial strains comprising 16S rDNA sequences having at least 97% sequence identity with sequences provided in PCT Publication No. WO 2013/080561, and a purified bacterial strain comprising a 16S rDNA sequence having at least 97% sequence identity with the nucleic acid sequence set forth as SEQ ID NO: 34.

In one aspect, the disclosure provides compositions consisting of purified bacterial strains comprising 16S rDNA sequences having at least 97% sequence identity with sequences provided in PCT Publication No. WO 2013/080561, and a purified bacterial strain comprising a 16S rDNA sequence having at least 97% sequence identity with the nucleic acid sequence set forth as SEQ ID NO: 34.

In one aspect, the disclosure provides compositions consisting essentially of purified bacterial strains comprising 16S rDNA sequences having at least 97% sequence identity with sequences provided in PCT Publication No. WO 2013/080561, and a purified bacterial strain comprising a 16S rDNA sequence having at least 97% sequence identity with the nucleic acid sequence set forth as SEQ ID NO: 34.

In some embodiments, the compositions described herein comprise a bacterial strain belonging to Escherichia species. Escherichia are Gram-negative, non-spore-forming bacteria that belong to the family Enterobacteriaceae. Many Escherichia species are commensal members of the intestinal microbiome. In some embodiments, the Escherichia species is Escherichia coli, Escherichia albertii, Escherichia fergusonii, Escherichia hermannii, Escherichia marmotae, or Escherichia vulneris. In some embodiments, the Escherichia species is Escherichia coli.

A non-limiting example of an Escherichia coli strain is, without limitation, Escherichia sp. 3_2_53FAA.

In some embodiments, the Escherichia species comprises genes that produce one or more bacteriocin. Bacteriocins are antibiotic compounds produced by bacteria that kill bacteria that are closely related to the bacteriocin-producing bacteria (e.g., closely related to Escherichia coli). In some embodiments, the bacteriocins are colicins, which kill bacteria that are closely related to Escherichia coli. In general, colicins are encoded on plasmids within an operon structure and typically involves three colicin-related genes: a toxin-encoding gene, an immunity gene that protects the producing cell, and a lysis gene. In some embodiments, the colicin plasmid is a high-copy-number plasmid or a low-copy-number plasmid. In some embodiments, the colicin produced by an Escherichia coli strain is colicin A, B, E1, E2, E7, Ia, Ib, N, M.

In some embodiments, the Escherichia coli does not have genes that are associated with genetic transfer (e.g., transformation). In general, many Escherichia coli strains are able to uptake exogenous DNA (e.g., drug resistance plasmids) from outside the cell through a protein complex in the cell membrane. Bacterial gene transfer may promote the phenomena such as acquired antibiotic resistance, nutrient-deprivation resistance, and temperature-resistance. In some embodiments, the Escherichia coli do not have one or more genes that encode one or more proteins associated with uptake of an exogenous plasmid (e.g., drug resistant plasmid. In some embodiments, the Escherichia coli do not have one or more genes that encode one or more proteins associated with uptake of an exogenous plasmid, such as one or more genes encoding SXY CRP, YHIR, YHGH, YRFB, YRFA, YHGI, HOFQ, YRFC, and/or YRFD.

In some embodiments, the compositions described herein comprise a bacterial strain belonging to Fusobacterium species. In some embodiments, the compositions provided herein comprise bacteria belonging to the phylum Fusobacteria. In some embodiments, the Fusobacterium species is Fusobacterium canifelinum, Fusobacterium equinum, Fusobacterium gonidiaformans, Fusobacterium mortiferum, Fusobacterium naviforme, Fusobacterium necrogenes, Fusobacterium necrophorum, Fusobacterium nucleatum, Fusobacterium perfoetens, Fusobacterium periodonticum, Fusobacterium prausnitzii, Fusobacterium russii, Fusobacterium simiae, Fusobacterium ulcerans, or Fusobacterium varium. In some embodiments, the Fusobacterium species is Fusobacterium mortiferum.

In some embodiments, the compositions disclosed herein comprise two or more bacterial strains. In some embodiments, the compositions described herein comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38 or more bacterial strains (e.g., purified bacterial strains).

As will be appreciated by one of skill in the art, any one or more bacterial strain identified as having pathogen-antagonizing activity may be included in any of the compositions described herein.

Aspects of the disclosure relate to bacterial strains with 16S rDNA sequences that have sequence identity to a nucleic acid sequence of any one of the sequences of the bacterial strains or species described herein. The terms “identical,” or percent “identity,” in the context of two or more nucleic acids or amino acid sequences, refer to two or more sequences or subsequences that are the same. Two sequences are “substantially identical” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% sequence identity) over a specified region of a nucleic acid or amino acid sequence or over the entire sequence, when compared and aligned for maximum correspondence over a comparison window, or designated region as measured using one of the following sequence comparison algorithms or by manual alignment and visual inspection. Optionally, the identity exists over a region that is at least about 50 nucleotides in length, or more preferably over a region that is 100 to 500 or 1000 or more nucleotides in length. In some embodiments, the identity exists over the length the 16S rRNA or 16S rDNA sequence.

In some embodiments, the bacterial strain has at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or up to 100% sequence identity relative to any of the strains or bacterial species described herein over a specified region or over the entire sequence. It would be appreciated by one of skill in the art that the term “sequence identity” or “percent sequence identity,” in the context of two or more nucleic acid sequences or amino acid sequences, refers to a measure of similarity between two or more sequences or portion(s) thereof.

In some embodiments, the composition includes two or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more) bacterial strains, wherein the two or more bacterial strains contain 16S rDNA sequences having at least 97% sequence identity with nucleic acid sequences selected from SEQ ID NOs:1-22.

Additionally, or alternatively, two or more sequences may be assessed for the alignment between the sequences. The terms “alignment” or percent “alignment” in the context of two or more nucleic acids or amino acid sequences, refer to two or more sequences or subsequences that are the same. Two sequences are “substantially aligned” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the following sequence comparison algorithms or by manual alignment and visual inspection. Optionally, the alignment exists over a region that is at least about 50 nucleotides in length, or more preferably over a region that is 100 to 500 or 1000 or more nucleotides in length. In some embodiments, the identity exists over the length the 16S rRNA or 16S rDNA sequence.

For sequence comparison, typically one sequence acts as a reference sequence, to which test sequences are compared. Methods of alignment of sequences for comparison are well known in the art. See, e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA 85:2444, 1988, by computerized implementations of these algorithms (GAP, BESTFIT, FASTA, and TFASTA in the Wisconsin Genetics Software Package, Genetics Computer Group. Madison. Wis.), or by manual alignment and visual inspection (see. e.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

Provided herein are bacterial strains and combinations of bacterial strains that are homologous or have a high percent of homology with bacterial strains comprising 16S rDNA sequences selected from SEQ ID NO: 1-36 and/or bacterial strains provided in PCT Publication Nos WO 2013/080561, WO 2017//218680, WO 2019/094837. It should be appreciated that the bacterial strains described herein have a 16S rDNA sequence selected from SEQ ID NO: 1-36 may also be homologous to other strains based on their whole genome sequence, or subset of their whole genome sequence.

In one aspect, the compositions comprise a fraction of a fecal sample. In some embodiments, the compositions comprise a non-spore forming fraction of a fecal sample. In some embodiments, the compositions comprise a spore forming fraction of a fecal sample.

In some embodiments, the compositions described herein comprise spore forming and non-spore forming bacterial strains. In some embodiments, the compositions described herein comprise spore forming bacterial strains. In some embodiments, the compositions described herein comprise only spore forming bacterial strains. In some embodiments, the compositions described herein comprise only non-spore forming bacterial strains. The spore-forming bacteria can be in spore form (i.e., as spores) or in vegetative form (i.e., as vegetative cells). In spore form, bacteria are generally more resistant to environmental conditions, such as heat, acid, radiation, oxygen, chemicals, and antibiotics. In contrast, in the vegetative state or actively growing state, bacteria are more susceptible to such environmental conditions, compared to in the spore form. In general, bacterial spores are able to germinate from the spore form into a vegetative/actively growing state, under appropriate conditions. For instance, bacteria in spore form may germinate when they are introduced in the intestine.

In some embodiments, at least one (e.g., 1, 2, 3, 4, 5, or more) of the bacterial strains in the composition is a spore former. In some embodiments, at least one (e.g., 1, 2, 3, 4, 5, or more) of the bacterial strains in the composition is in spore form. In some embodiments, at least one (e.g., 1, 2, 3, 4, 5, or more) of the bacterial strains in the composition is a non-spore former. In some embodiments, at least one (e.g., 1, 2, 3, 4, 5, or more) of the bacterial strains in the composition is in vegetative form. As discussed above, spore forming bacteria can also be in vegetative form. In some embodiments, at least one (e.g., 1, 2, 3, 4, 5, or more) of the bacterial strains in the composition is in spore form and at least one (e.g., 1, 2, 3, 4, 5, or more) of the bacterial strains in the composition is in vegetative form. In some embodiments, at least one bacterial strain that is considered able to form spores (i.e., a spore-former) but is present in the composition in vegetative form. In some embodiments, at least one bacterial strain that is considered able to form spores is present in the composition both in spore form and in vegetative form.

In some embodiments, the compositions comprise bacterial strains that are spore forming bacterial strains. In some embodiments, the compositions comprise bacterial strains that are non-spore forming bacterial strains. In some embodiments, the compositions comprise bacterial strains that are spore forming bacterial strains and bacterial strains that are non-spore forming bacterial strains. In some embodiments, the compositions comprise a mixture of bacterial strains wherein at least 10% of the bacterial strains are spore forming bacterial strains, at least 20% of the bacterial strains are spore forming bacterial strains, at least 30% of the bacterial strains are spore forming bacterial strains, at least 40% of the bacterial strains are spore forming bacterial strains, at least 50% of the bacterial strains are spore forming bacterial strains, at least 60% of the bacterial strains are spore forming bacterial strains, at least 70% of the bacterial strains are spore forming bacterial strains, at least 80% of the bacterial strains are spore forming bacterial strains, at least 90% of the bacterial strains are spore forming bacterial strains bacteria or up to 100% spore forming bacterial strains. Whether a bacterial strain is a spore forming strain can be determined for instance by evaluating the genome of the bacterial strain for the presence of sporulation genes. However, it should be appreciated that not all bacteria that are predicted to encode spore forming genes can be made to sporulate. In addition, whether a bacterial strain is a spore forming strain can be determined by exposing the bacterial strain to stress conditions, e.g., heat or exposure to chemicals (e.g., ethanol or chloroform), that are known to induce sporulation.

It should be appreciated that spore forming bacteria can be in spore form or in vegetative form. In some embodiments of the compositions provided herein, the spore forming bacteria are in spore form. In some embodiments, the spore forming bacteria are in vegetative form. In some embodiments, the spore forming bacteria are both present in spore form and in vegetative form. In some embodiments, compositions comprise spore forming bacteria and at least 10% of the spore forming bacteria are in spore format, at least 20% of the spore forming bacteria are in spore format, at least 30% of the spore forming bacteria are in spore format, at least 40% of the spore forming bacteria are in spore format, at least 50% of the spore forming bacteria are in spore format, at least 60% of the spore forming bacteria are in spore format, at least 70% of the spore forming bacteria are in spore format, at least 80% of the spore forming bacteria are in spore format, at least 90% of the spore forming bacteria are in spore format, or up to 100% of the spore forming bacteria are in spore format.

It is envisioned that the bacterial strains of the compositions provided herein are alive and will be alive when they reach the target area (e.g., the intestines). Bacterial spores are considered to be alive in this regard. In some embodiments, bacteria that are administered as spores may germinate in the target area (e.g., the intestines). It should further be appreciated that not all of the bacteria are alive, and the compositions can include a percentage (e.g., by weight) that is not alive. In addition, in some embodiments, the compositions include bacterial strains that are not alive when administered or at the time when the composition reaches the target area (e.g., the intestines). It is envisioned that non-living bacteria may still be useful by providing some nutrients and metabolites for the other bacterial strains in the composition.

Methods of inducing sporulation of spore-forming bacterial strains are well known in the art (See e.g., Paredes-Sabja et al., Trends Microbiol. (2011) 19(2):85-94). Generally, bacterial strains that are spore-formers can be made to go into spore form by stressing the bacterial strains. Non-limiting examples of stresses that can induce sporulation are an increase in temperature, change in the nutrients available and/or exposure to chemicals (e.g., ethanol or chloroform). It should be noted that bacteria that are non-spore formers, for instance because they are missing sporulation genes, cannot be made to sporulate by stress. To prepare compositions in which all the bacterial strains are in the spore form, the composition or bacterial cultures used to prepare the composition may be subjected to treatment to kill any bacteria not in spore form (e.g., in vegetative form), for example by exposing the composition to heat and chemically breaking down the non-spore bacteria. The bacteria in spore format can subsequently be separated from the non-spore bacteria for instance by filtration.

The amount of spores can be quantified using techniques known in the art. These techniques include phase contrast microscopy for enumerating spores using a hemocytometer. In addition, the viability of spores can be determined by plating the spores and growing the spores. For instance, spores can be plated in appropriate media and incubated in the anaerobic chamber for a period of time (e.g., 48-96 hrs.). Viability can subsequently be determined by quantifying the colony forming units which correspond to spores that germinated. For instance, spores can be plated on TCCFA plates (taurocholate, cycloserine, cefoxintin, fructose agar plates), in which taurocholate helps the spores to germinate. In addition, spores can be quantified using the dipicolinic assay (DPA assay). DPA is an agent that allows for spore selection and is a clear indicator of endospores. When complexed with terbium, bright green luminescence is observed.

In some embodiments, the compositions comprise bacterial strains that are non-spore forming bacterial strains. In some embodiments, the compositions comprise bacterial strains that are spore forming bacterial strains and bacterial strains that are non-spore forming bacterial strains. In some embodiments, the compositions comprise a mixture of bacterial strains wherein at least 10% of the bacterial strains are non-spore forming bacterial strains, at least 20% of the bacterial strains are non-spore forming bacterial strains, at least 30% of the bacterial strains are non-spore forming bacterial strains, at least 40% of the bacterial strains are non-spore forming bacterial strains, at least 50% of the bacterial strains are non-spore forming bacterial strains, at least 60% of the bacterial strains are non-spore forming bacterial strains, at least 70% of the bacterial strains are non-spore forming bacterial strains, at least 80% of the bacterial strains are non-spore forming bacterial strains, at least 90% of the bacterial strains are non-spore forming bacterial strains, or up to 100% non-spore forming bacterial strains.

In any of the compositions provided herein, the bacterial strains may be purified. In any of the compositions provided herein, the bacterial strains may be isolated. Any of the bacterial strains described herein may be isolated and/or purified, for example, from a source such as a culture or a microbiota sample (e.g., fecal matter). The bacterial strains used in the compositions provided herein generally are isolated from the microbiome of healthy individuals. However, bacterial strains can also be isolated from individuals that are considered not to be healthy. In some embodiments, the compositions include strains originating from multiple individuals.

As used herein, the term “isolated” refers to a bacteria or bacterial strain that has been separated from one or more undesired component, such as another bacterium or bacterial strain, one or more component of a growth medium, and/or one or more component of a sample, such as a fecal sample. In some embodiments, the bacteria are substantially isolated from a source such that other components of the source are not detected.

As also used herein, the term “purified” refers to a bacterial strain or composition comprising such that has been separated from one or more components, such as contaminants. In some embodiments, the bacterial strain is substantially free of contaminants. In some embodiments, one or more bacterial strains of a composition may be independently purified from one or more other bacteria produced and/or present in a culture or a sample containing the bacterial strain. In some embodiments, a bacterial strain is isolated or purified from a sample and then cultured under the appropriate conditions for bacterial replication, e.g., under anaerobic culture conditions. The bacteria that is grown under appropriate conditions for bacterial replication can subsequently be isolated/purified from the culture in which it is grown.

In some embodiments, the bacterial strains of the compositions provided herein are obligate anaerobes. In some embodiments, the bacterial strains of the compositions provided.

Aspects of the present disclosure are related to methods for suppressing undesired bacteria. As used herein, the term “suppressing” refers to any form of inhibiting undesired bacteria. In some embodiments, the methods described herein reduce/inhibit or prevent the colonization, replication, proliferation, and/or survival of the undesired bacteria (e.g., pathogenic organisms, multi-drug resistant organisms, oral microbiome bacteria). In some embodiments, the methods described herein directly or indirectly induce death of the undesired bacteria (e.g., pathogenic organism, multi-drug resistant organisms, oral microbiome bacteria).

Aspects of the present disclosure are related to methods for suppressing multi-drug resistant organisms in a subject. Aspects of the present disclosure are related to methods for suppressing a pathogenic organism (e.g., bacterium, virus, fungus, or parasite) in a subject. In some embodiments, the pathogenic organism is not multi-drug resistant. In some embodiments, the pathogenic organism is multi-drug resistant. Aspects of the present disclosure are related to methods of suppressing or preventing colonization of the intestine with oral microbiome bacteria. Aspects of the present disclosure are related to methods for treating a disease or disorder associated with bacterial colonization in a subject. Aspects of the present disclosure are related to methods for treating a disease or disorder associated with an immune response induced by bacteria in a subject. The methods described herein involve administering to a subject a therapeutically effective amount of any of the compositions described herein. As used herein, a “subject,” “individual,” and “patient” are used interchangeably, and refer to a vertebrate, preferably a mammal such as a human. Mammals include, but are not limited to, human primates, non-human primates or murine, bovine, equine, canine or feline species. In some embodiments, the subject is a human.

In some embodiments, any of the compositions described herein are effective in suppressing the replication, survival, and/or colonization of one or more pathogenic organism. In some instances, the pathogenic organism is susceptible to antibiotics, while in other instances, the pathogenic organism is resistant to antibiotics. In some embodiments, the pathogenic organism is a multi-drug resistant organism, which are described elsewhere herein. In some embodiments, the pathogenic organism is an oral microbiome bacterium. It should be noted that oral microbiome bacteria are not necessarily pathogenic, but may become so when located elsewhere, such as in the gastrointestinal tract. The amount of suppression of replication, survival, and/or colonization of the one or more pathogenic organism can be measured or identified using standard assays known in the art, some of which are further described and exemplified herein.

In some embodiments, administration of the compositions described herein reduces/inhibits or prevents the colonization, re-colonization, replication, proliferation, and/or survival of multi-drug resistant organisms. In some embodiments, administration of the compositions described herein allows for colonization of the gastrointestinal tract of the subject by the bacterial strain(s) of the compositions thereby preventing colonization by multi-drug resistant organisms.

In some embodiments, the pathogenic organism is Clostridium difficile. In some embodiments, the pathogenic organism is Klebsiella pneumoniae. In some embodiments, the Klebsiella pneumoniae is multi-drug resistant. In some embodiments, the multi-drug resistant Klebsiella pneumoniae is carbapenem-resistant Klebsiella pneumoniae. In some embodiments, the Klebsiella organism induces a Th1 response. In some embodiments, the Klebsiella pneumoniae is multi-drug resistant and induces a Th1 response. In some embodiments, the Klebsiella pneumoniae is one or more of those described in Atarashi et al. Science (2017) 358, 359-365, such as strain BAA-2552, strain KP-1, strain 700721, strain 13882, strain 34E1, strain BAA-1705, strain 700603, and/or strain Kp-2H7. In one particular embodiment, the Klebsiella pneumoniae is strain Kp-2H7.

In some embodiments, the pathogenic organism is a pathobiont, i.e., a potentially pathogenic organism which, under normal circumstances, lives as a symbiont.

In some embodiments, the subject is a carrier of a multi-drug resistant organism and is suffering from the effects of the infection. In some embodiments the subject is an asymptomatic carrier of a multi-drug resistant organism. In some embodiments, the subject has experienced recurrent or chronic colonization with a multi-drug resistant organism. In some embodiments, the subject is suffering from a first occurrence of a particular multi-drug resistant organism. In some embodiments, the subject is at risk of colonization with a multi-drug resistant organism, such as prior antibiotic use. In some embodiments, the subject has a risk factor associated with colonization with a multidrug resistant organism. In some embodiments, the subject has had a previous infection or colonization with a multi-drug resistant organism. In some embodiments, the subject has been treated with antibiotics which resulted in the recurrence of the multi-drug resistant organism.

In some embodiments, the subject is to undergo a procedure that puts the subject at a higher risk of colonization and the compositions are administered prophylactically. In some embodiments, the subject has a disease or disorder associated with use of a proton pump inhibitor, which may increase the likelihood of an oral bacterium migrating to the intestine. In some embodiments, the compositions provided herein are administered to a subject to lower the risk of becoming colonized with a multidrug resistant organism. In some embodiments, the bacterial compositions provided herein administered to a subject that is receiving a proton pump inhibitor.

Individuals may be at risk of acquiring a multi-drug resistant organism if they have recently received antimicrobials, are in an immunosuppressed state (e.g., on chemotherapy, have a malignancy, undergoing or received a transplant), have a chronic disease or inflammatory condition (such as diabetes, renal disease, etc.), are older, are undergoing hemodialysis, surgery or other invasive procedures, have indwelling device(s), and/or are living in a long-term care facility or are hospitalized. In some embodiments, the subject is colonized with a multi-drug resistant organism. Skin and mucosal colonization are common (Cassone et al., Curr Geriatr Rep. (2015) 4(1): 87-89), but multi-drug resistant organisms may also colonize the gastrointestinal (GI) tract and oral cavity, causing inflammation (Atarashi et al., Science (2017) 358, 359-365). Colonization can lead to significant infections, such as in the skin, lungs, urinary tract, or bloodstream, which may result in serious complications, including death (CDC, 2013). In some instances, multi-drug resistant organisms may be ingested, leading to consequences throughout the digestive system. In some embodiments, a multi-drug resistant organism may colonize the oral cavity.

In some embodiments, the multi-drug resistant organism is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella Typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, or Clindamycin resistant Group B Streptococcus. In some embodiments, the multi-drug resistant organism is Vancomycin Resistant Enterococci (VRE). In some embodiments, the multi-drug resistant organism is Carbapenem Resistant Enterobacteriaceae (CRE).

Aspects of the present disclosure are related to methods for suppressing pathogenic organisms in a subject. In some embodiments, the pathogenic organism is susceptible to antibiotics (e.g., not a multi-drug resistant organism). Pathogenic organisms are organisms that are capable of causing disease in a subject and may be viruses, bacteria, fungi, protozoa, and worms. Non-limiting examples of pathogenic organisms of the present disclosure include Salmonella, Shigella, Staphylococcus, Streptococcus, Enterococcus, Enterobacteriaceae, Neisseria gonorrheae, Acinetobacter, Campylobacter, Clostridium, Listeria, Escherichia, Pseudomonas aeruginosa, Salmonella, Shigella, Staphylococcus aureus, Streptococcus pneumoniae, Tuberculosis, Group A Streptococcus, or Group B Streptococcus, Toxoplasma, Cyclospora, Giardia, Cryptosporidium, and Trichinella.

In some embodiments, pathogenic organisms in the subject are resistant to treatment with a drug. In some embodiments, pathogenic organisms in the subject are resistant to treatment with multiple drugs (e.g., multi-drug resistant). Drugs that may be used to treat pathogenic organisms of the present disclosure may include antibiotics, antivirals, antifungals, and antiparasitics.

In some embodiments, a pathogenic organism that is susceptible to a drug may acquire resistance to one or more drugs. In some embodiments, a pathogenic organism that is susceptible to antibiotics may acquire resistance to one or more antibiotics. In some embodiments, a pathogenic organism is resistant to one or more drugs (e.g., antibiotics) and may acquire resistance to one or more drugs (e.g., antibiotics). In some embodiments, the pathogenic organism is resistant to one or more drugs (e.g., antibiotics) and may become a multi-drug resistant organism by acquiring resistance to more than one drugs (e.g., antibiotics).

In general, there are multiple means by which an organism may acquire drug resistance. In some embodiments, the organism may acquire drug resistance by acquiring mutations in its genome that promote survival of the organism in the presence of at least one drug. In some embodiments, the organism acquires DNA from an organism that is resistant to one or more drugs (e.g., antibiotics), thereby becoming resistant to the drug (e.g., antibiotic). In some embodiments, the organism acquires a plasmid, such as a plasmid containing one or more drug (e.g., antibiotic) resistance genes, from an organism that is resistant to one or more drugs (e.g., antibiotics), thereby becoming resistant to the drug (e.g., antibiotic).

In some embodiments, administration of the compositions described herein reduces/inhibits or prevents intestinal colonization with oral microbiome bacteria. In some embodiments, administration of the compositions described herein reduces/inhibits or prevents the colonization, replication, proliferation, and/or survival of oral microbiome bacteria in the intestinal tract of the subject. In some embodiments, administration of the compositions described herein allows for colonization of the gastrointestinal tract of the subject by the bacterial strain(s) of the compositions thereby preventing colonization by oral microbiome bacteria.

In some embodiments, the subject is a carrier of an oral bacterium and is suffering from the effects of the infection. In some embodiments the subject is an asymptomatic carrier of an oral bacterium. In some embodiments, the subject has experienced recurrent or chronic colonization with an oral bacterium. In some embodiments, the subject is at risk of colonization with an oral bacterium. In some embodiments, the subject has a risk factor associated with colonization with an oral bacterium. In some embodiments, the subject is taking a proton pump inhibitor. In some embodiments, the subject has had a previous infection or colonization with an oral bacterium.

In some embodiments, the subject is to undergo a procedure that puts the subject at a higher risk of colonization and the compositions are administered prophylactically. In some embodiments, the compositions provided herein are administered to a subject to lower the risk of becoming colonized with an oral bacterium.

Over 700 bacterial species or phylotypes have been found in the oral cavity; however, over 50% have not yet been cultivated. A number of phyla have been identified in the oral microbiome Actinobacteria, Arachnia, Bacteroidetes, Bifidobacterium, Chlamydiae, Chloroflexi, Eubacterium, Euryarchaeota, Fusobacterium, Firmicutes, Fusobacteria, Lactobacillus, Leptotrichia, Peptococcus, Peptostreptococcus, Propionibacterium, Proteobacteria, Selenomonas, Spirochaetes, SR1, Synergistetes, Tenericutes, Treponema, TM7, and Veillonella (Dewhirst et al., J. Bacteriology (2010) 192(19): 5002-5010). Examples of oral microbiome bacteria include, without limitation, Streptococcus sanguis, Streptococcus salivarius, Streptococcus mitis, Streptococcus mutans, Treponema denticola, Eikenella corrodens, Streptococcus gordonii, Streptococcus oralis, Acinomyces maeslundii, and Bacteroides melaningenicus. A list of example species found in the human oral cavity can be found on the Human Oral Microbiome Database (homd.org). In some embodiments, the oral microbiome bacteria may be pathogenic. In some embodiments, the oral microbiome bacteria may be pathogenic if the bacteria gain access to another site of the body. In some embodiments, the oral microbiome bacteria are not pathogenic.

In some embodiments, administration of the compositions described herein reduces/inhibits or prevents intestinal colonization with oral microbiome bacteria. In some embodiments, the oral microbiome bacteria are Fusobacterium nucleatum (See e.g., Yoneda et al. J Gastrointest Dig Syst (2016) 6:2). In some embodiments, the oral microbiome bacteria are Campylobacter concisus (See e.g., Yoneda et al. J Gastrointest Dig Syst (2016) 6:2). In some embodiments, the oral microbiome bacteria are Streptococcus mutans (See e.g., Yoneda et al. J Gastrointest Dig Syst (2016) 6:2). Additional oral microbiome bacteria are described in Table S1A of Atarashi et al. (Atarashi et al., Science (2017) 358, 359-365), such as Rothia mucilaginosa, Neisseria subflava, Granulicatella para-adiacens, Streptococcus salivarius, Streptococcus mitis, Fusobacterium sp. 1_1_41FAA, Streptococcus oralis, Streptococcus salivarius, Neisseria subflava, Prevotella scopos, Veillonella parvula, Streptococcus sp. M143, Haemophilus parainfluenzae, Prevotella sp. CD3_34, Neisseria macacae, Prevotella histicola, Prevotella pallens, Streptococcus infantis, Streptococcus parasanguinis, Porphyromonas CW034, Streptococcus sp. oral strain T1-E5, Gemella sp. 933-88, Veillonella parvula, and Prevotella sp. C561.

In some embodiments, intestinal colonization with an oral microbiome bacterium induces a Th1 immune response in the subject. Examples of oral microbiome bacteria that may induce Th1 immune responses have been isolated, showing significant similarity (≥96.3%) to the following species: Mogibacterium sp. CM96, Peptostreptococcus stomatis, Bifidobacterium sp. Group 111-3, Slackia exigua, Veillonella denticariosi, Atopobium parvulum, Veillonella sp. 2011_oral_VSA_A3, Campylobacter concisus, Actinomyces odontolyticus, Solobacterium moorei, Enterococcus faecium, Bifidobacterium dentium, Veillonella parvela, Fusobacterium sp. 3_1_33, Klebsiella aeromobilis, and Klebsiella pneumoniae (see, e.g., Atarashi et al., Science (2017) 358: 359-365, Schirmer et al., Cell Host and Microbe (2018) 24: 600-610).

In some embodiments, the bacterium that induces a Th1 immune response (e.g., IBD) in a subject is a pathobiont. “Pathobiont” refers to a potentially pathological (disease-causing) organism which, under normal circumstances, lives as a symbiont. Examples of pathobionts include a bacterium that is associated with chronic inflammatory conditions (e.g., IBD). Non-limiting examples of pathobionts include Shigella spp., Campylobacter spp., Cryptosporidium spp., Salmonella spp., Escherichia coli strains (e.g., Enteropathogenic E. coli, Enteroaggregative E. coli, Enterotoxigenic E. coli), Veillonella dispar, Aggregatibacter segnis, Campylobacter, Lachnospiraceae, Veillonella parvula, Haemophilus parainfluenzae, Megasphaera, Escherichia coli, Enterobacteriaceae spp., Enterococcus spp., Fusobacterium spp., Gemella spp., Veillonella spp., Pasteurella spp., Neisseria spp., Haemophilus spp., Campylobacter spp., and Bifidobacterium spp.

In some embodiments, the methods may involve determining whether an oral bacterium is present in the subject. In some embodiments, the methods may involve determining whether an oral bacterium colonizes the oral cavity of the subject. In some embodiments, a subject may be at risk of intestinal colonization if the oral bacterium is present in the oral cavity of the subject. In some embodiments, the methods involve administering the combinations described herein to the subject, if an oral bacterium is detected in the oral cavity of the subject.

In some embodiments, the methods may involve determining whether an oral bacterium is present in the intestine of the subject. In some embodiments, the methods involve administering the combinations described herein to the subject, if an oral bacterium is detected in the intestine of the subject.

In some embodiments, the methods are for treating a disease or disorder associated with bacterial colonization in a subject. In some embodiments, the methods are for treating a disease or disorder associated with an immune response induced by bacteria in a subject. In some embodiments, the methods are for treating a disease or disorder associated with an undesired immune response induced by bacteria in a subject.

In some embodiments, the methods may involve determining whether the subject is colonized with bacteria. In some embodiments, the methods may involve determining whether the subject has or is experiencing an immune response induced by bacterial colonization. In some embodiments, a subject may be at risk of an immune response induced by bacterial colonization if the subject is colonized by the bacteria. In some embodiments, the methods involve administering the combinations described herein to the subject, if the subject is determined to be colonized by the bacteria. In some embodiments, the methods involve administering the combinations described herein to the subject, if the subject is determined to be experiencing or have experienced an immune response induced by bacterial colonization.

In some embodiments, the immune response induced by bacterial colonization is a Th1 immune response. As will be evident to one of skill in the art, Th1 immune responses are mediated the Th1 population of CD4+ cells. Th1 cells produce IFN-γ and other pro-inflammatory factors. The differentiation of CD4+ cells to Th1 cells is promoted by the presence of IL-2 and/or IL-12 and activation of the transcription factors STAT4 and T-bet. In some embodiments, the immune responses induced by bacterial colonization are Th1 pro-inflammatory responses. Any direct or indirect measure of Th1 immune response, such as the amount of IFN-γ or the number of Th1 cells, may be used to assess the level or extent of the immune response in a sample from a subject.

In some embodiments, the compositions provided herein are administered to a subject if the subject has an autoimmune disease. Examples of autoimmune diseases include, without limitation, inflammatory bowel disease (IBD), ulcerative colitis, Crohn's disease, sprue, autoimmune arthritis, rheumatoid arthritis, graft versus host disease, Type I diabetes, multiple sclerosis, osteoarthritis, juvenile chronic arthritis, Lyme arthritis, psoriatic arthritis, reactive arthritis, spondyloarthropathy, systemic lupus erythematosus, insulin dependent diabetes mellitus, thyroiditis, asthma, psoriasis, dermatitis scleroderma, atopic dermatitis, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlejn purpurea, microscopic vasculitis of the kidneys, chronic active hepatitis, uveitis, cachexia, acute transverse myelitis, Huntington's chorea, Parkinson's disease, Alzheimer's disease, stroke, primary biliary cirrhosis, hemolytic anemia, polyglandular deficiency type I syndrome and polyglandular deficiency type II syndrome, Schmidt's syndrome, adult (acute) respiratory distress syndrome, alopecia, alopecia areata, seronegative arthopathy, arthropathy, Reiter's disease, psoriatic arthropathy, chlamydia, yersinia and salmonella associated arthropathy, spondyloarhopathy, atheromatous disease/arteriosclerosis, Pemphigus vulgaris, Pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, common varied immunodeficiency (common variable hypogammaglobulinaemia), dilated cardiomyopathy, fibrotic lung disease, cryptogenic fibrosing alveolitis, postinflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, discoid lupus, erythematosus, male infertility idiopathic or NOS, sperm autoimmunity, insulin-dependent diabetes mellitus, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, eosinophilic esophagitis, hypereosinophilic syndrome, eosinophilic gastroenteritis cutaneous lupus erythematosus, eosinophilic esophagitis, hypereosinophilic syndrome, and eosinophilic gastroenteritis, and diarrhea. In some embodiments, the autoimmune disease is inflammatory bowel disease (IBD). In some embodiments, the autoimmune disease is ulcerative colitis. In some embodiments, the autoimmune disease is Crohn's disease.

In some embodiments, the compositions provided herein are administered to a subject if the subject has non-alcoholic steatohepatitis (NASH), primary sclerosing cholangitis (PSC), non-alcoholic fatty liver disease (NAFLD), gastroesophageal reflux disease (GERD), or alcoholism.

In some embodiments, the compositions provided herein are administered to a subject if an immune response associated with bacterial colonization has been detected in the subject. In some embodiments, the methods involve determining whether the subject has an immune response induced by or associated with colonization with an undesired organism.

In some embodiments, the compositions provided herein are administered to a subject if the subject has a dysbiosis (e.g., has as microbiome associated with a disease state). In some embodiments, treatment with the compositions provided herein results in the change in the microbiome of the subject. In some embodiments, treatment with the compositions provided herein removes the dysbiosis in the subject resulting in a healthy microbiome. In some embodiments, treatment with the compositions provided herein removes the dysbiosis in the subject resulting in microbiome refractory or less susceptible to infection by a pathogen.

In some embodiments, the compositions and methods described herein are for the treatment and/or prevention of sepsis. In some embodiments, the compositions provided herein are administered to a subject if the subject has or is at risk for sepsis. In some embodiments, the compositions and methods described herein are for the treatment and/or prevention of sepsis in critically ill subjects, such as patients in an intensive care unit (ICU, also referred to as an intensive therapy unit or intensive treatment unit (ITU) or critical care unit (CCU)). In some embodiments, the compositions provided herein are administered to a subject if the subject is a critically ill subject and has or is at risk for sepsis. As will be evident to one of ordinary skill in the art, sepsis is a systemic inflammatory response that is typically due to infection with bacterium, fungus, virus, or protozoa. The severity of sepsis can range from mild (sepsis) to severe sepsis or even septic shock. Subjects of advanced age (e.g., over 65 years old), young age (e.g., under 1 year old), and/or having a compromised immune systems, including due to cancer, diabetes, burns, trauma, or other disease or disorder resulting in a critical illness, may be at increased risk of developing sepsis.

In some embodiments, the compositions and methods described herein are for the treatment and/or prevention of infection in critically ill subjects, such as patients in an intensive care unit (ICU). In some embodiments, the compositions provided herein are administered to a subject if the subject is a critically ill subject. In some embodiments, the subject may be a patient in an intensive care unit. In some embodiments, any of the compositions described herein may be administered to a subject to treat and/or prevent a bacterial infection, fungal infection, viral infection, or protozoan infection in a critically ill subject.

In some embodiments, the compositions and methods described herein are for the treatment and/or prevention of infection following a surgical procedure involving the gastrointestinal tract (i.e., gastrointestinal surgery). In some embodiments, the compositions and methods described herein are administered to a subject if the subject has undergone a surgical procedure involving the gastrointestinal tract. In some embodiments, the compositions and methods described herein are administered to a subject if the subject will be undergoing a surgical procedure involving the gastrointestinal tract (e.g., administered prior to the surgical procedure). In some embodiments, the subject has a disease or disorder associated with the gastrointestinal tract. In some embodiments, any of the compositions described herein may be administered to a subject to treat and/or prevent a bacterial infection, fungal infection, viral infection, or protozoan infection.

In some embodiments, the compositions and methods described herein are for the treatment and/or prevention of infection in subjects having cirrhosis (i.e., a cirrhosis patient). In some embodiments, the compositions and methods described herein are administered to a subject if the subject has or is at risk of cirrhosis. As will be evident to one of ordinary skill in the art, cirrhosis, also referred to as liver cirrhosis or hepatic cirrhosis, is a condition associated with liver dysfunction, predominantly due to long-term damage of the liver tissue and cells thereof. Cirrhosis may be caused by any of a variety of factors, for example, hepatitis (e.g., viral infection with Hepatitis B or Hepatitis C, autoimmune hepatitis), and excessive alcohol consumption. In some embodiments, any of the compositions described herein may be administered to a subject to treat and/or prevent a bacterial infection, fungal infection, viral infection, or protozoan infection in a cirrhosis patient.

In some embodiments, the compositions and methods described herein are for the treatment and/or prevention of infection in subjects who received a bone marrow transplant (i.e., bone marrow transplant patients). In some embodiments, the compositions and methods described herein are administered to a subject if the subject has undergone or will undergo a bone marrow transplant. In some embodiments, any of the compositions described herein may be administered to a subject to treat and/or prevent a bacterial infection, fungal infection, viral infection, or protozoan infection in a bone marrow transplant patient.

Any of the compositions described herein may be administered to a subject in a therapeutically effective amount or a dose of a therapeutically effective amount to treat or prevent a disease or disorder, for example associated with colonization with bacteria or an immune response associated with colonization with bacteria. The terms “treat” and “treatment” refer to reducing or alleviating one or more of the symptoms associated with colonization with bacteria or an immune response associated with colonization with bacteria. In some embodiments, any of the compositions described herein may be administered to a subject to prevent a disease or disorder. In some embodiments, any of the compositions described herein may be administered to a subject to prevent a Th1 related disease or disorder. In some embodiments, any of the compositions described herein may be administered to a subject to prevent IBD. The terms “prevent” and “prevention” encompass prophylactic administration and may reduce the incidence or likelihood of colonization with bacteria or an immune response associated with colonization with bacteria. For instance, in some embodiments, administration of the compositions provided herein result in a healthy microbiome that is refractory to pathogenic infection, thereby preventing the pathogenic infection or re-colonization with the pathogenic organism.

As used herein, a “therapeutically effective amount” may be used interchangeably with the term “effective amount.” A therapeutically effective amount or an effective amount of composition, such as a pharmaceutical composition, is any amount that results in a desired response or outcome in a subject, such as those described herein, including but not limited to reducing or preventing colonization with bacteria or an immune response associated with colonization with bacteria.

It should be appreciated that the term effective amount may be expressed as the number of bacteria or bacterial spores to be administered. It should further be appreciated that the bacteria can multiply once administered. Thus, administration of even a relatively small amount of bacteria may have therapeutic effects.

In some embodiments, the therapeutically effective amount of any of the compositions described herein is an amount sufficient to enhance survival of the subject, reduce or prevent bacterial colonization of the subject, and/or reduce or inhibit toxin production by the pathogenic infection. In some embodiments, colonization may be assessed by detecting and/or quantifying the bacteria in a sample from the subject, such as a fecal sample. In some embodiments, the therapeutically effective amount is an amount sufficient to reduce the colonization bacteria (e.g., pathogenic organisms, multi-drug resistant organisms, oral microbiome bacteria) in a fecal sample from the subject by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the bacterial burden in a subject that has not received any of the compositions described herein, or as compared to a fecal sample from the same subject that was collected prior to administration of any of the compositions.

In some embodiments, the compositions provided herein reduce an immune response associated with bacterial colonization or induced by bacterial colonization. In some embodiments, the therapeutically effective amount is an amount sufficient to reduce an immune response associated with bacterial colonization or induced by bacterial colonization by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the immune response associated with bacterial colonization or induced by bacterial colonization prior to administration of any of the compositions.

In some embodiments, the therapeutically effective amount is an amount sufficient to recolonize or repopulate the gastrointestinal tract of the subject with non-pathogenic bacteria. In some embodiments, the therapeutically effective amount is an amount sufficient to graft one or more of the bacterial strains of the composition in the gastrointestinal tract of the subject. In some embodiments, a fecal sample is obtained from the subject to assess the bacterial burden of undesired bacteria (e.g., pathogenic organisms, multi-drug resistant organisms, oral microbiome bacteria) and/or evaluate the efficacy of administration of the bacterial compositions described herein. In some embodiments, the microbiota of the subject (e.g., the identity and abundance of strains and/or species of the microbiota) may be assessed to determine a disease state of the subject and/or assess progress of the treatment. In some embodiments, the microbiota of the subject having a pathogenic infection is compared to the microbiota of a healthy subject, such as a subject that is not experiencing or has not experienced the pathogenic infection. In some embodiments, the microbiota of the subject having a pathogenic infection is compared to the microbiota of the same subject from a fecal sample obtained from the subject prior to the pathogenic infection.

In some embodiments, administration of the compositions provided herein results in a healthy microbiome that reduces or prevents colonization of the subject by any undesired organism. In some embodiments, administration of the compositions provided herein results in a healthy microbiome that reduces or prevents intestinal colonization of the subject by any undesired organism (e.g., pathogenic organisms, multi-drug resistant organisms, oral microbiome bacteria). In some embodiments, administration of the compositions provided herein results in a healthy microbiome that reduces an immune response associated with bacterial colonization, such as colonization with undesired bacteria. In some embodiments, administration of the compositions provided herein results in a healthy microbiome that reduces a Th1 immune response in the subject.

Any of the compositions described herein may be administered in combination with one or more additional compositions that can suppress a Th1 response and/or induces the accumulation and/or proliferation of regulatory T cells, and/or Th17 cells. In some embodiments, any of the compositions described herein may be administered in combination with a composition that induces the proliferation and/or accumulation of regulatory T cells (“Treg”).

In one aspect, the disclosure provides compositions comprises Treg inducing bacterial strains (such as VE202 or VE303), an Escherichia species (such as E. coli (e.g., Escherichia sp. 3_2_53FAA)) and/or a Fusobacterium species (e.g., Fusobacterium mortiferum).

In some embodiments, any of the compositions described herein may be administered in combination with VE-202, a Treg inducing composition of 17 bacterial strains, described for instance in Atarashi et al., Nature (2013) 500: 232-236. The 17 bacterial strains of VE-202 are represented by the following species: Clostridium saccharogumia, Flavonifractor plautii, Clostridium hathewayi, Blautia coccoides, Clostridium bolteae ATCC BAA-613, cf. Clostridium sp. MLGO55, Clostridium indolis, Anaerotruncus colihominis, Ruminococcus sp. ID8, Clostridium asparagiforme DSM 15981, Clostridium symbiosum, Clostridium ramosum, Eubacterium contortum, Lachnospiraceae bacterium 5_1_57FAA, Lachnospiraceae bacterium 3_1_57FAA_CT1, Clostridiales bacterium 1_7_47FAA, and Lachnospiraceae bacterium A4. It should be appreciated that subsets of the VE-202 bacteria can also induce Treg cells. Examples of subsets of VE202 that induce Treg cells are found for instance in Atarashi et al., Nature (2013) 500: 232-236 and corresponding Supplemental Information. In some embodiments, any of the compositions described herein may be administered in combination with any of the bacterial compositions as described in PCT Publication WO 2016/209806.

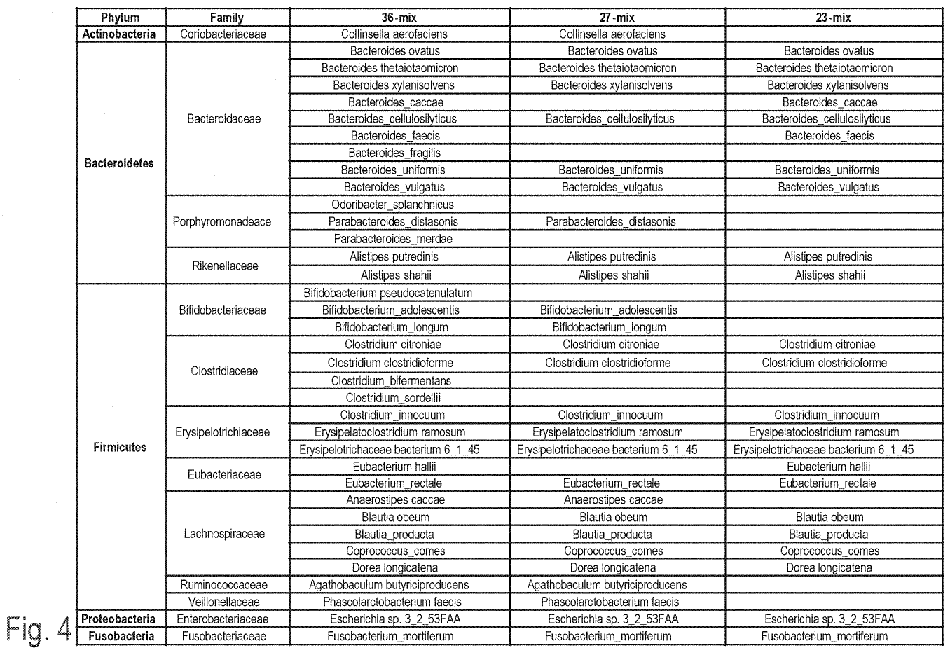

In some embodiments, any of the compositions described herein may be administered in combination with any of the bacterial compositions described in PCT Publication WO 2019/094837, which is incorporated herein by reference in its entirety. In some embodiments, any of the compositions described herein may be administered in combination with any of the bacterial compositions described in PCT Publication WO 2019/118515, which is incorporated herein by reference in its entirety. In some embodiments, any of the compositions described herein are administered in combination with a composition of 36 bacterial strains (e.g., the 36-mix, shown in FIGS. 4 and 5).

As used herein, the phrase “induces proliferation and/or accumulation of regulatory T cells” refers to an effect of inducing the differentiation of immature T cells into regulatory T cells, which differentiation leads to the proliferation and/or the accumulation of regulatory T cells. Further, the meaning of “induces proliferation and/or accumulation of regulatory T cells” includes in vivo effects, in vitro effects, and/or ex vivo effects. In some embodiments, the proliferation and/or accumulation of regulatory T cells may be assessed by detecting and/or quantifying the number of cells that express markers of regulatory T cells (e.g., Foxp3 and CD4), for example by flow cytometry. In some embodiments, the proliferation and/or accumulation of regulatory T cells may be assessed by determining the activity of the regulatory T cells, such as the production of cytokines (e.g., IL-10).

In some embodiments, any of the compositions described herein may be administered in combination with a composition that induces the accumulation and/or proliferation of Th17 cells, see e.g., compositions disclosed in PCT Publication WO 2015/156419, which is incorporated herein by reference in its entirety.

In some embodiments, suppressing live bacterial product also repopulates the microbiota of the subject.

In some embodiments, administration of the compositions described herein results in an increase in the proliferation and/or accumulation of regulatory T cells (e.g., total Tregs or pathogenic organism-specific Tregs) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the quantity of regulatory T cells in the subject (or particular site in the subject) prior to administration of the compositions. In some embodiments, administration of the compositions described herein results in an increase the proliferation and/or accumulation of regulatory T cells (e.g., total Tregs or pathogenic organism-specific Tregs) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the quantity of regulatory T cells in another subject (e.g., a reference subject) who did not receive the compositions.

In some embodiments, administration of the compositions described herein results in an increase the proliferation and/or accumulation of regulatory T cells (e.g., total Tregs or pathogenic organism-specific Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the quantity of regulatory T cells in the subject (or particular site in the subject) prior to administration of the compositions. In some embodiments, administration of the compositions described herein results in an increase the proliferation and/or accumulation of regulatory T cells (e.g., total Tregs or pathogenic organism-specific Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the quantity of regulatory T cells in another subject (e.g., a reference subject) who did not receive the compositions.

The induction of Treg cells and corresponding decolonization of pathogenic organisms are intricately related. In some embodiments, administration of the compositions described herein results in an increase the proliferation and/or accumulation of regulatory T cells (e.g., total Tregs or pathogenic organism-specific Tregs) by between 1% and 20%, 2% and 19%, 3% and 17%, 4% and 16%, 4% and 15%, 5% and 15%, 6% and 14%, 7% and 13%, 8% and 12%, 5% and 10%, 5% and 15%, 10% and 15%, or 8% and 15% as compared to the quantity of regulatory T cells in the subject (or particular site in the subject) prior to administration of the compositions. In some embodiments, administration of the compositions described herein results in an increase the proliferation and/or accumulation of regulatory T cells (e.g., total Tregs or pathogenic organism-specific Tregs) by between 1% and 20%, 2% and 19%, 3% and 17%, 4% and 16%, 4% and 15%, 5% and 15%, 6% and 14%, 7% and 13%, 8% and 12%, 5% and 10%, 5% and 15%, 10% and 15%, or 8% and 15% as compared to the quantity of regulatory T cells in another subject (e.g., a reference subject) who did not receive the compositions.

In some embodiments, administration of the compositions described herein results in an increase in activity of regulatory T cells (e.g., total Tregs or pathogenic organism-specific Tregs) at a particular site (e.g., the gastrointestinal tract) in the subject. In some embodiments, administration of the compositions described herein results in an increase in activity of regulatory T cells (e.g., total Tregs or pathogenic organism-specific Tregs) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the activity of regulatory T cells in the subject (or particular site in the subject) prior to administration of the compositions. In some embodiments, administration of the compositions described herein results in an increase in activity of regulatory T cells (e.g., total Tregs or pathogenic organism-specific Tregs) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the activity of regulatory T cells in another subject (e.g., a reference subject) who did not receive the compositions.

In some embodiments, administration of the compositions described herein results in an increase in the activity of regulatory T cells (e.g., total Tregs or pathogenic organism-specific Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the activity of regulatory T cells in the subject (or particular site in the subject) prior to administration of the compositions. In some embodiments, administration of the compositions described herein results in an increase in the activity of regulatory T cells (e.g., total Tregs or pathogenic organism-specific Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the activity of regulatory T cells in another subject (e.g., a reference subject) who did not receive the compositions.

The abundance of regulatory T cells (e.g., total Tregs or pathogenic organism-specific Tregs) can be assessed by any method known in the art, for example by detecting a cellular marker indicative of regulatory T cells (e.g., FoxP3), assessing a direct or indirect activity of regulatory T cells, and/or by measuring the production of one or more cytokines produced by regulatory T cells (e.g., IL-10).

Some aspects compositions and methods described herein increase production of short chain fatty acids (e.g., in the gastrointestinal tract of the subject). In some embodiments, the methods involve administering to a subject one or more compositions containing bacterial strains that produce short chain fatty acids. SCFAs are abundant in healthy subjects (e.g., subjects not having a pathogenic organism infection) and decreased in subjects having pathogenic organism infections (e.g., Clostridium difficile infections and rCDIs). Fecal matter transplant (FMT) have been shown to increase SCFA following rCDI (Seekatz, et al., Anaerobe (2018) 53: 64-73.

SCFA produced in the gastrointestinal tract are thought to function as signaling molecules between the gut microbiota and the host organism, with the SCFA playing a in local, intermediary and peripheral metabolism of the host. See, e.g., Morrison, et al. Gut Microbes (2016) 7(3): 189-200. In some embodiments, a damaged gut mucosal barrier can be repaired by providing SCFA.

Examples of SCFA include, without limitation, formic acid, acetic acid, butyric acid, isobutyric acid, valeric acid, or isovaleric acid. In some embodiments, the SCFA is butyric acid (butyrate).

The production of SCFAs and corresponding decolonization of pathogenic organisms are intricately related. In some embodiments, administration of the compositions described herein results in an increase in the production and/or accumulation of SCFAs (e.g., total SCFAs or pathogenic organism-specific SCFAs) by between 1% and 20%, 2% and 19%, 3% and 17%, 4% and 16%, 4% and 15%, 5% and 15%, 6% and 14%, 7% and 13%, 8% and 12%, 5% and 10%, 5% and 15%, 10% and 15%, or 8% and 15% as compared to the quantity of SCFAs in the subject (or particular site in the subject) prior to administration of the compositions. In some embodiments, administration of the compositions described herein results in an increase in the production and/or accumulation of SCFAs (e.g., total SCFAs or pathogenic organism-specific SCFAs) by between 1% and 20%, 2% and 19%, 3% and 17%, 4% and 16%, 4% and 15%, 5% and 15%, 6% and 14%, 7% and 13%, 8% and 12%, 5% and 10%, 5% and 15%, 10% and 15%, or 8% and 15% as compared to the quantity of SCFAs in another subject (e.g., a reference subject) who did not receive the compositions.

In some embodiments, the compositions and methods described herein result in an increase in the amount of SCFAs produced in the gastrointestinal tract of the subject. In some embodiments, the SCFAs are increased by 10-fold to 500-fold following administration of the composition as described herein. In some embodiments, SCFAs are increased by 20-fold to 250-fold following administration of the compositions described herein. In some embodiments, SCFAs are increased by 100-fold to 500-fold following administration of the compositions described herein. In some embodiments, SCFAs are increased by at least 2-fold, 5-fold, 10-fold, 20-fold, 30-fold, 40-fold, 50-fold, 60-fold, 70-fold, 80-fold, 90-fold, 100-fold, 200-fold, 300-fold, 400-fold, or 500-fold following administration of the compositions described herein.

In some embodiments, SCFAs comprise butyrate. In some embodiments, the level of butyrate (e.g., in the gastrointestinal tract of the subject) is increased by 10-fold to 500-fold following administration of the compositions described herein. In some embodiments, the level of butyrate is increased by 20-fold to 250-fold following administration of compositions described herein. In some embodiments, the level of butyrate is increased by 100-fold to 500-fold following administration of the compositions described herein. In some embodiments, the level of butyrate is increased by at least 2-fold, 5-fold 10-fold, 20-fold, 30-fold, 40-fold, 50-fold, 60-fold, 70-fold, 80-fold, 90-fold, 100-fold, 200-fold, 300-fold, 400-fold, or 500-fold following administration of the compositions described herein.

In some embodiments, SCFAs comprise propionate. In some embodiments, the level of propionate (e.g., in the gastrointestinal tract of the subject) is increased by 10-fold to 500-fold following administration of the compositions described herein. In some embodiments, the level of propionate is increased by 20-fold to 250-fold following administration of compositions described herein. In some embodiments, the level of propionate is increased by 100-fold to 500-fold following administration of the compositions described herein. In some embodiments, the level of propionate is increased by at least 2-fold, 5-fold 10-fold, 20-fold, 30-fold, 40-fold, 50-fold, 60-fold, 70-fold, 80-fold, 90-fold, 100-fold, 200-fold, 300-fold, 400-fold, or 500-fold following administration of the compositions described herein.

In some embodiments, SCFAs comprise acetate. In some embodiments, the level of acetate (e.g., in the gastrointestinal tract of the subject) is increased by 10-fold to 500-fold following administration of the compositions described herein. In some embodiments, the level of acetate is increased by 20-fold to 250-fold following administration of compositions described herein. In some embodiments, the level of acetate is increased by 100-fold to 500-fold following administration of the compositions described herein. In some embodiments, the level of acetate is increased by at least 2-fold, 5-fold 10-fold, 20-fold, 30-fold, 40-fold, 50-fold, 60-fold, 70-fold, 80-fold, 90-fold, 100-fold, 200-fold, 300-fold, 400-fold, or 500-fold following administration of the compositions described herein.

In some embodiments, SCFAs comprise formate. In some embodiments, the level of acetate (e.g., in the gastrointestinal tract of the subject) is increased by 10-fold to 500-fold following administration of the compositions described herein. In some embodiments, the level of formate is increased by 20-fold to 250-fold following administration of compositions described herein. In some embodiments, the level of formate is increased by 100-fold to 500-fold following administration of the compositions described herein. In some embodiments, the level of formate is increased by at least 2-fold, 5-fold 10-fold, 20-fold, 30-fold, 40-fold, 50-fold, 60-fold, 70-fold, 80-fold, 90-fold, 100-fold, 200-fold, 300-fold, 400-fold, or 500-fold following administration of the compositions described herein.

In some embodiments, the subject is infected with a pathogenic organism. In some embodiments, the subject has a Clostridium difficile infection (CDI). In some embodiments, the CDI is recurrent (rCDI). rCDI is CDI that occurs more than once in the same subject and is associated with reduced short chain fatty acids (SCFAs), increased primary bile acids, and decreased secondary bile acids in the gut microbiota of the subject.

Bile acids are steroid acids that allow the digestion of dietary fats and oils by acting as surfactants that turn the fats and oils into micelles. Bile acids also act as hormones utilizing the farnesoid X receptor and GBPAR1. Primary bile acids are synthesized in the liver from cholesterol and a conjugated with either taurine or glycine prior to secretion. When the primary bile acids are secreted into the lumen of the intestine, bacteria partially dehydroxylate and remove the glycine or taurine groups, forming secondary bile acids.

Non-limiting examples of primary bile acids are cholic acid (CA), chenodeoxycholic acid (CDCA), glycocholic acid (GCA), glycochenodeoxycholic acid (GCDCA), glycodeoxycholic acid (GDCA), taurocholic acid (TCA), and turochenodeoxycholic acid (TCDCA). Non-limiting examples of secondary bile acids are deoxycholic acid (DCA), lithocholic acid (LCA), ursodeoxycholic acid (UDCA), taurodeoxycholic acid (TDCA), taurolithocholic acid (TLCA), and tauroursodeoxycholic acid (TUDCA).

Pathogenic organism infection, including Clostridium difficile infection and rCDI, are associated with increased primary bile acids and reduced secondary bile acids. The primary bile acids are reduced, and the secondary bile acids are increased following fecal matter transplant (FMT) (Seekatz, et al., Anaerobe (2018) 53: 64-73). In some embodiments, administration of the bacterial strains or a pharmaceutical composition as described herein reduces primary bile acids and/or increases secondary bile acids.

In some embodiments, the levels of primary bile acids are reduced by 10-fold to 100,000-fold following administration of the bacterial strains or the pharmaceutical composition. In some embodiments, the levels of primary bile acids are reduced by 10-fold to 1,000-fold following administration of the bacterial strains or the pharmaceutical composition. In some embodiments, the levels of primary bile acids are reduced 20-fold to 10,000-fold following administration of the bacterial strains or the pharmaceutical composition. In some embodiments, the levels of primary bile acids are reduced by 10-fold, 100-fold, 200-fold, 300-fold, 400-fold, 500-fold, 600-fold, 700-fold, 800-fold, 900-fold, 1,000-fold, 10,000-fold, 20,000-fold, 30,000-fold, 40,000-fold, 50,000-fold, 60,000-fold, 70,000-fold, 80,000-fold, 90,000-fold, or 100,000-fold following administration of the bacterial strains or the pharmaceutical composition.

In some embodiments, the levels of secondary bile salts are increased by 10-fold to 10,000-fold following administration of the bacterial strains or the pharmaceutical composition. In some embodiments, the levels of secondary bile acids are increased by 10-fold to 1,000-fold following administration of the bacterial strains or the pharmaceutical composition. In some embodiments, the levels of secondary bile acids are increased by 20-fold to 100-fold following administration of the bacterial strains or the pharmaceutical composition. In some embodiments, the levels of secondary bile acids are increased by 10-fold, 20-fold, 50-fold, 100-fold, 200-fold, 300-fold, 400-fold, 500-fold, 600-fold, 700-fold, 800-fold, 900-fold, 1,000-fold, 2,000-fold, 3,000-fold, 4,000-fold, 5,000-fold, 6,000-fold, 7,000-fold, 8,000-fold, 9,000-fold, or 1,000-fold following administration of the bacterial strains or the pharmaceutical composition.

In general, short chain fatty acids (SCFAs) are fatty acids containing six or less carbon atoms. SCFAs are produced when dietary fiber is fermented in the intestine. Non-limiting examples of SCFAs include hexanoate, pentanoate, butyrate, propionate, acetate, and formate. SCFAs are primarily absorbed in the portal vein following lipid digestion, and can affect the production of lipids, energy, and vitamins. Further, SCFAs play a critical role in maintaining intestinal epithelial cell membrane integrity for preventing pathogenic organism (e.g., Clostridium difficile) infection.

In one aspect, the disclosure provides methods comprising administration of multiple doses of the pharmaceutical compositions. In some embodiments, the disclosure provides methods comprising administration of antibiotic (e.g., vancomycin) followed by multiple doses of the pharmaceutical compositions. In some embodiments, administration of multiple doses of the pharmaceutical compositions described herein provides enhanced colonization (engraftment) of one or more bacterial strains of the pharmaceutical compositions as compared to administration of a single dose of the pharmaceutical composition. In some embodiments, administration of multiple doses of the pharmaceutical compositions described herein provides enhanced recovery of one or more bacterial strains of the pharmaceutical compositions as compared to administration of a single dose of the pharmaceutical composition. In some embodiments, administration of multiple doses of the pharmaceutical compositions described herein provides increased abundance of one or more bacterial strains of the pharmaceutical compositions as compared to administration of a single dose of the pharmaceutical composition. In some embodiments, administration of multiple doses of the pharmaceutical compositions described herein provides an increase in the number of subjects that were colonized with of all of bacterial strains of the pharmaceutical compositions as compared to administration of a single dose of the pharmaceutical composition. In some embodiments, administration of multiple doses of the pharmaceutical compositions described herein provides durable colonization (e.g., up to 6 months) of one or more bacterial strains of the pharmaceutical compositions as compared to administration of a single dose of the pharmaceutical composition. In some embodiments, administration of multiple doses of the pharmaceutical compositions described herein provides durable colonization (e.g., up to 6 months) of all of the bacterial strains of the pharmaceutical compositions as compared to administration of a single dose of the pharmaceutical composition. It should further be appreciated that administration of multiple dose my results in a combination of the results described. Thus, for example, in some embodiments, administration of multiple doses of the pharmaceutical compositions described herein provides enhanced colonization (engraftment) and increased rate of recovery of one or more bacterial strains of the pharmaceutical compositions as compared to administration of a single dose of the pharmaceutical composition.

In some embodiments, administration of multiple doses of the pharmaceutical compositions described herein provides enhanced colonization (engraftment) of one or more bacterial strains of the pharmaceutical compositions as compared to administration of a single dose of the pharmaceutical composition. Administration of multiple doses of the pharmaceutical composition may result in enhanced colonization (engraftment) and an increased abundance of each of the bacterial strains of the pharmaceutical composition. In some embodiments, administration of a single dose of the pharmaceutical composition results in the same or a similar level of engraftment (e.g., total bacteria) as administration of multiple doses of the pharmaceutical composition, however the engraftment may be dominated by one bacterial strain or only a subset of the bacterial strains of the pharmaceutical compositions.

Any of the methods described herein may involve administering an antibiotic to the subject prior to administration of the pharmaceutical compositions described herein. In some embodiments, the antibiotic is vancomycin, fidaxomycin or ridinilazole. Non-limiting examples of antibiotics that may be used in any of the methods provided herein include cephalosporin antibiotics cephalexin, cefuroxime, cefadroxil, cefazolin, cephalothin, cefaclor, cefamandole, cefoxitin, cefprozil, ceftobiprole, clindamycin, ceftriaxone, cefotaxime, cefazolin, cefoperazone, cefuroxime, cefmetazole, fluoroquinolone, ciprofloxacin, Levaquin, floxin, tequin, avelox, norflox, tetracycline, minocycline, oxytetracycline, doxycycline, amoxicillin, ampicillin, penicillin V, dicloxacillin, benzylpenicillin, carbenicillin, vancomycin, and methicillin, ertapenem, doripenem, imipenem/cilastatin, meropenem, clavulanate, tazobactam, piperacillin, ceftriaxone, cefotaxime, cefazolin, fluoroquinolone, imipenem, meropenem, metronidazole, fidaxomyxin, or ridinilazole.

In some embodiments, any of the methods described herein may further comprise administering vancomycin to the subject prior to administration of the pharmaceutical compositions described herein. In some embodiments, the method does not comprise administering an antibiotic to the subject prior to administration of the pharmaceutical compositions described herein. In some embodiments, the method does not comprise administering vancomycin to the subject prior to administration of the pharmaceutical compositions described herein. Vancomycin administration has been found to alter the composition of human gut microbiota. See, e.g., Reijnders et al. Cell Metabolism (2016) 24(1): 63-72. Without wishing to be bound by any particular theory, it is thought that administration of vancomycin may aid engraftment of the bacterial strain(s) of the pharmaceutical compositions described herein, for example by removing other microbes present in the gastrointestinal tract.

In some embodiments, the antibiotic (e.g., vancomycin) is administered to the subject once, as a single dose. In some embodiments, the antibiotic (e.g., vancomycin) is administered to the subject in multiple doses. In some embodiments, the antibiotic (e.g., vancomycin) is administered to the subject in at least 2, 3, 4, 5, 6, 7, 8, 9, 10, 11, 12, 13, 14, 15 or more doses. The multiple doses of the antibiotic (e.g., vancomycin) may be administered to the subject at regular intervals prior to administering any of the pharmaceutical compositions described herein. In some embodiments, each of the multiple doses of the antibiotic (e.g., vancomycin) are administered on consecutive days (e.g., first dose on day 1, second dose of day 2, third dose on day 3, etc.). In some embodiments, the antibiotic (e.g., vancomycin) is administered to the subject for 2, 3, 4, 5, 6, 7, 8, 9, 10, 11, 12, 13, 14, 15, or more consecutive days. In some embodiments, the antibiotic (e.g., vancomycin) is administered to the subject each day for three consecutive days. In some embodiments, the antibiotic (e.g., vancomycin) administered to the subject each day for five consecutive days. In some embodiments, the antibiotic (e.g., vancomycin) administered to the subject each day for seven consecutive days. In some embodiments, the antibiotic (e.g., vancomycin) administered to the subject for one day. In any of the embodiments described herein, a subject may be administered one or more doses of a first antibiotic followed by one or more doses of a second antibiotic.

In some embodiments, a single dose, or the first dose in a treatment regimen of multiple doses, is administered, the same day as the administration of the final dose of the antibiotic (e.g., vancomycin). In some embodiments, a single dose, or the first dose in a treatment regimen of multiple doses, is administered, the day after administration of the final dose of the antibiotic (e.g., vancomycin). In some embodiments, a single dose, or the first dose in a treatment regimen of multiple doses, is administered, two days after administration of the final dose of the antibiotic (e.g., vancomycin). In some embodiments, the methods provided herein allow for a wash out day between the final dose of the antibiotic (e.g., vancomycin) and the first dose of the pharmaceutical composition. In some embodiments, a single dose, or the first dose in a treatment regimen of multiple doses, is administered, three days, four days, five days, six days, ten days or more, after administration of the final dose of the antibiotic (e.g., vancomycin). In some embodiments, the methods provided herein allow for multiple wash out days between the final dose of the antibiotic (e.g., vancomycin) and the first dose of the pharmaceutical composition.

Each dose of the antibiotic (e.g., vancomycin) may be the same amount of the antibiotic or may be a different amount of the antibiotic. In some embodiments, the antibiotic (e.g., vancomycin) is administered in an amount sufficient to allow for colonization of one or more of the bacterial strains of the pharmaceutical compositions described herein. In some embodiments, the subject is administered between about 50 mg and 1 g, 100 mg and 750 mg, 100 mg and 500 mg, 200 mg and 750 mg, 200 mg and 500 mg, 300 mg and 750 mg, 300 mg and 500 mg, 100 mg and 400 mg, 100 mg and 300 mg, 100 mg and 200 mg, 200 mg and 400 mg, 200 mg and 300 mg, or 450 mg to 550 mg of the antibiotic per day. As will be appreciated by one of skill in the art, the total amount of vancomycin administered to the subject per day may be administered in a single dose or between multiple doses, which in sum results in the total amount of the antibiotic per day.

In some example, the subject is administered about 500 mg vancomycin per day prior to administration of any of the pharmaceutical compositions described herein. In some embodiments, 500 mg of vancomycin per day is administered in a single dose (e.g., 500 mg). In some embodiments, 500 mg of vancomycin per day is administered in multiple doses (e.g., 2, 3, 4, 5 or more), which in sum results in 500 mg vancomycin per day. In some embodiments, 500 mg vancomycin is administered in 4 doses of 125 mg vancomycin per day. In some embodiments, 500 mg of vancomycin is administered to the subject for one day. In some embodiments, 500 mg of vancomycin is administered to the subject per day for two days. In some embodiments, 500 mg vancomycin is administered to the subject per day for three days. In some embodiments, 500 mg vancomycin is administered to the subject per day for four days. In some embodiments, 500 mg vancomycin is administered to the subject per day for five days. In some embodiments, 500 mg vancomycin is administered to the subject per day for six days. In some embodiments, 500 mg vancomycin is administered to the subject per day for seven days. In some embodiments, 500 mg vancomycin is administered to the subject per day for eight days. In some embodiments, 500 mg vancomycin is administered to the subject per day for nine days. In some embodiments, 500 mg vancomycin is administered to the subject per day for ten days.

In some embodiments, the subject is administered about 250 mg vancomycin per day prior to administration of any of the pharmaceutical compositions described herein. In some embodiments, 250 mg vancomycin per day is administered in a single dose (e.g., 250 mg). In some embodiments, 250 mg vancomycin per day is administered in multiple doses (e.g., 2, 3, 4, 5 or more), which in sum results in 250 mg vancomycin per day. In some embodiments, 250 mg vancomycin is administered in 2 doses of 125 mg vancomycin per day. In some embodiments, 250 mg vancomycin is administered to the subject for one day. In some embodiments, 250 mg vancomycin is administered to the subject per day for two days. In some embodiments, 250 mg vancomycin is administered to the subject per day for three days. In some embodiments, 250 mg vancomycin is administered to the subject per day for four days. In some embodiments, 250 mg vancomycin is administered to the subject per day for five days. In some embodiments, 250 mg vancomycin is administered to the subject per day for six days. In some embodiments, 250 mg vancomycin is administered to the subject per day for seven days. In some embodiments, 250 mg vancomycin is administered to the subject per day for eight days. In some embodiments, 250 mg vancomycin is administered to the subject per day for nine days. In some embodiments, 250 mg vancomycin is administered to the subject per day for ten days.

In some embodiments, the subject is administered about 125 mg vancomycin per day prior to administration of any of the pharmaceutical compositions described herein. In some embodiments, the 125 mg vancomycin per day is administered in a single dose (e.g., 125 mg). In some embodiments, the 125 mg vancomycin per day is administered in multiple doses (e.g., 2, 3, 4, 5 or more), which in sum results in 125 mg vancomycin per day. In some embodiments, 125 mg vancomycin is administered to the subject for one day. In some embodiments, 125 mg vancomycin is administered to the subject per day for two days. In some embodiments, 125 mg vancomycin is administered to the subject per day for three days. In some embodiments, 125 mg vancomycin is administered to the subject per day for four days. In some embodiments, 125 mg vancomycin is administered to the subject per day for five days. In some embodiments, 125 mg vancomycin is administered to the subject per day for six days. In some embodiments, 125 mg vancomycin is administered to the subject per day for seven days. In some embodiments, 125 mg vancomycin is administered to the subject per day for eight days. In some embodiments, 125 mg vancomycin is administered to the subject per day for nine days. In some embodiments, 125 mg vancomycin is administered to the subject per day for ten days.

In some embodiments, the disclosure provides methods comprising administering one or more antibiotics to the subject and subsequently administering any of the bacterial compositions to the subject once, twice, 3 times, 4 times, 5 times, 6 times, 7 times, 8 times, 9 times, or at least 10 times, or more. In some embodiments, the disclosure provides methods comprising administering one or more antibiotics to the subject and subsequently administering any of the bacterial compositions described herein to the subject in multiple doses at a regular interval, such as every 2 weeks, every month, every 2 months, every 3 months, every 4 months, every 5 months, every 6 months, or more. In some embodiments, one dose of any of the compositions described herein is administered and a second dose of the composition is administered the following day (e.g., consecutive day). In some embodiments, one dose of any of the compositions described herein is administered and each of the additional doses of the composition are administered on consecutive days (e.g., first dose on day 1, second dose of day 2, third dose on day 3, etc.).

In one aspect, the disclosure provides methods comprising administering one or more antibiotics to the subject and subsequently administering any of the bacterial compositions as multiple daily doses of the pharmaceutical compositions. In some embodiments, the pharmaceutical compositions are administered on a daily basis for 2 days, 3 days, 4, days, 5, days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, 12 months or more.

In some embodiments, the antibiotic (e.g., vancomycin) is administered according to a pulse tapered regime. See e.g., Sirbu et al., Clinical Infectious Diseases (2017) 65: 1396-1399.

In some embodiments, the antibiotic (e.g., vancomycin) is administered to the subject at least 1, 2, 3, 4, 5, 6, 7 days or more prior to administration of the pharmaceutical compositions described herein. In some embodiments, administration of antibiotic (e.g., vancomycin) is terminated at least one day (e.g., 1, 2, 3, 4, 5, or more) prior to administration of any of the pharmaceutical compositions described herein.

In some embodiments, additional antibiotics are administered in combination with the vancomycin regimes provided herein.

It should be appreciated, in some embodiments, that any of the vancomycin doses or administration regimens may be combined with any of the pharmaceutical composition doses or administration regimens provided herein.

In one aspect the disclosure provides methods comprising the administration of an antibiotic (e.g., vancomycin) followed by the administration of a pharmaceutical composition provided herein, wherein the administration of an antibiotic (e.g., vancomycin) is followed by the administration of a single dose or multiple doses of the pharmaceutical composition. In some embodiments, administration of an antibiotic (e.g., vancomycin) followed by the administration of a single dose or multiple doses of the pharmaceutical composition results in an increase in the abundance of bacterial strains of the pharmaceutical compositions in the microbiome of the subject (engraftment) compared to the administration of a pharmaceutical composition without the administration of the antibiotic. In some embodiments, administration of an antibiotic (e.g., vancomycin) followed by the administration of a single dose or multiple doses of the pharmaceutical composition results in an increase in the duration of the colonization of bacterial strains of the pharmaceutical composition in the microbiome of the subject (e.g., up to 6 months) compared to the administration of a pharmaceutical composition without the administration of the antibiotic.

In some embodiments, administration of an antibiotic (e.g., vancomycin) followed by the administration of a single dose or multiple doses of the pharmaceutical composition results in an increase in the rate of engraftment of the initial amount of the bacterial strains of the pharmaceutical composition in the microbiome of the subject by between ten- to one hundred-fold (e.g., within the first 48 hours) compared to the administration of a pharmaceutical composition without the administration of the antibiotic.

In some embodiments, administration of an antibiotic (e.g., vancomycin) followed by the administration of a single dose or multiple doses of the pharmaceutical composition results in a greater number (amount) of subjects having all of the bacterial strains of the pharmaceutical composition present in their microbiome as compared to compared to the administration of a pharmaceutical composition without the administration of the antibiotic.

In some embodiments, administration of an antibiotic (e.g., vancomycin) followed by the administration of multiple doses of the pharmaceutical composition results in an increase in the abundance of bacterial strains of the pharmaceutical composition in the microbiome of the subject (engraftment) compared to the administration of a single dose of the pharmaceutical composition. In some embodiments, the disclosure provides methods comprising the administration of a pharmaceutical composition provided herein, wherein the administration of multiple doses of the pharmaceutical composition increases the abundance of bacterial strains in the microbiota of the subject (engraftment) of the pharmaceutical composition in the microbiome of the subject compared to the administration of a single dose of the pharmaceutical composition.

In some embodiments, administration of an antibiotic (e.g., vancomycin) followed by the administration of multiple doses of the pharmaceutical composition results in an increase in the rate of engraftment of the initial amount of the bacterial strains of the pharmaceutical composition in the microbiome of the subject as compared to the administration of a single dose of the pharmaceutical composition. In some embodiments, the disclosure provides methods comprising the administration of a pharmaceutical composition provided herein, wherein the administration of multiple doses of the pharmaceutical composition increases the rate of engraftment of the initial amount of the bacterial strains of the pharmaceutical composition in the microbiome of the subject compared to the administration of a single dose of the pharmaceutical composition.

In some embodiments, administration of an antibiotic (e.g., vancomycin) followed by the administration of multiple doses of the pharmaceutical composition results in a higher abundance of the bacterial strains of the pharmaceutical composition in the microbiome of the subject as compared to the administration of a single dose of the pharmaceutical composition. In some embodiments, the disclosure provides methods comprising the administration of a pharmaceutical composition provided herein, wherein the administration of multiple doses of the pharmaceutical composition results in higher abundance of the bacterial strains of the pharmaceutical composition in the microbiome of the subject compared to the administration of a single dose of the pharmaceutical composition.

In some embodiments, administration of an antibiotic (e.g., vancomycin) followed by the administration of multiple doses of the pharmaceutical composition results in a greater number (amount) of subjects having all of the bacterial strains of the pharmaceutical composition present in their microbiome as compared to the administration of a single dose of the pharmaceutical composition. In some embodiments, the disclosure provides methods comprising the administration of a pharmaceutical composition provided herein, wherein the administration of multiple doses of the pharmaceutical results in a greater number (amount) of subject having all of the bacterial strains of the pharmaceutical composition in their microbiome as compared to the administration of a single dose of the pharmaceutical composition.

In some embodiments, administration of an antibiotic (e.g., vancomycin) followed by the administration of multiple doses of the pharmaceutical composition results in an accelerated recovery of the microbiome (e.g., increase in bacterial species of Bacteroidetes and/or Firmicutes, and/or decrease in Proteobacteria) as compared to the administration of a single dose of the pharmaceutical composition. In some embodiments, the disclosure provides methods comprising the administration of a pharmaceutical composition provided herein, wherein the administration of multiple doses of the pharmaceutical results in an accelerated recovery of the microbiome (e.g., increase in bacterial species of Bacteroidetes and/or Firmicutes, and/or decrease in Proteobacteria) as compared the administration of a single dose of the pharmaceutical composition.

In some embodiments, administration of an antibiotic (e.g., vancomycin) followed by the administration of a single dose or multiple doses of the pharmaceutical composition results in an accelerated recovery of the microbiome (e.g., increase in bacterial species of Bacteroidetes and/or Firmicutes, and/or decrease in Proteobacteria) as compared to the administration of an antibiotic (e.g., vancomycin) without the administration of a pharmaceutical composition.

In some embodiments, the methods described herein may involve subjecting the subject to a bowel lavage (bowel irrigation, whole bowel irrigation, gastrointestinal lavage, gastric lavage) prior to administration of the compositions described herein. In some embodiments, a bowel lavage may remove or aid in removing microbiota from the gastrointestinal tract of the subject, creating a niche for the bacterial strains of the compositions described herein. In some embodiments, the bowel lavage may be an oral bowel lavage or a rectal bowel lavage.

Methods of performing a bowel lavage are known in the art, and generally involve the rapid administration of large volumes of a solution, such as polyethylene glycol or a balanced electrolyte solution. A rectal bowel lavage can involve the administration of a solution or a suppository containing the pharmaceutical composition. A bowel lavage may be performed under doctor supervision, hospitalization, or at home.

Any of the compositions described herein, including the pharmaceutical compositions and food products comprising the compositions, may contain bacterial strains in any form, for example in an aqueous form, such as a solution or a suspension, embedded in a semi-solid form, in a powdered form or freeze dried form. In some embodiments, the composition or the bacterial strains of the composition are lyophilized. In some embodiments, a subset of the bacterial strains in a composition is lyophilized. Methods of lyophilizing compositions, specifically compositions comprising bacteria, are well known in the art. See, e.g., U.S. Pat. Nos. 3,261,761; 4,205,132; PCT Publications WO 2014/029578 and WO 2012/098358, herein incorporated by reference in their entirety. The bacteria may be lyophilized as a combination and/or the bacteria may be lyophilized separately and combined prior to administration. A bacterial strain may be combined with a pharmaceutical excipient prior to combining it with the other bacterial strain or multiple lyophilized bacteria may be combined while in lyophilized form and the mixture of bacteria, once combined may be subsequently be combined with a pharmaceutical excipient. In some embodiments, the bacterial strain is a lyophilized cake. In some embodiments, the compositions comprising the one or more bacterial strains are a lyophilized cake.

In some embodiments, one or more of the bacterial strains of the compositions, including pharmaceutical compositions and food products, has been spray-dried. In some embodiments, a subset of the bacterial strains is spray-dried. The process of spray-drying refers to production of dry powder from a liquid comprising bacterial compositions (See, e.g., Ledet, et al., Spray Draying of Pharmaceuticals in “Lyophilized Biologics and Vaccines” pages 273-294, Springer). In general, the process involves rapidly drying the bacterial compositions with a hot gas. A bacterial strain may be combined with a pharmaceutical excipient prior to combining it with the other bacterial strains or multiple spray-dried bacterial strains may be combined while in spray-dried form and the mixture of bacterial strains, once combined, may be subsequently combined with a pharmaceutical excipient.

The bacterial strains of the composition can be manufactured using fermentation techniques well known in the art. In some embodiments, the active ingredients are manufactured using anaerobic fermenters, which can support the rapid growth of anaerobic bacterial species. The anaerobic fermenters may be, for example, stirred tank reactors or disposable wave bioreactors. Culture media such as BL media and EG media, or similar versions of these media devoid of animal components, can be used to support the growth of the bacterial species. The bacterial product can be purified and concentrated from the fermentation broth by traditional techniques, such as centrifugation and filtration, and can optionally be dried and lyophilized by techniques well known in the art.

In some embodiments, the composition of bacterial strains may be formulated for administration as a pharmaceutical composition. The term “pharmaceutical composition” as used herein means a product that results from the mixing or combining of at least one active ingredient, such as any two or more purified bacterial strains described herein, and one or more inactive ingredients, which may include one or more pharmaceutically acceptable excipient.

An “acceptable” excipient refers to an excipient that must be compatible with the active ingredient and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Pharmaceutical compositions disclosed herein can be prepared in accordance with methods well known and routinely practiced in the art (see e.g., Remington: The Science and Practice of Pharmacy, Mack Publishing Co. 20th ed. 2000). The pharmaceutical compositions described herein may further comprise any carriers or stabilizers in the form of a lyophilized formulation or an aqueous solution. Acceptable excipients, carriers, or stabilizers may include, for example, buffers, antioxidants, preservatives, polymers, chelating reagents, and/or surfactants. Pharmaceutical compositions are preferably manufactured under GMP conditions. The pharmaceutical compositions can be used orally, nasally or parenterally, for instance, in the form of capsules, tablets, pills, sachets, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like. In some embodiments, the pharmaceutical compositions can be used by injection, such as by intravenous, intramuscular, subcutaneous, or intradermal administration.

In some embodiments, the compositions comprising bacterial strains are formulated for delivery to the intestines (e.g., the small intestine and/or the colon). In some embodiments, the compositions comprising bacterial strains are formulated with an enteric coating that increases the survival of the bacteria through the harsh environment in the stomach. The enteric coating is one which resists the action of gastric juices in the stomach so that the bacteria of the composition therein will pass through the stomach and into the intestines. The enteric coating may readily dissolve when in contact with intestinal fluids, so that the bacteria enclosed in the coating will be released in the intestinal tract. Enteric coatings may consist of polymer and copolymers well known in the art, such as commercially available EUDRAGIT (Evonik Industries). (See e.g., Zhang, AAPS PharmSciTech (2016) 17(1): 56-67).

The compositions comprising bacterial strains may also be formulated for rectal delivery to the intestine (e.g., the colon). Thus, in some embodiments, compositions comprising bacterial strains may be formulated for delivery by suppository, colonoscopy, endoscopy, sigmoidoscopy or enema. A pharmaceutical preparation or formulation and particularly a pharmaceutical preparation for oral administration, may include an additional component that enables efficient delivery of the compositions of the disclosure to the intestine (e.g., the colon). A variety of pharmaceutical preparations that allow for the delivery of the compositions to the intestine (e.g., the colon) can be used. Examples thereof include pH sensitive compositions, more specifically, buffered sachet formulations or enteric polymers that release their contents when the pH becomes alkaline after the enteric polymers pass through the stomach. When a pH sensitive composition is used for formulating the pharmaceutical preparation, the pH sensitive composition is preferably a polymer whose pH threshold of the decomposition of the composition is between about 6.8 and about 7.5. Such a numeric value range is a range in which the pH shifts toward the alkaline side at a distal portion of the stomach, and hence is a suitable range for use in the delivery to the colon. It should further be appreciated that each part of the intestine (e.g., the duodenum, jejunum, ileum, cecum, colon and rectum), has different biochemical and chemical environment. For instance, parts of the intestines have different pHs, allowing for targeted delivery by compositions that have a specific pH sensitivity. Thus, the compositions provided herein may be formulated for delivery to the intestine or specific parts of the intestine (e.g., the duodenum, jejunum, ileum, cecum, colon and rectum) by providing formulations with the appropriate pH sensitivity. (See e.g., Villena et al., Int J Pharm (2015) 487 (1-2): 314-9).

Another embodiment of a pharmaceutical preparation useful for delivery of the compositions to the intestine (e.g., the colon) is one that ensures the delivery to the colon by delaying the release of the contents (e.g., the bacterial strains) by approximately 3 to 5 hours, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Additional examples of pharmaceutical compositions that allow for the delivery to the intestine (e.g., the colon) include bioadhesive compositions which specifically adhere to the colonic mucosal membrane (for example, a polymer described in the specification of U.S. Pat. No. 6,368,586) and compositions into which a protease inhibitor is incorporated for protecting particularly a biopharmaceutical preparation in the gastrointestinal tracts from decomposition due to an activity of a protease.

Another example of a system enabling the delivery to the intestine (e.g., the colon) is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

A further example of a system enabling the delivery of a composition to the intestine (e.g., the colon), is a composition that includes a coating that can be removed by an enzyme present in the gut (e.g., the colon), such as, for example, a carbohydrate hydrolase or a carbohydrate reductase. Such a system is not particularly limited, and more specific examples thereof include systems which use food components such as non-starch polysaccharides, amylose, xanthan gum, and azopolymers.

The compositions provided herein can also be delivered to specific target areas, such as the intestine, by delivery through an orifice (e.g., a nasal tube) or through surgery. In addition, the compositions provided herein that are formulated for delivery to a specific area (e.g., the cecum or the colon), may be administered by a tube (e.g., directly into the small intestine). Combining mechanical delivery methods such as tubes with chemical delivery methods such as pH specific coatings, allow for the delivery of the compositions provided herein to a desired target area (e.g., the cecum or the colon).

The compositions comprising bacterial strains are formulated into pharmaceutically acceptable dosage forms by conventional methods known to those of skill in the art. Dosage regimens are adjusted to provide the optimum desired response (e.g., the prophylactic or therapeutic effect). In some embodiments, the dosage form of the composition is a tablet, pill, capsule, powder, granules, solution, or suppository. In some embodiments, the pharmaceutical composition is formulated for oral administration. In some embodiments, the pharmaceutical composition is formulated such that the bacteria of the composition, or a portion thereof, remain viable after passage through the stomach of the subject. In some embodiments, the pharmaceutical composition is formulated for rectal administration, e.g. as a suppository. In some embodiments, the pharmaceutical composition is formulated for delivery to the intestine or a specific area of the intestine (e.g., the colon) by providing an appropriate coating (e.g., a pH specific coating, a coating that can be degraded by target area specific enzymes, or a coating that can bind to receptors that are present in a target area).

Dosages of the active ingredients in the pharmaceutical compositions disclosed herein can be varied so as to obtain an amount of the active ingredient which is effective to achieve the desired pharmaceutical response for a particular subject, composition, and mode of administration, without being toxic or having an adverse effect on the subject. The selected dosage level depends upon a variety of factors including the activity of the particular compositions employed, the route of administration, the time of administration, the duration of the treatment, other drugs, compounds and/or materials used in combination with the particular compositions employed, the age, sex, weight, condition, general health and prior medical history of the subject being treated, and like factors.

A physician, veterinarian or other trained practitioner, can start doses of the pharmaceutical composition at levels lower than that required to achieve the desired therapeutic effect and gradually increase the dosage until the desired effect is achieved. In general, effective doses of the compositions, for the prophylactic or therapeutic treatment of groups of people as described herein vary depending upon many different factors, including routes of administration, physiological state of the subject, whether the subject is human or an animal, other medications administered, and the therapeutic effect desired. Dosages need to be titrated to optimize safety and efficacy. In some embodiments, the dosing regimen entails oral administration of a dose of any of the compositions described herein. In some embodiments, the dosing regimen entails oral administration of multiple doses of any of the compositions described herein. In some embodiments, the composition is administered orally the subject once, twice, 3 times, 4 times, 5 times, 6 times, 7 times, 8 times, 9 times, or at least 10 times. In some embodiments, any of the compositions described herein are administered the subject in multiple doses at a regular interval, such as every 2 weeks, every month, every 2 months, every 3 months, every 4 months, every 5 months, every 6 months, or more.

The compositions, including the pharmaceutical compositions disclosed herein, include compositions that contain selected bacterial strains. The amount of bacteria, including the amount of bacteria of each of the bacterial strains, in the compositions, including pharmaceutical compositions, may be expressed in weight, number of bacteria and/or CFUs (colony forming units). In some embodiments, the compositions, including pharmaceutical compositions, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more of each of the bacterial strains per dosage amount. In some embodiments, the compositions, including pharmaceutical compositions, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more total bacteria per dosage amount. It should further be appreciated that bacteria of each of the bacterial strains may be present in different amounts. Thus, for instance, as a non-limiting example, composition may include 10³ of bacteria A, 10⁴ of bacteria B and 10⁶ of bacteria C. In some embodiments, compositions, including pharmaceutical composition, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more CFUs of each of the bacterial strains per dosage amount. In some embodiments, compositions, including pharmaceutical compositions, comprise about 10¹, about 10², about 10³, about 10⁴, about 10⁵, about 10⁶, about 10⁷, about 10⁸, about 10⁹, about 10¹⁰, about 10¹¹, about 10¹², about 10¹³ or more CFUs in total for all of the bacterial strains combined per dosage amount. As discussed above, bacteria of each of the bacterial strains may be present in different amounts. In some embodiments, the compositions, including pharmaceutical compositions, contain about 10⁻⁷, about 10⁻⁶, about 10⁻⁵, about 10⁴, about 10⁻³, about 10⁻², about 10⁻¹ or more grams of bacteria of each of the bacterial strains in the composition per dosage amount. In some embodiments, the compositions, including pharmaceutical compositions, contain about 10⁻⁷, about 10⁻⁶, about 10⁻⁵, about 10⁴, about 10⁻³, about 10⁻², about 10⁻¹ or more grams of bacteria in total for all of the bacterial strains combined per dosage amount.

In some embodiments, the dosage amount is one administration device (e.g., one table, pill or capsule). In some embodiments, the dosage amount is the amount administered at one time, which may be in the form of more than one administration device (e.g., more than one table, pill or capsule). In some embodiment, the dosage amount is the amount that is administered in a particular period (e.g., one day or one week).

As described herein, any of the pharmaceutical compositions described herein may be administered once, as a single dose. In some embodiments, the pharmaceutical compositions described herein are administered in multiple doses. In some embodiments, each dose is administered in the form of one or more capsules. In some embodiments, each dose comprises administration of multiple capsules. In some embodiments, each dose is administered in the form of 1, 2, 3, 4, 5, 6, 7, 8, 9, 10, 11, 12, 13, 14, 15, or more capsules.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² of each of the bacterial strains per capsule.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria per capsule. In some embodiments, each capsule contains between 10⁷ and 10⁹, between 10⁷ and 10⁸, or between 10⁸ and 10⁹ total bacteria. In some embodiments, each capsule contains about 1.0×10⁷, 2.0×10⁷, 3.0×10⁷, 4.0×10⁷, 5.0×10⁷, 6.0×10⁷, 7.0×10⁷, 8.0×10⁷, 9.0×10⁷, 1.0×10⁸, 2.0×10⁸, 3.0×10⁸, 4.0×10⁸, 5.0×10⁸, 6.0×10⁸, 7.0×10⁸, 8.0×10⁸, 9.0×10⁸, 1.0×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2.0×10⁹, 2.1×10⁹, 2.2×10⁹, 2.3×10⁹, 2.4×10⁹, 2.5×10⁹, 2.6×10⁹, 2.7×10⁹, 2.8×10⁹, 2.9×10⁹, 3.0×10⁹, 3.1×10⁹, 3.2×10⁹, 3.3×10⁹, 3.4×10⁹, 3.5×10⁹, 3.6×10⁹, 3.7×10⁹, 3.8×10⁹, 3.9×10⁹, 4.0×10⁹, 4.1×10⁹, 4.2×10⁹, 4.3×10⁹, 4.4×10⁹, 4.5×10⁹, 4.6×10⁹, 4.7×10⁹, 4.8×10⁹, 4.9×10⁹, 5.0×10⁹ total bacteria. In some embodiments, each capsule contains about 8.0×10⁸ total bacteria. In some embodiments, each capsule contains about 1.6×10⁹ total bacteria. In some embodiments, each capsule contains about 8.0×10⁸ CFUs. In some embodiments, each capsule contains about 1.6×10⁹ CFUs.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² of each bacterial strain per capsule.

In some embodiments, the pharmaceutical compositions contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² CFUs of each of the bacterial strains per dosage amount. In some embodiments, the pharmaceutical compositions contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total CFUs per dosage amount.

In some embodiments, the pharmaceutical compositions contain at least about 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3.0×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4.0×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5.0×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6.0×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7.0×10⁸, 7.1×10⁸, 7.2×10⁸, 7.3×10⁸, 7.4×10⁸, 7.5×10⁸, 7.6×10⁸, 7.7×10⁸, 7.8×10⁸, 7.9×10⁸, 8.0×10⁸, 8.1×10⁸, 8.2×10⁸, 8.3×10⁸, 8.4×10⁸, 8.5×10⁸, 8.6×10⁸, 8.7×10⁸, 8.8×10⁸, 8.9×10⁸, 9.0×10⁸, 9.1×10⁸, 9.2×10⁸, 9.3×10⁸, 9.4×10⁸, 9.5×10⁸, 9.6×10⁸, 9.7×10⁸, 9.8×10⁸, 9.9×10⁸, 1.0×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2.0×10⁹, 2.1×10⁹, 2.2×10⁹, 2.3×10⁹, 2.4×10⁹, 2.5×10⁹, 2.6×10⁹, 2.7×10⁹, 2.8×10⁹, 2.9×10⁹, 3.0×10⁹, 3.1×10⁹, 3.2×10⁹, 3.3×10⁹, 3.4×10⁹, 3.5×10⁹, 3.6×10⁹, 3.7×10⁹, 3.8×10⁹, 3.9×10⁹, 4.0×10⁹, 4.1×10⁹, 4.2×10⁹, 4.3×10⁹, 4.4×10⁹, 4.5×10⁹, 4.6×10⁹, 4.7×10⁹, 4.8×10⁹, 4.9×10⁹, 5.0×10⁹, 5.1×10⁹, 5.2×10⁹, 5.3×10⁹, 5.4×10⁹, 5.5×10⁹, 5.6×10⁹, 5.7×10⁹, 5.8×10⁹, 5.9×10⁹, 6.0×10⁹, 6.1×10⁹, 6.2×10⁹, 6.3×10⁹, 6.4×10⁹, 6.5×10⁹, 6.6×10⁹, 6.7×10⁹, 6.8×10⁹, 6.9×10⁹, 7.0×10⁹, 7.1×10⁹, 7.2×10⁹, 7.3×10⁹, 7.4×10⁹, 7.5×10⁹, 7.6×10⁹, 7.7×10⁹, 7.8×10⁹, 7.9×10⁹, 8.0×10⁹, 8.1×10⁹, 8.2×10⁹, 8.3×10⁹, 8.4×10⁹, 8.5×10⁹, 8.6×10⁹, 8.7×10⁹, 8.8×10⁹, 8.9×10⁹, 9.0×10⁹, 9.1×10⁹, 9.2×10⁹, 9.3×10⁹, 9.4×10⁹, 9.5×10⁹, 9.6×10⁹, 9.7×10⁹, 9.8×10⁹, 9.9×10⁹, 1.0×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2.0×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3.0×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4.0×10¹⁰, 4.1×10¹⁰, 4.2×10¹⁰, 4.3×10¹⁰, 4.4×10¹⁰, 4.5×10¹⁰, 4.6×10¹⁰, 4.7×10¹⁰, 4.8×10¹⁰, 4.9×10¹⁰, 5.0×10¹⁰, 5.1×10¹⁰, 5.2×10¹⁰, 5.3×10¹⁰, 5.4×10¹⁰, 5.5×10¹⁰, 5.6×10¹⁰, 5.7×10¹⁰, 5.8×10¹⁰, 5.9×10¹⁰, 6.0×10¹⁰, 6.1×10¹⁰, 6.2×10¹⁰, 6.3×10¹⁰, 6.4×10¹⁰, 6.5×10¹⁰, 6.6×10¹⁰, 6.7×10¹⁰, 6.8×10¹⁰, 6.9×10¹⁰, 7.0×10¹⁰, 7.1×10¹⁰, 7.2×10¹⁰, 7.3×10¹⁰, 7.4×10¹⁰, 7.5×10¹⁰, 7.6×10¹⁰, 7.7×10¹⁰, 7.8×10¹⁰, 7.9×10¹⁰, 8.0×10¹⁰, 8.1×10¹⁰, 8.2×10¹⁰, 8.3×10¹⁰, 8.4×10¹⁰, 8.5×10¹⁰, 8.6×10¹⁰, 8.7×10¹⁰, 8.8×10¹⁰, 8.9×10¹⁰, 9.0×10¹⁰, 9.1×10¹⁰, 9.2×10¹⁰, 9.3×10¹⁰, 9.4×10¹⁰, 9.5×10¹⁰, 9.6×10¹⁰, 9.7×10¹⁰, 9.8×10¹⁰, 9.9×10¹⁰, 1.0×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2.0×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3.0×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4.0×10¹¹, 4.1×10¹¹, 4.2×10¹¹, 4.3×10¹¹, 4.4×10¹¹, 4.5×10¹¹, 4.6×10¹¹, 4.7×10¹¹, 4.8×10¹¹, 4.9×10¹¹, 5.0×10¹¹, 5.1×10¹¹, 5.2×10¹¹, 5.3×10¹¹, 5.4×10¹¹, 5.5×10¹¹, 5.6×10¹¹, 5.7×10¹¹, 5.8×10¹¹, 5.9×10¹¹, 6.0×10¹¹, 6.1×10¹¹, 6.2×10¹¹, 6.3×10¹¹, 6.4×10¹¹, 6.5×10¹¹, 6.6×10¹¹, 6.7×10¹¹, 6.8×10¹¹, 6.9×10¹¹, 7.0×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8.0×10¹¹, 8.1×10¹¹, 8.2×10¹¹, 8.3×10¹¹, 8.4×10¹¹, 8.5×10¹¹, 8.6×10¹¹, 8.7×10¹¹, 8.8×10¹¹, 8.9×10¹¹, 9.0×10¹¹, 9.1×10¹¹, 9.2×10¹¹, 9.3×10¹¹, 9.4×10¹¹, 9.5×10¹¹, 9.6×10¹¹, 9.7×10¹¹, 9.8×10¹¹, 9.9×10¹¹, or 1.0×10¹² total CFUs.

In some embodiments, the pharmaceutical composition comprises at least 1.6×10⁹ total CFUs. In some embodiments, the pharmaceutical composition comprises at least 1.6×10⁹ total CFUs and is administered as a single dose. In some embodiments, the pharmaceutical composition comprises at least 1.6×10⁹ total CFUs and is administered as multiple (e.g., 2, 3, 4, 5, or more) doses. In some embodiments, the pharmaceutical composition comprises at least 1.6×10⁹ total CFUs and is administered as two, three, four, five, six, seven, eight, nine, ten, eleven, twelve, thirteen, fourteen, fifteen or more doses. In some embodiments, each of the multiple doses are administered at regular intervals. In some embodiments, each of the multiple doses are on consecutive days (e.g., first dose on day 1, second dose of day 2, third dose on day 3, etc.).

In some embodiments, the pharmaceutical composition comprises at least 4.0×10⁹ total CFUs. In some embodiments, the pharmaceutical composition comprises at least 4.0×10⁹ total CFUs and is administered as a single dose. In some embodiments, the pharmaceutical composition comprises at least 4.0×10⁹ total CFUs and is administered as multiple (e.g., 2, 3, 4, 5, or more) doses. In some embodiments, the pharmaceutical composition comprises at least 4.0×10¹⁰ total CFUs and is administered as two, three, four, five, six, seven, eight, nine, ten, eleven, twelve, thirteen, fourteen, fifteen or more doses. In some embodiments, each of the multiple doses are administered at regular intervals. In some embodiments, each of the multiple doses are on consecutive days (e.g., first dose on day 1, second dose of day 2, third dose on day 3, etc.).

In some embodiments, the pharmaceutical composition comprises at least 8.0×10⁹ total CFUs. In some embodiments, the pharmaceutical composition comprises at least 8.0×10⁹ total CFUs and is administered as a single dose. In some embodiments, the pharmaceutical composition comprises at least 8.0×10⁹ total CFUs and is administered as multiple (e.g., 2, 3, 4, 5, or more) doses. In some embodiments, the pharmaceutical composition comprises at least 8.0×10⁹ total CFUs and is administered as two, three, four, five, six, seven, eight, nine, ten, eleven, twelve, thirteen, fourteen, fifteen or more doses. In some embodiments, each of the multiple doses are administered at regular intervals. In some embodiments, each of the multiple doses are on consecutive days (e.g., first dose on day 1, second dose of day 2, third dose on day 3, etc.).

In some embodiments, the pharmaceutical composition comprises at least 2.8×10¹⁰ total CFUs. In some embodiments, the pharmaceutical composition comprises at least 2.8×10¹⁰ total CFUs and is administered as a single dose. In some embodiments, the pharmaceutical composition comprises at least 2.8×10¹⁰ total CFUs and is administered as multiple (e.g., 2, 3, 4, 5, or more) doses. In some embodiments, the pharmaceutical composition comprises at least 2.8×10¹⁰ total CFUs and is administered as two, three, four, five, six, seven, eight, nine, ten, eleven, twelve, thirteen, fourteen, fifteen or more doses. In some embodiments, the pharmaceutical composition comprises at least 2.8×10¹⁰ total CFUs and is administered as seven doses. In some embodiments, each of the multiple doses are administered at regular intervals. In some embodiments, each of the multiple doses are on consecutive days (e.g., first dose on day 1, second dose of day 2, third dose on day 3, etc.).

In some embodiments, the pharmaceutical composition comprises at least 4.0×10¹⁰ total CFUs. In some embodiments, the pharmaceutical composition comprises at least 4.0×10¹⁰ total CFUs and is administered as a single dose. In some embodiments, the pharmaceutical composition comprises at least 4.0×10¹⁰ total CFUs and is administered as multiple (e.g., 2, 3, 4, 5, or more) doses. In some embodiments, the pharmaceutical composition comprises at least 4.0×10¹⁰ total CFUs and is administered as two, three, four, five, six, seven, eight, nine, ten, eleven, twelve, thirteen, fourteen, fifteen or more doses. In some embodiments, the pharmaceutical composition comprises at least 4.0×10¹⁰ total CFUs and is administered as five doses. In some embodiments, each of the multiple doses are administered at regular intervals. In some embodiments, each of the multiple doses are on consecutive days (e.g., first dose on day 1, second dose of day 2, third dose on day 3, etc.). In some embodiments, the pharmaceutical composition comprises at least 4.0×10¹⁰ total CFUs and is administered as five doses, each of which are administered on five consecutive days.

In some embodiments, the pharmaceutical composition comprises at least 5.6×10¹⁰ total CFUs. In some embodiments, the pharmaceutical composition comprises at least 5.6×10¹⁰ total CFUs and is administered as a single dose. In some embodiments, the pharmaceutical composition comprises at least 5.6×10¹⁰ total CFUs and is administered as multiple (e.g., 2, 3, 4, 5, or more) doses. In some embodiments, the pharmaceutical composition comprises at least 5.6×10¹⁰ total CFUs and is administered as two, three, four, five, six, seven, eight, nine, ten, eleven, twelve, thirteen, fourteen, fifteen or more doses. In some embodiments, the pharmaceutical composition comprises at least 5.6×10¹⁰ total CFUs and is administered as fourteen doses. In some embodiments, each of the multiple doses are administered at regular intervals. In some embodiments, each of the multiple doses are on consecutive days (e.g., first dose on day 1, second dose of day 2, third dose on day 3, etc.). In some embodiments, the pharmaceutical composition comprises at least 5.6×10¹⁰ total CFUs and is administered as fourteen doses, each of which are administered on fourteen consecutive days.

In some embodiments, the pharmaceutical composition comprises at least 1.1×10¹¹ total CFUs. In some embodiments, the pharmaceutical composition comprises at least 1.1×10¹¹ total CFUs and is administered as a single dose. In some embodiments, the pharmaceutical composition comprises at least 1.1×10¹¹ total CFUs and is administered as multiple (e.g., 2, 3, 4, 5, or more) doses. In some embodiments, the pharmaceutical composition comprises at least 1.1×10¹¹ total CFUs and is administered as two, three, four, five, six, seven, eight, nine, ten, eleven, twelve, thirteen, fourteen, fifteen or more doses. In some embodiments, the pharmaceutical composition comprises at least 1.1×10¹¹ total CFUs and is administered as fourteen doses. In some embodiments, each of the multiple doses are administered at regular intervals. In some embodiments, each of the multiple doses are on consecutive days (e.g., first dose on day 1, second dose of day 2, third dose on day 3, etc.). In some embodiments, the pharmaceutical composition comprises at least 1.1×10¹¹ total CFUs and is administered as fourteen doses, each of which are administered on fourteen consecutive days.

In some embodiments, the pharmaceutical composition comprises at least 2.1×10¹⁰ total CFUs. In some embodiments, the pharmaceutical composition comprises at least 2.1×10¹⁰ total CFUs and is administered as a single dose. In some embodiments, the pharmaceutical composition comprises at least 2.1×10¹⁰ total CFUs and is administered as multiple (e.g., 2, 3, 4, 5, or more) doses. In some embodiments, the pharmaceutical composition comprises at least 2.1×10¹⁰ total CFUs and is administered as two, three, four, five, six, seven, eight, nine, ten, eleven, twelve, thirteen, fourteen, fifteen or more doses. In some embodiments, the pharmaceutical composition comprises at least 2.1×10¹⁰ total CFUs and is administered as five doses. In some embodiments, each of the multiple doses are administered at regular intervals. In some embodiments, each of the multiple doses are on consecutive days (e.g., first dose on day 1, second dose of day 2, third dose on day 3, etc.). In some embodiments, the pharmaceutical composition comprises at least 2.1×10¹⁰ total CFUs and is administered as five doses, each of which are administered on five consecutive days.

As described herein, any of the pharmaceutical compositions described herein may be administered to a subject in one dose or in multiple doses (e.g., initial administration), which may be followed by one or more additional doses of any of the pharmaceutical compositions described herein. In some embodiments, any of pharmaceutical composition described herein may be administered to a subject in one dose or in multiple doses in an initial administration, followed by one or more additional doses of a pharmaceutical composition comprising the same one or more bacterial strains as the pharmaceutical composition of the initial administration. In some embodiments, any of pharmaceutical composition described herein may be administered to a subject in one dose or in multiple doses in an initial administration, followed by one or more additional doses of a pharmaceutical composition comprising more total bacteria (colony-forming units) relative to the initial administration of the pharmaceutical composition. In some embodiments, any of pharmaceutical composition described herein may be administered to a subject in one dose or in multiple doses in an initial administration, followed by one or more additional doses of a pharmaceutical composition comprising fewer total bacteria (colony-forming units) relative to the initial administration of the pharmaceutical composition. In some embodiments, the initial administration includes at least 1, 2, 3, 4, 5, 6, 7, 8, 9, 10, 11, 12, 13, 14, 15 or more doses of any of the pharmaceutical compositions described herein. In some embodiments, the additional administration includes at least 1, 2, 3, 4, 5, 6, 7, 8, 9, 10, 11, 12, 13, 14, 15 or more doses of any of the pharmaceutical compositions described herein. In some embodiments, the initial administration comprises two doses of any of the pharmaceutical composition and the additional administration comprises three doses of any of the pharmaceutical compositions described herein.

In some embodiments, any of pharmaceutical composition described herein may be administered to a subject in one dose or in multiple doses in an initial administration, followed by one or more additional doses of a pharmaceutical composition comprising fewer total bacteria (colony-forming units) relative to the initial administration of the pharmaceutical composition. In such embodiments, the dose(s) of the initial administration may be referred to as a “high dose” and the dose(s) of the additional administration may be referred to as a “low dose. In some embodiments, the high dose is at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold or more higher than the low dose. In some embodiments, the high dose is 8.0×10⁹ CFUs. In some embodiments, the low dose is 1.6×10⁹ CFUs. In some embodiments, the initial administration comprises multiple doses (e.g., 2, 3, 4, 5 or more) of 8.0×10⁹ CFUs and the additional administration comprises multiple doses (e.g., 2, 3, 4, 5 or more) of 1.6×10⁹ CFUs. In some embodiments, the low dose is 1.6×10⁹ CFUs. In some embodiments, the initial administration comprises two doses of 8.0×10⁹ CFUs and the additional administration comprises three doses of 1.6×10⁹ CFUs.

In some embodiments, the one or more additional administrations is performed on the day following the initial administration (e.g., consecutive days). In some embodiments, the one or more additional administrations is performed at least 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks or longer following the initial administration. In some embodiments, the one or more additional administrations is performed at least 6 weeks after the initial administration. In some embodiments, the one or more additional administrations is performed at least 12 weeks after the initial administration.

In some embodiments, the compositions, including pharmaceutical compositions, contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² CFUs of each of the bacterial strains per dosage amount. In some embodiments, the compositions, including pharmaceutical compositions contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total CFUs per dosage amount.

In some embodiments, the compositions, including pharmaceutical compositions, contain between 10⁻⁷ and 10⁻¹, between 10⁻⁶ and 10⁻¹, between 10⁻⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁻⁵ and 10⁻², between 10⁻⁴ and 10⁻², between 10⁻³ and 10⁻², between 10⁻⁷ and 10⁻³, between 10⁻⁶ and 10⁻³, between 10⁻⁵ and 10⁻³, between 10⁻⁴ and 10⁻³, between 10⁻⁷ and 10⁻⁴, between 10⁻⁶ and 10⁻⁴, between 10⁻⁵ and 10⁻⁴, between 10⁻⁷ and 10⁻⁵′ between 10⁻⁶ and 10⁻⁵, or between 10⁻⁷ and 10⁻⁶ grams of bacteria of each of the bacterial strains in the composition per dosage amount. In some embodiments, the compositions, including pharmaceutical compositions, disclosed herein contain between 10⁻⁷ and 10⁻¹, between 10⁻⁶ and 10⁻¹, between 10⁻⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁻⁵ and 10⁻², between 10⁻⁴ and 10⁻², between 10⁻³ and 10⁻², between 10⁻⁷ and 10⁻³, between 10⁻⁶ and 10⁻³, between 10⁻⁵ and 10⁻³, between 10⁻⁴ and 10⁻³, between 10⁻⁷ and 10⁻⁴, between 10⁻⁶ and 10⁻⁴, between 10⁻⁵ and 10⁻⁴, between 10⁻⁷ and 10⁻⁵′ between 10⁻⁶ and 10⁻⁵, or between 10⁻⁷ and 10⁻⁶ grams of all of the bacteria combined (total) per dosage amount.

Aspects of the present disclosure also provide food products comprising any of the compositions described herein and a nutrient. Also with the scope of the present disclosure are food products comprising any of the bacterial strains described herein and a nutrient. Food products are, in general, intended for the consumption of a human or an animal. Any of the bacterial strains described herein may be formulated as a food product. In some embodiments, the bacterial strains are formulated as a food product in spore form. In some embodiments, the bacterial strains are formulated as a food product in vegetative form. In some embodiments, the food product comprises both vegetative bacteria and bacteria in spore form. The compositions disclosed herein can be used in a food or beverage, such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed.

Non-limiting examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products such as Western confectionery products including biscuits, cookies, and the like, Japanese confectionery products including steamed bean-jam buns, soft adzuki-bean jellies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

Food products containing bacterial strains described herein may be produced using methods known in the art and may contain the same amount of bacteria (e.g., by weight, amount or CFU) as the pharmaceutical compositions provided herein. Selection of an appropriate amount of bacteria in the food product may depend on various factors, including for example, the serving size of the food product, the frequency of consumption of the food product, the specific bacterial strains contained in the food product, the amount of water in the food product, and/or additional conditions for survival of the bacteria in the food product.

Examples of food products which may be formulated to contain any of the bacterial strains described herein include, without limitation, a beverage, a drink, a bar, a snack, a dairy product, a confectionery product, a cereal product, a ready-to-eat product, a nutritional formula, such as a nutritional supplementary formulation, a food or beverage additive.

TABLE 3
Examples of bacterial species of the bacterial strains disclosed herein

| | | % Identity by | SEQ ID | |
|---|---|---|---|---|
| 36-mix | Similarity based on Whole Genome Sequencing | 16S | NO: | Strain |
| Bacteroides caccae | Bacteroides caccae | 99.7 | 1 | 1 |
| Bacteroides intestinalis/Bacteroides cellulosilyticus | Bacteroides intestinalis/Bacteroides cellulosilyticus | 99.2 | 2 | 2 |
| Bacteroides_faecis | Bacteroides sp. 1_1_14/Bacteroides | 99.3 | 3 | 3 |
| | thetaiotaomicron | | | |
| Bacteroides_ovatus | Bifidobacterium angulatum | 99.1 | 4 | 4 |
| Bacteroides_thetaiotaomicron | Bacteroides sp. 1_1_14/Bacteroides | 98.4 | 5 | 5 |
| | thetaiotaomicron | | | |
| Bacteroides uniformis | Bacteroides uniformis | 99.9 | 6 | 6 |
| Bacteroides vulgatus | Bacteroides vulgatus/Bacteroides_B vulgatus | 99.7 | 7 | 7 |
| Bifidobacterium_adolescentis | Gulbenkiania mobilis/Bacillus paralicheniformis | 99.324 | 8 | 8 |
| Bifidobacterium_longum | Bifidobacterium breve/Bifidobacterium longum | 99.6 | 9 | 9 |
| Bifidobacterium_pseudocatenulatum | Bifidobacterium pseudocatenulatum | 99.1 | 10 | 10 |
| Clostridiales bacterium VE202-06 /Blautia coccoides/ | Clostridiales bacterium VE202-06/Blautia producta | 100 | 11 | 11 |
| Blautia producta | | | | |
| Clostridium_citroniae | Clostridium sp. FS41/Clostridium_M citroniae | 99.7 | 12 | 12 |
| Clostridium sp. C105KSO14/Clostridium | Clostridium sp. C105KSO14/Clostridium_M | 99.77 | 13 | 13 |
| clostridioforme | clostridioformis | | | |
| Clostridiales bacterium VE202-21/Clostridium | Clostridiales bacterium VE202-21/Eubacterium_D | 98.6 | 14 | 14 |
| innocuum/Eubacterium contortum | innocuum | | | |
| Erysipelotrichaceae bacterium 6_1_45/Clostridium | Erysipelotrichaceae bacterium 6_1_45/ | 98.7 | 15 | 15 |
| innocuum | Eubacterium_D innocuum | | | |
| Paeniclostridium sordellii/Clostridium sordelli | Paeniclostridium sordellii | 99.0 | 16 | 16 |
| Coprococcus comes | Coprococcus comes | 95.3 | 17 | 17 |
| Dorea longicatena | Dorea longicatena/Dorea longicatena_B | 99.7 | 18 | 18 |
| Erysipelatoclostridium_ramosum | Clostridia bacterium UC5.1-2H6/ | 98.9 | 19 | 19 |
| | Erysipelatoclostridium ramosum | | | |
| Eubacterium_rectale | Eubacterium_rectale | 100 | 20 | 20 |
| Odoribacter sp. UNK.MGS-12/Odoribacter | Odoribacter sp. UNK.MGS-12/Odoribacter | 99.6 | 21 | 21 |
| splanchnicus | splanchnicus | | | |
| Bacteroides sp. 1_1_14 /Parabacteroides merdae/ | Bacteroides sp. 3_1_19/Parabacteroides distasonis | 96.9 | 22 | 22 |
| Parabacteroides distasonis | | | | |
| Bacteroides sp. UNK.MGS-14/Parabacteroides | Bacteroides sp. UNK.MGS-14/Parabacteroides | 98.1 | 23 | 23 |
| merdae | merdae | | | |
| Bacteroides xylanisolvens | Bacteroides sp. 3 113/Bacteroides xylanisolvens_B | 99.708 | 24 | 24 |
| Blautia obeum | Blautia obeum/Blautia_A obeum | 98.8 | 25 | 25 |
| Alistipes putredinis | Alistipes putredinis | 100.0 | 26 | 26 |
| Collinsella aerofaciens | Collinsella aerofaciens | 99.5 | 27 | 27 |
| Eubacterium hallii/Bacteroides faecis | Eubacterium hallii/Eubacterium_E hallii | 99.932 | 28 | 28 |
| Alistipes shahii | Alistipes shahii | 89.0 | 29 | 29 |
| Anaerostipes caccae | | 99.4 | 30 | 30 |
| Phascolarctobacterium faecis/Phascolarctobacterium | | 99.3 | 31 | 31 |
| faecium | | | | |
| Agathobaculum/Agathobaculum butyriciproducens | | 98.0 | 32 | 32 |
| Bacteroides sp. 2_1_56FAA (Bacteroides fragilis) | Bacteroides sp. 2_1_56FAA/Bacteroides fragilis | 99.9 | 33 | 33 |
| Fusobacterium mortiferum | Fusobacterium mortiferum/Fusobacterium_A | 99.022 | 34 | 34 |
| Paraclostridium bifermentans/Paraclostridium | Paraclostridium bifermentans | 100.0 | 35 | 35 |
| benzoelyticum/Clostridium bifermentans | | | | |
| Escherichia sp. 3_2_53FAA/Escherichia_fergusonii | Escherichia sp. 3_2_53FAA/Escherichia coli | 99.87 | 36 | 36 |

SEQUENCES

SEQ ID NO: 1 Bacteroides caccae Strain 1
TGGCTCAGGATGAACGCTAGCTACAGGCTTAACACATGCAAGTCGAGGGGCATCAGTTTGGTTTGCTTGCAAACC
AAAGCTGGCGACCGGCGCACGGGTGAGTAACACGTATCCAACCTGCCTCATACTCGGGGATAGCCTTTCGAAAGA
AAGATTAATATCCGATAGCATATATTTCCCGCATGGGTTTTATATTAAAGAAATTCGGTATGAGATGGGGATGCG
TTCCATTAGTTTGTTGGGGGGGTAACGGCCCACCAAGACTACGATGGATAGGGGTTCTGAGAGGAAGGTCCCCCA
CATTGGAACTGAGACACGGTCCAAACTCCTACGGGAGGCAGCAGTGAGGAATATTGGTCAATGGACGCGAGTCTG
AACCAGCCAAGTAGCGTGAAGGATGACTGCCCTATGGGTTGTAAACTTCTTTTATATGGGAATAAAGTTGTCCAC
GTGTGGATTTTTGTATGTACCATATGAATAAGGATCGGCTAACTCCGTGCCAGCAGCCGCGGTAATACGGAGGAT
CCGAGCGTTATCCGGATTTATTGGGTTTAAAGGGAGCGTAGGCGGATTGTTAAGTCAGTTGTGAAAGTTTGCGGC
TCAACCGTAAAATTGCAGTTGATACTGGCAGTCTTGAGTGCAGTAGAGGTGGGCGGAATTCGTGGTGTAGCGGTG
AAATGCTTAGATATCACGAAGAACTCCGATTGCGAAGGCAGCCACTGGAGTGTAACTGACGCTGATGCTCGAAAG
TGTGGGTATCAAACAGGATTAGATACCCTGGTAGTCCACACAGTAAACGATGAATACTCGCTGTTTGCGATATAC
AGTAAGCGGCCAAGCGAAAGCATTAAGTATTCCACCTGGGGAGTACGCCGGCAACGGTGAAACTCAAAGGAATTG
ACGGGGGCCCGCACAAGCGGAGGAACATGTGGTTTAATTCGATGATACGCGAGGAACCTTACCCGGGCTTAAATT
GCAAATGAATTATGGGGAAACCCATAGGCCGCAAGGCATTTGTGAAGGTGCTGCATGGTTGTCGTCAGCTCGTGC
CGTGAGGTGTCGGCTTAAGTGCCATAACGAGCGCAACCCTTATCTTCAGTTACTAACAGGTCATGCTGAGGACTC
TGGAGAGACTGCCGTCGTAAGATGTGAGGAAGGTGGGGATGACGTCAAATCAGCACGGCCCTTACGTCCGGGGCT
ACACACGTGTTACAATGGGGGGTACAGAAGGCCGCTACCTGGTGACAGGATGCCAATCCCAAAAACCTCTCTCAG
TTCGGATCGAAGTCTGCAACCCGACTTCGTGAAGCTGGATTCGCTAGTAATCGCGCATCAGCCATGGCGCGGTGA
ATACGTTCCCGGGCCTTGTACACACCGCCCGTCAAGCCATGAAAGCCGGGGGTACCTGAAGTACGTAACCGCAAG
GAGCGTCCTAGGGTAAAACTGGTAATTGGGGCTAAGTCGTAACAAGGTA
SEQ ID NO: 2 Bacteroides cellulosilyticus/Bacteroides intestinalis Strain 2
AGAGTTTGATCCTGGCTCAGGATGAACGCTAGCTACAGGCTTAACACATGCAAGTCGAGGGGCAGCATGACCTAG
CAATAGGTTGATGGCGACCGGCGCACGGGTGAGTAACACGTATCCAACCTACCGGTTATTCCGGGATAGCCTTTC
GAAAGAAAGATTAATACCGGATAGTATAACGAGAAGGCATCTTTTTGTTATTAAAGAATTTCGATAACCGATGGG
GATGCGTTCCATTAGTTTGTTGGCGGGGTAACGGCCCACCAAGACATCGATGGATAGGGGTTCTGAGAGGAAGGT
CCCCCACATTGGAACTGAGACACGGTCCAAACTCCTACGGGAGGCAGCAGTGAGGAATATTGGTCAATGGACGAG
AGTCTGAACCAGCCAAGTAGCGTGAAGGATGACTGCCCTATGGGTTGTAAACTTCTTTTATATGGGAATAAAGTG
AGCCACGTGTGGCTTTTTGTATGTACCATACGAATAAGGATCGGCTAACTCCGTGCCAGCAGCCGCGGTAATACG
GAGGATCCGAGCGTTATCCGGATTTATTGGGTTTAAAGGGAGCGTAGGCGGACTATTAAGTCAGCTGTGAAAGTT
TGCGGCTCAACCGTAAAATTGCAGTTGATACTGGTCGTCTTGAGTGCAGTAGAGGTAGGCGGAATTCGTGGTGTA
GCGGTGAAATGCTTAGATATCACGAAGAACTCCGATTGCGAAGGCAGCTTACTGGACTGTAACTGACGCTGATGC
TCGAAAGTGTGGGTATCAAACAGGATTAGATACCCTGGTAGTCCACACAGTAAACGATGAATACTCGCTGTTTGC
GATATACGGCAAGCGGCCAAGCGAAAGCATTAAGTATTCCACCTGGGGAGTACGCCGGCAACGGTGAAACTCAAA
GGAATTGACGGGGGCCCGCACAAGCGGAGGAACATGTGGTTTAATTCGATGATACGCGAGGAACCTTACCCGGGC
TTAAATTGCAAATGAATATAGTGGAAACATTATAGCCGCAAGGCATTTGTGAAGGTGCTGCATGGTTGTCGTCAG
CTCGTGCCGTGAGGTGTCGGCTTAAGTGCCATAACGAGCGCAACCCTTATCTTTAGTTACTAACAGGTCATGCTG
AGGACTCTAGAGAGACTGCCGTCGTAAGATGTGAGGAAGGTGGGGATGACGTCAAATCAGCACGGCCCTTACGTC
CGGGGCTACACACGTGTTACAATGGGGGGTACAGAAGGCAGCTACACAGCGATGTGATGCTAATCCCAAAAGCCT
CTCTCAGTTCGGATTGGAGTCTGCAACCCGACTCCATGAAGCTGGATTCGCTAGTAATCGCGCATCAGCCACGGC
GCGGTGAATACGTTCCCGGGCCTTGTACACACCGCCCGTCAAGCCATGAAAGCCGGGGGTACCTGAAGTCCGTAA
CCGTAAGGAGCGGCCTAGGGTAAAACTGGTAATTGGGGCTAAGTCGTA
SEQ ID NO: 3 Bacteroides faecis Strain 3
CTCAGGATGAACGCTAGCTACAGGCTTAACACATGCAAGTCGAGGGGCAGCATTTCAGTTTGCTTGCAAACTGGA
GATGGCGACCGGCGCACGGGTGAGTAACACGTATCCAACCTGCCGATAACTCGGGGATAGCCTTTCGAAAGAAAG
ATTAATACCCGATGGCATAATAGAACCGCATGGTTTTTTTATTAAAGAATTTCGGTTATCGATGGGGATGCGTTC
CATTAGGCAGTTGGTGAGGTAACGGCTCACCAAACCTTCGATGGATAGGGGTTCTGAGAGGAAGGTCCCCCACAT
TGGAACTGAGACACGGTCCAAACTCCTACGGGAGGCAGCAGTGAGGAATATTGGTCAATGGACGAGAGTCTGAAC
CAGCCAAGTAGCGTGAAGGATGACTGCCCTATGGGTTGTAAACTTCTTTTATATGGGAATAAAGTTTTCCACGTG
TGGAATTTTGTATGTACCATATGAATAAGGATCGGCTAACTCCGTGCCAGCAGCCGCGGTAATACGGAGGATCCG
AGCGTTATCCGGATTTATTGGGTTTAAAGGGAGCGTAGGTGGACAGTTAAGTCAGTTGTGAAAGTTTGCGGCTCA
ACCGTAAAATTGCAGTTGATACTGGCTGTCTTGAGTACAGTAGAGGTGGGCGGAATTCGTGGTGTAGCGGTGAAA
TGCTTAGATATCACGAAGAACTCCGATTGCGAAGGCAGCTCACTGGACTGCAACTGACACTGATGCTCGAAAGTG
TGGGTATCAAACAGGATTAGATACCCTGGTAGTCCACACAGTAAACGATGAATACTCGCTGTTTGCGATATACAG
TAAGCGGCCAAGCGAAAGCATTAAGTATTCCACCTGGGGAGTACGCCGGCAACGGTGAAACTCAAAGGAATTGAC
GGGGGCCCGCACAAGCGGAGGAACATGTGGTTTAATTCGATGATACGCGAGGAACCTTACCCGGGCTTAAATTGC
ATTTGAATATATTGGAAACAGTATAGTCGTAAGACAAATGTGAAGGTGCTGCATGGTTGTCGTCAGCTCGTGCCG
TGAGGTGTCGGCTTAAGTGCCATAACGAGCGCAACCCTTATCTTTAGTTACTAACAGGTCATGCTGAGGACTCTA
GAGAGACTGCCGTCGTAAGATGTGAGGAAGGTGGGGATGACGTCAAATCAGCACGGCCCTTACGTCCGGGGCTAC
ACACGTGTTACAATGGGGGGTACAGAAGGCAGCTACCTGGTGACAGGATGCTAATCCCAAAAGCCTCTCTCAGTT
CGGATCGAAGTCTGCAACCCGACTTCGTGAAGCTGGATTCGCTAGTAATCGCGCATCAGCCATGGCGCGGTGAAT
ACGTTCCCGGGCCTTGTACACACCGCCCGTCAAGCCATGAAAGCCGGGGGTACCTGAAGTACGTAACCGCAAGGA
GCGTCCTAGGGTAAAACTGGTAATTGGGGCTAAGTCGTAACAAGGTA
SEQ ID NO: 4 Bacteroides ovatus Strain 4
CGATATCCGGATTTATTGGAGTTT-
AAGGGAGCGTAGGTGGATTGTTAAGTCAGTTGTGAAAGTTTGCGGCTCAACCGTAAAATTGCAGTTGAAACTGGC
AGTCTTGAGTACAGTAGAGGTGGGCGGAATTCGTGGTGTAGCGGTGAAATGCTTAGATATCACGAAGAACTCCGA
TTGCGAAGGCAGCTCACTAGACTGTCACTGACACTGATGCTCGAAAGTGTGGGTATCAAACAGGATTAGATACCC
TGGTAGTCCACACAGTAAACGATGAATACTCGCTGTTTGCGATATACAGTAAGCGGCCAAGCGAAAGCATTAAGT
ATTCCACCTGGGGAGTACGCCGGCAACGGTGAAACTCAAAGGAATTGACGGGGGCCCGCACAAGCGGAGGAACAT
GTGGTTTAATTCGATGATACGCGAGGAACCTTACCCGGGCTTAAATTGCAACAGAATATATTGGAAACAGTATAG
CCGTAAGGCTGTTGTGAAGGTGCTGCATGGTTGTCGTCAGCTCGTGCCGTGAGGTGTCGGCTTAAGTGCCATAAC
GAGCGCAACCCTTATCTTTAGTTACTAACAGGTKATGCTGAGGACTCTAGAGAGACTGCCGTCGTAAGATGTGAG
GAAGGTGGGGATGACGTCAAATCAGCACGGCCCTTACGTCCGGGGCTACACACGTGTTACAATGGGGGGTACAGA
AGGCSGCTACCTGGTGACAGGATGCTAATCCCAAAAACCTCTCTCAGTTCGGATCGAAGTCTGCAACCCGACTTC
GTGAAGCTGGATTCGCTAGTAATCGCGCATCAGCCATGGCGCGGTGAATACGTTCCCGGGCCTTGTACACACCGC
CCGTCAAGCCATGAAAGCCGGGGGTACCTGAAGTACGTAACCGCAAGGAGCGTCCTAGGGGTAAAACTGGTAATT
GGGGC
SEQ ID NO: 5 Bacteroides thetaiotaomicron Strain 5
TTTAAGGGAGCGTAGGTGGACAGTTAAGTCAGTTGTGAAAGTTTGCGGCTCAACCGTAAAATTGCAGTTGATACT
GGCTGTCTTGAGTACAGTAGAGGTGGGCGGAATTCGTGGTGTAGCGGTGAAATGCTTAGATATCACGAAGAACTC
CGATTGCGAAGGCAGCTCACTGGACTGCAACTGACACTGATGCTCGAAAGTGTGGGTATCAAACAGGATTAGATA
CCCTGGTAGTCCACACAGTAAACGATGAATACTCGCTGTTTGCGATATACAGTAAGCGGCCWAGCGAAAGCATTA
AGTATTCCACCTGGGGAGTACGCCGGCAACGGTGAAACTCAAAGGAATTGACGGGGGCCCGCACAAGCGGAGGAA
CATGTGGTTTAATTCGATGATACGCGAGGAACCTTACCCGGGCTTAAATTGCAWWTGAATAWWYTGGAAACAGKW
TAGYCGYAAGRCAWWTGTGAAGGTGCTGCATGGTTGTCGTCAGCTCGTGCCGTGAGGTGTCGGCTTAAGTGCCAT
AACGAGCGCAACCCTTATCTTTAGTTACTAACAGGTCATGCTGAGGACTCTAGAGAGACTGCCGTCGTAAGATGT
GAGGAAGGTGGGGATGACGTCAAATCAGCACGGCCCTTACGTCCGGGGCTACACACGTGTTACAATGGGGGGTAC
AGAAGGCAGCTACCTGGTGACAGGATGCTAATCCCAAAAGCCTCTCTCAGTTCGGATCGAAGTCTGCAACCCGAC
TTCGTGAAGCTGGATTCGCTAGTAATCGCGCATCAGCCATGGCGCGGTGAATACGTTCCCGGGCCTTGTACACAC
CGCCCGTCAAGCCATGAAAGCCGGGGGTACCTGAAGTACGTAACCGCA
SEQ ID NO: 6 Bacteroides uniformis Strain 6
CTGGCTCAGGATGAACGCTAGCTACAGGCTTAACACATGCAAGTCGAGGGGCAGCATGAACTTAGCTTGCTAAGT
TTGATGGCGACCGGCGCACGGGTGAGTAACACGTATCCAACCTGCCGATGACTCGGGGATAGCCTTTCGAAAGAA
AGATTAATACCCGATGGCATAGTTCTTCCGCATGGTAGAACTATTAAAGAATTTCGGTCATCGATGGGGATGCGT
TCCATTAGGTTGTTGGCGGGGTAACGGCCCACCAAGCCTTCGATGGATAGGGGTTCTGAGAGGAAGGTCCCCCAC
ATTGGAACTGAGACACGGTCCAAACTCCTACGGGAGGCAGCAGTGAGGAATATTGGTCAATGGACGAGAGTCTGA
ACCAGCCAAGTAGCGTGAAGGATGACTGCCCTATGGGTTGTAAACTTCTTTTATACGGGAATAAAGTGAGGCACG
TGTGCCTTTTTGTATGTACCGTATGAATAAGGATCGGCTAACTCCGTGCCAGCAGCCGCGGTAATACGGAGGATC
CGAGCGTTATCCGGATTTATTGGGTTTAAAGGGAGCGTAGGCGGACGCTTAAGTCAGTTGTGAAAGTTTGCGGCT
CAACCGTAAAATTGCAGTTGATACTGGGTGTCTTGAGTACAGTAGAGGCAGGCGGAATTCGTGGTGTAGCGGTGA
AATGCTTAGATATCACGAAGAACTCCGATTGCGAAGGCAGCTTGCTGGACTGTAACTGACGCTGATGCTCGAAAG
TGTGGGTATCAAACAGGATTAGATACCCTGGTAGTCCACACAGTAAACGATGAATACTCGCTGTTTGCGATATAC
AGTAAGCGGCCAAGCGAAAGCGTTAAGTATTCCACCTGGGGAGTACGCCGGCAACGGTGAAACTCAAAGGAATTG
ACGGGGGCCCGCACAAGCGGAGGAACATGTGGTTTAATTCGATGATACGCGAGGAACCTTACCCGGGCTTGAATT
GCAACTGAATGATGTGGAGACATGTCAGCCGCAAGGCAGTTGTGAAGGTGCTGCATGGTTGTCGTCAGCTCGTGC
CGTGAGGTGTCGGCTTAAGTGCCATAACGAGCGCAACCCTTATCGATAGTTACCATCAGGTTATGCTGGGGACTC
TGTCGAGACTGCCGTCGTAAGATGTGAGGAAGGTGGGGATGACGTCAAATCAGCACGGCCCTTACGTCCGGGGCT
ACACACGTGTTACAATGGGGGGTACAGAAGGCAGCTACACGGCGACGTGATGCTAATCCCTAAAGCCTCTCTCAG
TTCGGATTGGAGTCTGCAACCCGACTCCATGAAGCTGGATTCGCTAGTAATCGCGCATCAGCCACGGCGCGGTGA
ATACGTTCCCGGGCCTTGTACACACCGCCCGTCAAGCCATGAAAGCCGGGGGTACCTGAAGTGCGTAACCGCGAG
GAGCGCCCTAGGGTAAAACTGGTGATTGGGGCTAAGTCGTAACAAGGTA
SEQ ID NO: 7 Bacteroides vulgatus Strain 7
ATGAAGAGTTTGATCCTGGCTCAGGATGAACGCTAGCTACAGGCTTAACACATGCAAGTCGAGGGGCAGCATGGT
CTTAGCTTGCTAAGGCCGATGGCGACCGGCGCACGGGTGAGTAACACGTATCCAACCTGCCGTCTACTCTTGGAC
AGCCTTCTGAAAGGAAGATTAATACAAGATGGCATCATGAGTTCACATGTTCACATGATTAAAGGTATTCCGGTA
GACGATGGGGATGCGTTCCATTAGATAGTAGGCGGGGTAACGGCCCACCTAGTCTTCGATGGATAGGGGTTCTGA
GAGGAAGGTCCCCCACATTGGAACTGAGACACGGTCCAAACTCCTACGGGAGGCAGCAGTGAGGAATATTGGTCA
ATGGGCGAGAGCCTGAACCAGCCAAGTAGCGTGAAGGATGACTGCCCTATGGGTTGTAAACTTCTTTTATAAAGG
AATAAAGTCGGGTATGCATACCCGTTTGCATGTACTTTATGAATAAGGATCGGCTAACTCCGTGCCAGCAGCCGC
GGTAATACGGAGGATCCGAGCGTTATCCGGATTTATTGGGTTTAAAGGGAGCGTAGATGGATGTTTAAGTCAGTT
GTGAAAGTTTGCGGCTCAACCGTAAAATTGCAGTTGATACTGGATATCTTGAGTGCAGTTGAGGCAGGCGGAATT
CGTGGTGTAGCGGTGAAATGCTTAGATATCACGAAGAACTCCGATTGCGAAGGCAGCCTGCTAAGCTGCAACTGA
CATTGAGGCTCGAAAGTGTGGGTATCAAACAGGATTAGATACCCTGGTAGTCCACACGGTAAACGATGAATACTC
GCTGTTTGCGATATACGGCAAGCGGCCAAGCGAAAGCGTTAAGTATTCCACCTGGGGAGTACGCCGGCAACGGTG
AAACTCAAAGGAATTGACGGGGGCCCGCACAAGCGGAGGAACATGTGGTTTAATTCGATGATACGCGAGGAACCT
TACCCGGGCTTAAATTGCAGATGAATTACGGTGAAAGCCGTAAGCCGCAAGGCATCTGTGAAGGTGCTGCATGGT
TGTCGTCAGCTCGTGCCGTGAGGTGTCGGCTTAAGTGCCATAACGAGCGCAACCCTTGTTGTCAGTTACTAACAG
GTTCCGCTGAGGACTCTGACAAGACTGCCATCGTAAGATGTGAGGAAGGTGGGGATGACGTCAAATCAGCACGGC
CCTTACGTCCGGGGCTACACACGTGTTACAATGGGGGGTACAGAGGGCCGCTACCACGCGAGTGGATGCCAATCC
CAAAAACCTCTCTCAGTTCGGACTGGAGTCTGCAACCCGACTCCACGAAGCTGGATTCGCTAGTAATCGCGCATC
AGCCACGGCGCGGTGAATACGTTCCCGGGCCTTGTACACACCGCCCGTCAAGCCATGGGAGCCGGGGGTACCTGA
AGTGCGTAACCGCGAGGAGCGCCCTAGGGTAAAACTGGTGACTGGGGCTAAGTCGTAACAAGGTAGCCGTACCGG
AAG
SEQ ID NO: 8 Bifidobacterium adolescentis Strain 8
GGGCTCGTAGKCGGTTCGTCGCGTCCGGTGTGAAAGTCCAYCGCTTAACGGTGGATCCGCGCCGGGTACGGGCGG
GCTTGAGTGCGGTAGGGGAGACTGGAATTCCCGGTGTAACGGTGGAATGTGTAGATATCGGGAAGAACACCAATG
GCGAAGGCAGGTCTCTGGGCCGTCACTGACGCTGAGGAGCGAAAGCGTGGGGAGCGAACAGGATTAGATACCCTG
GTAGTCCACGCCGTAAACGGTGGATGCTGGATGTGGGGACCATTCCACGGTCTCCGTGTCGGAGCCAACGCGTTA
AGCATCCCGCCTGGGGAGTACGGCCGCAAGGCTAAAACTCAAAGAAATTGACGGGGGCCCGCACAAGCGGCGGAG
CATGCGGATTAATTCGATGCAACGCGAAGAACCTTACCTGGGCTTGACATGTTCCCGACAGCCCCAGAGATGGGG
CCTCCCTTCGGGGCGGGTTCACAGGTGGTGCATGGTCGTCGTCAGCTCGTGTCGTGAGATGTTGGGTTAAGTCCC
GCAACGAGCGCAACCCTCGCCCTGTGTTGCCAGCACGTCGTGGTGGGAACTCACGGGGGACCGCCGGGGTCAACT
CGGAGGAAGGTGGGGATGACGTCAGATCATCATGCCCCTTACGTCCAGGGCTTCACGCATGCTACAATGGCCGGT
ACAACGGGATGCGACACT-
GTGAGGTGGAGCGGATCCCTTAAAACCGGTCTCAGTTCGGATTGGAGTCTGCAACCCGACTCCATGAAGGCGGAG
TCGCTAGTAATCGCGGATCAGCAACGCCGCGGTGAATGCGTTCCCGGGCCTTGTACACACCGCCCGTCAAGTCAT
GAAAGTGGGTAGCACCCGAAGCCGGTGGCCCATCCTTTTTGGGG
SEQ ID NO: 9 Bifidobacterium longum Strain 9
TGGCTCAGGATGAACGCTGGCGGCGTGCTTAACACATGCAAGTCGAACGGGATCCATCAGGCTTTGCTTGGTGGT
GAGAGTGGCGAACGGGTGAGTAATGCGTGACCGACCTGCCCCATACACCGGAATAGCTCCTGGAAACGGGTGGTA
ATGCCGGATGCTCCAGTTGATCGCATGGTCTTCTGGGAAAGCTTTCGCGGTATGGGATGGGGTCGCGTCCTATCA
GCTTGACGGCGGGGTAACGGCCCACCGTGGCTTCGACGGGTAGCCGGCCTGAGAGGGCGACCGGCCACATTGGGA
CTGAGATACGGCCCAGACTCCTACGGGAGGCAGCAGTGGGGAATATTGCACAATGGGCGCAAGCCTGATGCAGCG
ACGCCGCGTGAGGGATGGAGGCCTTCGGGTTGTAAACCTCTTTTATCGGGGAGCAAGCGAGAGTGAGTTTACCCG
TTGAATAAGCACCGGCTAACTACGTGCCAGCAGCCGCGGTAATACGTAGGGTGCAAGCGTTATCCGGAATTATTG
GGCGTAAAGGGCTCGTAGGCGGTTCGTCGCGTCCGGTGTGAAAGTCCATCGCTTAACGGTGGATCCGCGCCGGGT
ACGGGCGGGCTTGAGTGCGGTAGGGGAGACTGGAATTCCCGGTGTAACGGTGGAATGTGTAGATATCGGGAAGAA
CACCAATGGCGAAGGCAGGTCTCTGGGCCGTTACTGACGCTGAGGAGCGAAAGCGTGGGGAGCGAACAGGATTAG
ATACCCTGGTAGTCCACGCCGTAAACGGTGGATGCTGGATGTGGGGCCCGTTCCACGGGTTCCGTGTCGGAGCTA
ACGCGTTAAGCATCCCGCCTGGGGAGTACGGCCGCAAGGCTAAAACTCAAAGAAATTGACGGGGGCCCGCACAAG
CGGCGGAGCATGCGGATTAATTCGATGCAACGCGAAGAACCTTACCTGGGCTTGACATGTTCCCGACGGTCGTAG
AGATACGGCTTCCCTTCGGGGCGGGTTCACAGGTGGTGCATGGTCGTCGTCAGCTCGTGTCGTGAGATGTTGGGT
TAAGTCCCGCAACGAGCGCAACCCTCGCCCCGTGTTGCCAGCGGATTATGCCGGGAACTCACGGGGGACCGCCGG
GGTTAACTCGGAGGAAGGTGGGGATGACGTCAGATCATCATGCCCCTTACGTCCAGGGCTTCACGCATGCTACAA
TGGCCGGTACAACGGGATGCGACGCGGCGACGCGGAGCGGATCCCTGAAAACCGGTCTCAGTTCGGATCGCAGTC
TGCAACTCGACTGCGTGAAGGCGGAGTCGCTAGTAATCGCGAATCAGCAACGTCGCGGTGAATGCGTTCCCGGGC
CTTGTACACACCGCCCGTCAAGTCATGAAAGTGGGCAGCACCCGAAGCCGGTGGCCTAACCCCTTGTGGGATGGA
GCCGTCTAAGGTGAGGCTCGTGATTGGGACTAAGTCGTAACAAGGTAGCCGTACCGGAAGGTGCGGCTGG
SEQ ID NO: 10 Bifidobacterium pseudocatenulatum Strain 10
GGTTCGTCGCGTCCGGTGTGAAAGTCCATCGTTTAACGGTGGATCTGCGCCGGGTACGGGCGGGCTGGAGTGCGG
TAGGGGAGACTGGAATTCCCGGTGTAACGGTGGAATGTGTAGATATCGGGAAGAACACCAATGGCGAAGGCAGGT
CTCTGGGCCGTTACTGACGCTGAGGAGCGAAAGCGTGGGGAGCGAACAGGATTAGATACCCTGGTAGTCCACGCC
GTAAACGGTGGATGCTGGATGTGGGGCCCGTTCCACGGGTTCCGTGWCGGAGCTAACGCGTTAAGCATCCCGCCT
GGGGAGTACGGCCGCAAGGCTAAAACWMAAAKAAATTGACGGGGGCCCGCACAAGCGGCGGAGCATGCGGATTAA
TTCGATGCAACGCGAAGAACCTTACCTGGGCTTGACATGTTCCCGACAGCCGTAGAGATATGGCCTCCCTTCGGG
GCGGGTTCACAGGTGGTGCATGGTCGTCGTCAGCTCGTGTCGTGAGATGTTGGGTTAAGTCCCGCAACGAGCGCA
ACCCTCGCCCTGTGTTGCCAGCACGTCATGGTGGGAACTCACGGGGGACCGCCGGGGTCAACTCGGAGGAAGGTG
GGGATGACGTCAGATCATCATGCCCCTTACGTTCAGGGCTTCACGCATGCTACAATGGCCGGTACAACGGGATGC
GACACGGCGACGTG
SEQ ID NO: 11 Blautia coccoides/Blautia producta Strain 11
TGTGACTGAGCGGCGGACGGGTGAGTAACGCGTGGGTAACCTGCCTCATACAGGGGGATAACAGTTAGAAATGAC
TGCTAATACCGCATAAGCGCACAGGACCGCATGGTCTGGTGTGAAAAACTCCGGTGGTATGAGATGGACCCGCGT
CTGATTAGCTAGTTGGAGGGGTAACGGCCCACCAAGGCGACGATCAGTAGCCGGCCTGAGAGGGTGAACGGCCAC
ATTGGGACTGAGACACGGCCCAGACTCCTACGGGAGGCAGCAGTGGGGAATATTGCACAATGGGGGAAACCCTGA
TGCAGCGACGCCGCGTGAAGGAAGAAGTATCTCGGTATGTAAACTTCTATCAGCAGGGAAGAAAATGACGGTACC
TGACTAAGAAGCCCCGGCTAACTACGTGCCAGCAGCCGCGGTAATACGTAGGGGGCAAGCGTTATCCGGATTTAC
TGGGTGTAAAGGGAGCGTAGACGGAAGAGCAAGTCTGATGTGAAAGGCTGGGGCTTAACCCCAGGACTGCATTGG
AAACTGTTGTTCTAGAGTGCCGGAGAGGTAAGCGGAATTCCTAGTGTAGCGGTGAAATGCGTAGATATTAGGAGG
AACACCAGTGGCGAAGGCGGCTTACTGGACGGTAACTGACGTTGAGGCTCGAAAGCGTGGGGAGCAAACAGGATT
AGATACCCTGGTAGTCCACGCCGTAAACGATGAATACTAGGTGTCGGGTGGCAAAGCCATTCGGTGCCGCAGCAA
ACGCAATAAGTATTCCACCTGGGGAGTACGTTCGCAAGAATGAAACTCAAAGGAATTGACGGGGACCCGCACAAG
CGGTGGAGCATGTGGTTTAATTCGAAGCAACGCGAAGAACCTTACCAAGTCTTGACATCCCTCTGACCGTCCCGT
AACGGGGGCTTCCCTTCGGGGCAGAGGAGACAGGTGGTGCATGGTTGTCGTCAGCTCGTGTCGTGAGATGTTGGG
TTAAGTCCCGCAACGAGCGCAACCCTTATCCTTAGTAGCCAGCACATGATGGTGGGCACTCTAGGGAGACTGCCG
GGGATAACCCGGAGGAAGGCGGGGACGACGTCAAATCATCATGCCCCTTATGATTTGGGCTACACACGTGCTACA
ATGGCGTAAACAAAGGGAAGCGAGACAGCGATGTTGAGCGAATCCCAAAAATAACGTCCCAGTTCGGACTGCAGT
CTGCAACTCGACTGCACGAAGCTGGAATCGCTAGTAATCGCGGATCAGAATGCCGCGGTGAATACGTTCCCGGGT
CTTGTACACACCGCCCGTCACACCATGGGAGTCAGTAACGCCCGAAGTCAGTGACCTAACCGAAAGGAAGGAGCT
GCCGAAGGCGGGACCGATAACTGGGGTGAAGTCGTAACAAGGTA
SEQ ID NO: 12 Clostridium citroniae Strain 12
TCCGGATTTACTGGAGTAGT-
AAGGGAGCGTAGACGGCGAAGCAAGTCTGGAGTGAAAACCCAGGGCTCAACCCTGGGACTGCTTTGGAAACTGTT
TTGCTAGAGTGTCGGAGAGGTAAGTGGAATTCCTAGTGTAGCGGTGAAATGCGTAGATATTAGGAGGAACACCAG
TGGCGAAGGCGGCTTACTGGACGATAACTGACGTTGAGGCTCGAAAGCGTGGGGAGCAAACAGGATTAGATACCC
TGGTAGTCCACGCCGTAAACGATGAATGCTAGGTGTTGGGGGGCAAAGCCCTTCGGTGCCGTCGCAAACGCAATA
AGCATTCCACCTGGGGAGTACGTTCGCAAGAATGAAACTCAAAGGAATTGACGGGGACCCGCACAAGCGGTGGAG
CATGTGGTTTAATTCGAAGCAACGCGAAGAACCTTACCAAGTCTTGACATCCCCCTGACCGGTCAGTAAAGTGAC
CTTTCCTTCGGGACAGGGGAGACAGGTGGTGCATGGTTGTCGTCAGCTCGTGTCGTGAGATGTTGGGTTAAGTCC
CGCAACGAGCGCAACCCTTATCCTTAGTAGCCAGCAGGTAAAGCTGGGCACTCTAGGGAGACTGCCAGGGATAAC
CTGGAGGAAGGTGGGGATGACGTCAAATCATCATGCCCCTTATGATTTGGGCTACACACGTGCTACAATGGCGTA
AACAAAGGGAAGCGACCCTGCGAAGGCAAGCAAATCCCAAAAATAACGTCCCAGTTCGGACTGTAGTCTGCAACC
CGACTACACGAAGCTGGAATCGCTAGTAATCGCGAATCAGAATGTCGCGGTGAATACGTTCCCGGGTCTTGTACA
CACCGCCCGTCACACCATGGGAGTCAGCAACGCCCGAAGTCAGTGACCCAACCGAAAGGAGGGAGCTGCCGAA
SEQ ID NO: 13 Clostridium clostridioforme Strain 13
TAATACCGCATAAGCGCACAGTGCCGCATGGCAGTGTGTGAAAAACTCCGGTGGTGTGAGATGGATCCGCGTCTG
ATTAGCCAGTTGGCGGGGTAACGGCCCACCAAAGCGACGATCAGTAGCCGACCTGAGAGGGTGACCGGCCACATT
GGGACTGAGACACGGCCCAAACTCCTACGGGAGGCAGCAGTGGGGAATATTGCACAATGGGCGAAAGCCTGATGC
AGCGACGCCGCGTGAGTGAAGAAGTATTTCGGTATGTAAAGCTCTATCAGCAGGGAAGAAAATGACGGTACCTGA
CTAAGAAGCCCCGGCTAACTACGTGCCAGCAGCCGCGGTAATACGTAGGGGGCAAGCGTTATCCGGATTTACTGG
GTGTAAAGGGAGCGTAGACGGCGAAGCAAGTCTGAAGTGAAAACCCGGGGCTCAACCCTGGGACTGCTTTGGAAA
CTGTTTTGCTAGAGTGTCGGAGAGGTAAGTGGAATTCCTAGTGTAGCGGTGAAATGCGTAGATATTAGGAGGAAC
ACCAGTGGCGAAGGCGGCTTACTGGACGATAACTGACGTTGAGGCTCGAAAGCGTGGGGAGCAAACAGGATTAGA
TACCCTGGTAGTCCACGCCGTAAACGATGAATGCTAGGTGTTGGGGGGCAAAGCCCTTCGGTGCCGCCGCAAACG
CAGTAAGCATTCCACCTGGGGAGTACGTTCGCAAGAATGAAACTCAAAGGAATTGACGGGGACCCGCACAAGCGG
TGGAGCATGTGGTTTAATTCGAAGCAACGCGAAGAACCTTACCAAGTCTTGACATCCCCCTGACGGGCCGGTAAC
GCGGCCTTTCCTTCGGGACAGGGGAGACAGGTGGTGCATGGTTGTCGTCAGCTCGTGTCGTGAGATGTTGGGTTA
AGTCCCGCAACGAGCGCAACCCTTATCCTTAGTAGCCAGCAGGTAGAGCCGGGCACTCTAGGGAGACTGCCAGGG
ATAACCTGGAGGAAGGTGGGGATGACGTCAAATCATCATGCCCCTTATGATTTGGGCTACACACGTGCTACAATG
GCGTAAACAAAGGGAAGCGAGACAGTGATGTGGAGCAAATCCCAAAAATAACGTCCCAGTTCGGACTGTAGTCTG
CAACCCGACTACACGAAGCTGGAATCGCTAGTAATCGCGAATCAGAATGTCGCGGTGAATACGTTCCCGGGTCTT
GTACACACCGCCCGTCACACCATGGGAGTCAGCAACGCCCGAAGTCAGTGACCCAACCGAAAGGAGGGAGCTGCC
GAAGGCGGGGCAGGTAACTGGGGTGAAGTCGT
SEQ ID NO: 14 Clostridium innocuum Strain 14
ATGGAGAGTTTGATCCTGGCTCAGGATGAACGCTGGCGGCATGCCTAATACATGCAAGTCGAACGAAGT-
TTCGAGGAAGCTTGCTTCCAAAGAGACTTAGTGGCGAACGGGTGAGTAACACGTAGGTAACCTGCCCATGTGTCC
GGGATAACTGCTGGAAACGGTAGCTAAAACCGGATAGGTATACAGAGCGCATGCTCAGTATATTAAAGCGCCCAT
CAAGGCGTGAACATGGATGGACCTGCGGCGCATTAGCTAGTTGGTGAGGTAACGGCCCACCAAGGCGATGATGCG
TAGCCGGCCTGAGAGGGTAAACGGCCACATTGGGACTGAGACACGGCCCAAACTCCTACGGGAGGCAGCAGTAGG
GAATTTTCGTCAATGGGGGAAACCCTGAACGAGCAATGCCGCGTGAGTGAAGAAGGTCTTCGGATCGTAAAGCTC
TGTTGTAAGTGAAGAACGGCTCATAGAGGAAATGCTATGGGAGTGACGGTAGCTTACCAGAAAGCCACGGCTAAC
TACGTGCCAGCAGCCGCGGTAATACGTAGGTGGCAAGCGTTATCCGGAATCATTGGGCGTAAAGGGTGCGTAGGT
GGCGTACTAAGTCTGTAGTAAAAGGCAATGGCTCAACCATTGTAAGCTATGGAAACTGGTATGCTGGAGTGCAGA
AGAGGGCGATGGAATTCCATGTGTAGCGGTAAAATGCGTAGATATATGGAGGAACACCAGTGGCGAAGGCGGTCG
CCTGGTCTGTAACTGACACTGAGGCACGAAAGCGTGGGGAGCAAATAGGATTAGATACCCTAGTAGTCCACGCCG
TAAACGATGAGAACTAAGTGTTGGAGGAATTCAGTGCTGCAGTTAACGCAATAAGTTCTCCGCCTGGGGAGTATG
CACGCAAGTGTGAAACTCAAAGGAATTGACGGGGGCCCGCACAAGCGGTGGAGTATGTGGTTTAATTCGAAGCAA
CGCGAAGAACCTTACCAGGCCTTGACATGGAAACAAATACCCTAGAGATAGGGGGATAATTATGGATCACACAGG
TGGTGCATGGTTGTCGTCAGCTCGTGTCGTGAGATGTTGGGTTAAGTCCCGCAACGAGCGCAACCCTTGTCGCAT
GTTACCAGCATCAAGTTGGGGACTCATGCGAGACTGCCGGTGACAAACCGGAGGAAGGTGGGGATGACGTCAAAT
CATCATGCCCCTTATGGCCTGGGCTACACACGTACTACAATGGCGGCCACAAAGAGCAGCGACACAGTGATGTGA
AGCGAATCTCATAAAGGTCGTCTCAGTTCGGATTGAAGTCTGCAACTCGACTTCATGAAGTCGGAATCGCTAGTA
ATCGCAGATCAGCATGCTGCGGTGAATACGTTCTCGGGCCTTGTACACACCGCCCGTCAAACCATGGGAGTCAGT
AATACCCGAAGCCGGTGGCATAACCGTAAGGAGTGAGCCGTCGAAGGTAGGACCGA
SEQ ID NO: 15 Clostridium innocuum/Erysipelotrichaceae bacterium 6_1_45 Strain 15
ATGGAGAGTTTGATCCTGGCTCAGGATGAACGCTGGCGGCATGCCTAATACATGCAAGTCGAACGAAGT-
TTCGAGGAAGCTTGCTTCCAAAGAGACTTAGTGGCGAACGGGTGAGTAACACGTAGGTAACCTGCCCATGTGTCC
GGGATAACTGCTGGAAACGGTAGCTAAAACCGGATAGGTATACAGAGCGCATGCTCAGTATATTAAAGCGCCCAT
CAAGGCGTGAACATGGATGGACCTGCGGCGCATTAGCTAGTTGGTGAGGTAACGGCCCACCAAGGCGATGATGCG
TAGCCGGCCTGAGAGGGTAAACGGCCACATTGGGACTGAGACACGGCCCAAACTCCTACGGGAGGCAGCAGTAGG
GAATTTTCGTCAATGGGGGAAACCCTGAACGAGCAATGCCGCGTGAGTGAAGAAGGTCTTCGGATCGTAAAGCTC
TGTTGTAAGTGAAGAACGGCTCATAGAGGAAATGCTATGGGAGTGACGGTAGCTTACCAGAAAGCCACGGCTAAC
TACGTGCCAGCAGCCGCGGTAATACGTAGGTGGCAAGCGTTATCCGGAATCATTGGGCGTAAAGGGTGCGTAGGT
GGCGTACTAAGTCTGTAGTAAAAGGCAATGGCTCAACCATTGTAAGCTATGGAAACTGGTATGCTGGAGTGCAGA
AGAGGGCGATGGAATTCCATGTGTAGCGGTAAAATGCGTAGATATATGGAGGAACACCAGTGGCGAAGGCGGTCG
CCTGGTCTGTAACTGACACTGAGGCACGAAAGCGTGGGGAGCAAATAGGATTAGATACCCTAGTAGTCCACGCCG
TAAACGATGAGAACTAAGTGTTGGAGGAATTCAGTGCTGCAGTTAACGCAATAAGTTCTCCGCCTGGGGAGTATG
CACGCAAGTGTGAAACTCAAAGGAATTGACGGGGGCCCGCACAAGCGGTGGAGTATGTGGTTTAATTCGAAGCAA
CGCGAAGAACCTTACCAGGCCTTGACATGGAAACAAATACCCTAGAGATAGGGGGATAATTATGGATCACACAGG
TGGTGCATGGTTGTCGTCAGCTCGTGTCGTGAGATGTTGGGTTAAGTCCCGCAACGAGCGCAACCCTTGTCGCAT
GTTACCAGCATCAAGTTGGGGACTCATGCGAGACTGCCGGTGACAAACCGGAGGAAGGTGGGGATGACGTCAAAT
CATCATGCCCCTTATGGCCTGGGCTACACACGTACTACAATGGCGACCACAAAGAGCAGCGACACAGTGATGTGA
AGCGAATCTCATAAAGGTCGTCTCAGTTCGGATTGAAGTCTGCAACTCGACTTCATGAAGTCGGAATCGCTAGTA
ATCGCAGATCAGCATGCTGCGGTGAATACGTTCTCGGGCCTTGTACACACCGCCCGTCAAACCATGGGAGTCAGT
AATACCCGAAGCCGGTGGCATAACCGTAAGGAGTGAGCCGTCGAAGGTAGGACCGA
SEQ ID NO: 16 Clostridium sordellii Strain 16
ACACATGCAAGTCGAGCGAACCCTTCGGGGTGAGCGGCGGACGGGTGAGTAACGCGTGGGTAACCTGCCCTGTAC
ACACGGATAACATACCGAAAGGTATGCTAATACGGGATRAYATATGAGAGTCGCATGGCTTTTGTATCAAAGCTC
CGGCGGTACAGGATGGACCCGCGTCTGATTAGCTAGTTGGTAAGGTAACGGCTTACCAAGGCAACGATCAGTAGC
CGACCTGAGAGGGTGATCGGCCACATTGGAACTGAGACACGGTCCAAACTCMTACGGGAGGCAGCAGTGGGGAAT
ATTGCACAATGGGCGAAAGCCTGATGCAGCAACGCCGCGTGAGCGATGAAGGCCTTCGGGTCGTAAAGCTCTGTC
CTCAAGGAAGATAATGACGGTACTTGAGGAGGAAGCCCCGGCTAACTACGTGCCAGCAGCCGCGGTAATACGTAG
GGGGCTAGCGTTATCCGGAATTACTGGGCGTAAAGGGTGCGTAGGCGGTCTTTCAAGCCAGAAGTGAAAGGCTAC
GGCTCAACCGTAGTAAGCTTTTGGAACTGTAGGACTTGAGTGCAGGAGAGGAGAGTGGAATTCCTAGTGTAGCGG
TGAAATGCGTAGATATTAGGAGGAACACCAGTAGCGAAGGCGGCTCTCTGGACTGTAACTGACGCTGAGGCACGA
AAGCGTGGGGAGCAAACAGGATTAGATACCCTGGTAGTCCACGCCGTAAACGATGAGTACTAGGTGTCGGGGGTT
ACCCCCCTCGGTGCCGCAGCTAACGCATTAAGTACTCCGCCTGGGAAGTACGCTCGCAAGAGTGAAACTCAAAGG
AATTGACGGGGACCCGCACAAGTAGCGGAGCATGTGGTTTAATTCGAAGCAACGCGAAGAACCTTATCTAARCTT
GACATC
SEQ ID NO: 17 Coprococcus comes Strain 17
ACRGGAGGCAGCAGTGGGGAATATTGCACAATGGGGGAAACCCTGATGCAGCGACGCCRCGTGAGCGAAGAAGTA
TTKCGGTATGTAAAGCTCTATCAGCAGGGAAGAAAATGACGGTACCTGACTAAGAAGCACCGGCTAAATACGTGC
CAGCAGCCGCGGTAATACGTATGGTGCAAGCGTTATCCGGATTTACTGGGTGTAAAGGGAGCGTAGACGGCTGTG
TAAGTCTGAAGTGAAAGGCGGGGGCTCCCCCCCGGGGACTGCTTTGGAAACTATGCAGCTAGACTGTCGGACAGG
TAAGTGGAATTCCCAGTGTAGCGGTGAAATGCGTAGATATTGGGAGGAACAGCAKTGGGTAAGGCTSCTTACAGG
ACRAT
SEQ ID NO: 18 Dorea longicatena Strain 18
TAACGCGTGGGTAACCTGCCTCATACAGGGGGATAACAGTTAGAAATGACTGCTAATACCGCATAAGACCACGGT
ACCGCATGGTACAGTGGTAAAAACTCCGGTGGTATGAGATGGACCCGCGTCTGATTAGGTAGTTGGTGGGGTAAC
GGCCTACCAAGCCGACGATCAGTAGCCGACCTGAGAGGGTGACCGGCCACATTGGGACTGAGACACGGCCCAGAC
TCCTACGGGAGGCAGCAGTGGGGAATATTGCACAATGGAGGAAACTCTGATGCAGCGACGCCGCGTGAAGGATGA
AGTATTTCGGTATGTAAACTTCTATCAGCAGGGAAGAAAATGACGGTACCTGACTAAGAAGCCCCGGCTAACTAC
GTGCCAGCAGCCGCGGTAATACGTAGGGGGCAAGCGTTATCCGGATTTACTGGGTGTAAAGGGAGCGTAGACGGC
ACGGCAAGCCAGATGTG-
AAAGCCCGGGGCTCAACCCCGGGACTGCATTTGGAACTGCTGAGCTAGAGTGTCGGAGAGGCAAGTGGAATTCCT
AGTGTAGCGGTGAAATGCGTAGATATTAGGAGGAACACCAGTGGCGAAGGCGGCTTGCTGGACGATGACTGACGT
TGAGGCTCGAAAGCGTGGGGAGCAAACAGGATTAGATACCCTGGTAGTCCACGCCGTAAACGATGACTGCTAGGT
GTCGGGTGGCAAAGCCATTCGGTGCCGCAGCTAACGCAATAAGCAGTCCACCTGGGGAGTACGTTCGCAAGAATG
AAACTCAAAGGAATTGACGGGGACCCGCACAAGCGGTGGAGCATGTGGTTTAATTCGAAGCAACGCGAAGAACCT
TACCTGATCTTGACATCCCGATGACCGCTTCGTAATGGAAGCTTTTCTTCGGAACATCGGTGACAGGTGGTGCAT
GGTTGTCGTCAGCTCGTGTCGTGAGATGTTGGGTTAAGTCCCGCAACGAGCGCAACCCCTATCTTCAGTAGCCAG
CAGGTTAAGCTGGGCACTCTGGAGAGACTGCCAGGGATAACCTGGAGGAAGGTGGGGATGACGTCAAATCATCAT
GCCCCTTATGACCAGGGCTACACACGTGCTACAATGGCGTAAACAAAGAGAAGCGAACTCGCGAGGGTAAGCAAA
TCTCAAAAATAACGTCTCAGTTCGGATTGTAGTCTGCAACTCGACTACATGAAGCTGGAATCGCTAGTAATCGCA
GATCAGAATGCTGCGGTGAATACGTTCCCGGGTCTTGTACACACCGCCCGTCACACCATGGGAGTCAGTAACGCC
CGAAGTCAGTGACCCAACCGTAAGG
SEQ ID NO: 19 Erysipelatoclostridium ramosum Strain 19
GAGGGAGCAGGCGGCAGCAAGGGTCTGTGGTGAAAGCCTGAAGTTAAACTTCAGTAAGCCATAGAAACCAGGCAG
CTAGAGTGCAGGAGAGGAKCGTGGAATTCCATGTGTAGCGGTGAAATGCGTAGATATATGGAGGAACACCAGTGG
CGAAGGCGACGATCTGGCCTGCAACTGACGCTCAGTCCCGAAAGCGTGGGGAGCAAATAGGATTAGATACCCTAG
TAGTCCACGCCGTAAACGATGAGTACTRAGTGTTGGATGTCAAAGTTCAGTGCTGCAGTTAACGCAATAAGTACT
CCGCCTGAGTAGTACGTTCGCAAGAATGAAACTCAAAGGAATTGACGGGGGCCCGCACAAGCGGTGGAGCATGTG
GTTTAATTCGAAGCAACGCGAAGAACCTTACCAGGTCTTGACATACTCATAAAGGCTCCAGAGATGGAGAGATAG
CTATATGAGATACAGGTGGTGCATGGTTGTCGTCAGCTCGTGTCGTGAGATGTTGGGTTAAGTCCCGCAACGAGC
GCAACCCTTATCGTTAGTTACCATCATTAAGTTGGGGACTCTAGCGAGACTGCCAGTGACAAGCTGGAGGAARGC
GGGGATGACGTCAAATCATCATGCCCCTTATGACCTGGGCTACACACGTGCTACAATGGATGGTGCAGAGGGAAG
CGAAGCCGCGAGGTGAAGCAAAACCCATAAAACCATTCTCAGTTCGGATTGTAGTCTGCARCTCGACTACATGAA
GTTGGAATCGCTAGTAATCGCGAATCARCATGTCGCGATGAATAMGTTCTCGGGCCTT
SEQ ID NO: 20 Eubacterium rectale Strain 20
AGAGTTTGATCCTGGCTCAGGATGAACGCTGGCGGCGTGCTTAACACATGCAAGTCGAACGAAGCACTTTATTTG
ATTTCCTTCGGGACTGATTATTTTGTGACTGAGTGGCGGACGGGTGAGTAACGCGTGGGTAACCTGCCTTGTACA
GGGGGATAACAGTTGGAAACGGCTGCTAATACCGCATAAGCGCACGGCATCGCATGATGCAGTGTGAAAAACTCC
GGTGGTATAAGATGGACCCGCGTTGGATTAGCTAGTTGGTGAGGTAACGGCCCACCAAGGCGACGATCCATAGCC
GACCTGAGAGGGTGACCGGCCACATTGGGACTGAGACACGGCCCAAACTCCTACGGGAGGCAGCAGTGGGGAATA
TTGCACAATGGGCGAAAGCCTGATGCAGCGACGCCGCGTGAGCGAAGAAGTATTTCGGTATGTAAAGCTCTATCA
GCAGGGAAGATAATGACGGTACCTGACTAAGAAGCACCGGCTAAATACGTGCCAGCAGCCGCGGTAATACGTATG
GTGCAAGCGTTATCCGGATTTACTGGGTGTAAAGGGAGCGCAGGCGGTGCGGCAAGTCTGATGTGAAAGCCCGGG
GCTCAACCCCGGTACTGCATTGGAAACTGTCGTACTAGAGTGTCGGAGGGGTAAGCGGAATTCCTAGTGTAGCGG
TGAAATGCGTAGATATTAGGAGGAACACCAGTGGCGAAGGCGGCTTACTGGACGATAACTGACGCTGAGGCTCGA
AAGCGTGGGGAGCAAACAGGATTAGATACCCTGGTAGTCCACGCCGTAAACGATGAATACTAGGTGTTGGGAAGC
ATTGCTTCTCGGTGCCGTCGCAAACGCAGTAAGTATTCCACCTGGGGAGTACGTTCGCAAGAATGAAACTCAAAG
GAATTGACGGGGACCCGCACAAGCGGTGGAGCATGTGGTTTAATTCGAAGCAACGCGAAGAACCTTACCAAGTCT
TGACATCCTTCTGACCGGTACTTAACCGTACCTTCTCTTCGGAGCAGGAGTGACAGGTGGTGCATGGTTGTCGTC
AGCTCGTGTCGTGAGATGTTGGGTTAAGTCCCGCAACGAGCGCAACCCTTATCTTTAGTAGCCAGCGGTTCGGCC
GGGCACTCTAGAGAGACTGCCAGGGATAACCTGGAGGAAGGCGGGGATGACGTCAAATCATCATGCCCCTTATGA
CTTGGGCTACACACGTGCTACAATGGCGTAAACAAAGGGAAGCAAAGCTGTGAAGCCGAGCAAATCTCAAAAATA
ACGTCTCAGTTCGGACTGTAGTCTGCAACCCGACTACACGAAGCTGGAATCGCTAGTAATCGCAGATCAGAATGC
TGCGGTGAATACGTTCCCGGGTCTTGTACACACCGCCCGTCACACCATGGGAGTTGGGAATGCCCGAAGCCAGTG
ACCTAACCGAAAGGAAGGAGCTGTCGAAGGCAGGCTCGATAACTGGGGTGAAGTCGTAACAAGGTAGCCGTATCG
GAAGGTGCGGCTGGATCACCT
SEQ ID NO: 21 Odoribacter splanchnicus Strain 21
AGAGTTTGATCCTGGCTCAGGATGAACGCTAGCGACAGGCTTAACACATGCAAGTCGAGGGGCATCATGAGGTAG
CAATACCTTGATGGCGACCGGCGCACGGGTGAGTAACGCGTATGCAACCTGCCCGATACCGGGGTATAGCCCATG
GAAACGTGGATTAACACCCCATAGTACTTTTATCCTGCATGGGATGTGAGTTAAATGTTTAAGGTATCGGATGGG
CATGCGTCCTATTAGTTAGTTGGCGGGGTAACAGCCCACCAAGACGATGATAGGTAGGGGTTCTGAGAGGAAGGT
CCCCCACATTGGAACTGAGACACGGTCCAAACTCCTACGGGAGGCAGCAGTGAGGAATATTGGTCAATGGACGAG
AGTCTGAACCAGCCAAGTCGCGTGAGGGAAGACTGCCCTATGGGTTGTAAACCTCTTTTATAAGGGAAGAATAAG
TTCTACGTGTAGAATGATGCCTGTACCTTATGAATAAGCATCGGCTAACTCCGTGCCAGCAGCCGCGGTAATACG
GAGGATGCGAGCGTTATCCGGATTTATTGGGTTTAAAGGGTGCGTAGGCGGTTTATTAAGTTAGTGGTTAAATAT
TTGAGCTAAACTCAATTGTGCCATTAATACTGGTAAACTGGAGTACAGACGAGGTAGGCGGAATAAGTTAAGTAG
CGGTGAAATGCATAGATATAACTTAGAACTCCGATAGCGAAGGCAGCTTACCAGACTGTAACTGACGCTGATGCA
CGAGAGCGTGGGTAGCGAACAGGATTAGATACCCTGGTAGTCCACGCCGTAAACGATGCTCACTGGTTCTGTGCG
ATATATTGTACGGGATTAAGCGAAAGTATTAAGTGAGCCACCTGGGGAGTACGTCGGCAACGATGAAACTCAAAG
GAATTGACGGGGGCCCGCACAAGCGGAGGAACATGTGGTTTAATTCGATGATACGCGAGGAACCTTACCTGGGTT
TAAATGGGAAATGTCGTATTTGGAAACAGATATTCTCTTCGGAGCGTTTTTCAAGGTGCTGCATGGTTGTCGTCA
GCTCGTGCCGTGAGGTGTCGGGTTAAGTCCCATAACGAGCGCAACCCTTACCGTTAGTTGCTAGCATGTAATGAT
GAGCACTCTAACGGGACTGCCACCGTAAGGTGAGAGGAAGGCGGGGATGACGTCAAATCAGCACGGCCCTTACAC
CCAGGGCTACACACGTGTTACAATGGCCGGTACAGAGGGCCGCTACCAGGTGACTGGATGCCAATCTCAAAAGCC
GGTCGTAGTTCGGATTGGAGTCTGTAACCCGACTCCATGAAGTTGGATTCGCTAGTAATCGCGCATCAGCCATGG
CGCGGTGAATACGTTCCCGGGCCTTGTACACACCGCCCGTCAAGCCATGGAAGCCGGGGGTGCCTGAAGTCCGTA
ACCGCGAGGATCGGCCTAGGGCAAAACTGGTAACTGGGGCTAAGTCGTAACAAGGTA
SEQ ID NO: 22 Parabacteroides distasonis Strain 22
CGAGGGGCAGCRCAGGAGT-TAGCAATAC-
CSGGTGGCGACCGGCGCACGGGTGAGTAACGCGTATGCAACTTRCCTATCAGAGGGGGATAACCCGGCGAAAGTC
GGACTAATACCGCATGAAGCAGGGATYCCGCATGGGRATATTTGCTAAAGATTCATCGCTGATAGATAGGCATGC
GTTCCATTAGGCAGTTGGCGGGGTAACRGCCCACCAAACCGACGATGGATAGGGGTTCTGAGAGGAAGGTCCCCC
ACATTGGTACTGAGACACGGACCAAACTCCTACGGGAGGCAGCAGTGAGGAATATTGGTCAATGGSCGWRAGSCT
GAACCAGCCAAGTCGCGTGAGGGATGAAGGTTCTATGGATCGTAAACCTCTTTTATAAGGGAATAAAGTGCGGGA
CGTGTCCYRTTTTGTATGTACCTTATGAATAAGGATCGGCTAACTCCGTGCCAGCAGCCGCGGTAATACGGAGGA
TCCGAGCGTTATCCGGATTTATTGGGTTTAAAGGGTGCGTAGGCGGCCTTTTAAGTCAGCGGTGAAAGTCTGTGG
CTCAACCATAGAATTGCCGTTGAAACTGGGGGGCTTGAGTATGTTTGAGGCAGGCGGAATGCGTGGTGTAGCGGT
GAAATGCATAGATATCACGCAGAACCCCGWTTGCGAAGGCAGCCTGCCAAGCCGTAACTGACGCGGATGCACGAA
AGCGTGGGGATCAAACAGGATTAGATACCCTGGTA
SEQ ID NO: 23 Parabacteroides merdae Strain 23
CATGCAAGTCGAGGGGCAGCATGATTTGTAGCAATACAGATTGATGGCGACCGGCGCACGGGTGAGTAACGCGTA
TGCAACTTACCTATCAGAGGGGGATAGCCCGGCGAAAGTCGGATTAATACCCCATAAAACAGGGGTCCCGCATGG
GAATATTTGTTAAAGATTCATCGCTGATAGATAGGCATGCGTTCCATTAGGCAGTTGGCGGGGTAACGGCCCACC
AAACCGACGATGGATAGGGGTTCKGAGAGGAAGGTCCCCCACATTGGTACTGAGACACGGACCAAACTCCTACGG
GAGGCAGCAGTGAGGAATATTGGTCAATGGCCGAGAGGCTGAACCAGCCAAGTCGCGTGAAGGAAGAAGGATCTA
TGGTTTGTAAACTTCTTTTATAGGGGAATAAAGTGGAGGACGTGTCCTTTTTTGTATGTACCCTATGAATAAGCA
TCGGCTAACTCCGTGMSARCMGCCGCGGGAATACGGAAGATGCAGAGCGTTATCCGGATWTATTGGGGTTA
SEQ ID NO: 24 Bacteroides xylanisolvens Strain 24
CATGCAAGTCGAGGGGCAGCATTTTAGTTTGCTTGCAAACTAAAGATGGCGACCGGCGCACGGGTGAGTAACACG
TATCCAACCTGCCGATAACTCGGGGATAGCCTTTCGAAAGAAAGATTAATATCCGATAGTATATTAAAACCGCAT
GGTTTTACTATTAAAGAATTTCGGTTATCGATGGGGATGCGTTCCATTAGTTTGTTGGCGGGGTAACGGCCCACC
AAGACTACGATGGATAGGGGTTCTGAGAGGAAGGTCCCCCACATTGGAACTGAGACACGGTCCAAACTCCTACGG
GAGGCAGCAGTGAGGAATATTGGTCAATGGACGAGAGTCTGAACCAGCCAAGTAGCGTGAAGGATGACTGCCCTA
TGGGTTGTAAACTTCTTTTATATGGGAATAAAGTATTCCACGTGTGGGATTTTGTATGTACCATATGAATAAGGA
TCGGCTAACTCCGTGCCAGCAGCCGCGGTAATACGGAGGATCCGAGCGTTATCCGGATTTATTGGGTTTAAAGGG
AGCGTAGGTGGATTGTTAAGTCAGTTGTGAAAGTTTGCGGCTCAACCGTAAAATTGCAGTTGAAACTGGCAGTCT
TGAGTACAGTAGAGGTGGGCGGAATTCGTGGTGTAGCGGTGAAATGCTTAGATATCACGAAGAACTCCGATTGCG
AAGGCAGCTCACTAGACTGCAACTGACACTGATGCTCGAAAGTGTGGGTATCAAACAGGATTAGATACCCTGGTA
GTCCACACAGTAAACGATGAATACTCGCTGTTTGCGATATACAGTAAGCGGCCAAGCGAAAGCATTAAGTATTCC
ACCTGGGGAGTACGCCGGCAACGGTGAAACTCAAAGGAATTGACGGGGGCCCGCACAAGCGGAGGAACATGTGGT
TTAATTCGATGATACGCGAGGAACCTTACCCGGGCTTAAATTGCATTTGAATAATCTGGAAACAGGTTAGCCGCA
AGGCAAATGTGAAGGTGCTGCATGGTTGTCGTCAGCTCGTGCCGTGAGGTGTCGGCTTAAGTGCCATAACGAGCG
CAACCCTTATCTTTAGTTACTAACAGGTTATGCTGAGGACTCTAGAGAGACTGCCGTCGTAAGATGTGAGGAAGG
TGGGGATGACGTCAAATCAGCACGGCCCTTACGTCCGGGGCTACACACGTGTTACAATGGGGGGTACAGAAGGCA
GCTACCTGGCGACAGGATGCTAATCCCAAAAACCTCTCTCAGTTCGGATCGAAGTCTGCAACCCGACTTCGTGAA
GCTGGATTCGCTAGTAATCGCGCATCAGCCATGGCGCGGTGAATACGTTCCCGGGCCTTGTACACACCGCCCGTC
AAGCCATGAAAGCCGGGG
SEQ ID NO: 25 Blautia obeum Strain 25
GGCGTGCTTAACACATGCAAGTCGAACGGGAAACCTTTTATTGAAGCTTCGGCAGATTTAG-
CTGGTTTCTAGTGGCGGACGGGTGAGTAACGCGTGGGTAACCTGCCTTATACAGGGGGATAACAACCAGAAATGG
TTGCTAATACCGCATAAGCGCACAGGACCGCATGGTCCGGTGTGAAAAACTCCGGTGGTATAAGATGGACCCGCG
TTGGATTAGCTAGTTGGCAGGGTAACGGCCTACCAAGGCGACGATCCATAGCCGGCCTGAGAGGGTGAACGGCCA
CATTGGGACTGAGACACGGCCCAGACTCCTACGGGAGGCAGCAGTGGGGAATATTGCACAATGGGGGAAACCCTG
ATGCAGCGACGCCGCGTGAAGGAAGAAGTATCTCGGTATGTAAACTTCTATCAGCAGGGAAGATAGTGACGGTAC
CTGACTAAGAAGCCCCGGCTAACTACGTGCCAGCAGCCGCGGTAATACGTAGGGGGCAAGCGTTATCCGGATTTA
CTGGGTGTAAAGGGAGCGTAGACGGACTGGCAAGTCTGATGTGAAAGGCGGGGGCTCAACCCCTGGACTGCATTG
GAAACTGTTAGTCTTGAGTGCCGGAGAGGTAAGCGGAATTCCTAGTGTAGCGGTGAAATGCGTAGATATTAGGAG
GAACACCAGTGGCGAAGGCGGCTTACTGGACGGTAACTGACGTTGAGGCTCGAAAGCGTGGGGAGCAAACAGGAT
TAGATACCCTGGTAGTCCACGCCGTAAACGATGAATACTAGGTGTTGGGGAGCAAAGCTCTTCGGTGCCGCCGCA
AACGCATTAAGTATTCCACCTGGGGAGTACGTTCGCAAGAATGAAACTCAAAGGAATTGACGGGGACCCGCACAA
GCGGTGGAGCATGTGGTTTAATTCGAAGCAACGCGAAGAACCTTACCAAGTCTTGACATCCCTCTGACCGTTCCT
TAACCGGAACTTTCCTTCGGGACAGGGGAGACAGGTGGTGCATGGTTGTCGTCAGCTCGTGTCGTGAGATGTTGG
GTTAAGTCCCGCAACGAGCGCAACCCCTATCCCCAGTAGCCAGCAGTCCGGCTGGGCACTCTGAGGAGACTGCCA
GGGATAACCTGGAGGAAGGCGGGGATGACGTCAAATCATCATGCCCCTTATGATTTGGGCTACACACGTGCTACA
ATGGCGTAAACAAAGGGAAGCAAGCCTGCGAAGGTAAGCAAATCCCAAAAATAACGTCCCAGTTCGGACTGCAGT
CTGCAACTCGACTGCACGAAGCTGGAATCGCTAGTAATCGCGGATCAGAATGCCGCGGTGAATACGTTCCCGGGT
CTTGTACACACCGCCCGTCACACCATGGGAGTCAGTAACGCCCGAAGTCAGTGACCTAACTGC
SEQ ID NO: 26 Alistipes putredinis Strain 26
AGAGTTTGATCCTGGCTCAGGATGAACGCTAGCGGCAGGCTTAACACATGCAAGTCGAGGGGCAGCATAATGGAT
AGCAATATCTATGGTGGCGACCGGCGCACGGGTGCGTAACGCGTATGCAACCTACCTTTAACAGGGGGATAACAC
TGAGAAATTGGTACTAATACCCCATAATATCATAGAAGGCATCTTTTATGGTTGAAAATTCCGATGGTTAGAGAT
GGGCATGCGTTGTATTAGCTAGTTGGTGGGGTAACGGCTCACCAAGGCGACGATACATAGGGGGACTGAGAGGTT
AACCCCCCACACTGGTACTGAGACACGGACCAGACTCCTACGGGAGGCAGCAGTGAGGAATATTGGTCAATGGAC
GCAAGTCTGAACCAGCCATGCCGCGTGCAGGATGACGGCTCTATGAGTTGTAAACTGCTTTTGTACGAGGGTAAA
CGCAGATACGTGTATCTGTCTGAAAGTATCGTACGAATAAGGATCGGCTAACTCCGTGCCAGCAGCCGCGGTAAT
ACGGAGGATTCAAGCGTTATCCGGATTTATTGGGTTTAAAGGGTGCGTAGGCGGTTTGATAAGTTAGAGGTGAAA
TTTCGGGGCTCAACCCTGAACGTGCCTCTAATACTGTTGAGCTAGAGAGTAGTTGCGGTAGGCGGAATGTATGGT
GTAGCGGTGAAATGCTTAGAGATCATACAGAACACCGATTGCGAAGGCAGCTTACCAAACTATATCTGACGTTGA
GGCACGAAAGCGTGGGGAGCAAACAGGATTAGATACCCTGGTAGTCCACGCAGTAAACGATGATAACTCGTTGTC
GGCGATACACAGTCGGTGACTAAGCGAAAGCGATAAGTTATCCACCTGGGGAGTACGTTCGCAAGAATGAAACTC
AAAGGAATTGACGGGGGCCCGCACAAGCGGAGGAACATGTGGTTTAATTCGATGATACGCGAGGAACCTTACCCG
GGCTTGAAAGTTAGCGACGATTCTTGAAAGAGGATTTCCCTTCGGGGCGCGAAACTAGGTGCTGCATGGTTGTCG
TCAGCTCGTGCCGTGAGGTGTCGGGTTAAGTCCCATAACGAGCGCAACCCCTACCGTTAGTTGCCATCAGGTGAA
GCTGGGCACTCTGGCGGGACTGCCGGTGTAAGCCGAGAGGAAGGTGGGGATGACGTCAAATCAGCACGGCCCTTA
CGTCCGGGGCTACACACGTGTTACAATGGTAGGTACAGAGGGCAGCTACCCAGCGATGGGATGCGAATCTCGAAA
GCCTATCTCAGTTCGGATTGGAGGCTGAAACCCGCCTCCATGAAGTTGGATTCGCTAGTAATCGCGCATCAGCCA
TGGCGCGGTGAATACGTTCCCGGGCCTTGTACACACCGCCCGTCAAGCCATGGGAGCCGGGGGTGCCTGAAGTTC
GTGACCGCAAGGAGCGACCTAGGGCAAAACTGGTGACTGGGGCTAAGTCGTAACAAGGTA
SEQ ID NO: 27 Collinsella aerofaciens Strain 27
AGAGTTCGATCCTGGCTCAGGATGAACGCTGGCGGCGCGCCTAACACATGCAAGTCGAACGGCACCTATCTTCGG
ATAGAAGCGAGTGGCGAACGGCTGAGTAACACGTGGAGAACCTGCCCCCTCCCCCGGGATAGCCGCCCGAAAGGA
CGGGTAATACCGGATACCCCGGGGTGCCGCATGGCACCCCGGCTAAAGCCCCGACGGGAGGGGATGGCTCCGCGG
CCCATCAGGTAGACGGCGGGGTGACGGCCCACCGTGCCGACAACGGGTAGCCGGGTTGAGAGACCGACCGGCCAG
ATTGGGACTGAGACACGGCCCAGACTCCTACGGGAGGCAGCAGTGGGGAATCTTGCGCAATGGGGGGAACCCTGA
CGCAGCGACGCCGCGTGCGGGACGGAGGCCTTCGGGTCGTAAACCGCTTTCAGCAGGGAAGAGTCAAGACTGTAC
CTGCAGAAGAAGCCCCGGCTAACTACGTGCCAGCAGCCGCGGTAATACGTAGGGGGCGAGCGTTATCCGGATTCA
TTGGGCGTAAAGCGCGCGTAGGCGGCCCGGCAGGCCGGGGGTCGAAGCGGGGGGCTCAACCCCCCGAAGCCCCCG
GAACCTCCGCGGCTTGGGTCCGGTAGGGGAGGGTGGAACACCCGGTGTAGCGGTGGAATGCGCAGATATCGGGTG
GAACACCGGTGGCGAAGGCGGCCCTCTGGGCCGAGACCGACGCTGAGGCGCGAAAGCTGGGGGAGCGAACAGGAT
TAGATACCCTGGTAGTCCCAGCCGTAAACGATGGACGCTAGGTGTGGGGGGACGATCCCCCCGTGCCGCAGCCAA
CGCATTAAGCGTCCCGCCTGGGGAGTACGGCCGCAAGGCTAAAACTCAAAGGAATTGACGGGGGCCCGCACAAGC
AGCGGAGCATGTGGCTTAATTCGAAGCAACGCGAAGAACCTTACCAGGGCTTGACATATGGGTGAAGCGGGGGAG
ACCCCGTGGCCGAGAGGAGCCCATACAGGTGGTGCATGGCTGTCGTCAGCTCGTGTCGTGAGATGTTGGGTTAAG
TCCCGCAACGAGCGCAACCCCCGCCGCGTGTTGCCATCGGGTGATGCCGGGAACCCACGCGGGACCGCCGCCGTC
AAGGCGGAGGAGGGCGGGGACGACGTCAAGTCATCATGCCCCTTATGCCCTGGGCTGCACACGTGCTACAATGGC
CGGTACAGAGGGATGCCACCCCGCGAGGGGGAGCGGATCCCGGAAAGCCGGCCCCAGTTCGGATTGGGGGCTGCA
ACCCGCCCCCATGAAGTCGGAGTTGCTAGTAATCGCGGATCAGCATGCCGCGGTGAATGCGTTCCCGGGCCTTGT
ACACACCGCCCGTCACACCACCCGAGTCGTCTGCACCCGAAGTCGCCGGCCCAACCGAGAGGGGGGAGGCGCCGA
AGGTGTGGAGGGTGAGGGGGGTGAAGTCGTAACAAGGTA
SEQ ID NO: 28 Bacteroides faecis/Eubacterium hallii Strain 28
CTCAGGATGAACGCTAGCTACAGGCTTAACACATGCAAGTCGAGGGGCAGCATTCCAGTTTGCTTGCAAACTGGA
GATGGCGACCGGCGCACGGGTGAGTAACACGTATCCAACCTGCCGATAACTCGGGGATAGCCTTTCGAAAGAAAG
ATTAATACCCGATGGCATAATAGAACCGCATGGTTTGATTATTAAAGAATTTCGGTTATCGATGGGGATGCGTTC
CATTAGGCAGTTGGTGGGGTAACGGCCCACCAAACCTTCGATGGATAGGGGTTCTGAGAGGAAGGTCCCCCACAT
TGGAACTGAGACACGGTCCAAACTCCTACGGGAGGCAGCAGTGAGGAATATTGGTCAATGGACGAGAGTCTGAAC
CAGCCAAGTAGCGTGAAGGATGACTGCCCTATGGGTTGTAAACTTCTTTTATATGGGAATAAAGTGGTCCACGTG
TGGATTTTTGTATGTACCATATGAATAAGGATCGGCTAACTCCGTGCCAGCAGCCGCGGTAATACGGAGGATCCG
AGCGTTATCCGGATTTATTGGGTTTAAAGGGAGCGTAGGTGGACAGTTAAGTCAGTTGTGAAAGTTTGCGGCTCA
ACCGTAAAATTGCAGTTGATACTGGCTGTCTTGAGTACAGTAGAGGCGGGCGGAATTCGTGGTGTAGCGGTGAAA
TGCTTAGATATCACGAAGAACTCCGATTGCGAAGGCAGCTCACTGGACTGCAACTGACACTGATGCTCGAAAGTG
TGGGTATCAAACAGGATTAGATACCCTGGTAGTCCACACAGTAAACGATGAATACTCGCTGTTTGCGATATACAG
TAAGCGGCCAAGCGAAAGCATTAAGTATTCCACCTGGGGAGTACGCCGGCAACGGTGAAACTCAAAGGAATTGAC
GGGGGCCCGCACAAGCGGAGGAACATGTGGTTTAATTCGATGATACGCGAGGAACCTTACCCGGGCTTAAATTGC
ATTTGAATATATTGGAAACAGTATAGTCGTAAGACAAATGTGAAGGTGCTGCATGGTTGTCGTCAGCTCGTGCCG
TGAGGTGTCGGCTTAAGTGCCATAACGAGCGCAACCCTTATCTTTAGTTACTAACAGGTCATGCTGAGGACTCTG
GAGAGACTGCCGTCGTAAGATGTGAGGAAGGTGGGGATGACGTCAAATCAGCACGGCCCTTACGTCCGGGGCTAC
ACACGTGTTACAATGGGGGGTACAGAAGGCCGCTACCTGGTGACAGGATGCTAATCCCAAAAGCCTCTCTCAGTT
CGGATCGAAGTCTGCAACCCGACTTCGTGAAGCTGGATTCGCTAGTAATCGCGCATCAGCCATGGCGCGGTGAAT
ACGTTCCCGGGCCTTGTACACACCGCCCGTCAAGCCATGAAAGCCGGGGGTACCTGAAGTACGTAACCGCAAGGA
GCGTCCTAGGGTAAAACTGGTAATTGGGGCTAAGTCGTAACAAGGTA
SEQ ID NO: 29 Alistipes shahii Strain 29
ACATAGGGGGWSTGWKAGGTTWRCCSCCCACATTSRTACTGAGMCA-
TGAWCMAACTCTMTACGGGARGSAGSAGTGAGGAATATTGGTCRRTGGACGCAAGTCTGAACCAGCCATGCCGSG
TGCRGGAAGACGGCTCKATGAGTKGKAAACTGCTTTTGTACRARRGTAAACGCTCTTACGTGTAAGAGCCTGAAA
GTATSGTACRAATGAGGATCGGCTAACTCCGTGCCAGCAGCCGCGGTAATACGGAGGATCCAAGCGTTATCCGGA
TTTATTGGGTTTAAAGGGTGCGTAGGCGGGTTGATAAAGTTAGRGG
SEQ ID NO: 30 Anaerostipes caccae Strain 30
GCTT-ACACATG-
CAAGTCGAACGAAGCATTTARGATTGAAGTTTTCGGATGGATTTCCTATATGACTGAGTGGCGGACGGGTGAGTA
ACGCGTGGGGAACCTGCCCTATACAGGGGGATAACAGCTGGAAACGGCTGCTAATACCGCATAAGCGCACAGAAT
CGCATGATTCAGTGTGAAAAGCCCTGGCAGTATAGGATGGTCCCGCGTCTGATTAGCTGGTTGGTGAGGTAACGG
CTCACCAAGGCGACGATCAGTAGCCGGCTTGAGAGAGTGAACGGCCACATTGGGACTGAGACACGGCCCAAACTC
CTACGGGAGGCAGCAGTGGGGAATATTGCACAATGGGGG-
AAACCCTGATGCAGCGACGCCGCGTGAGTGAAGAAGTATTTCGGTATGTAAAGCTCTATCAGCAGGGAAGAAAAC
AGACGGTACCTGACTAAGAAGCCCCGGCTAACTACGTGCCAGCAGCCGCGGTAATACGTAGGGGGCAAGCGTTAT
CCGGAATTACTGGGTGTAAAGGGTGCGTAGGTGGCATGGTAAGTCAGAAGTGAAAGCCCGGGGCTTAACCCCGGG
ACTGCTTTTGAAACTGTCATGCTGGAGTGCAGGAGAGGTAAGCGGAATTCCTAGTGTAGCGGTGAAATGCGTAGA
TATTAGGAGGAACACCAGTGGCGAAGGCGGCTTACTGGACTGTCACTGACACTGATGCACGAAAGCGTGGGGAGC
AAACAGGATTAGATACCCTGGTAGTCCACGCCGTAAACGATGAATACTAGGTGTCGGGGCCGTAGAGGCTTCGGT
GCCGCAGCAAA
SEQ ID NO: 31 Phascolarctobacterium faecium Strain 31
CGGAGAATTTTCATTTCGGTAGAATTCTTAGTGGCGAACGGGTGAGTAACGCGTAGGCAACCTGCCCTTTAGACG
GGGACAACATTCCGAAAGGAGTGCTAATACCGGATGTGATCATCGTGCCGCATGGCAGGATGAAGAAAGATGGCC
TCTACAAGTAAGCTATCGCTAAAGGATGGGCCTGCGTCTGATTAGCTAGTTGGTAGTGTAACGGACTACCAAGGC
GATGATCAGTAGCCGGTCTGAGAGGATGAACGGCCACATTGGGACTGAGACACGGCCCAAACTCCTACGGGAGGC
AGCAGTGGGGAATCTTCCGCAATGGACGAAAGTCTGACAGAGCAACGCCGCGTGAGTGATGAAGGATTTCGGTCT
GTAAAGCTCTGTTGTTTATGACGAACGTGCAGTGTGTGAACAATGCATTGCAATGACGGTAGTAAACGAGGAAGC
CACGGCTAACTACGTGCCAGCAGCCGCGGTAATACGTAGGTGGCGAGCGTTGTCCGGAATTATTGGGCGTAAAGA
GCATGTAGGCGGCTTAATAAGTCGAGCGTGAAAAATGCGGGGCTCAACCCCGTATGGCGCTGGAAACTGTTAGGC
TTGAGTGCAGGAGAGGAAAGGGGAATTCCCAGTGTAGCGGTGAAATGCGTAGATATTGGGAGGAACACCAGTGGC
GAAGGCGCCTTTCTGGACTGTGTTTGACGCTGAGATGCGAAAGCCAGGGTAGC
SEQ ID NO: 32 Agathobaculum butyriciproducens Strain 32
TAGTGGCGGACGGGTGAGTAACGCGTGAGCAATCTGCCTTTAAGAGGGGGATAACAGTCGGAAACGGCTGCTAAT
ACCGCATAAAGCATTGAATTCGCATGTTTTCGATGCCAAAGGAGCAATCCGCTTTTAGATGAGCTCGCGTCTGAT
TAGCTAGTTGGCGGGGTAACGGCCCACCAAGGCGACGATCAGTAGCCGGACTGAGAGGTTGAACGGCCACATTGG
GACTGAGACACGGCCCAGACTCCTACGGGAGGCAGCAGTGGGGAATATTGCGCAATGGGGGRAACCCTGACGCAG
CAACGCCGCGTGATTGAAGAAGGCCTTCGGGTTGTAAAGATCTTTAATCAGGGACGAA
AMATGACGGTACCTGAAGAATAAGCTCCGGCTAACTACGTGCCAGCAGCCGCGGTAATACGTAGGGAGCAAGCGT
TATCCGGATTTACTGGGTGTAAAGGGCGCGCAGGCGGGCCGGCAAGTTGGAAGTGAAATCCGGGGGCTTAACCCC
CGAACTGCTTTCAAAACTGCTGGTCTTGAGTGATGGAGAGGCAGGCGGAATTCCGTGTGTAGCGGTGAAATGCGT
AGATATACGGAGGAACACCAGTGGCGAAGGCGGCCTGCTGGACATTAACTGACGCTGAGGCGCGAAAGCGTGGGG
AGCAAACAGGATTAGATACCCTGGTAGTCCACGCCGTAAACGATGGATA
SEQ ID NO: 33 Bacteroides fragilis Strain 33
ATGAAGAGTTTGATCCTGGCTCAGGATGAACGCTAGCTACAGGCTTAACACATGCAAGTCGAGGGGCATCAGGAA
GAAAGCTTGCTTTCTTTGCTGGCGACCGGCGCACGGGTGAGTAACACGTATCCAACCTGCCCTTTACTCGGGGAT
AGCCTTTCGAAAGAAAGATTAATACCCGATGGCATAATGATTCCGCATGGTTTCATTATTAAAGGATTCCGGTAA
AGGATGGGGATGCGTTCCATTAGGTTGTTGGTGAGGTAACGGCTCACCAAGCCTTCGATGGATAGGGGTTCTGAG
AGGAAGGTCCCCCACATTGGAACTGAGACACGGTCCAAACTCCTACGGGAGGCAGCAGTGAGGAATATTGGTCAA
TGGGCGCTAGCCTGAACCAGCCAAGTAGCGTGAAGGATGAAGGCTCTATGGGTCGTAAACTTCTTTTATATAAGA
ATAAAGTGCAGTATGTATACTGTTTTGTATGTATTATATGAATAAGGATCGGCTAACTCCGTGCCAGCAGCCGCG
GTAATACGGAGGATCCGAGCGTTATCCGGATTTATTGGGTTTAAAGGGAGCGTAGGTGGACTGGTAAGTCAGTTG
TGAAAGTTTGCGGCTCAACCGTAAAATTGCAGTTGATACTGTCAGTCTTGAGTACAGTAGAGGTGGGCGGAATTC
GTGGTGTAGCGGTGAAATGCTTAGATATCACGAAGAACTCCGATTGCGAAGGCAGCTCACTGGACTGCAACTGAC
ACTGATGCTCGAAAGTGTGGGTATCAAACAGGATTAGATACCCTGGTAGTCCACACAGTAAACGATGAATACTCG
CTGTTTGCGATATACAGTAAGCGGCCAAGCGAAAGCATTAAGTATTCCACCTGGGGAGTACGCCGGCAACGGTGA
AACTCAAAGGAATTGACGGGGGCCCGCACAAGCGGAGGAACATGTGGTTTAATTCGATGATACGCGAGGAACCTT
ACCCGGGCTTAAATTGCAGTGGAATGATGTGGAAACATGTCAGTGAGCAATCACCGCTGTGAAGGTGCTGCATGG
TTGTCGTCAGCTCGTGCCGTGAGGTGTCGGCTTAAGTGCCATAACGAGCGCAACCCTTATCTTTAGTTACTAACA
GGTTATGCTGAGGACTCTAGAGAGACTGCCGTCGTAAGATGTGAGGAAGGTGGGGATGACGTCAAATCAGCACGG
CCCTTACGTCCGGGGCTACACACGTGTTACAATGGGGGGTACAGAAGGCAGCTAGCGGGTGACCGTATGCTAATC
CCAAAAGCCTCTCTCAGTTCGGATCGAAGTCTGCAACCCGACTTCGTGAAGCTGGATTCGCTAGTAATCGCGCAT
CAGCCACGGCGCGGTGAATACGTTCCCGGGCCTTGTACACACCGCCCGTCAAGCCATGGGAGCCGGGGGTACCTG
AAGTACGTAACCGCAAGGATCGTCCTAGGGTAAAACTGGTGACTGGGGCTAAGTCGTAACAAGGTAGCCGTACCG
GAAGGTGCGGCTGGAACACCTCCTT
SEQ ID NO: 34 Fusobacterium mortiferum Strain 34
TGGCTCAGGATGAACGCTGACAGAATGCTTAACACATGCAAGTCTACTTGATCCTTCGGGTGATGGTGGCGGACG
GGTGAGTAACGCGTAAAGAACTTGCCCTGCAGTCTGGGACAACATTTGGAAACGAATGCTAATACCGGATATTAT
GT-
ATTTCTCGCATGAGTTTTACATGAAAGCTATATGCGCTGCAGGAGAGCTTTGCGTCCTATTAGCTAGTTGGTGAG
GTAACGGCTCACCAAGGCCATGATAGGTAGCCGGCCTGAGAGGGTGAACGGCCACAAGGGGACTGAGACACGGCC
CTTACTCCTACGGGAGGCAGCAGTGGGGAATATTGGACAATGGACCAAAAGTCTGATCCAGCAATTCTGTGTGCA
CGATGAAGTTTTTCGGAATGTAAAGTGCTTTCAGTTGGGACGAAGTAAGTGACGGTACCAACAGAAGAAGCGACG
GCTAAATACGTGCCAGCAGCCGCGGTAATACGTATGTCGCAAGCGTTATCCGGATTTATTGGGCGTAAAGCGCGT
CTAGGCGGTTTGGTAAGTCTGATGTGAAAATGCGGGGCTCAACTCCGTATTGCGTTGGAAACTGCTAAACTAGAG
TACTGGAGAGGTGGGCGGAACTACAAGTGTAGAGGTGAAATTCGTAGATATTTGTAGGAATGCCGATGGGGAAGC
CAGCCCACTGGACAGATACTGACGCTAAAGCGCGAAAGCGTGGGTAGCAAACAGGATTAGATACCCTGGTAGTCC
ACGCCGTAAACGATGATTACTAGGTGTTGGGGGTCGAACCTCAGCGCCCAAGCTAACGCGATAAGTAATCCGCCT
GGGGAGTACGTACGCAAGTATGAAACTCAAAGGAATTGACGGGGACCCGCACAAGCGGTGGAGCATGTGGTTTAA
TTCGACGCAACGCGAGGAACCTTACCAGCGTTTGACATCCTAAGAAATTAGCAGAGATGCTTTTGTGCCCCTTCG
GGGGAACTTAGTGACAGGTGGTGCATGGCTGTCGTCAGCTCGTGT
SEQ ID NO: 35 Paraclostridium benzoelyticum/Clostridium bifermantans Strain 35
AGAGTTTGATCCTGGCTCAGGATGAACGCTGGCGGCGTGCCTAACACATGCAAGTCGAGCGATCTCTTCGGAGAG
AGCGGCGGACGGGTGAGTAACGCGTGGGTAACCTGCCCTGTACACACGGATAACATACCGAAAGGTATACTAATA
CGGGATAACATACGAAAGTCGCATGGCTTTTGTATCAAAGCTCCGGCGGTACAGGATGGACCCGCGTCTGATTAG
CTAGTTGGTAAGGTAATGGCTTACCAAGGCAACGATCAGTAGCCGACCTGAGAGGGTGATCGGCCACACTGGAAC
TGAGACACGGTCCAGACTCCTACGGGAGGCAGCAGTGGGGAATATTGCACAATGGGCGAAAGCCTGATGCAGCAA
CGCCGCGTGAGCGATGAAGGCCTTCGGGTCGTAAAGCTCTGTCCTCAAGGAAGATAATGACGGTACTTGAGGAGG
AAGCCCCGGCTAACTACGTGCCAGCAGCCGCGGTAATACGTAGGGGGCTAGCGTTATCCGGAATTACTGGGCGTA
AAGGGTGCGTAGGTGGTTTTTTAAGTCAGAAGTGAAAGGCTACGGCTCAACCGTAGTAAGCTTTTGAAACTAGAG
AACTTGAGTGCAGGAGAGGAGAGTAGAATTCCTAGTGTAGCGGTGAAATGCGTAGATATTAGGAGGAATACCAGT
AGCGAAGGCGGCTCTCTGGACTGTAACTGACACTGAGGCACGAAAGCGTGGGGAGCAAACAGGATTAGATACCCT
GGTAGTCCACGCCGTAAACGATGAGTACTAGGTGTCGGGGGTTACCCCCCTCGGTGCCGCAGCTAACGCATTAAG
TACTCCGCCTGGGAAGTACGCTCGCAAGAGTGAAACTCAAAGGAATTGACGGGGACCCGCACAAGTAGCGGAGCA
TGTGGTTTAATTCGAAGCAACGCGAAGAACCTTACCTAAGCTTGACATCCCACTGACCTCTCCCTAATCGGAGAT
TTCCCTTCGGGGACAGTGGTGACAGGTGGTGCATGGTTGTCGTCAGCTCGTGTCGTGAGATGTTGGGTTAAGTCC
CGCAACGAGCGCAACCCTTGCCTTTAGTTGCCAGCATTAAGTTGGGCACTCTAGAGGGACTGCCGAGGATAACTC
GGAGGAAGGTGGGGATGACGTCAAATCATCATGCCCCTTATGCTTAGGGCTACACACGTGCTACAATGGGTGGTA
CAGAGGGTTGCCAAGCCGCGAGGTGGAGCTAATCCCTTAAAGCCATTCTCAGTTCGGATTGTAGGCTGAAACTCG
CCTACATGAAGCTGGAGTTACTAGTAATCGCAGATCAGAATGCTGCGGTGAATGCGTTCCCGGGTCTTGTACACA
CCGCCCGTCACACCATGGAAGTTGGGGGCGCCCGAAGCCGGTTAGCTAACCTTTTAGGAAGCGGCCGTCGAAGGT
GAAACCAATGACTGGGGTGAAGTCGTAACAAGGTAGCCGTATCGGAAGGTGCGGCTGGATCACCT
SEQ ID NO: 36 Escherichia fergusonii/Escherichia coli Strain 36
TTGAAGAGTTTGATCATGGCTCAGATTGAACGCTGGCGGCAGGCCTAACACATGCAAGTCGAACGGTAACAGGAA
GCAGCTTGCTGCTTTGCTGACGAGTGGCGGACGGGTGAGTAATGTCTGGGAAACTGCCTGATGGAGGGGGATAAC
TACTGGAAACGGTAGCTAATACCGCATAACGTCGCAAGACCAAAGAGGGGGACCTTCGGGCCTCTTGCCATCGGA
TGTGCCCAGATGGGATTAGCTAGTAGGTGGGGTAACGGCTCACCTAGGCGACGATCCCTAGCTGGTCTGAGAGGA
TGACCAGCCACACTGGAACTGAGACACGGTCCAGACTCCTACGGGAGGCAGCAGTGGGGAATATTGCACAATGGG
CGCAAGCCTGATGCAGCCATGCCGCGTGTATGAAGAAGGCCTTCGGGTTGTAAAGTACTTTCAGCGGGGAGGAAG
GGAGTAAAGTTAATACCTTTGCTCATTGACGTTACCCGCAGAAGAAGCACCGGCTAACTCCGTGCCAGCAGCCGC
GGTAATACGGAGGGTGCAAGCGTTAATCGGAATTACTGGGCGTAAAGCGCACGCAGGCGGTTTGTTAAGTCAGAT
GTGAAATCCCCGGGCTCAACCTGGGAACTGCATCTGATACTGGCAAGCTTGAGTCTCGTAGAGGGGGGTAGAATT
CCAGGTGTAGCGGTGAAATGCGTAGAGATCTGGAGGAATACCGGTGGCGAAGGCGGCCCCCTGGACGAAGACTGA
CGCTCAGGTGCGAAAGCGTGGGGAGCAAACAGGATTAGATACCCTGGTAGTCCACGCCGTAAACGATGTCGACTT
GGAGGTTGTGCCCTTGAGGCGTGGCTTCCGGAGCTAACGCGTTAAGTCGACCGCCTGGGGAGTACGGCCGCAAGG
TTAAAACTCAAATGAATTGACGGGGGCCCGCACAAGCGGTGGAGCATGTGGTTTAATTCGATGCAACGCGAAGAA
CCTTACCTGGTCTTGACATCCACGGAAGTTTTCAGAGATGAGAATGTGCCTTCGGGAACCGTGAGACAGGTGCTG
CATGGCTGTCGTCAGCTCGTGTTGTGAAATGTTGGGTTAAGTCCCGCAACGAGCGCAACCCTTATCCTTTGTTGC
CAGCGGTCCGGCCGGGAACTCAAAGGAGACTGCCAGTGATAAACTGGAGGAAGGTGGGGATGACGTCAAGTCATC
ATGGCCCTTACGACCAGGGCTACACACGTGCTACAATGGCGCATACAAAGAGAAGCGACCTCGCGAGAGCAAGCG
GACCTCATAAAGTGCGTCGTAGTCCGGATTGGAGTCTGCAACTCGACTCCATGAAGTCGGAATCGCTAGTAATCG
TGGATCAGAATGCCACGGTGAATACGTTCCCGGGCCTTGTACACACCGCCCGTCACACCATGGGAGTGGGTTGCA
AAAGAAGTAGGTAGCTTAACCTTCGGGAGGGCGCTTACCACTTTGTGATTCATGACTGGGGTGAAGTCGTAACAA
GGTAACCGTAGGGGAACCTGCGGTTGGATCACCTCCTT

The invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Unless otherwise defined herein, scientific and technical terms used in connection with the present disclosure shall have the meanings that are commonly understood by those of ordinary skill in the art. Further, unless otherwise required by context, singular terms shall include pluralities and plural terms hall include the singular. The methods and techniques of the present disclosure are generally performed according to conventional methods well-known in the art. Generally, nomenclatures used in connection with, and techniques of biochemistry, enzymology, molecular and cellular biology, microbiology, virology, cell or tissue culture, genetics and protein and nucleic chemistry described herein are those well-known and commonly used in the art. The methods and techniques of the present disclosure are generally performed according to conventional methods well known in the art and as described in various general and more specific references that are cited and discussed throughout the present specification unless otherwise indicated.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Example 1: Efficacy of Bacterial Compositions to Decolonize CRE

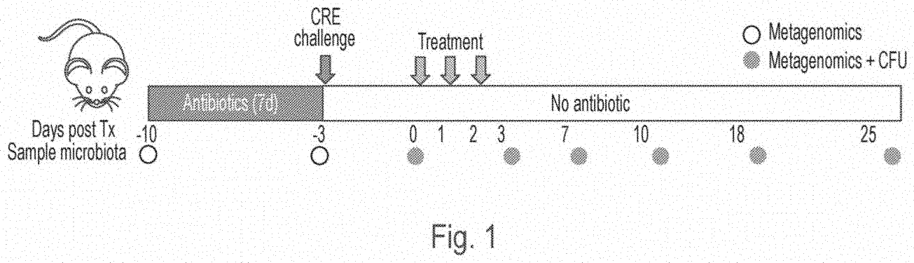

As shown in FIG. 1, C57BL/6J mice were treated with antibiotics (e.g., 0.5 g/L ampicillin) for 7 days prior to challenge with Klebsiella pneumoniae challenge (Klebsiella pneuomoniae ATCC BAA-2814, “CRE”). On day −3, mice were challenged with CRE. On days 0, 1, and 2, mice were administered treatment (e.g., PBS (control), the 36-mix live biotherapeutic product (36-mix), the 36-mix without E. coli (36-Ec), the 36-mix without Fusobacterium mortiferum (36-Fuso), the 36-mix without E. coli or Fusobacterium mortiferum (36-Ec&Fuso), or stool fraction library (SFL)). Fecal samples (pellets) were collected at various time points post-treatment to quantify colony forming units (CFU) as measure of CRE colonization levels. The efficacy of a treatment was assessed based on the criteria in Table 4.

TABLE 4
Efficacy Level

| Efficacy Level | CFU decrease (log) | Clearance (%) |
|---|---|---|
| High | ≥3 | ≥25 |
| Intermediate | 0-2 | 0-20 |
| Low | 2-3 | 0-25 |

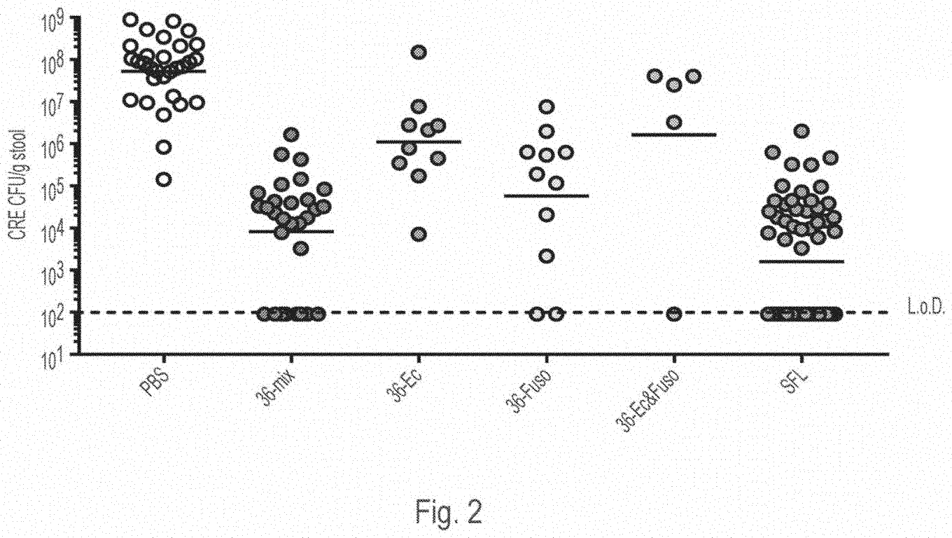

As shown in FIG. 2 and Table 5, removal of E. coli and Fusobacterium mortiferum, separately or together, resulted in an increase in CRE colonization as compared to the 36-mix composition. The bacterial compositions without E. coli and/or Fusobacterium mortiferum were characterized by reduced efficacy in CRE clearance, relative to the highly efficacious 36-mix.

TABLE 5
Decolonization efficacy of bacterial compositions

| | Klebsiella pneumoniae |
|---|---|
| | (CRE) Clearance |
| LBP/SFL | Efficacy |
| 36-mix | High |
| 36-mix without E. coli | Low |
| 36-mix without | Intermediate |
| Fusobacterium | |
| 36-mix without E. coli | Low |
| and Fusobacterium | |
| SFL (17014) | High |

### Example 2: Efficacy of 36-Mix Derivatives Against CRE Decolonization

As shown in FIG. 1, C57BL/6J mice were treated with antibiotics (e.g., 0.5 g/L ampicillin) for 7 days prior to challenge with Klebsiella pneuomoniae challenge (Klebsiella pneuomoniae ATCC BAA-2814, “CRE”). On day −3, mice were challenged with CRE. On days 0, 1, and 2, mice were administered treatment (e.g., PBS (control), the 36-mix live biotherapeutic product (36-mix), the 33-mix (33-mix B), the 32-mix, the 27-mix, the 23-mix, or stool fraction library (SFL)). Fecal samples (pellets) were collected at various time points post-treatment to quantify colony forming units (CFU) as measure of CRE colonization levels. The efficacy of a treatment was assessed based on the criteria in Table 4.

As shown in FIG. 3 and Table 6, several of the bacterial compositions were able to reduce CRE colonization to a comparable level as the 36-mix composition.

TABLE 6
Decolonization efficacy of bacterial compositions

| | Klebsiella pneumoniae |
|---|---|
| | (CRE) Clearance |
| LBP/SFL | Efficacy |
| 36-mix | High |
| 33-mix | High |
| 32-mix | Low |
| 27-mix | High |
| 23-mix | High |
| SFL (17014) | High |

### Example 3: Efficacy of Bacteria Compositions to Promote CRE Decolonization

As shown in FIG. 1, C57BL/6J mice are treated with antibiotics (e.g., 0.5 g/L ampicillin) for 7 days prior to challenge with Klebsiella pneuomoniae challenge (Klebsiella pneuomoniae ATCC BAA-2814, “CRE”). On day −3, mice are challenged with CRE. On days 0, 1, and 2, mice are administered the following treatments:

a) PBS (negative control);

b) stool fraction library (SFL);

c) bacterial composition containing: Clostridium bolteae, Anaerotruncus colihominis, Sellimonas intestinalis, Clostridium symbiosum, Blautia producta, Dorea longicatena, Erysipelotrichaceae bacterium, and

d) bacterial composition containing: Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and Escherichia sp. 3_2_53FAA;

e) bacterial composition containing: Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and Fusobacterium mortiferum;

f) bacterial composition containing: Clostridium bolteae, Anaerotruncus colihominis, Sellimonas intestinalis, Clostridium symbiosum, Blautia producta, Dorea longicatena, Erysipelotrichaceae bacterium, Flavinofractor plautii, Escherichia sp. 3_2_53FAA, and Fusobacterium mortiferum;

e) bacterial composition containing: Clostridium bolteae, Anaerotruncus colihominis, Sellimonas intestinalis, Clostridium symbiosum, Blautia producta, Dorea longicatena, Erysipelotrichaceae bacterium, Flavinofractor plautii, Escherichia sp. 3_2_53FAA, and Fusobacterium mortiferum and two Bacteroides strains;

Fecal samples (pellets) are collected at various time points post-treatment to quantify colony forming units (CFU) as measure of CRE colonization levels.

### Example 4: In Vitro Competition Assays with Campylobacter jejuni 81-176

Individual bacterial strains were evaluated for their ability to kill Campylobacter jejuni strain 81-176 (C. jejuni) by an in vitro mixed culture competition assay. Each of bacterial strains 1-36 and VE303 strains 1-8 were assessed relative to controls: Lactococcus sp. (JL17), Escherichia coli ATCC 25922 (Ec 25922), Campylobacter in Thioglycollate broth (Campy thio), Campylobacter in Thioglycollate broth supplemented with chopped meat (Campy CM-thio), Campylobacter in Thioglycollate broth plus succinate (Campy thio+S), and VE303 strain 6.

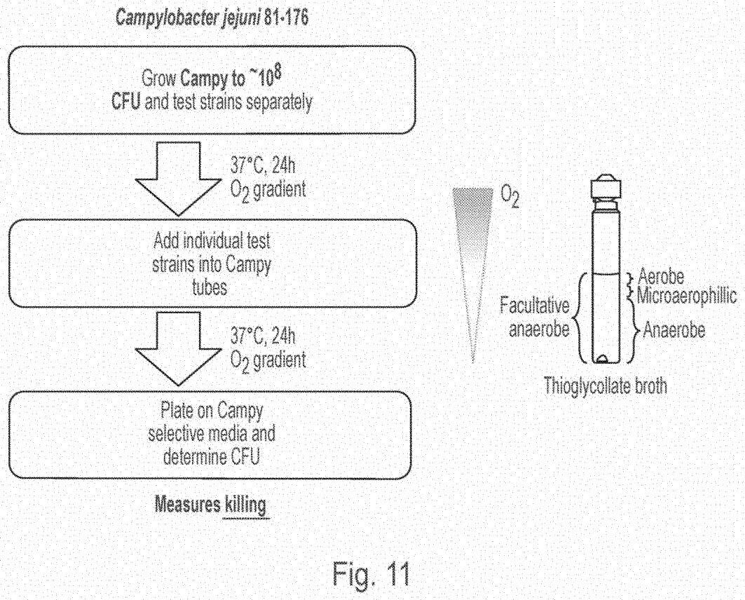

Each of the bacterial strains were assessed according to the workflow diagram shown in FIG. 11B. Briefly, each of the bacterial strains and C. jejuni were grown separately to ˜10⁸ colony forming units (CFUs) at 37° C. for 24 hours in an oxygen concentration gradient in Thioglycollate broth, Campy CM-thio, or Campy thio+S. The cultures of the individual bacterial strains were then added to the C. jejuni culture and incubated at 37° C. for 24 hours. The combined culture was then plated on C. jejuni selective media for CFU enumeration by counting colonies for the positive and negative controls, as well as each of the competition experiments. Each of the strains was compared to the average Campylobacter input to evaluate killing of C. jejuni in the presence of the individual bacterial strains.

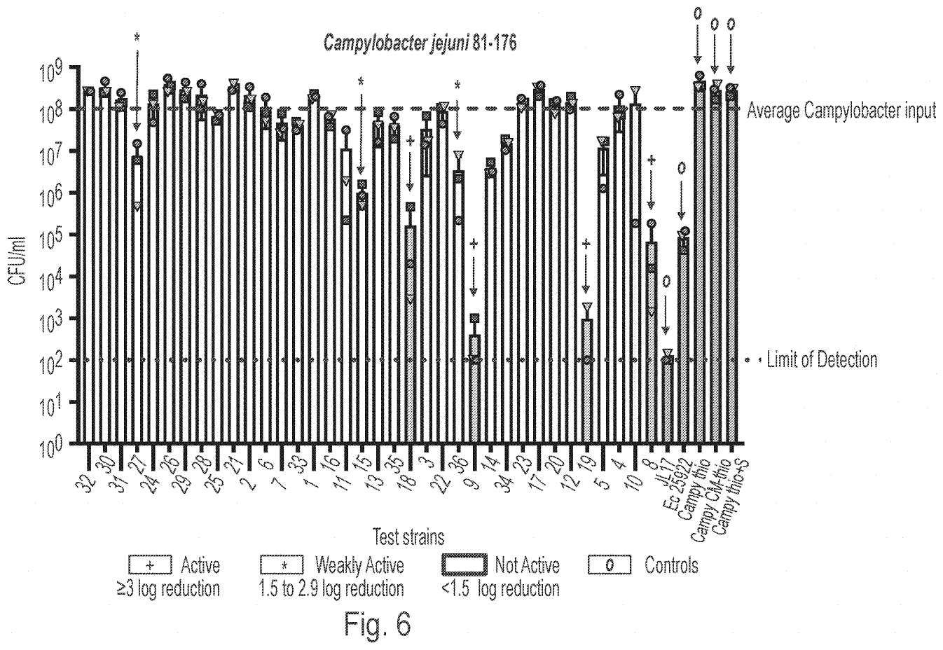

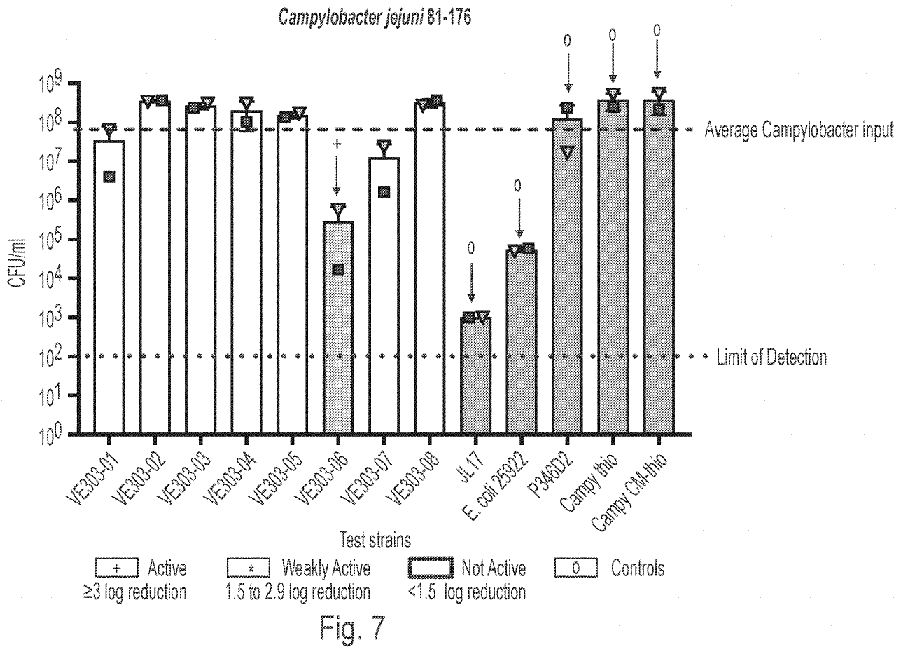

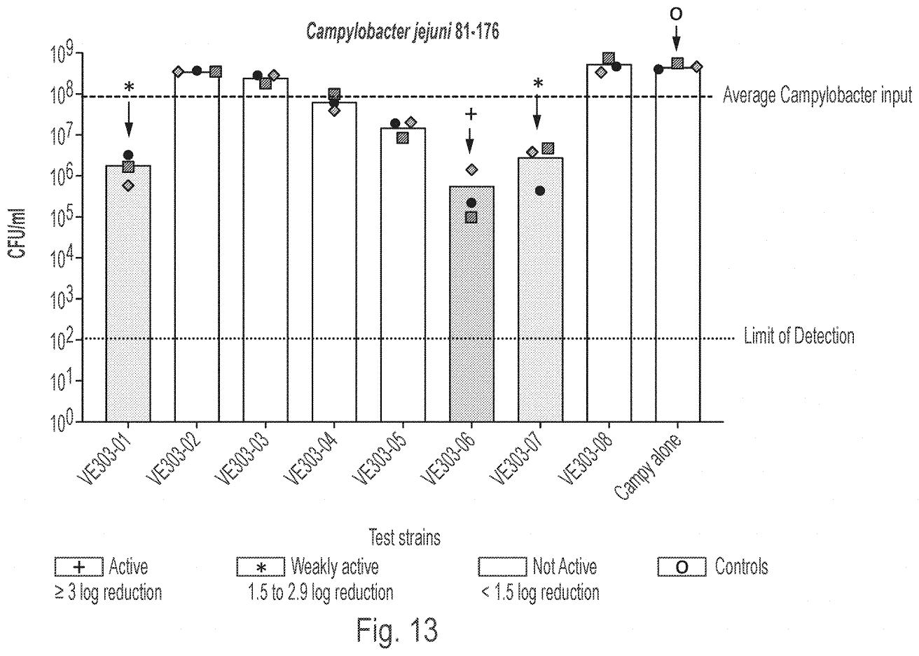

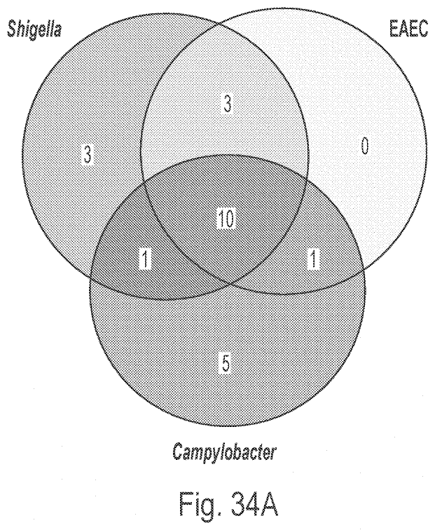

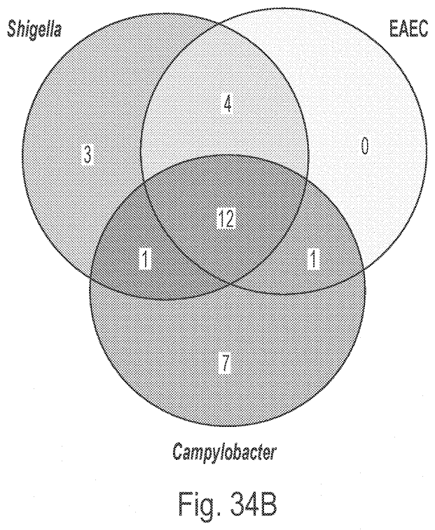

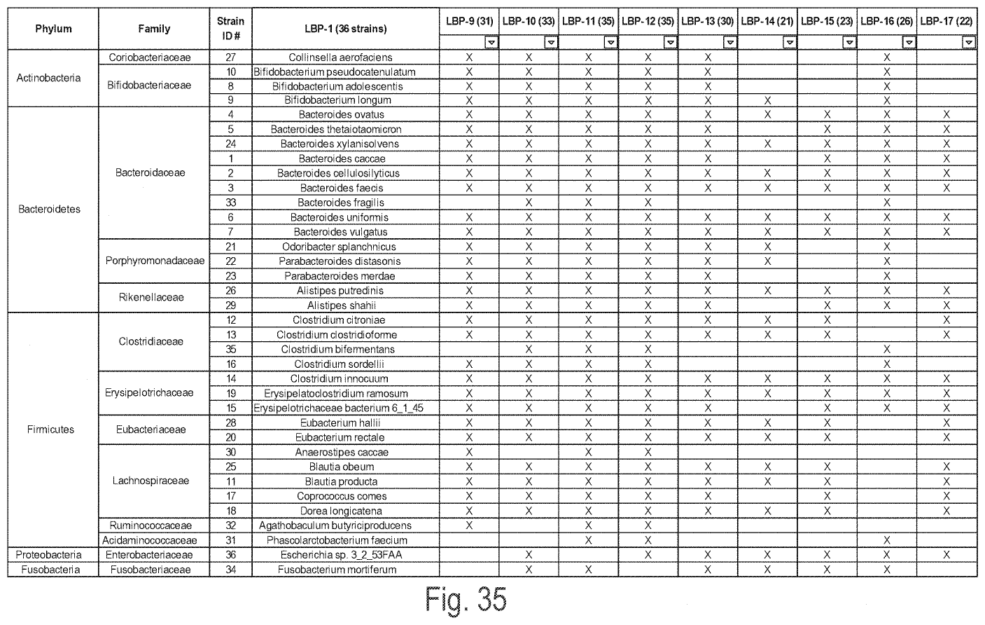

The results are shown in FIGS. 6, 7, 13, 17-21, 34A and 34B, and Tables 7 and 8, below. Bacterial strains 8, 9, 18, and 19, and VE303 strain 6 (VE303-06) were considered “active,” and bacterial strains 11, 14, 15, 27, 36, VE303 strain 1 (VE303-01) and VE303 strain 7 (VE303-07) were weakly active in decreasing C. jejuni survival.

To be considered “active” the bacterial strain reduced C. jejuni survival by ≥3 log, to be considered “weakly active” the bacterial strain reduced C. jejuni survival by between 1.5 to 2.9 log, and “not active” means bacterial strain reduced C. jejuni survival by <1.5 log.

TABLE 7
C. jejuni survival

| Strain | Classification | Activity |
|---|---|---|
| 8 | Bifidobacterium adolescentis | Active |
| 9 | Bifidobacterium longum | Active |
| 18 | Dorea longicatena | Active |
| 19 | Erysipelatoclostridium ramosum | Active |
| 15 | Clostridium innocuum | Weakly Active |
| 27 | Collinsella aerofaciens | Weakly Active |
| 36 | Escherichia coli | Weakly Active |
| 11 | Blautia producta | Weakly Active |
| 14 | Clostridium innocuum | Weekly Active |

TABLE 8
C. jejuni survival

| Strain | Classification | Activity |
|---|---|---|
| VE303-6 | Dorea longicatena | Active |
| VE303-1 | Clostridium bolteae | Weakly Active |
| VE303-7 | Clostridium innocuum | Weakly Active |

### Example 5: In Vitro Competition Assays with Shigella flexneri 2457T

Individual bacterial strains were evaluated for their ability to suppress growth of Shigella flexneri strain 2457T (S. flexneri) by an in vitro mixed culture competition assay. Each of bacterial strains 1-36 and VE303 strains 1-8 were assessed relative to controls: Lactococcus sp. (JL17), Escherichia coli ATCC 25922 (Ec 25922), Shigella in peptone-yeast-glucose medium (Shigella PYG), Shigella in chopped meat with carbohydrates broth (Shigella CMC), Shigella in yeast casitone fatty acids with carbohydrates broth (Shigella YCFAC), Shigella in peptone-yeast-glucose medium with succinate (Shigella PYG+S), and strain 24.

Each of the bacterial strains were assessed according to the workflow diagram in FIG. 11A. Briefly, each of the bacterial strains and S. flexneri were grown separately at 37° C. for 18 hours under anaerobic conditions. The cultures of the individual bacterial strains were then added to S. flexneri cultures (˜10² CFU) and incubated at 37° C. for 18 hours under anaerobic conditions. The co-cultures were then plated on Shigella selective media for CFU enumeration by counting colonies for the positive and negative controls, as well as each of the competition experiments. Each of the strains was compared to the average Shigella input and controls to evaluate growth suppression of S. flexneri in the presence of the individual bacterial strains.

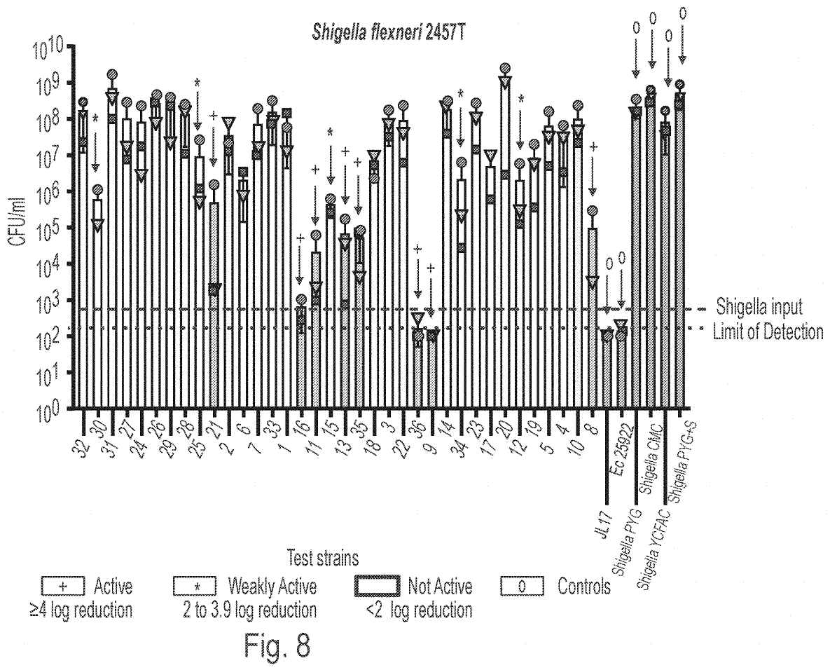

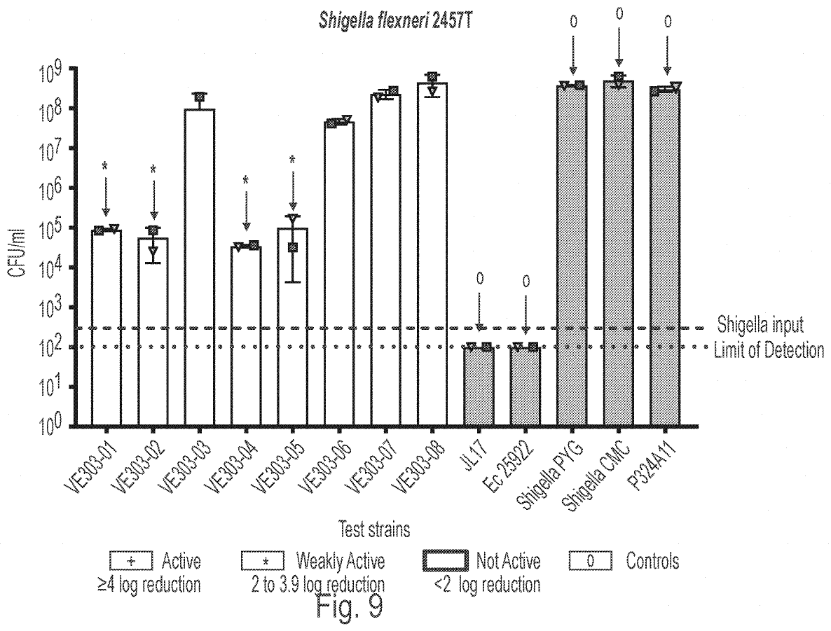

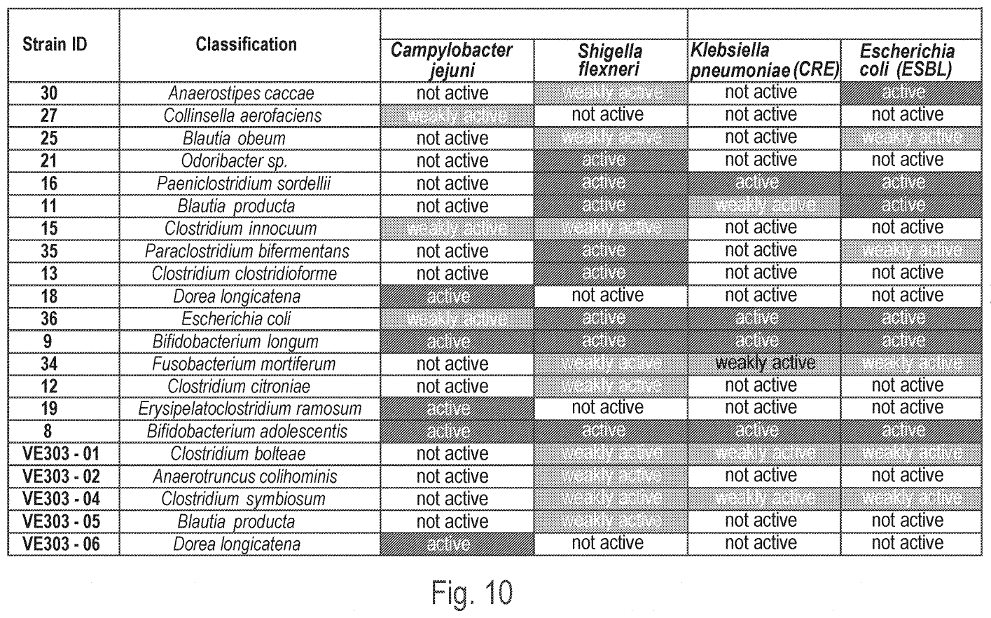

The results are shown in FIGS. 8, 9, 17-21, 34A and 34B, and Tables 9 and 10, below. Bacterial strains 8, 9, 11, 16, 21, and 36 were considered “active,” and bacterial strains 12, 13, 14, 15, 25, 30, 34, 35, VE303 strain 1 (VE303-01), VE303 strain 4 (VE303-04), and VE303 strain 5 (VE303-05) were considered “weakly active” in suppressing the growth of S. flexneri in vitro.

To be considered “active” the bacterial strain reduced S. flexneri by ≥4 log, to be considered “weakly active” the bacterial strain reduced S. flexneri by between 2 to 3.9 log, and “not active” means bacterial strain reduced S. flexneri by <2 log.

TABLE 9
S. flexneri suppression

| Strain | Classification | Activity |
|---|---|---|
| 8 | Bifidobacterium adolescentis | Active |
| 9 | Bifidobacterium longum | Active |
| 11 | Blautia producta | Active |
| 16 | Paeniclostridium sordellii | Active |
| 21 | Odoribacter sp. | Active |
| 36 | Escherichia coli | Active |
| 12 | Clostridium citroniae | Weakly Active |
| 15 | Clostridium innocuum | Weakly Active |
| 25 | Blautia obeum | Weakly Active |
| 30 | Anaerostipes caccae | Weakly Active |
| 34 | Fusobacterium mortiferum | Weakly Active |
| 35 | Paraclostridium bifermentans | Weakly Active |
| 13 | Clostridium clostridioforme | Weakly Active |
| 14 | Clostridium innocuum | Weakly Active |

TABLE 10
S. flexneri suppression

| Strain | Classification | Activity |
|---|---|---|
| 1 | Clostridium bolteae | Weakly Active |
| 4 | Clostridium symbiosum | Weakly Active |
| 5 | Blautia producta | Weakly Active |

### Example 6: In Vitro Competition Assays with Enteroaggregative Escherichia coli

Individual bacterial strains were evaluated for their ability to suppress growth of enteroaggregative Escherichia coli (EAEC) by an in vitro mixed culture competition assay. Each of bacterial strains 1-36 and VE303 strains 1-8 were assessed relative to controls.

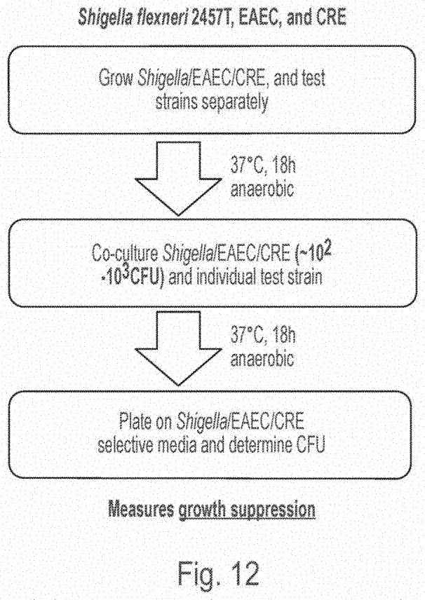

Each of the bacterial strains were assessed according to the workflow diagram in FIG. 12. Briefly, each of the bacterial strains and EAEC were grown separately at 37° C. for 18 hours under anaerobic conditions. The cultures of the individual bacterial strains were then added to EAEC cultures (˜10² CFU) and incubated at 37° C. for 18 hours under anaerobic conditions. The co-cultures were then plated on EAEC selective media for CFU enumeration by counting colonies for the positive and negative controls, as well as each of the competition experiments.

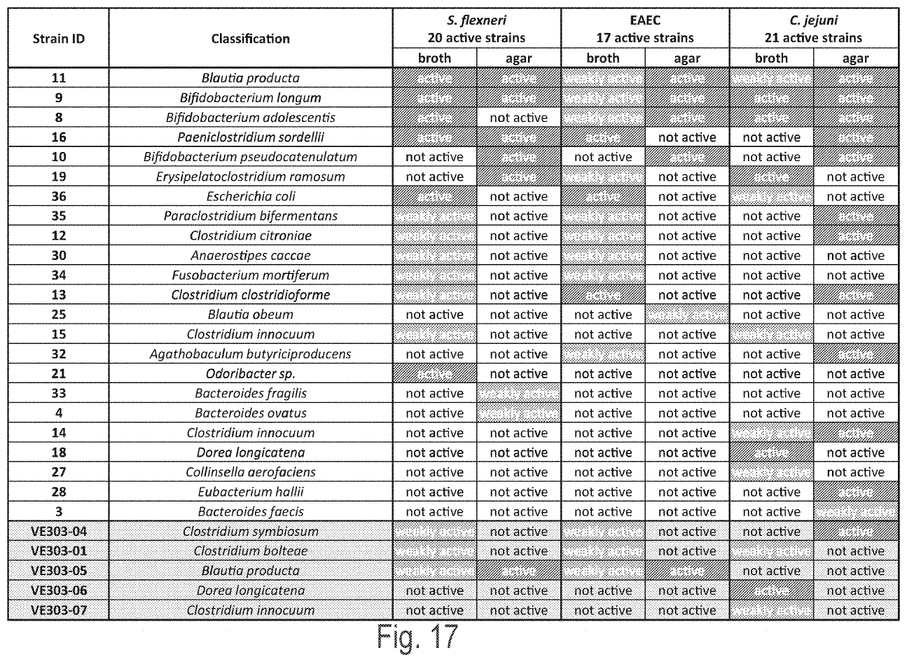

The results are shown in FIGS. 17-21 and Tables 11 and 12, below. Bacterial strains 9, 13, 16, and 36 were considered “active,” and bacterial strains 8, 9, 11, 12, 19, 30, 32, 34, 35, VE303 strain 1 (VE303-1), VE303 strain 4 (VE303-4), and VE303 strain 5 (VE303-5) were considered “weakly active” in suppressing the growth of EAEC in vitro. To be considered “active” the bacterial strain reduced EAEC by ≥4 log, to be considered “weakly active” the bacterial strain reduced EAEC by between 2 to 3.9 log, and “not active” means bacterial strain reduced EAEC by <2 log.

TABLE 11
EAEC suppression

| Strain | Classification | Activity |
|---|---|---|
| 9 | Bifidobacterium longum | Active |
| 13 | Clostridium clostridioforme | Active |
| 16 | Paeniclostridium sordellii | Active |
| 36 | Escherichia coli | Active |
| 8 | Bifidobacterium adolescentis | Weakly Active |
| 9 | Bifidobacterium longum | Weakly Active |
| 11 | Blautia producta | Weakly Active |
| 12 | Clostridium citroniae | Weakly Active |
| 19 | Erysipelatoclostridium ramosum | Weakly Active |
| 30 | Anaerostipes caccae | Weakly Active |
| 32 | Agathobaculum butyriciproducens | Weakly Active |
| 34 | Fusobacterium mortiferum | Weakly Active |
| 35 | Paraclostridium bifermantans | Weakly Active |

TABLE 12
EAEC suppression

| Strain | Classification | Activity |
|---|---|---|
| VE303-1 | Clostridium bolteae | Weakly Active |
| VE303-4 | Clostridium symbiosum | Weakly Active |
| VE303-5 | Blautia producta | Weakly Active |

### Example 7: In Vitro Screening of Bacterial Strains Against Pathogenic Bacteria

Various in vitro screening assays can be utilized to identify bacterial strains that are active against pathogens (e.g., Campylobacter, Shigella, and Enteroaggregative E. coli (EAEC)).

As discussed above, broth competition assays assess bacteria survival or growth suppression, using colony forming units on selective media as a readout. Potential mechanisms of action include pH, nutrient competition, production of metabolites (e.g., short chain fatty acids), and/or other inhibitory molecules (e.g., bacteriocins).

Additionally, a soft agar overlay assay may be used to measure bacterial survival or growth suppression using zone of inhibition as a readout. Potential mechanisms of action include production of soluble inhibitor molecules (e.g., bacteriocins).

### Example 8: In Vitro Soft Agar Overlay Assays

Individual bacterial strains were evaluated for their ability to suppress growth of Campylobacter jejuni strain 81-176 (C. jejuni), Shigella flexneri strain 2457T (S. flexneri), enteroaggregative Escherichia coli (EAEC), or Klebsiellaa pneumoniae ATCC BAA-2814 (CRE) by an in vitro soft agar overlay assay. Each of bacterial strains 1-36 and VE303 strains 1-8 were assessed relative to controls.

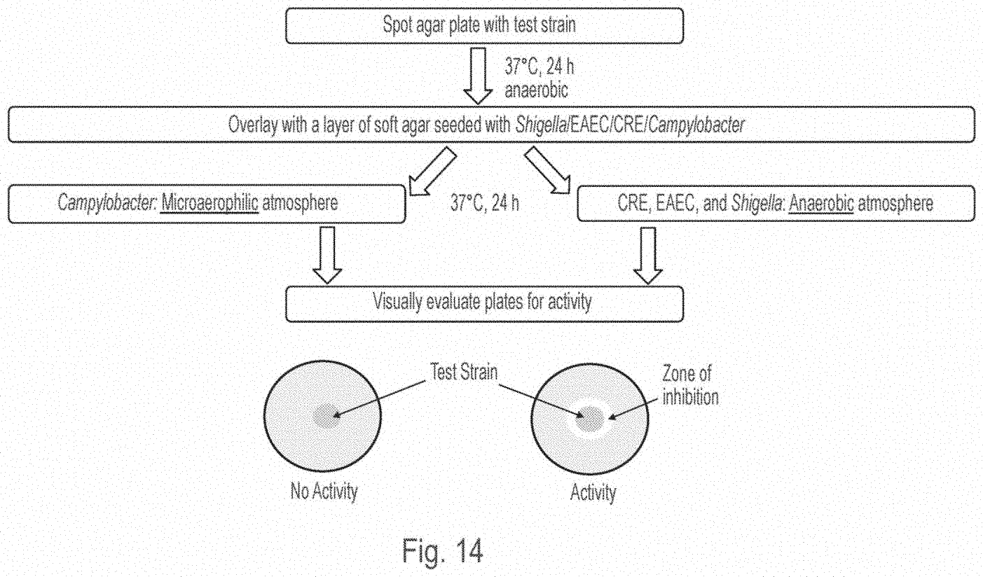

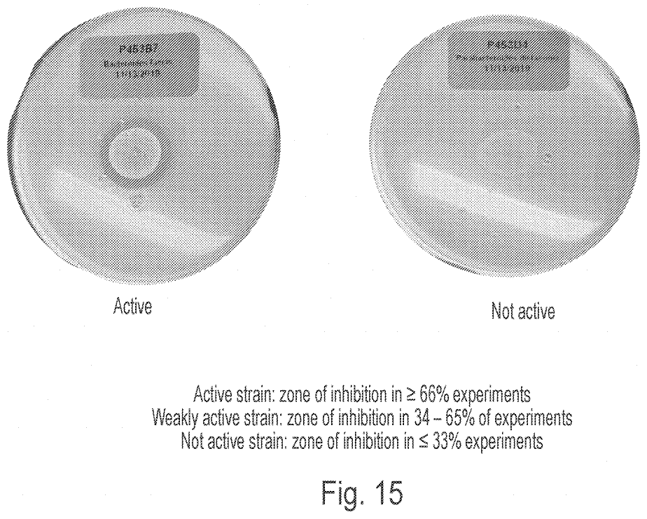

Each of the bacterial strains were assessed according to the workflow diagram in FIG. 14. Briefly, an agar plate was spotted with one of the bacterial strains and grown at 37° C. for 24 hours under anaerobic conditions. The agar plates of the individual bacterial strains were then overlayed with a soft agar seeded with C. jejuni, S. flexneri, EAEC, or CRE and incubated at 37° C. for 24 hours under anaerobic conditions (S. flexneri, EAEC, and CRE) or microaerophilic conditions (C. jejuni). The plates are then evaluated for a zone of inhibition for the controls, as well as each of the bacterial strain experiments. Each of the strains was compared to controls to evaluate growth suppression and/or killing of C. jejuni, S. flexneri, EAEC, or CRE in the presence of the individual bacterial strains (FIG. 15).

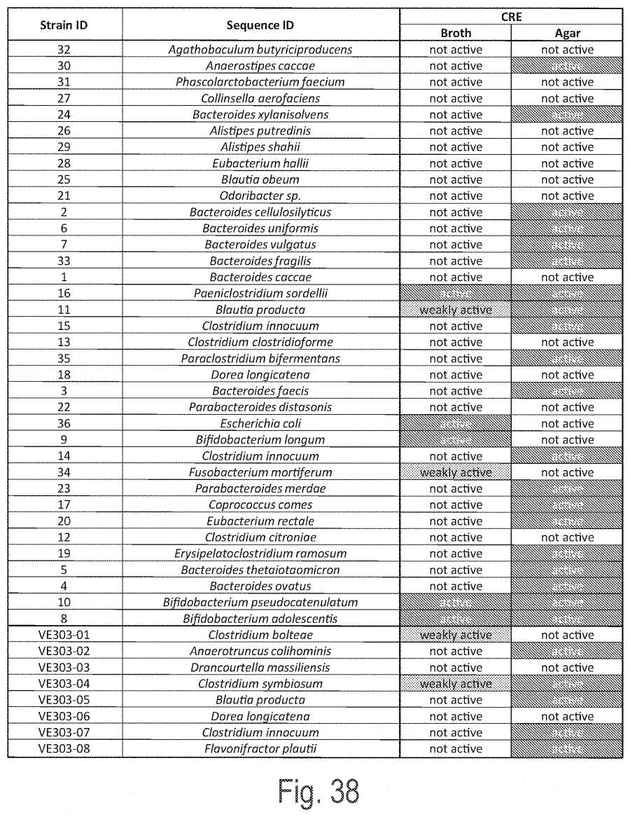

Each experiment was performed at least 3 times per bacterial strain, and plates were independently scored by two individuals. The results are shown FIGS. 17-21, 34A, 34B, and 38 and Tables 13-18, and 23-24, below. To be considered “active” the bacterial strain produced a zone of inhibition in greater than 66% of replicates; to be considered “weakly active,” the bacterial strain produced a zone of inhibition in 33%-66% of replicates; and to be considered “not active,” the bacterial strain produced s zone of inhibition in less than 33% of replicates.

Bacterial strains 8, 9, 10, 11, 12, 13, 14, 15, 16, 28, 32, 35, VE303 strain 4 (VE303-4), and VE303 strain 7 (VE303-7) were considered “active,” in suppressing the growth of C. jejuni in vitro, and bacterial strain 3 was considered “weakly active.”

TABLE 13
C. jejuni suppression

| Strain | Classification | Activity |
|---|---|---|
| 8 | Bifidobacterium adolescentis | Active |
| 9 | Bifidobacterium longum | Active |
| 10 | Bifidobacterium pseudocatenulatum | Active |
| 11 | Blautia producta | Active |
| 12 | Clostridium citroniae | Active |
| 14 | Clostridium innocuum | Active |
| 15 | Clostridium innocuum | Active |
| 16 | Paeniclostridium sordellii | Active |
| 28 | Eubacterium hallii | Active |
| 32 | Agathobaculum butyriciproducens | Active |
| 35 | Paraclostridium bifermentans | Active |
| 3 | Bacteroides faecis | Weakly Active |

TABLE 14
C. jejuni suppression

| Strain | Classification | Activity |
|---|---|---|
| VE303-4 | Clostridium symbiosum | Active |
| VE303-7 | Clostridium innocuum | Active |

Bacterial strains 9, 10, 11, 16, 19, and VE303-5, were considered “active,” and bacterial strains 4 and 33 were considered “weakly active” in suppressing the growth of S. flexneri in vitro.

TABLE 15
S. flexneri suppression

| Strain | Classification | Activity |
|---|---|---|
| 9 | Bifidobacterium longum | Active |
| 10 | Bifidobacterium pseudocatenulatum | Active |
| 11 | Blautia producta | Active |
| 16 | Paeniclostridium sordellii | Active |
| 19 | Erysipelatoclostridium ramosum | Active |
| 4 | Bacteroides ovatus | Weakly Active |
| 33 | Bacteroides fragilis | Weakly Active |

TABLE 16
S. flexneri suppression

| Strain | Classification | Activity |
|---|---|---|
| VE303-5 | Blautia producta | Active |

Bacterial strains 8, 9, 10, 11, and VE303-5, were considered “active,” and bacterial strain 25 was considered “weakly active” in suppressing the growth of EAEC in vitro.

TABLE 17
EAEC suppression

| Strain | Classification | Activity |
|---|---|---|
| 8 | Bifidobacterium adolescentis | Active |
| 9 | Bifidobacterium longum | Active |
| 10 | Bifidobacterium pseudocatenulatum | Active |
| 11 | Blautia producta | Active |
| 25 | Blautia obeum | Weakly Active |

TABLE 18
EAEC suppression

| Strain | Classification | Activity |
|---|---|---|
| VE303-5 | Blautia producta | Active |

Bacterial strains 2, 3, 4, 5, 6, 7, 8, 10, 11, 15, 14, 16, 17, 19, 20, 23, 24, 30, 33, 35, VE303 strain 2 (VE303-2), VE303 strain 4 (VE303-4), VE303 strain 5 (VE303-5), VE303 strain 7 (VE303-7), and VE303 strain 8 (VE303-8) were considered “active,” in suppressing the growth of CRE in vitro.

TABLE 23
CRE suppression

| Strain | Classification | Activity |
|---|---|---|
| 30 | Anaerostipes caccae | Active |
| 24 | Bacteroides xylanisolvens | Active |
| 2 | Bacteroides cellulosilyticus | Active |
| 6 | Bacteroides uniformis | Active |
| 7 | Bacteroides vulgatus | Active |
| 33 | Bacteroides fragilis | Active |
| 16 | Paeniclostridium sordellii | Active |
| 11 | Blautia producta | Active |
| 15 | Clostridium innocuum | Active |
| 35 | Paraclostridium bifermentans | Active |
| 3 | Bacteroides faecis | Active |
| 14 | Clostridium innocuum | Active |
| 23 | Parabacteroides merdae | Active |
| 17 | Coprococcus comes | Active |
| 20 | Eubacteria rectale | Active |
| 19 | Erysipelatoclostridium ramosum | Active |
| 5 | Bacteroides thetaiotamicron | Active |
| 4 | Bacteroides ovatus | Active |
| 10 | Bifidobacterium pseudocatenulatum | Active |
| 8 | Bifidobacterium adolescentis | Active |

TABLE 24
CRE suppression

| Strain | Classification | Activity |
|---|---|---|
| VE303-2 | Anaerotruncus colihominis | Active |
| VE303-4 | Clostridium symbiosum | Active |
| VE303-5 | Blautia producta | Active |
| VE303-7 | Clostridium innocuum | Active |
| VE303-8 | Flavonifractor plautii | Active |

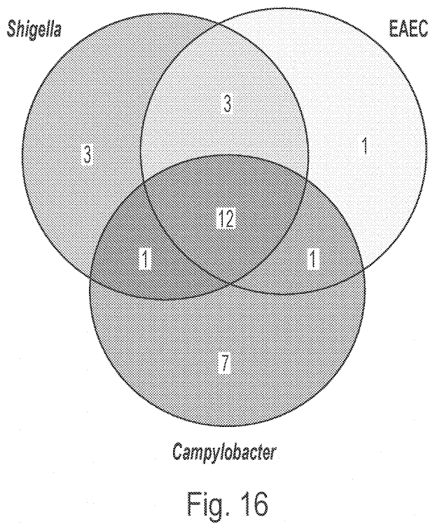

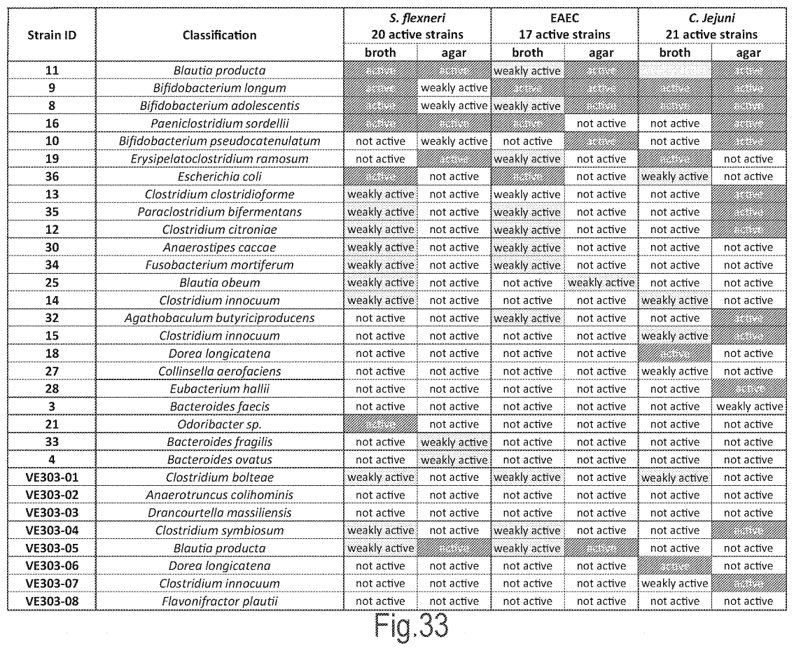

A summary of all the active strains in the in vitro broth competition assays and soft agar overlay assays is shown in FIGS. 16, 32, and 33. Overall, 38 of 44 strains tested were active at suppressing at least one pathogen (C. jejuni, S. flexneri, or EAEC). Of these 38 strains, 31 were from the 36-mix and 7 were from the VE303 composition. 21 bacterial strains were active against C. jejuni, 20 bacterial strains were active against S. flexneri, 17 bacterial strains were active against EAEC, and 29 bacterial strains were active against CRE.

Of the 38 active strains, the activity of 26 strains was assay-specific: 12 bacterial strains were active at suppressing at least one pathogen only in the broth competition assay, and 14 were active at suppressing at least one pathogen only in the soft agar overlay assay.

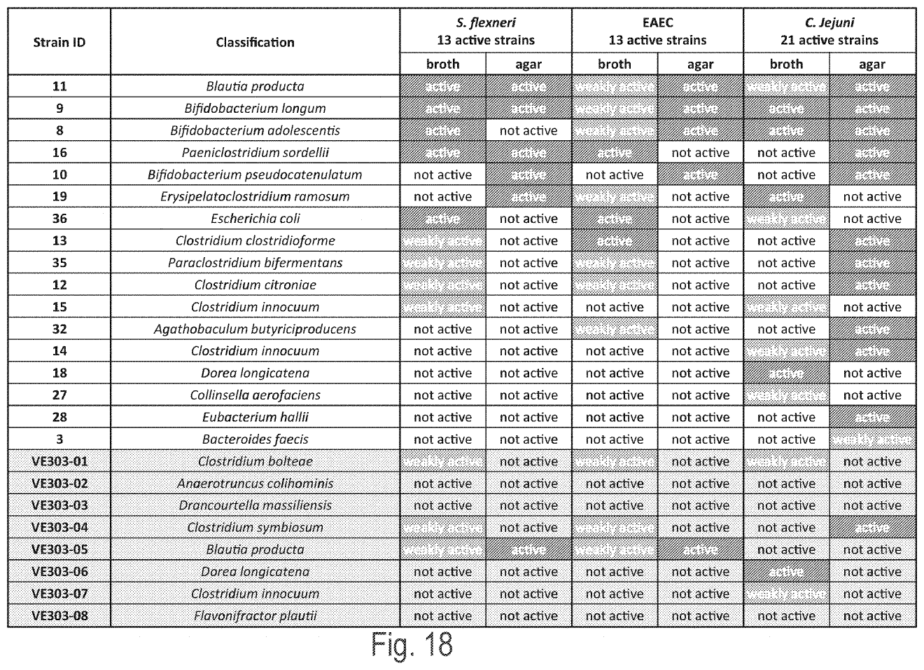

Of the 36-mix composition, 5 bacterial strains were found to be inactive against all three pathogens tested. Of the VE303 composition, 1 bacterial strain was inactive. The bacterial strains considered to be “inactive” are shown in Table 19. Two bacterial families were not represented in the compositions shown in FIGS. 18-21: Acidaminococcoceae and Rikenellaceae.

TABLE 19
Inactive strains

| Strain | Classification |
|---|---|
| 31 | Phascolarctobacterium faecium |
| 26 | Alistipes putredinis |
| 29 | Alistipes shahii |
| 1 | Bacteroides caccae |
| 22 | Parabacteroides distasonis |
| VE303-03 | Drancourtella massiliensis |

### Example 9: Efficacy of Bacteria Compositions to Promote CRE Decolonization

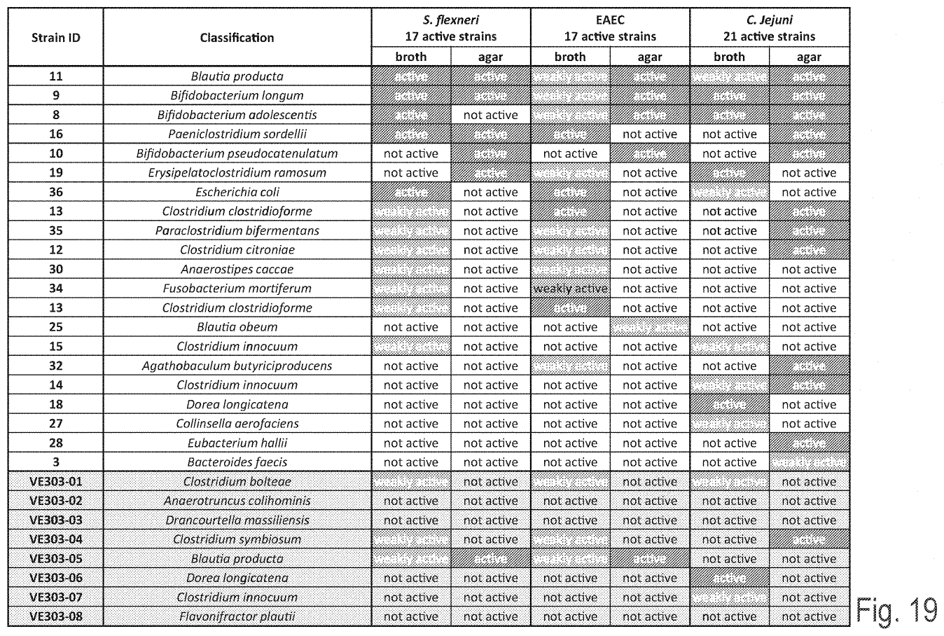

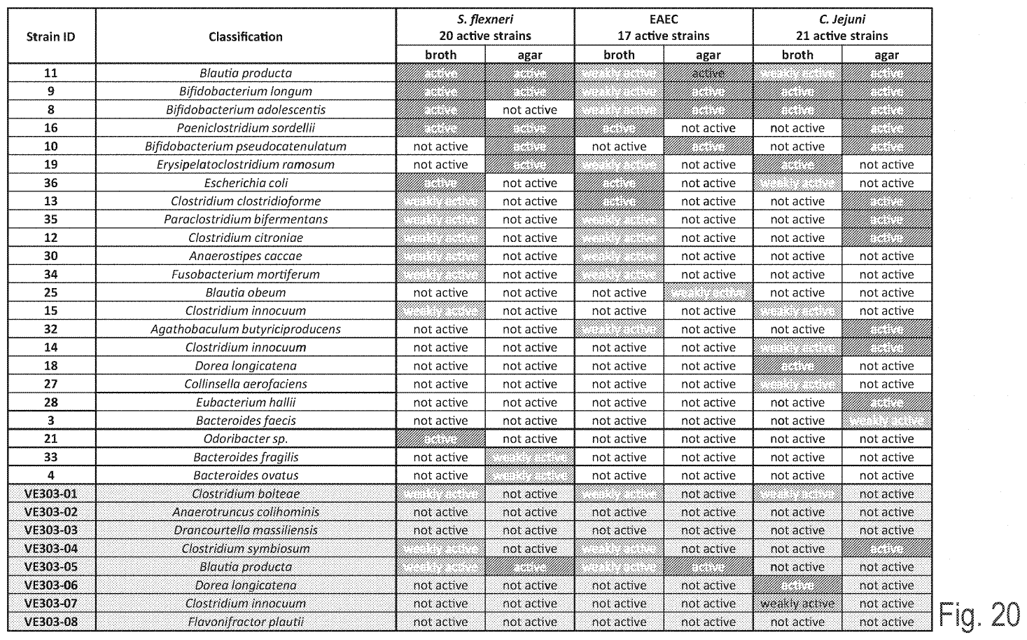

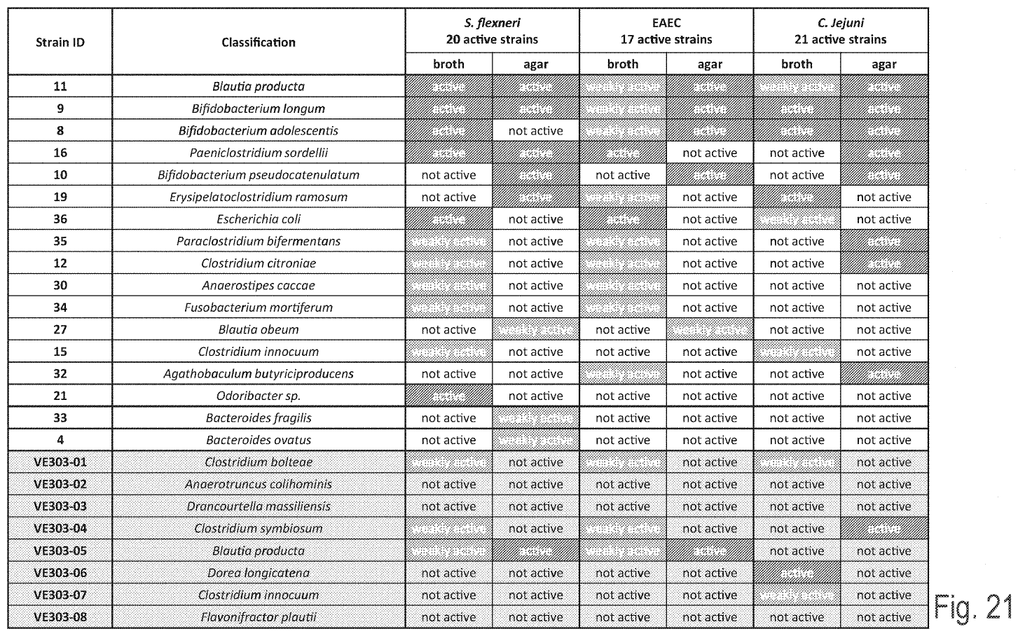

The in vitro results were used to assemble bacterial compositions for further testing. Of the 25 bacterial strains shown in FIG. 18, 9 bacterial families are represented and 15 genera. Of the 29 bacterial strains shown in FIG. 19, 10 bacterial families are represented and 17 genera. Of the 31 bacterial strains shown in FIG. 20, 13 bacterial families are represented and 18 genera. Of the 25 bacterial strains shown in FIG. 21, 12 bacterial families are represented and 16 genera.

As shown in FIG. 1, C57BL/6J mice are treated with antibiotics (e.g., 0.5 g/L ampicillin) for 7 days prior to challenge with Klebsiella pneuomoniae challenge (Klebsiella pneuomoniae ATCC BAA-2814, “CRE”). On day −3, mice are challenged with CRE. On days 0, 1, and 2, mice are administered the following treatments:

a) PBS (negative control);

b) stool fraction library (SFL);

c) bacterial composition containing: Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Clostridium innocuum, and Flavonifractor plautii;

d) bacterial composition containing: Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Clostridium clostridioforme, Blautia obeum, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Clostridium innocuum, and Flavonifractor plautii;

e) bacterial composition containing: Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Blautia obeum, Clostridium innocuum, Agathobaculum butyriciproducens, Clostridium innocuum, Dorea longicatena, Collinsella aerofaciens, Eubacterium hallii, Bacteroides faecis, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Clostridium innocuum, and Flavonifractor plautii; and

f) bacterial composition containing: Blautia producta, Bifidobacterium longum, Bifidobacterium adolescentis, Paeniclostridium sordellii, Bifidobacterium pseudocatenulatum, Erysipelatoclostridium ramosum, Escherichia coli, Paraclostridium bifermentans, Clostridium citroniae, Anaerostipes caccae, Fusobacterium mortiferum, Blautia obeum, Clostridium innocuum, Agathobaculum butyriciproducens, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Clostridium innocuum, and Flavonifractor plautii.

Fecal samples (pellets) are collected at various time points post-treatment to quantify colony forming units (CFU) as measure of CRE colonization levels.

### Example 10: Efficacy of Bacteria Compositions Derived from LBP-1 (“36-Mix”) to Promote CRE Decolonization

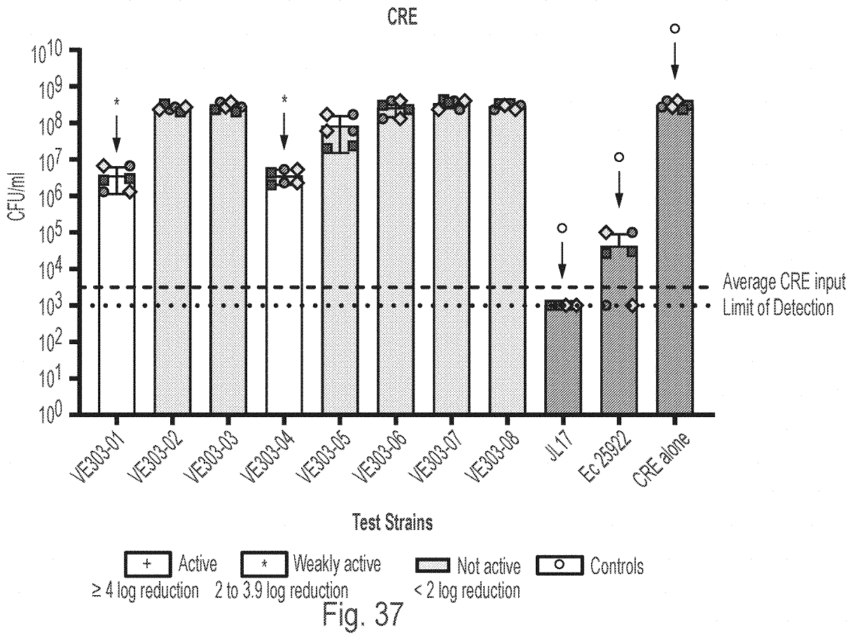

C57BL/6J mice were administered Klebsiella pneuomoniae (Klebsiella pneuomoniae ATCC BAA-2814, “CRE”) prior treatment with the live biotherapeutic products (LBP): LBP-1 (“36-mix”), LBP-9, LBP-10, LBP-11, LBP-12, LBP-13, LBP-14, LBP-15, LBP-16, LBP-17, LBP-18, LBP-19, LBP-19, LBP-20, LBP-21, LBP-22, LBP-23, LBP-24, LBP-25, LBP-26, LBP-27, LBP-28, LBP-29, LBP-30, LBP-31, LBP-32, and LBP-33, or PBS (control). The bacteria strains present in the compositions are shown in FIG. 36. Fecal samples (pellets) were collected at 15 days post-treatment to quantify colony forming units (CFU) as a measure of CRE colonization levels.

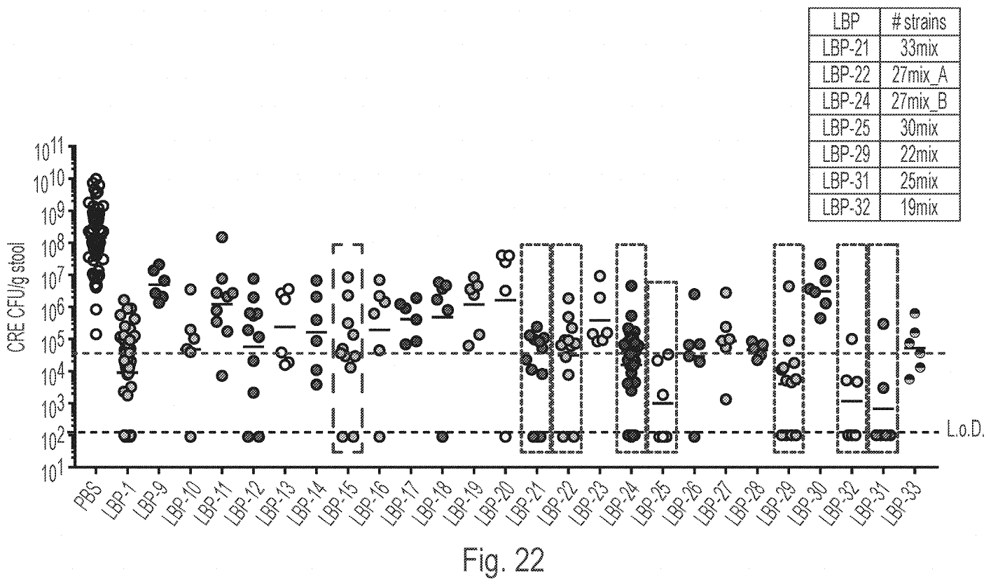

As shown in FIG. 22 and Table 20, several of the bacterial compositions were able to reduce CRE colonization to at least a comparable level as the LBP-1 composition (36-mix).

TABLE 20
Decolonization efficacy of bacterial compositions using criteria in Table 4

| | Klebsiella pneumoniae (CRE) |
|---|---|
| LBP | Clearance Efficacy |
| LBP-1 | High |
| LBP-9 | Low |
| LBP-10 | Intermediate |
| LBP-11 | Low |
| LBP-12 | Intermediate |
| LBP-13 | Intermediate |
| LBP-14 | Intermediate |
| LBP-15 | Intermediate |
| LBP-16 | Intermediate |
| LBP-17 | Intermediate |
| LBP-18 | Intermediate |
| LBP-19 | Intermediate |
| LBP-20 | Low |
| LBP-21 | High |
| LBP-22 | High |
| LBP-23 | Intermediate |
| LBP-24 | High |
| LBP-25 | High |
| LBP-26 | Intermediate |
| LBP-27 | Intermediate |
| LBP-28 | Intermediate |
| LBP-29 | High |
| LBP-30 | Low |
| LBP-31 | High |
| LBP-32 | High |
| LBP-33 | Intermediate |

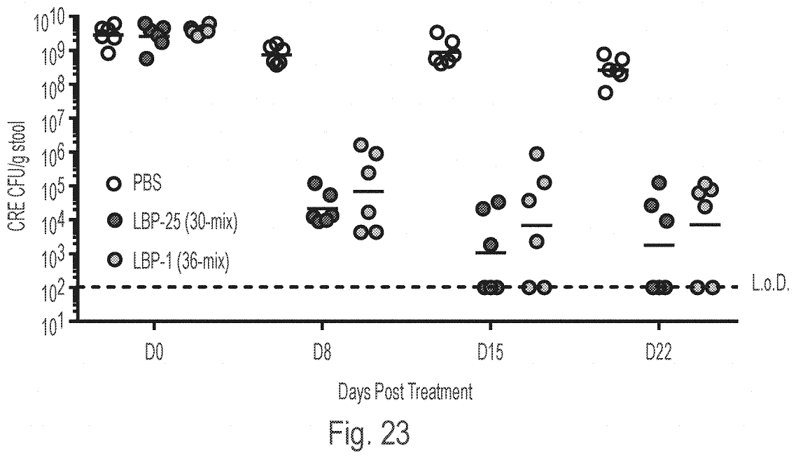

Exclusion experiments were performed with to evaluate subsets of bacterial species from LBP-1 to promote CRE decolonization. There are 9 Bacteroides species present in the LBP-1 composition. The LBP-25 composition excludes 6 of the 9 Bacteroides species present in the LBP-1 composition (Bacteroides thetaiotaomicron, Bacteroides caccae, Bacteroides intestinalis, Bacteroides faecis, Bacteroides fragilis, and Bacteroides uniformis) and has similar CRE decolonization activity to LBP-1 (FIG. 23). Thus, 6 of 9 Bacteroides species are not required for CRE decolonization activity and can be excluded from the composition without compromising activity.

Various compositions were assembled to assess the importance of Escherichia coli and Fusobacterium mortiferum in CRE decolonization activity. As shown in FIGS. 2 and 3, E. coli was required for full decolonization activity and F. mortiferum was beneficial but may not be required for full decolonization activity.

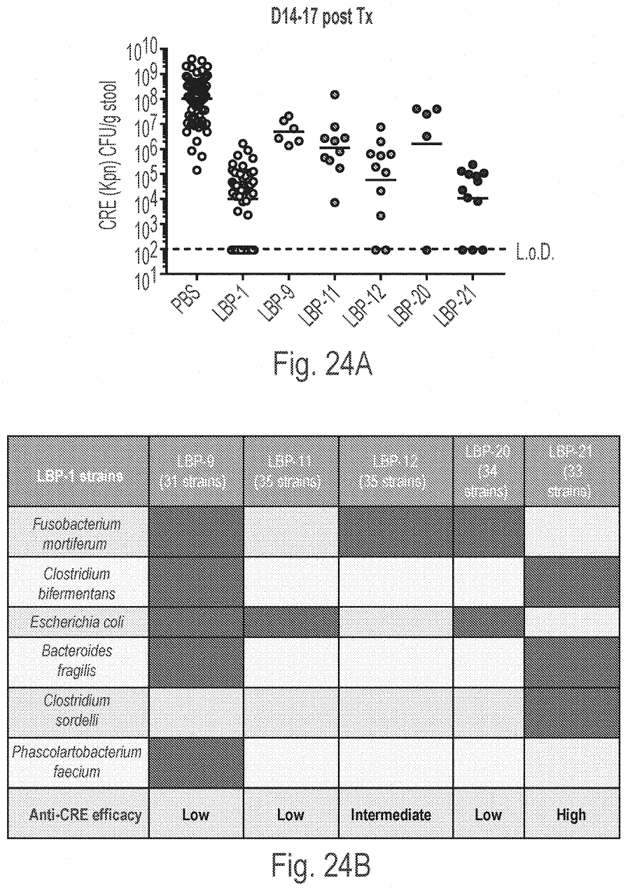

Additional compositions were assembled to evaluate exclusion of other bacterial strains (FIG. 24B). As shown in FIG. 24A, C. bifermentans, B. fragilis, C. sordellii could be excluded from the compositions without impacting decolonization activity.

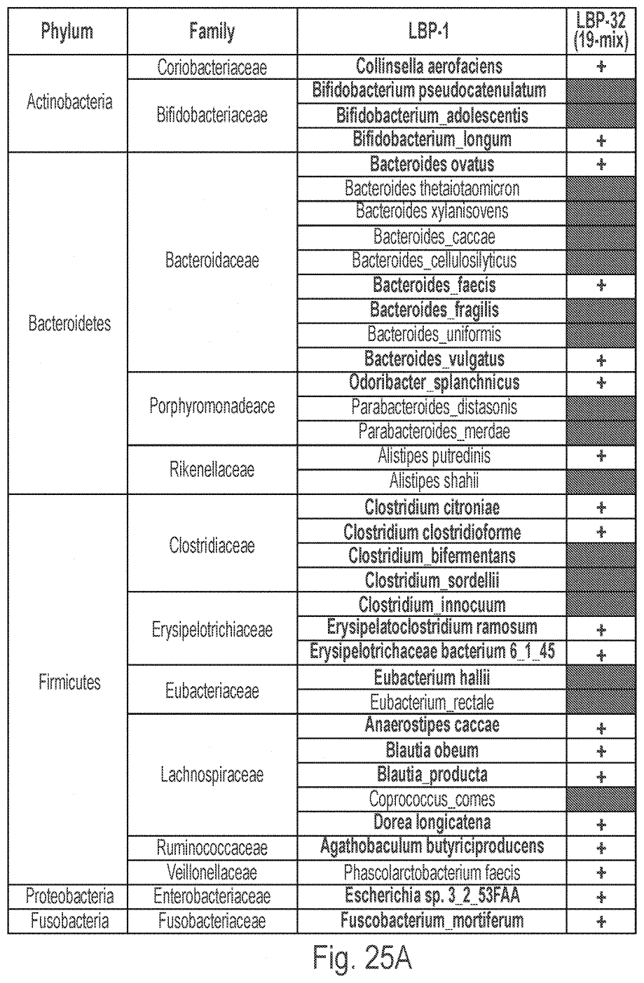

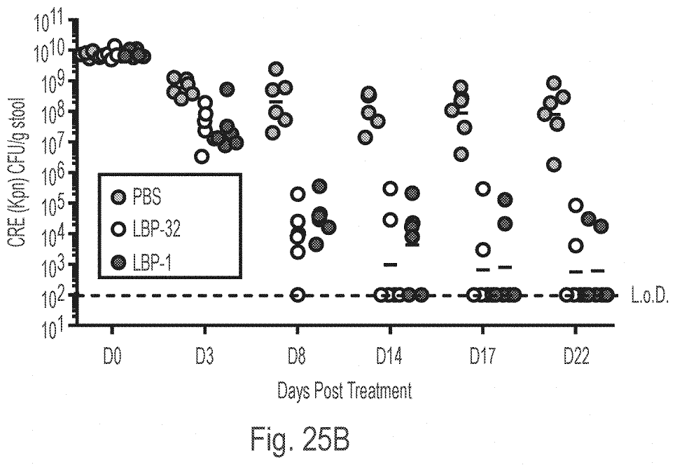

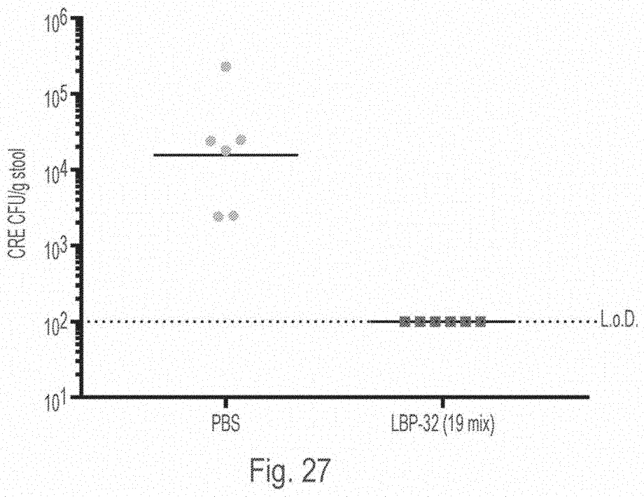

Composition LBP-32 contains 19 bacterial strains that are active in vitro against at least one multi-drug resistant organism (MDRO) or other pathogens evaluated herein minus strains excluded in previous experiments (17 strains) with the addition of Alistipes putredinis and Phascolarctobacterium faecium (FIG. 25A). Mice colonized with CRE were administered LBP-32, LBP-1 (“36-mix”), or PBS control. Fecal samples (pellets) were collected at 0, 3, 8, 14, 17, and 22 days post-treatment to quantify colony forming units (CFU) as a measure of CRE colonization levels. As shown in FIG. 25B, LBP-32 was found to result in a similar level of CRE decolonization activity as LBP-1. Complete CRE decolonization was also seen in ileal samples obtained from the mice at 22 days post-treatment (FIG. 27). Thus, exclusion of 2/3 Bifidobacterium species (Bifidobacterium pseudocatenulatum and Bifidobacterium adolescentis) and Eubacteriaceae (Eubacterium hallii and Eubacterium rectale) did not compromise CRE decolonization activity.

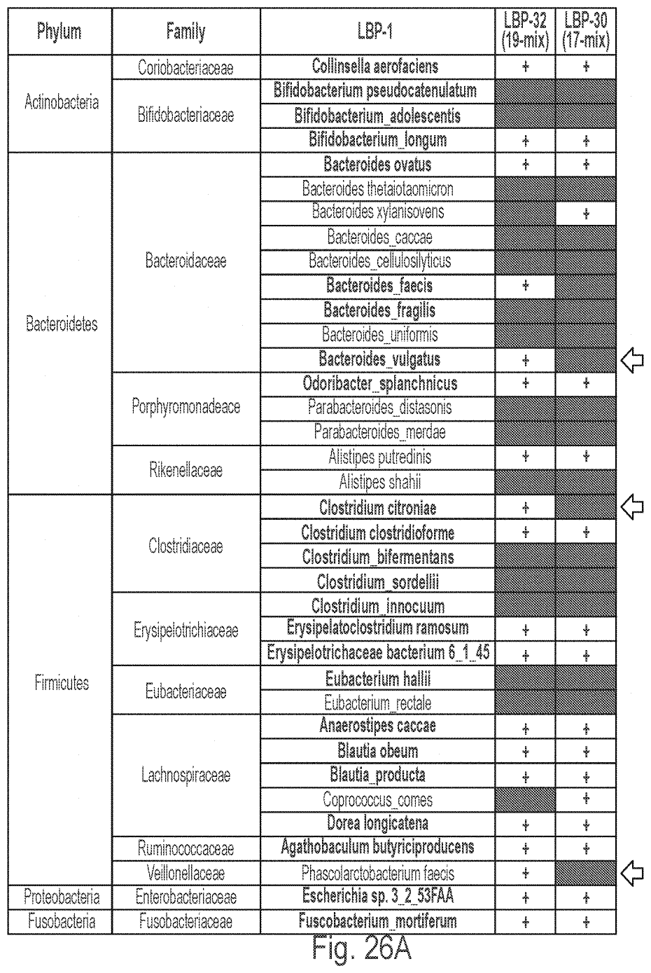

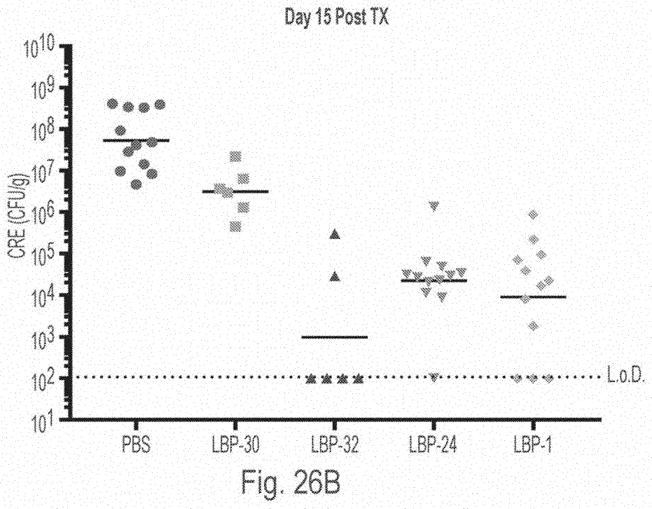

Composition LBP-30 is a modified version of LBP-32 and contains 17 bacterial strains. In particular, Bacteroides vulgatus, Clostridium citroniae, and Phascolarctobacterium faecis are excluded (FIG. 26A). Mice colonized with CRE were administered LBP-30, LBP-32, LBP-1, or PBS control. Fecal samples (pellets) were collected at 8 days post-treatment to quantify colony forming units (CFU) as a measure of CRE colonization levels. LBP-30 was found to result in decreased CRE decolonization activity compared to LBP-32 (FIG. 26B). These results indicate that Bacteroides vulgatus, Clostridium citroniae, and Phascolarctobacterium faecis are required to full CRE decolonization activity.

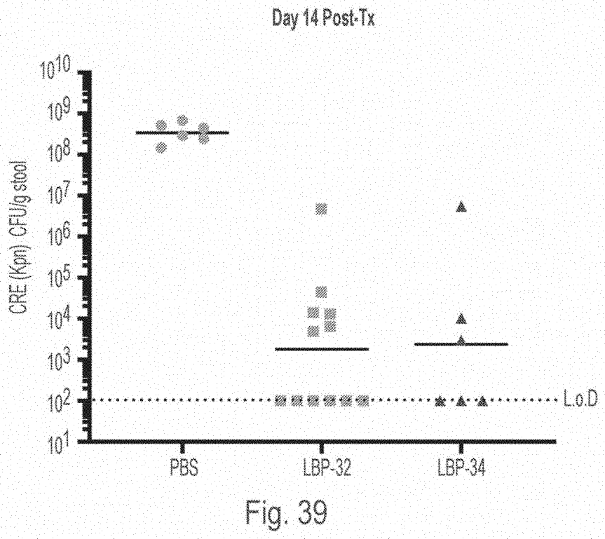

Composition LBP-34 is a modified version of LBP-32 and contains 15 bacterial strains. In particular, Bacteroides faecis, Odoribacter splanchnicus, Alistipes shahii, Clostridium innocuum, Anaerostipes caccae, and Agathobaculum butyriciproducens were excluded and Erysipelotrichaceae bacterium 6_1_45 and Dorea longicatena were included in LBP-34 (FIG. 35). Fecal samples (pellets) were collected at 14 days post-treatment to quantify colony forming units (CFU) as a measure of CRE colonization levels. LBP-34 was found to result in reduced CRE colonization, comparable to LBP-32 (FIG. 39). These results suggest that Bacteroides faecis, Odoribacter splanchnicus, Alistipes shahii, Clostridium innocuum, Anaerostipes caccae, Agathobaculum butyriciproducens, Erysipelotrichaceae bacterium 6_1_45 and Dorea longicatena are not required to promote full LBP-32-mediated CRE decolonization activity.

### Example 11: Co-Colonization Model to Accelerate Live Biotherapeutic Product Identification

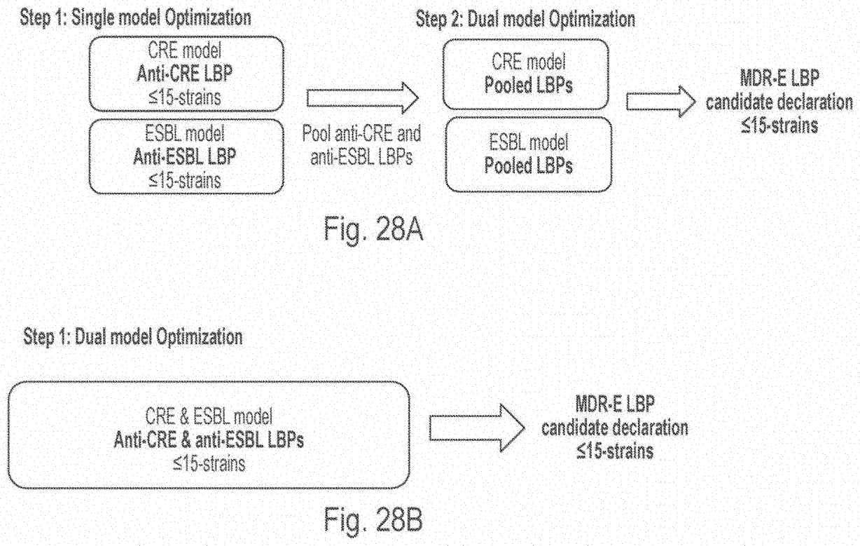

Live biotherapeutic products (LBPs) have previously been tested in mouse models mono-colonized with carbapenem resistant Klebsiella pneumoniae (CRE) or extended spectrum beta-lactamase (ESBL) producing Enterobacteriaceae. The LBPs are then optimized and combined and tested in the mono-colonized CRE and ESBL mouse models (FIG. 28A). This process is time-consuming and long, and there is a risk for diminished activity when the LBPs are combined.

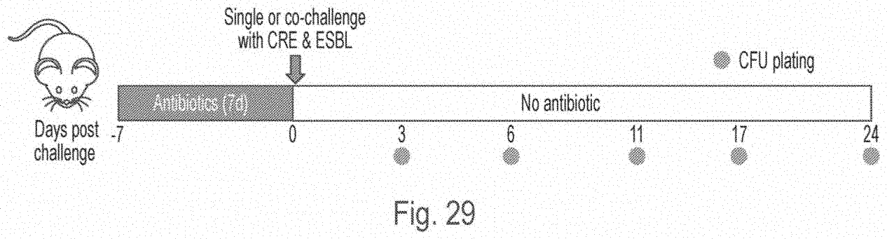

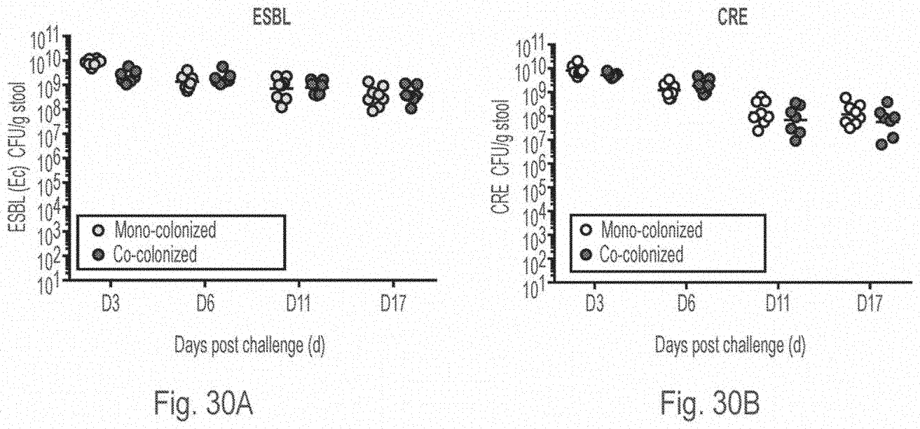

A mouse model that is dual-colonized with CRE and ESBL has been developed to test LBPs for efficacy in promoting CRE and ESBL decolonization, potentially providing simpler and faster strategy for LBP evaluation (FIG. 28B). As shown in FIG. 29, C57BL/6J mice were treated with antibiotics (e.g., 0.5 g/L ampicillin) for 7 days prior to single challenge with Klebsiella pneumoniae (Klebsiella pneuomoniae, “CRE”) or extended spectrum beta-lactamase (ESBL) producing Enterobacteriaceae, or co-challenged with both CRE and ESBL. Fecal samples (pellets) were collected at various time points post-challenge to quantify colony forming units (CFU) as measure of CRE and ESBL colonization levels. CRE and ESBL were found to co-colonize the gastrointestinal tract to the similar levels as mono-colonized mice (FIGS. 30A and 30B).

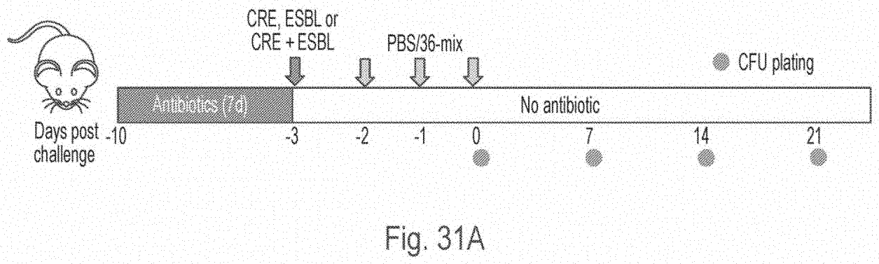

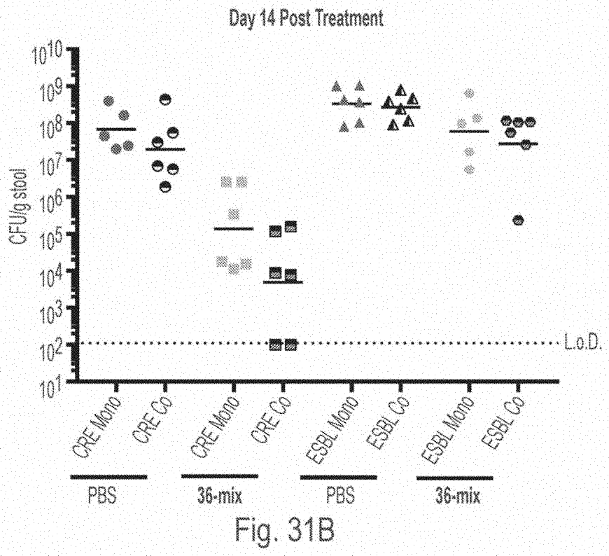

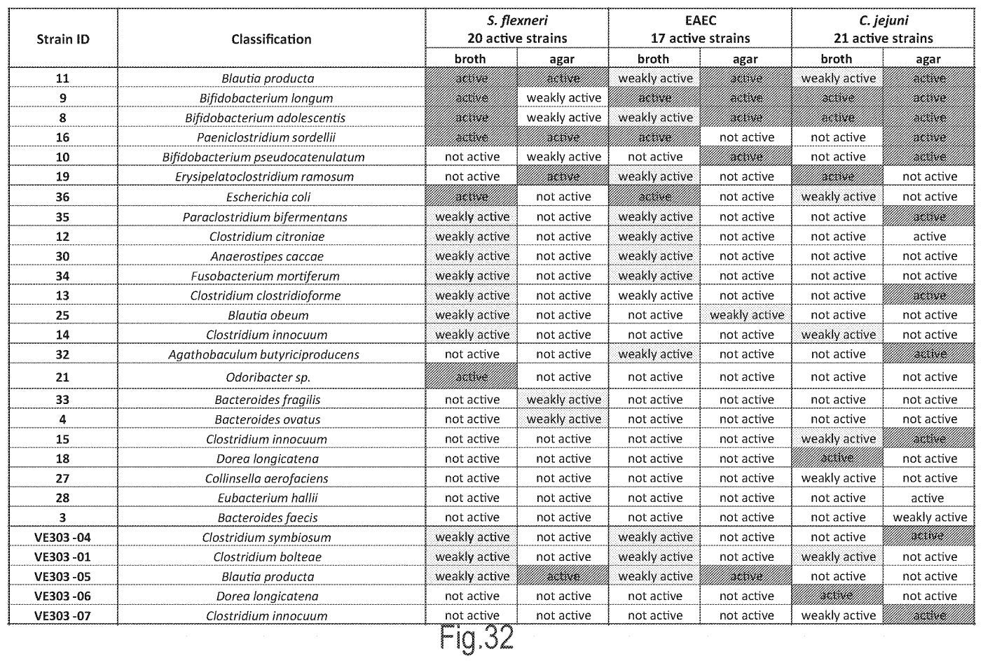

Similarly, the mouse model that is dual-colonized with CRE and ESBL was utilized to test the LBP-1 (“36-mix”) composition. As shown in FIG. 31A, C57BL/6J were treated with antibiotics (e.g., 0.5 g/L ampicillin) for 7 days prior to single challenge with CRE or ESBL, or co-challenged with both CRE and ESBL. The mice were then treated with the 36-mix or control (PBS). Fecal samples (pellets) were collected at various time points post-challenge and treatment to quantify colony forming units (CFU) as a measure of CRE and ESBL colonization levels. The 36-mix composition exhibits similar CRE and ESBL decolonization in mono-colonized (e.g., CRE Mono, ESBL Mono) and co-colonized (e.g., CRE Co, ESBL Co) mice (FIG. 31B).

### Example 12: Screening a Diverse Library of Human Commensals for Antagonistic Activity Against Multidrug-Resistant Gram-Negative Bacteria

Development of defined bacterial consortia to remodel the microbiome and treat infectious diseases is a new therapeutic modality. A well-characterized library of bacterial strains was generated consisting of >90,000 fecal isolates from 275 healthy donors from 12 different geographies. Described herein are rationally-designed bacterial consortia to promote the decolonization of multidrug-resistant (MDR) Gram-negative organisms as a strategy to prevent infection. To identify commensal bacterial strains with inhibitory activity against several MDR Klebsiella pneumoniae strains, a high throughput fluorescence-based bacterial competition assay and a soft agar overlay assay were developed.

Commensal strains were co-cultured with several species of MDR Enterobacteriaceae to identify commensal strains with antagonistic activity in a variety of conditions. First, a high-throughput competition assay was developed to enable large scale screening. Following co-culture, samples were added to chromogenic selective media, and commensal strains that prevented K. pneumoniae expansion were defined as being antagonistic. As different screening procedures may capture different mechanisms of bacterial inhibition, a soft agar overlay competition assay in which inhibitory activity was revealed as a zone of clearance in a lawn of K. pneumoniae was also performed.

13% of the commensal library of 1500 strains were found to have antagonistic activity against K. pneumoniae in the high-throughput liquid assay, of which 184 were active against strains with multiple resistance mechanisms including OXA-48 (carbapenemase), NDM-1 (metallo-beta-lactamase), ESBL (extended-spectrum beta-lactamases), and KPC (Klebsiella pneumoniae carbapenemase). While there was some concordance (˜30%) of strains that were active in both liquid and solid media, there were several commensal strains that had activity in one assay but not the other, highlighting the importance of using multiple assays for comprehensive screening. Strains active against all five K. pneumoniae strains span across 14 genera including Bacteroides, Bifidobacterium, Clostridium, and Lactobacillus.

Combining a novel liquid high-throughput fluorescence-based screening assay with a medium-throughput solid agar assay provided an effective platform to identify bacterial strains active against MDR Enterobacteriaceae and facilitated the development of microbiome-based products to target these organisms.

### Example 13: A Dual In Vitro Screening Platform for the Identification of Pathogen-Antagonistic Gut Commensals

As described herein, a defined bacterial consortia was developed against intestinal pathogens including multidrug-resistant Enterobacteriaceae, Campylobacter jejuni, Shigella flexneri and enteroaggregative Escherichia coli to prevent infection with these organisms. To aid in consortia design, broth competition and soft agar overlay assays were developed to identify commensal bacteria with mechanistically diverse inhibitory activity against these pathogens. Assays were run by co-culturing each commensal strain with a target pathogen followed by selective plating (broth assay), or by spotting each commensal strain and overlaying with soft agar seeded with the target organism (agar-based assay). Strains that resulted in pathogen killing/suppression (broth assay) or formation of a clear zone (agar-based assay) were deemed active. Of the 44 strains tested, 70% showed activity against at least one pathogen, and the strains spanned across 10 different bacterial families. Half of the strains found to be active were assay-specific, highlighting the importance of a combinatorial in vitro approach for assessing bacterial antagonism to support microbiome product development against gut pathogens.

### Example 14: Development of a Live Bacterial Product (LBP) for Decolonization of Multidrug-Resistant Gram Negative Bacteria as a Strategy to Prevent Healthcare-Associated Infections

Intestinal colonization with multidrug-resistant organisms (MDRO) is a risk factor for infection. Described herein are defined bacterial consortia for MDRO decolonization to prevent infection and colonization recurrence while restoring a healthy microbiota. Fecal matter transplant (FMT) donor material was tested in mouse models of carbapenem-resistant Klebsiella pneumoniae (CRE) colonization. Bacterial species from FMT donors with the highest CRE decolonization efficacy were isolated. Using a combinatorial approach of in vitro, in vivo and in silico tools, selected strains were assembled into 35 bacterial consortia of various sizes for in vivo testing. While control mice remained densely colonized, all mice treated with 1 of the 35 consortia exhibited ≥4-log reduction in CRE colonization in the feces with ≥25% exhibiting complete CRE clearance, which was comparable to the efficacy observed with human FMT. These findings demonstrate that a bacterial consortium is an effective microbiome-based approach to eliminate MDRO colonization and reduce the risk of infection.

### Example 15: In Vitro Competition Assays with Carbapenem-Resistant Klebsiella pneumoniae ATCC BAA-2814

Individual bacterial strains were evaluated for their ability to kill carbapenem-resistant Klebsiella pneumoniae strain ATCC BAA-2814 (CRE) by an in vitro mixed culture competition assay. Each of the bacterial strains 1-36 and VE303 strains 1-8 were assessed relative to controls: Lactococcus sp. (JL17), Escherichia coli ATCC 25922 (Ec 25922), VE303 strain 6 (VE303-06), and CRE by itself (CRE alone).

Each of the bacterial strains were assessed according to the workflow diagram in FIG. 12. Briefly, each of the bacterial strains and CRE were grown separately at 37° C. for 18 hours under anaerobic conditions. The cultures of the individual bacterial strains were then added to CRE cultures (— 10³ CFU) and incubated at 37° C. for 18 hours under anaerobic conditions. The co-cultures were then plated on CRE selective media for CFU enumeration by counting colonies for the positive and negative controls, as well as each of the competition experiments. Each of the strains was compared to the average CRE input and controls to evaluate growth suppression of CRE in the presence of the individual bacterial strains.

The results are shown in FIGS. 36-38 and Tables 21 and 22, below. Bacterial strains 8, 9, 10, 16, and 36 were considered “active,” and bacterial strains 11, 34, VE303 strain 1 (VE303-01) and VE303 strain 4 (VE303-07) were “weakly active” in decreasing CRE survival.

To be considered “active” the bacterial strain reduced C. jejuni survival by ≥4.0 log, to be considered “weakly active” the bacterial strain reduced C. jejuni survival by between 2.0 to 3.9 log, and “not active” means bacterial strain reduced C. jejuni survival by <2.0 log.

TABLE 21
CRE survival

| Strain | Classification | Activity |
|---|---|---|
| 8 | Bifidobacterium adolescentis | Active |
| 9 | Bifidobacterium longum | Active |
| 10 | Bifidobacterium pseudocatenulatum | Active |
| 16 | Paeniclostridium sordellii | Active |
| 36 | Escherichia coli | Active |
| 11 | Blautia producta | Weakly Active |
| 34 | Fusobacterium mortiferum | Weakly Active |

TABLE 22
CRE survival

| Strain | Classification | Activity |
|---|---|---|
| VE303-1 | Clostridium bolteae | Weakly Active |
| VE303-4 | Clostridium symbiosum | Weakly Active |

What is claimed is:

1. A composition comprising
(1) one or more purified bacterial strains belonging to the phylum Firmicutes or Bacteroidetes and
(2) one or more purified bacterial strains selected from the group consisting of an Escherichia species and a Fusobacterium species.

2. A composition comprising
(1) one or more purified bacterial strains belonging to the phylum Firmicutes and
(2) one or more purified bacterial strains of Escherichia species.

3. A composition comprising
(1) one or more purified bacterial strains belonging to the phylum Firmicutes and
(2) one or more purified bacterial strains of Fusobacterium species.

4. A composition comprising
(1) one or more purified bacterial strains belonging to the phylum Bacteroidetes and
(2) one or more purified bacterial strains of Escherichia species.

5. A composition comprising
(1) one or more purified bacterial strains belonging to the phylum Bacteroidetes and
(2) one or more purified bacterial strains of Fusobacterium species.

6. The composition of any one of claims 1-3, wherein the one or more purified bacterial strains belonging to the phylum Firmicutes are bacterial strains belonging to the Clostridiaceae family.

7. The composition of any one of claim 1-3 or 6, wherein the one or more purified bacterial strains belonging to the phylum Firmicutes are bacterial strains belonging to Clostridium cluster IV and/or Clostridium cluster XIVa.

8. The composition of any one of claim 1, 4, or 5, wherein the one or more purified bacterial strains belonging to the phylum Bacteroidetes are bacterial strains belonging to the Bacteroidaceae family.

9. The composition of any one of claims 1-8, wherein one or more of the purified bacterial strains belonging to the phylum Firmicutes or Bacteroidetes are bacterial strains that produce short chain fatty acids.

10. The composition of claim 9, wherein one or more of the purified bacterial strains belonging to the phylum Firmicutes or Bacteroidetes are bacterial strains that produce butyrate.

11. The composition of any one of claim 1, 2, 4, or 6-10, wherein the Escherichia species is Escherichia coli (E. coli).

12. The composition of claim 11, wherein the E. coli is Escherichia sp. 3_2_53FAA.

13. The composition of any one of claim 1, 3, or 5-10, wherein the Fusobacterium species is Fusobacterium mortiferum.

14. A composition comprising a purified bacterial strain belonging to Escherichia species, a purified bacterial strain belonging to Fusobacterium species, and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis.

15. A composition comprising a purified bacterial strain belonging to Escherichia species, a purified bacterial strain belonging to Fusobacterium species, and one or more purified bacterial strains of species selected from the group consisting of Collinsella aerofaciens, Bifidobacterium longum, Bacteroides ovatus, Bacteroides vulgatus, Alistipes putredinis, Clostridium citroniae, Clostridium clostridioforme, Erysipelatoclostridium ramosum, Erysipelotrichaceae bacterium 6_1_45, Blautia obeum, Blautia producta, Dorea longicatena, and Phascolarctobacterium faecium.

16. A composition comprising a purified bacterial strain belonging to Escherichia species and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis.

17. A composition comprising purified bacterial strain belonging to Fusobacterium species and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis.

18. A composition comprising a purified bacterial strain belonging to Escherichia species, a purified bacterial strain belonging to Fusobacterium species, and one or more purified bacterial strains of species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii.

19. A composition comprising a purified bacterial strain belonging to Escherichia species and one or more purified bacterial strains of species selected from the group consisting of Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii.

20. A composition comprising a purified bacterial strain belonging to Fusobacterium species and one or more purified bacterial strains of species selected from the group consisting of Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii.

21. A composition comprising Clostridium bolteae, Anaerotruncus colihominis, Sellimonas intestinalis, Clostridium symbiosum, Blautia producta, Dorea longicatena, Erysipelotrichaceae bacterium, Flavinofractor plautii, a purified bacterial strain belonging to Escherichia species, and a purified bacterial strain belonging to Fusobacterium species.

22. A composition comprising Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain belonging to Escherichia species.

23. A composition comprising Clostridium bolteae, Anaerotruncus colihominis, Sellimonas intestinalis, Clostridium symbiosum, Blautia producta, Dorea longicatena, Erysipelotrichaceae bacterium, Flavinofractor plautii, and a purified bacterial strain belonging to Fusobacterium species.

24. A composition comprising Anaerotruncus colihominis, Sellimonas intestinalis, Clostridium symbiosum, Dorea longicatena, Erysipelotrichaceae bacterium, Flavinofractor plautii, a purified bacterial strain belonging to Escherichia species, and a purified bacterial strain belonging to Fusobacterium species.

25. A composition comprising Anaerotruncus colihominis, Sellimonas intestinalis, Clostridium symbiosum, Dorea longicatena, Erysipelotrichaceae bacterium, Flavinofractor plautii, and a purified bacterial strain belonging to Escherichia species.

26. A composition comprising Anaerotruncus colihominis, Sellimonas intestinalis, Clostridium symbiosum, Dorea longicatena, Erysipelotrichaceae bacterium, Flavinofractor plautii, and a purified bacterial strain belonging to Fusobacterium species.

27. A composition comprising Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), a purified bacterial strain belonging to Escherichia species, and a purified bacterial strain belonging to Fusobacterium species.

28. A composition comprising Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), and a purified bacterial strain belonging to Escherichia species.

29. A composition comprising Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLGO55 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), and a purified bacterial strain belonging to Fusobacterium species.

30. A composition comprising Clostridium bolteae, Anaerotruncus colihominis, Sellimonas intestinalis, Clostridium symbiosum, Blautia producta, Erysipelotrichaceae bacterium, Flavinofractor plautii, a purified bacterial strain belonging to Escherichia species, and a purified bacterial strain belonging to Fusobacterium species.

31. A composition comprising Clostridium bolteae, Anaerotruncus colihominis, Sellimonas intestinalis, Clostridium symbiosum, Blautia producta, Erysipelotrichaceae bacterium, Flavinofractor plautii, and a purified bacterial strain belonging to Escherichia species.

32. A composition comprising Clostridium bolteae, Anaerotruncus colihominis, Sellimonas intestinalis, Clostridium symbiosum, Blautia producta, Erysipelotrichaceae bacterium, Flavinofractor plautii, and a purified bacterial strain belonging to Fusobacterium species.

33. A composition comprising Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecis, a purified bacterial strain belonging to Escherichia species, and a purified bacterial strain belonging to Fusobacterium species.

34. A composition comprising Collinsella aerofaciens, Bacteroides ovatus, Bacteroides thetaiotamicron, Bacteroides xyalinosolvens, Bacteroides cellulosilyticus, Bacteroides uniformis, Bacteroides vulgatus, Parabacteroides distasonis, Alistipes putredinis, Alistipes shahii, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium rectale, Anaerostipes caccae, Blautia obeum, Blautia producta, Coprococcus comes, Dorea longicatena, Agathobaculum butyriciproducens, Phascolarctobacterium faecis, a purified bacterial strain belonging to Escherichia species, and a purified bacterial strain belonging to Fusobacterium species.

35. A composition comprising Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, a purified bacterial strain belonging to Escherichia species, and a purified bacterial strain belonging to Fusobacterium species.

36. A composition comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

37. A composition comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Clostridium clostridioforme, Blautia obeum, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

38. A composition comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Blautia obeum, Clostridium innocuum, Agathobaculum butyriciproducens, Clostridium innocuum, Dorea longicatena, Collinsella aerofaciens, Eubacterium hallii, Bacteroides faecis, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

39. A composition comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Paraclostridium bifermentans, Clostridium citroniae, Anaerostipes caccae, Fusobacterium mortiferum, Blautia obeum, Clostridium innocuum, Agathobaculum butyriciproducens, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, and Flavonifractor plautii.

40. A composition comprising Bacteroides cellulosilyticus, Bacteroides ovatus, Bacteroides thetaiotaomicron, Bacteroides uniformis, Bacteroides vulgatus, Bifidobacterium adolescentis, Bifidobacterium longum, Blautia producta, Clostridium citroniae, Clostridium clostridioforme, Clostridium innocuum, Clostridium innocuum, Coprococcus comes, Dorea longicatena, Erysipelatoclostridium ramosum, Eubacterium rectale, Parabacteroides distasonis, Bacteroides xylanisolvens, Blautia obeum, Alistipes putredinis, Collinsella aerofaciens, Eubacterium hallii, Anaerostipes caccae, Phascolarctobacterium faecium, Agathobaculum butyriciproducens, Fusobacterium mortiferum, and Escherichia coli.

41. A composition comprising Bacteroides caccae, Bacteroides cellulosilyticus, Bacteroides ovatus, Bacteroides thetaiotaomicron, Bacteroides uniformis, Bacteroides vulgatus, Bifidobacterium longum, Blautia producta, Clostridium clostridioforme, Clostridium innocuum, Coprococcus comes, Dorea longicatena, Erysipelatoclostridium ramosum, Clostridium citroniae, Odoribacter splanchnicus, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Agathobaculum butyriciproducens, Bacteroides fragilis, Fusobacterium mortiferum, and Escherichia coli.

42. A composition comprising Bacteroides ovatus, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Clostridium innocuum, Paeniclostridium sordellii, Coprococcus comes, Dorea longicatena, Erysipelatoclostridium ramosum, Eubacterium rectale, Odoribacter splanchnicus, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, Phascolarctobacterium faecium, Agathobaculum butyriciproducens, Fusobacterium mortiferum, Paraclostridium bifermentans, and Escherichia coli.

43. A composition comprising Bacteroides ovatus, Bacteroides vulgatus, Bifidobacterium longum, Blautia producta, Clostridium citroniae, Clostridium clostridioforme, Clostridium innocuum, Coprococcus comes, Dorea longicatena, Erysipelatoclostridium ramosum, Odoribacter splanchnicus, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Fusobacterium mortiferum, and Escherichia coli.

44. A composition comprising Bacteroides faecis, Bacteroides ovatus, Bacteroides vulgatus, Bifidobacterium adolescentis, Bifidobacterium longum, Blautia producta, Clostridium citroniae, Clostridium clostridioforme, Clostridium innocuum, Clostridium innocuum, Paeniclostridium sordellii, Dorea longicatena, Erysipelatoclostridium ramosum, Odoribacter splanchnicus, Blautia obeum, Alistipes putredinis, Collinsella aerofaciens, Eubacterium hallii, Anaerostipes caccae, Phascolarctobacterium faecium, Agathobaculum butyriciproducens, Bacteroides fragilis, Fusobacterium mortiferum, Paraclostridium bifermentans, and Escherichia coli.

45. A composition comprising Bacteroides faecis, Bacteroides ovatus, Bacteroides vulgatus, Bifidobacterium longum, Blautia producta, Clostridium citroniae, Clostridium clostridioforme, Clostridium innocuum, Dorea longicatena, Erysipelatoclostridium ramosum, Odoribacter splanchnicus, Alistipes putredinis, Collinsella aerofaciens, Blautia obeum, Anaerostipes caccae, Phascolarctobacterium faecium, Agathobaculum butyriciproducens, Fusobacterium mortiferum, and Escherichia coli.

46. A composition comprising bacterial strains of species Bacteroides vulgatus, Clostridium citroniae, and Phascolarctobacterium faecium, and one or more purified bacterial strains of species selected from the group consisting of Bacteroides faecis, Bacteroides ovatus, Bifidobacterium longum, Blautia producta, Clostridium clostridioforme, Clostridium innocuum, Dorea longicatena, Erysipelatoclostridium ramosum, Odoribacter splanchnicus, Alistipes putredinis, Collinsella aerofaciens, Blautia obeum, Anaerostipes caccae, Agathobaculum butyriciproducens, Fusobacterium mortiferum, and Escherichia coli.

47. A composition comprising Collinsella aerofaciens, Bifidobacterium longum, Bacteroides ovatus, Bacteroides faecis, Bacteroides vulgatus, Odoribacter splanchnicus, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Dorea longicatena, Agathobaculum butyriciproducens, Phascolarctobacterium faecium, Escherichia coli, and Fusobacterium mortiferum.

48. A composition comprising Collinsella aerofaciens, Bifidobacterium longum, Bacteroides ovatus, Bacteroides vulgatus, Parabacteroides distasonis, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Agathobaculum butyriciproducens, Phascolarctobacterium faecium, Escherichia coli, and Fusobacterium mortiferum.

49. A composition comprising Collinsella aerofaciens, Bifidobacterium longum, Bacteroides ovatus, Bacteroides vulgatus, Alistipes putredinis, Clostridium citroniae, Clostridium clostridioforme, Erysipelatoclostridium ramosum, Erysipelotrichaceae bacterium 6_1_45, Blautia obeum, Blautia producta, Dorea longicatena, Phascolarctobacterium faecium, Escherichia coli, and Fusobacterium mortiferum.

50. The composition of any one of claim 14-16, 18, 19, 21, 22, 24, 25, 27, 28, 30, or 33-48, wherein the Escherichia species is Escherichia coli (E. coli).

51. The composition of claim 50, wherein the E. coli is Escherichia sp. 3_2_53FAA.

52. The composition of any one of claim 36-49, 48, or 49, wherein the E. coli encodes one or more genes associated with bacteriocin production.

53. The composition of any one of claims 36-49, 48, and 49, wherein the E. coli does not encode one or more genes associated with plasmid uptake.

54. The composition of any one of claims 14, 15, 17, 18, 20, 21, 23, 24, 26, 27, 29, 30, and 33-35, wherein the Fusobacterium species is Fusobacterium mortiferum.

55. The composition of any one of claims 1-54, wherein the composition comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 purified bacterial strains.

56. The composition of any one of claims 1-55, wherein the composition comprises bacterial strains that originate from more than one human donor.

57. The composition of any one of claims 1-56, wherein the composition is effective in suppressing the replication, survival, and/or colonization of one or more pathogenic organisms.

58. The composition of any one of claims 1-57, wherein the composition is effective in treating an infection by a pathogenic organism in a subject.

59. The composition of claim 58, wherein the pathogenic organism is susceptible to antibiotics.

60. The composition of claim 58, wherein the pathogenic organism is resistant to one or more antibiotics.

61. The composition of any one of claim 57, 58, or 60, wherein the pathogenic organism is a multi-drug resistant organism.

62. The composition of claim 61, wherein the multi-drug resistant organism is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, or Clindamycin resistant Group B Streptococcus.

63. The composition of any one of claims 1-62, wherein the composition is effective in inducing the production of regulatory T cells (Tregs) in the intestine.

64. The composition of any one of claims 1-63, wherein the composition is effective in inducing the production of short chain fatty acids (SCFAs) in the intestine.

65. The composition of any one of claims 1-64, wherein the composition is effective in suppressing the replication, survival, and/or colonization of the intestine by one or more bacteria associated with induction of a Th1 immune response.

66. The composition of any one of claims 1-65, wherein the composition is effective in suppressing the replication, survival, and/or colonization of the intestine by one or more oral microbiome bacteria.

67. The composition of any one of claims 1-66, wherein the bacterial strains are lyophilized.

68. The composition of any one of claims 1-67, wherein the bacterial strains are spray-dried.

69. The composition of any one of claims 1-68, wherein one or more of the bacterial strains are in spore form.

70. The composition of any one of claims 1-69, wherein each of the bacterial strains is in spore form.

71. The composition of any one of claims 1-69, wherein one or more of the bacterial strains are in vegetative form.

72. The composition of any one of claim 1-68 or 71, wherein each of the bacterial strains is in vegetative form.

73. A pharmaceutical composition comprising the composition of any one of claims 1-72, further comprising a pharmaceutically acceptable excipient.

74. The pharmaceutical composition of claim 73, wherein the pharmaceutical composition is formulated for oral delivery.

75. The pharmaceutical composition of claim 73, wherein the pharmaceutical composition is formulated for rectal delivery.

76. The pharmaceutical composition of any one of claims 73-75, wherein the pharmaceutical composition is formulated for delivery to the intestine.

77. The pharmaceutical composition of any one of claims 73-76, wherein the pharmaceutical composition is formulated for delivery to the colon.

78. The pharmaceutical composition of any one of claims 73-77, wherein the pharmaceutical composition is administered as one dose.

79. The pharmaceutical composition of any one of claims 73-77, wherein the pharmaceutical composition is administered as multiple doses.

80. The pharmaceutical composition of claim 78 or 79, wherein each dose comprises the administration of multiple capsules.

81. A food product comprising the composition of any one of claims 1-72 and a nutrient.

82. A method of suppressing infection by a pathogenic organism in a subject, comprising administering to the subject a therapeutically effective amount of the composition of any one of claims 1-72 or the food product of claim 81.

83. The method of claim 82, wherein the pathogenic organism is susceptible to antibiotics.

84. The method of claim 82, wherein the pathogenic organism is resistant to antibiotics.

85. The method of any one of claims 82-84, wherein the pathogenic organism is Clostridium difficile.

86. The method of any one of claim 82, 83, or 85, wherein the pathogenic organism is a multi-drug resistant organism.

87. The method of claim 82-85, wherein the pathogenic organism is Klebsiella pneumoniae.

88. The method of claim 87, wherein the Klebsiella pneumoniae is multi-drug resistant.

89. The method of claim 89, wherein the multi-drug resistant Klebsiella pneumoniae is carbapenem-resistant Klebsiella pneumoniae.

90. The method of any one of claims 85-87, wherein the Klebsiella pneumoniae induces a Th1 response.

91. The method of claim 87, wherein the Klebsiella pneumoniae is strain BAA-2552, strain KP-1, strain 700721, strain 13882, strain 34E1, strain BAA-1705, strain 700603, or strain Kp-2H7.

92. The method of claim 87, wherein the Klebsiella pneumoniae is strain Kp-2H7.

93. The method of any one of claims 82-92, wherein the subject is human.

94. The method of any one of claims 82-93, wherein the composition is administered to the subject more than once.

95. The method of any one of claims 82-94, wherein the composition is administered to the subject by oral administration.

96. The method of any one of claims 82-94, wherein the composition is administered to the subject by rectal administration.

97. The method of any one of claims 82-96, wherein the administering suppresses the replication, survival, and/or colonization of the pathogenic organism.

98. The method of any one of claims 82, 83, and 93-97, wherein the pathogenic organism is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, or Clindamycin resistant Group B Streptococcus.

99. The method of any one of claims 82-98, further comprising administering one or more additional compositions comprising bacteria.

100. The method of any one of claims 82-99, wherein the administration of the pharmaceutical composition is not preceded by administration of an antibiotic.

101. The method of any one of claims 82-100, wherein the administration of the pharmaceutical composition is not preceded by administration of vancomycin.

102. The method of any one of claims 82-99, further comprising administering an antibiotic to the subject prior to administration of the pharmaceutical composition.

103. The method of claim 102, wherein the antibiotic is vancomycin.

104. A method of treating infection by a pathogenic organism in a subject, comprising administering to the subject a therapeutically effective amount of the composition of any one of claims 1-80, or the food product of claim 81.

105. The method of claim 104, wherein the pathogenic organism is susceptible to antibiotics.

106. The method of claim 104, wherein the pathogenic organism is resistant to antibiotics.

107. The method of claim 104, wherein the pathogenic organism is Clostridium difficile.

108. The method of any one of claim 104, 106, or 107, wherein the pathogenic organism is a multi-drug resistant organism.

109. The method of claim 108, wherein the pathogenic organism is Klebsiella pneumoniae.

110. The method of claim 109, wherein the Klebsiella pneumoniae is multi-drug resistant.

111. The method of claim 110, wherein the multi-drug resistant Klebsiella pneumoniae is carbapenem-resistant Klebsiella pneumoniae.

112. The method of any one of claims 109-111, wherein the Klebsiella pneumoniae induces a Th1 response.

113. The method of any one of claims 109-112, wherein the Klebsiella pneumoniae is strain BAA-2552, strain KP-1, strain 700721, strain 13882, strain 34E1, strain BAA-1705, strain 700603, or strain Kp-2H7.

114. The method of claim 113, wherein the Klebsiella pneumoniae is strain Kp-2H7.

115. The method of any one of claims 104-114, wherein the subject is human.

116. The method of any one of claims 104-115, wherein the composition is administered to the subject more than once.

117. The method of any one of claims 104-116, wherein the composition is administered to the subject by oral administration.

118. The method of any one of claims 104-116, wherein the composition is administered to the subject by rectal administration.

119. The method of any one of claims 104-118, wherein the administering suppresses the replication, survival, and/or colonization of the pathogenic organism.

120. The method of any one of claims 104, 106, and 115-119, wherein the pathogenic organism is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, or Clindamycin resistant Group B Streptococcus.

121. The method of any one of claims 104-120, further comprising administering one or more additional compositions comprising bacteria.

122. The method of any one of claims 104-121, wherein the administration of the pharmaceutical composition is not preceded by administration of an antibiotic.

123. The method of any one of claims 104-122, wherein the administration of the pharmaceutical composition is not preceded by administration of vancomycin.

124. The method of any one of claims 104-121, further comprising administering an antibiotic to the subject prior to administration of the pharmaceutical composition.

125. The method of claim 124, wherein the antibiotic is vancomycin.

126. A method of treating a disease or disorder associated with bacterial colonization in a subject, comprising administering to the subject a therapeutically effective amount of the composition of any one of claims 1-80, or the food product of claim 81.

127. The method of claim 126, wherein the pathogenic organism is susceptible to antibiotics.

128. The method of claim 126, wherein the pathogenic organism is resistant to antibiotics.

129. The method of claim 126, wherein the pathogenic organism is Clostridium difficile.

130. The method of claim 126, wherein the pathogenic organism is a multi-drug resistant organism.

131. The method of claim 126, wherein the pathogenic organism is Klebsiella pneumoniae.

132. The method of claim 131, wherein the Klebsiella pneumoniae is multi-drug resistant.

133. The method of claim 132, wherein the multi-drug resistant Klebsiella pneumoniae is carbapenem-resistant Klebsiella pneumoniae.

134. The method of any one of claims 131-133, wherein the Klebsiella pneumoniae induces a Th1 response.

135. The method of claim 134, wherein the Klebsiella pneumoniae is strain BAA-2552, strain KP-1, strain 700721, strain 13882, strain 34E1, strain BAA-1705, strain 700603, or strain Kp-2H7.

136. The method of any one of claims 131-135, wherein the Klebsiella pneumoniae is strain Kp-2H7.

137. The method of any one of claims 126-136, wherein the subject is human.

138. The method of any one of claims 126-137, wherein the composition is administered to the subject more than once.

139. The method of any one of claims 126-138, wherein the composition is administered to the subject by oral administration.

140. The method of any one of claims 126-138, wherein the composition is administered to the subject by rectal administration.

141. The method of any one of claims 126-140, wherein the administering suppresses the replication, survival, and/or colonization of the pathogenic organism.

142. The method of claim 126, wherein the pathogenic organism is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella Typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, or Clindamycin resistant Group B Streptococcus.

143. The method of any one of claims 126-142, further comprising administering one or more additional compositions comprising bacteria.

144. The method of any one of claims 126-143, wherein the administration of the pharmaceutical composition is not preceded by administration of an antibiotic.

145. The method of any one of claims 126-144, wherein the administration of the pharmaceutical composition is not preceded by administration of vancomycin.

146. The method of any one of claims 126-143, further comprising administering an antibiotic to the subject prior to administration of the pharmaceutical composition.

147. The method of claim 146, wherein the antibiotic is vancomycin.

148. A method of suppressing colonization of the intestine of a subject with oral microbiome bacteria, comprising administering to the subject a therapeutically effective amount of the composition of any one of claims 1-80 or the food product of claim 81.

149. The method of claim 148, wherein the oral microbiome bacteria are susceptible to antibiotics.

150. The method of claim 148, wherein the oral microbiome bacteria are resistant to antibiotics.

151. The method of any one of claims 148-158, wherein the subject is human.

152. The method of any one of claims 148-151, wherein the composition is administered to the subject more than once.

153. The method of any one of claims 148-152, wherein the composition is administered to the subject by oral administration.

154. The method of any one of claims 148-152, wherein the composition is administered to the subject by rectal administration.

155. The method of any one of claims 148-154, wherein the administering suppresses the replication, survival, and/or colonization of the oral microbiome bacteria.

156. The method of any one of claims 148-155, further comprising administering one or more additional compositions comprising bacteria.

157. The method of any one of claims 148-156, wherein the administration of the pharmaceutical composition is not preceded by administration of an antibiotic.

158. The method of any one of claims 148-157, wherein the administration of the pharmaceutical composition is not preceded by administration of vancomycin.

159. The method of any one of claims 148-156, further comprising administering an antibiotic to the subject prior to administration of the pharmaceutical composition.

160. The method of claim 159, wherein the antibiotic is vancomycin.
